(19) United States
(12) Patent Application Publication
(10) Pub. No.: US 2026/0207501 A1
(43) Pub. Date: Jul. 23, 2026

(54) LIPID COMPOUNDS AND METHODS OF MAKING AND USE THEREOF

(71) Applicants: OHIO STATE INNOVATION FOUNDATION, Columbus, OH (US); ICAHN SCHOOL OF MEDICINE AT MOUNT SINAI, New York, NY (US)

(72) Inventors: Yizhou DONG, New York, NY (US); Yuebao ZHANG, Columbus, OH (US); Shi DU, New York, NY (US); Siyu WANG, New York, NY (US); Yichen ZHONG, New York, NY (US); Miriam MERAD, New York, NY (US); Brian BROWN, New York, NY (US)

(21) Appl. No.: 19/138,866

(22) PCT Filed: Dec. 15, 2023
(86) PCT No.: PCT/US23/84236

§ 371 Date: Jun. 13, 2025

Related U.S. Application Data
(60) Provisional application No. 63/387,546, filed on Dec. 15, 2022.

(51) Int. Cl. A61K 9/1271; A61K 9/00; A61K 9/06; A61K 9/51; A61K 31/7105; A61K 38/20; A61P 17/02; C07C 323/12; C12N 15/88

(52) U.S. Cl. CPC A61K 9/1271; A61K 9/0014; A61K 9/06; A61K 9/5123; A61K 31/7105; A61K 38/2026; A61P 17/02; C07C 323/12; C12N 15/88

(57) ABSTRACT

Disclosed herein are lipid compounds and compositions comprising lipid compounds and methods of making and use thereof. The present disclosure also relates to methods for delivering an agent into a cell by introducing to the cell a therapeutically effective amount of the compositions, the lipid nanoparticles, the pharmaceutically acceptable compositions, or the hydrogel matrices disclosed herein. Also disclosed herein are methods for promoting wound repair in a subject by administering to the subject a therapeutically effective amount of the compositions, the lipid nanoparticles, the pharmaceutically acceptable compositions, or the hydrogel matrices disclosed herein.

### CROSS REFERENCE TO RELATED APPLICATIONS

This application claims the benefit of U.S. Provisional Patent Application Ser. No. 63/387,546 filed Dec. 15, 2022, the disclosure of which is expressly incorporated herein by reference.

### STATEMENT REGARDING FEDERALLY SPONSORED RESEARCH

This invention was made with Government Support under Grant No. GM144117 awarded by the National Institutes of Health. The Government has certain rights in the invention.

### TECHNICAL FIELD

This application generally relates to lipid formulations that can be used in drug delivery and screening.

### BACKGROUND

Efficient delivery of vaccines, gene therapeutics, mRNA, and drug delivery is a key step and challenge for the application of mRNA therapeutics. Despite promising data from ongoing clinical trials, there are currently problems restricting the widespread use of oligonucleotides in therapeutic and diagnostic contexts. First, free RNAs are susceptible to nuclease digestion in plasma, facilitating degradation of the therapeutic agent. Second, these oligonucleotides are often unable to access the intracellular compartment where the relevant translation machinery resides.

As a result, lipid nanoparticles formed from cationic lipids with other lipid components have been used to as a possible way to traverse these barriers in delivery and increase the cellular uptake of oligonucleotides. However, the efficacy of these delivery systems typically stems from the compositional structure of the base lipid molecule, and new compositions and methods are needed for delivering mRNA to cells for treating various disease states. Thus, there remains a need for improved cationic lipids and lipid nanoparticles for the delivery of oligonucleotides.

### SUMMARY

In accordance with the purposes of the disclosed compounds and methods as embodied and broadly described herein, the disclosed subject matter relates to compounds and methods of making and use thereof.

In some examples, the compound is defined by Formula I, or a pharmaceutically acceptable salt thereof:

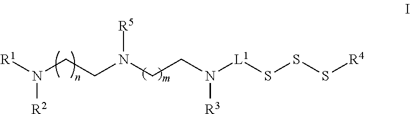

wherein
p is an integer from 0 to 5;
n is an integer from 1 to 10;
each m, when present, is independently an integer from 1 to 10;
R¹, R², and R³ are independently OH, a substituted or unsubstituted C₁-C₅ alkyl, substituted or unsubstituted C₁-C₅ alkyl alcohol, or -L¹-S—S—S—R^{a};
R⁴ is a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl; and
L¹ in each case is independently a substituted or unsubstituted alkyl, a carbonyl, an ester, an amide, a carbamate ester, an amine, an ether, a carbonate ester, a thioether, a thioester, or a urea.

In some examples, the compound is defined by Formula II, or a pharmaceutically acceptable salt thereof:

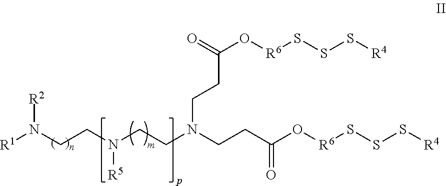

wherein
p is an integer from 0 to 5;
n is an integer from 1 to 10;
m, when present, is an integer from 1 to 10;
R¹ and R² are independently OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

In some examples, the compound is defined by Formula III, or a pharmaceutically acceptable salt thereof:

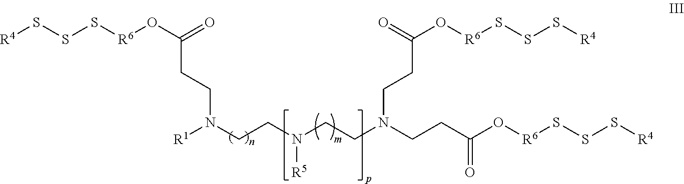

wherein
p is an integer from 0 to 5;
n is an integer from 1 to 10;
m, when present, is an integer from 1 to 10;
R¹ is OH, substituted or unsubstituted C₁-C₅ alkyl, substituted or unsubstituted C₁-C₅ alkyl alcohol, or -L¹-S—S—S—R^{a}
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl;
L¹, when present, is a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

In some examples, the compound is defined by Formula IV, or a pharmaceutically acceptable salt thereof:

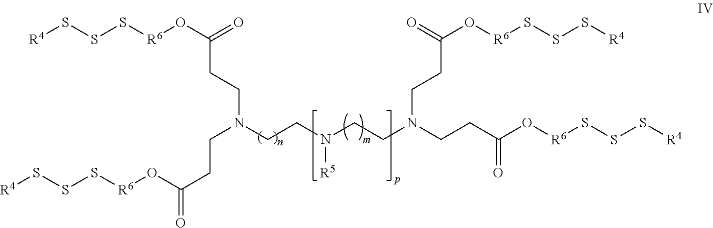

wherein
p is an integer from 0 to 5;
n is an integer from 1 to 10;
m, when present, is an integer from 1 to 10;
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl; and
each R⁶ is independently a substituted or unsubstituted C₁-C₇ alkyl.

In some examples, the compound is defined by Formula V, or a pharmaceutically acceptable salt thereof:

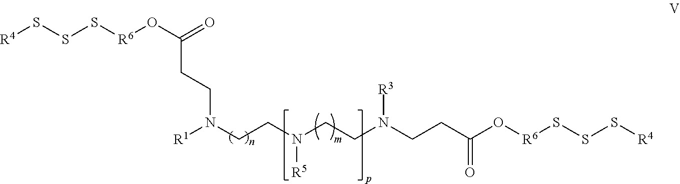

wherein
p is an integer from 0 to 5;
n is an integer from 1 to 10;
each m, when present, is independently an integer from 1 to 10;
R¹ and R³ are independently OH, substituted or unsubstituted C₁-C₅ alkyl, substituted or unsubstituted C₁-C₅ alkyl alcohol, or -L¹-S—S—S—R^{a}
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

Also disclosed herein are compositions comprising any of the compounds disclosed herein. Also disclosed herein are compositions comprising any of the compounds disclosed herein further comprising an agent (e.g. mRNA). In some examples, the agent comprises an RNA. In some examples, the agent comprises an mRNA. In some examples, the agent comprises a polynucleotide encoding a macrophage polarizing factor. In some examples, the agent comprises a polynucleotide encoding interleukin-4. In some examples, the agent is encapsulated by the nanoparticle.

Also disclosed herein are methods of making any of the compounds and compositions disclosed herein.

Also disclosed herein are lipid nanoparticles comprising any of the compounds disclosed herein, a non-cationic lipid; a polyethylene glycol-lipid; and a sterol. In some examples, the non-cationic lipid comprises 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In some examples, the sterol comprises a cholesterol-based lipid. In some examples, a molar ratio of the non-cationic lipid is from 20% to 50%. In some examples, a molar ratio of the compound is from 5% to 60%. In some examples, a molar ratio of the sterol is from 20% to 50%. In some examples, a molar ratio of the PEG-lipid is from 0.1% to 2%. In some examples, a molar ratio of the compound is from 20% to 30%, a molar ratio of the non-cationic lipid is from 35% to 45%, a molar ratio of the sterol is from 35% to 45%, and a molar ratio of the polyethylene glycol-lipid is from 0.1% to 1%. In some examples, a weight fraction of the agent is from 5% to 20%.

Also disclosed herein are pharmaceutical compositions comprising a pharmaceutically acceptable carrier and an effective amount of any of the compounds and compositions disclosed herein. Also disclosed herein is a hydrogel matrix encapsulating the lipid nanoparticle and the agents disclosed herein.

Also disclosed herein are methods for delivering an agent into a cell, comprising: introducing into the cell any of the compositions, nanoparticles, pharmaceutically acceptable compositions, or hydrogel matrices described herein.

Also disclosed herein are methods for promoting wound repair in a subject, comprising: administering to the subject any of the compositions, nanoparticles, pharmaceutically acceptable compositions, or hydrogel matrices described herein.

Additional advantages of the disclosed compounds, compositions and methods will be set forth in part in the description which follows, and in part will be obvious from the description. The advantages of the disclosed compounds, compositions and methods will be realized and attained by means of the elements and combinations particularly pointed out in the appended claims. It is to be understood that both the foregoing general description and the following detailed description are exemplary and explanatory only and are not restrictive of the disclosed compounds, compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIG. 1 shows in vitro delivery efficiency of trisulfide lipid nanoparticles in Hep3B cells using firefly luciferase.

FIG. 2 shows in vivo delivery efficacy of trisulfide lipid nanoparticles in C57BL/6 mice using firefly luciferase.

FIGS. 3A-3B show the design and synthesis of a library of trisulfide-derived ionizable lipids for the treatment of diabetic wounds. (FIG. 3A) Representative synthetic route to TS2, a trisulfide-derived ionizable lipid. List of headgroup amines and acrylate tails used in the combinatorial Michael addition reactions. Acrylates tails were named according to the length of hydrocarbon domains and ester types. (FIG. 3B) Illustration of synthetic TS2-IL4 Lipid nanoparticle (LNP) loaded into a hydrogel accelerates diabetic wound healing by scavenging ROS and modulating the phenotype of macrophages at the wound site.

FIGS. 4A-4H show the screening, optimization, and characterization of trisulfide-derived lipid nanoparticles (TS LNPs). (FIG. 4A) mRNA delivery efficacy of TS LNPs in macrophage. The bioluminescence intensity was normalized to the MC3 LNP (n=3). (FIG. 4B) Table for first round optimal formulations of TS2 LNP. (FIG. 4C) Orthogonal assay to determine impact trends of each lipid component in TS2 LNP at four levels (n=3). (FIG. 4D) Relative bioluminescence intensity of the first-round optimization (n=3). (FIG. 4E) Table for second round optimal formulations of TS2 LNP. (FIG. 4F) Relative bioluminescence intensity of the second round of optimization (n=3). (FIG. 4G) Characterization of TS2 LNP, including size, PDI, encapsulation efficiency, and zeta potential (n=3). (FIG. 4H) Cryo-TEM image of TS2 LNP. Scale bar, 50 nm. All data are presented as mean±SD. Statistical significance was calculated by unpaired two-tailed Student's t test a f and one-way ANOVA with the Tukey post hoc test d. *P<0.05, **P<0.01, ***P<0.001, ****P<0.0001.

FIGS. 5A-5H show that TS2-IL4 LNP induces macrophage polarization and enhances fibroblast survival by scavenging ROS in vitro. (FIG. 5A) Representative flow cytometric analysis and (FIG. 5B) quantification of the expression of M1 macrophage marker CD86⁺ after different treatments, (n=3). (FIG. 5C) Representative flow cytometric analysis and (FIG. 5D) quantification of the expression of M2 macrophage markers CD206⁺ after different treatments, (n=3). (FIG. 5E) Representative Live/dead cell staining CLSM images of fibroblasts after different treatments. Green: live; Red: dead. Scale bar: 100 m. (FIG. 5F) Quantification of cell viability after different treatments, (n=3). (FIG. 5G) CLSM images of intracellular ROS level in fibroblasts after different treatments. Scale bar: 100 m. (FIG. 5H) Flow cytometry analysis of intracellular ROS generation after different treatments. All data are presented as mean±SD. Statistical significance was calculated by one-way ANOVA with the Tukey post hoc test. ns P>0.05, *P<0.05, **P<0.01, ***P<0.001, ****P<0.0001.

FIGS. 6A-6G show that TS2-IL4 LNP loaded in a hydrogel accelerated wound healing in diabetic mice. (FIG. 6A) Representative bioluminescence imaging of wounds in different groups. (FIG. 6B) Quantification of bioluminescence intensity at the wound sites in different groups, (n=6). (FIG. 6C) Representative photographic images of the diabetic wounds with different treatments on Day 0, 3, 6, 9, 12, 15, and 18, respectively. (FIG. 6D) Relative wound area in each group (n=6). (FIG. 6E) The complete wound closure time in different treatment groups. (FIG. 6F) Representative images of H&E staining (upper) and Masson staining (lower) of the wounds on day 18 post-treatment. Scale bar: 500 m. (FIG. 6G) Quantification analysis of epidermal thickness for each treatment group 18 days post-treatment, (n=6). All data are presented as mean±SD. Statistical significance was calculated by one-way ANOVA with the Tukey post hoc test in FIGS. 6B, 6D, 6G, and Log-rank test in e. **P<0.01, ***P<0.001, ****P<0,0001.

FIGS. 7A-7H show flow cytometry analysis and immunofluorescence staining of wound tissues on day 18 post-treatment. (FIG. 7A) Representative flow cytometry analysis of CD86 expression in the wounds 18 days post-treatment, gated on F4/80⁺, CD86⁺ cells. (FIG. 7B) Quantification of percentage of M1 phenotype macrophage (F4/80⁺ CD86+), (n=3). (FIG. 7C) Representative flow cytometry analysis of CD206 expression in the wound 18 days post-treatment, gated on F4/80+, CD206⁺ cells. (FIG. 7D) Quantification of the percentage of M2 phenotype macrophage (F4/80⁺ CD206⁺), (n=3). (FIG. 7E) Representative images and (FIG. 7F) quantification of M1 phenotype macrophage immunofluorescence staining in the wounds of the different groups (CD86⁺: green, nuclei: blue), (n=6). Scale bar: 50 m. (FIG. 7G) Representative images and (FIG. 7H) quantification of M2 phenotype macrophage immunofluorescence staining in the wounds of the different groups (CD206+: green, nuclei: blue), (n=6). Scale bar: 50 m. All data are presented as mean±SD. Statistical significance was calculated by one-way ANOVA with the Tukey post hoc test, **P<0.01, ***P<0.001, ****P<0.0001.

FIGS. 8A-8E show that TS2-IL4 LNP loaded in a hydrogel accelerated wound healing in diabetic mice. (FIG. 8A) Representative bioluminescence imaging of mouse wounds in different groups. (FIG. 8B) Quantification of bioluminescence intensity at the wound sites in different groups (n=6). (FIG. 8C) Representative photographic images of the diabetic wounds with different treatments on day 0, 3, 6, 9, 12, 15, and 18, respectively. (FIG. 8D) Relative wound area in each group (n=6). (FIG. 8E) The complete wound closure time in different treatments. All data are presented as mean±SD. Statistical significance was calculated by one-way ANOVA with the Tukey post hoc test in FIGS. 8B, 8D and Log-rank test in 8E. **P<0.01, ***P<0.001, ****P<0.0001.

FIG. 9 shows the expression levels of IL4 after incubating TS2-IL4 LNP with RAW264.7 cells at different time points (n=3).

FIG. 10 shows quantitative analysis of the mean fluorescence intensity of ROS with different treatments (n=3).

FIG. 11 shows mRNA delivery efficiency of TS2 LNPs, ALC 0315 LNP, and SM102 LNP in macrophages. The bioluminescence intensity was normalized to MC3 LNP group (n=3).

FIGS. 12A-12B show (a) Representative images and quantification of α-SMA⁺ immunofluorescence staining in the wounds of different groups (α-SMA*: green, nuclei: blue). (b) Representative images and quantification of CD31⁺ immunofluorescence staining in the wounds of different groups (CD31⁺: red, nuclei: blue). Scale bar: 50 μm.

### DETAILED DESCRIPTION

The compounds, compositions and methods described herein may be understood more readily by reference to the following detailed description of specific aspects of the disclosed subject matter and the Examples included therein.

Before the present compounds, compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1a promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of 20%, ±10%, ±5%, or ±1% from the measurable value.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

The term “molar ratio” as used herein relates to the stoichiometric amount of moles of components. For example, the molar ratio can be used to define the relative amounts of individual lipid components within a lipid nanoparticle.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “toll-like receptor” (TLR) refers to a member of a family of receptors that bind to pathogen associated molecular patterns (PAMPs) and facilitate an immune response in a mammal. Ten mammalian TLRs are known, e.g., TLR 1-10. The term “toll-like receptor agonist” (TLR agonist) refers to a molecule that binds to a TLR. Synthetic TLR agonists are chemical compounds that are designed to bind to a TLR and activate the receptor. Exemplary synthetic TLR agonists provided herein include “TLR-7 agonist”, “TLR” agonist”, “TLR-3 agonist” and “TLR-9 agonist.” TLR agonists include imiquimod, resiquimod, broprimine and loxoribine, gardiquimod, CL075, SM324405, UC1V150, CU-T12-9, or derivatives thereof.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkyl alcohol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above. Examples of alkyl-alcohols include methanol, ethanol, 1-propanol, 2-propanol, 1-butanol, 2-butanol, tert-butyl alcohol, and the like.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula C(O)H. Throughout this specification “C(O)” is a shorthand notation for C═O.

The terms “amine” or “amino” as used herein are represented by the formula NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

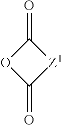

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —Z¹OC(O)Z² or —Z¹C(O)OZ², where Z¹ and Z² can independently be be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

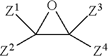

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds and methods of making and use thereof. For example, disclosed herein are compositions comprising a compound defined by Formula I, or a pharmaceutically acceptable salt thereof:

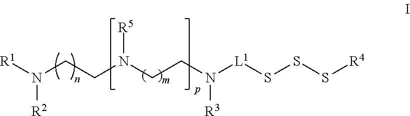

wherein
p is an integer from 0 to 5;
n is an integer from 1 to 10;
each m, when present, is independently an integer from 1 to 10;
R¹, R², and R³ are independently OH, a substituted or unsubstituted C₁-C₅ alkyl, substituted or unsubstituted C₁-C₅ alkyl alcohol, or -L¹-S—S—S—R^{a};
R⁴ is a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl; and
L¹ in each case is independently a substituted or unsubstituted alkyl, a carbonyl, an ester, an amide, a carbamate ester, an amine, an ether, a carbonate ester, a thioether, a thioester, or a urea.

In some examples of Formula I, n is from 1 to 7. In some examples of Formula I, n is from 1 to 6. In some examples of Formula I, n is from 1 to 5. In some examples of Formula I, n is from 1 to 4. In some examples of Formula I, n is from 1 to 3. In some examples of Formula I, n is 1.

In some examples of Formula I, m is from 1 to 7. In some examples of Formula I, m is from 1 to 6. In some examples of Formula I, m is from 1 to 5. In some examples of Formula I, m is from 1 to 4. In some examples of Formula I, m is from 1 to 3. In some examples of Formula I, m is 1. In some examples of Formula I, R⁵ is a substituted or unsubstituted C₁-C₅ alkyl. In some examples of Formula I, R⁵ is methyl. In some examples of Formula I, R⁵ is a substituted or unsubstituted C₁-C₅ alkyl alcohol. In some examples of Formula I, R⁵ is an unsubstituted C₁-C₅ alkyl alcohol. In some examples of Formula I, R⁵ is —CH₂OH. In some examples of Formula I, R⁵ is OH. In some examples of Formula I, R⁵ is hydrogen. In some examples of Formula I, R⁵ is -L¹-S—S—S—R^{a}.

In some examples of Formula I, R⁴ is a substituted or unsubstituted C₈-C₁₈ alkyl (e.g., C₁₀-C₁₄ alkyl or C₁₂ alkyl). In some examples of Formula I, R⁴ is a substituted C₈-C₁₈ alkyl (e.g., C₁₀-C₁₄ alkyl or C₁₂ alkyl). In some examples of Formula I, R⁴ is a C₈-C₁₈ alkyl substituted with one or more substituents selected from the group consisting of amine, amide, ester, ether, and carbonate ester. In some examples of Formula I, R⁴ is an unsubstituted C₈-C₁₈ alkyl (e.g., C₁₀-C₁₄ alkyl or C₁₂ alkyl). In some examples of Formula I, R⁴ is an unsubstituted branched C₈-C₁₈ alkyl (e.g., C₁₀-C₁₄ alkyl or C₁₂ alkyl). In some examples of Formula I, R⁴ is an unsubstituted linear C₈-C₁₈ alkyl (e.g., C₁₀-C₁₄ alkyl or C₁₂ alkyl).

In some examples of Formula I, p is from 0 to 4. In some examples of Formula I, p is from 0 to 3. In some examples of Formula I, p is from 0 to 2. In some examples of Formula I, p is from 0 to 1. In some examples of Formula I, p is 0. In some examples of Formula I, p is 1. In some examples of Formula I, p is 2 or more (e.g., 2, 3, 4, 5). In some examples of Formula I, where p is 2 or more, each R⁵ is the same. In some examples of Formula I, where p is 2 or more, at least one R⁵ is different. In some examples of Formula I, where p is 2 or more, each m is the same. In some examples of Formula I, where p is 2 or more, at least one m is different.

In some examples of Formula I, R₁ is OH. In some examples of Formula I, R₁ is a substituted or unsubstituted C₁-C₅ alkyl. In some examples of Formula I, R₁ is a substituted C₁-C₅ alkyl. In some examples of Formula I, R₁ is an unsubstituted C₁-C₅ alkyl. In some examples of Formula I, R₁ is a substituted or unsubstituted C₁-C₅ alkyl alcohol. In some examples of Formula I, R₁ is a substituted C₁-C₅ alkyl alcohol. In some examples of Formula I, R₁ is an unsubstituted C₁-C₅ alkyl alcohol. In some examples of Formula I, R₁ is -L¹-S—S—S—R^{a}.

In some examples of Formula I, R₂ is OH. In some examples of Formula I, R₂ is a substituted or unsubstituted C₁-C₅ alkyl. In some examples of Formula I, R₂ is a substituted C₁-C₅ alkyl. In some examples of Formula I, R₂ is an unsubstituted C₁-C₅ alkyl. In some examples of Formula I, R₂ is a substituted or unsubstituted C₁-C₅ alkyl alcohol. In some examples of Formula I, R₂ is a substituted C₁-C₅ alkyl alcohol. In some examples of Formula I, R₂ is an unsubstituted C₁-C₅ alkyl alcohol. In some examples of Formula I, R₂ is -L¹-S—S—S—R^{a}.

In some examples of Formula I, R₃ is OH. In some examples of Formula I, R₃ is a substituted or unsubstituted C₁-C₅ alkyl. In some examples of Formula I, R₃ is a substituted C₁-C₅ alkyl. In some examples of Formula I, R₃ is an unsubstituted C₁-C₅ alkyl. In some examples of Formula I, R₃ is a substituted or unsubstituted C₁-C₅ alkyl alcohol. In some examples of Formula I, R₃ is a substituted C₁-C₅ alkyl alcohol. In some examples of Formula I, R₃ is an unsubstituted C₁-C₅ alkyl alcohol. In some examples of Formula I, R₃ is -L¹-S—S—S—R^{a}.

In some examples of Formula I, each R^{a}, when present, is independently a substituted or unsubstituted C₈-C₁₈ alkyl (e.g., C₁₀-C₁₄ alkyl or C₁₂ alkyl). In some examples of Formula I, each R^{a}, when present, is independently a substituted C₈-C₁₈ alkyl. In some examples of Formula I, each R^{a}, when present, is independently an unsubstituted C₈-C₁₈ alkyl.

In some examples of Formula I, L¹ in each case is independently a substituted or unsubstituted alkyl. In some examples of Formula I, L¹ in each case is independently a carbonyl. In some examples of Formula I, L¹ in each case is independently an ester. In some examples of Formula I, L¹ in each case is independently an amide. In some examples of Formula I, L¹ in each case is independently a carbamate ester. In some examples of Formula I, L¹ in each case is independently an amine. In some examples of Formula I, L¹ in each case is independently an ether. In some examples of Formula I, L¹ in each case is independently a carbonate ester. In some examples of Formula I, L¹ in each case is independently a thioether. In some examples of Formula I, L¹ in each case is independently a thioester. In some examples of Formula I, L¹ in each case is independently a urea. In some examples of Formula I having more than one L¹ groups, the compound includes a combination of the above (e.g., both a carbonate ester and a substituted or unsubstituted alkyl).

In some examples of Formula I, each L¹ is an ester represented by the formula —Z¹C(O)OZ²—, wherein Z¹ and Z² are each independently a substituted or unsubstituted C₁-C₁₀ alkyl. In some examples of Formula I, Z¹ and Z² are each independently a substituted or unsubstituted C₁-C₅ alkyl. In some examples of Formula I, Z¹ is —(C₂H₄)—. In some examples of Formula I, Z² is an unsubstituted C₁-C₇ alkyl. In some examples of Formula I, Z² is selected from;

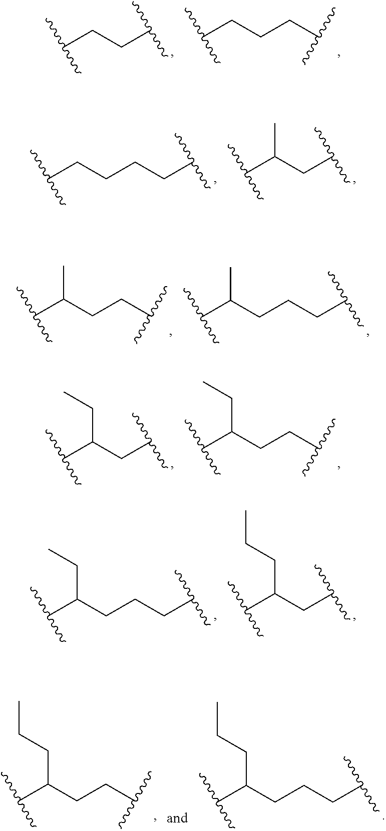

In some examples, the compound is defined by Formula II, or a pharmaceutically acceptable salt thereof:

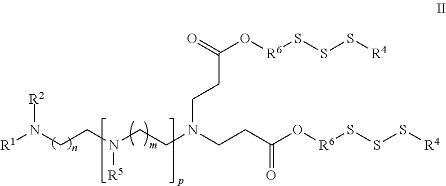

wherein
p is an integer from 0 to 5;
n is an integer from 1 to 10;
m, when present, is an integer from 1 to 10;
R¹ and R² are independently OH, substituted or unsubstituted C₁-C₅ alkyl, substituted or unsubstituted C₁-C₅ alkyl alcohol, or -L¹-S—S—S—R^{a}
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a}
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

In some examples of Formula II, n is from 1 to 7. In some examples of Formula II, n is from 1 to 6. In some examples of Formula II, n is from 1 to 5. In some examples of Formula II, n is from 1 to 4. In some examples of Formula II, n is from 1 to 3. In some examples of Formula II, n is 1.

In some examples of Formula II, m is from 1 to 7. In some examples of Formula II, m is from 1 to 6. In some examples of Formula II, m is from 1 to 5. In some examples of Formula II, m is from 1 to 4. In some examples of Formula II, m is from 1 to 3. In some examples of Formula II, m is 1. In some examples of Formula II, R⁵ is a substituted or unsubstituted C₁-C₅ alkyl. In some examples of Formula II, R⁵ is methyl. In some examples of Formula II, R⁵ is a substituted or unsubstituted C₁-C₅ alkyl alcohol. In some examples of Formula II, R⁵ is an unsubstituted C₁-C₅ alkyl alcohol. In some examples of Formula II, R⁵ is —CH₂OH. In some examples of Formula II, R⁵ is OH. In some examples of Formula II, R⁵ is hydrogen. In some examples of Formula II, R⁵ is -L¹-S—S—S—R^{a}.

In some examples of Formula II, each R⁴ is independently a substituted or unsubstituted C₈-C₁₈ alkyl (e.g., C₁₀-C₁₄ alkyl or C₁₂ alkyl). In some examples of Formula II, each R⁴ is independently a substituted C₈-C₁₈ alkyl (e.g., C₁₀-C₁₄ alkyl or C₁₂ alkyl). In some examples of Formula II, each R⁴ is independently a C₈-C₁₈ alkyl substituted with one or more substituents selected from the group consisting of amine, amide, ester, ether, and carbonate ester. In some examples of Formula II, each R⁴ is independently an unsubstituted C₈-C₁₈ alkyl (e.g., C₁₀-C₁₄ alkyl or C₁₂ alkyl). In some examples of Formula II, each R⁴ is independently an unsubstituted branched C₈-C₁₈ alkyl (e.g., C₁₀-C₁₄ alkyl or C₁₂ alkyl). In some examples of Formula II, each R⁴ is independently an unsubstituted linear C₈-C₁₈ alkyl (e.g., C₁₀-C₁₄ alkyl or C₁₂ alkyl). In some examples of Formula II, each R⁴ is the same. In some examples of Formula II, at least one R⁴ is different.

In some examples of Formula II, p is from 0 to 4. In some examples of Formula II, p is from 0 to 3. In some examples of Formula II, p is from 0 to 2. In some examples of Formula II, p is from 0 to 1. In some examples of Formula II, p is 0. In some examples of Formula II, p is 1. In some examples of Formula II, p is 2 or more (e.g., 2, 3, 4, 5). In some examples of Formula II, where p is 2 or more, each R⁵ is the same. In some examples of Formula II, where p is 2 or more, at least one R⁵ is different. In some examples of Formula II, where p is 2 or more, each m is the same. In some examples of Formula II, where p is 2 or more, at least one m is different.

In some examples of Formula II, R₁ is OH. In some examples of Formula II, R₁ is a substituted or unsubstituted C₁-C₅ alkyl. In some examples of Formula II, R₁ is a substituted C₁-C₅ alkyl. In some examples of Formula II, R₁ is an unsubstituted C₁-C₅ alkyl. In some examples of Formula II, R₁ is a substituted or unsubstituted C₁-C₅ alkyl alcohol. In some examples of Formula II, R₁ is a substituted C₁-C₅ alkyl alcohol. In some examples of Formula II, R₁ is an unsubstituted C₁-C₅ alkyl alcohol. In some examples of Formula II, R₁ is -L¹-S—S—S—R^{a}.

In some examples of Formula II, R₂ is OH. In some examples of Formula II, R₂ is a substituted or unsubstituted C₁-C₅ alkyl. In some examples of Formula II, R₂ is a substituted C₁-C₅ alkyl. In some examples of Formula II, R₂ is an unsubstituted C₁-C₅ alkyl. In some examples of Formula II, R₂ is a substituted or unsubstituted C₁-C₅ alkyl alcohol. In some examples of Formula II, R₂ is a substituted C₁-C₅ alkyl alcohol. In some examples of Formula II, R₂ is an unsubstituted C₁-C₅ alkyl alcohol. In some examples of Formula II, R₂ is -L¹-S—S—S—R^{a}.

In some examples of Formula II, each R^{a}, when present, is independently a substituted or unsubstituted C₈-C₁₈ alkyl (e.g., C₁₀-C₁₄ alkyl or C₁₂ alkyl). In some examples of Formula II, each R^{a}, when present, is independently a substituted C₈-C₁₈ alkyl. In some examples of Formula II, each R^{a}, when present, is independently an unsubstituted C₈-C₁₈ alkyl.

In some examples of Formula II, L¹ in each case is independently a substituted or unsubstituted alkyl. In some examples of Formula II, L¹ in each case is independently a carbonyl. In some examples of Formula II, L¹ in each case is independently an ester. In some examples of Formula II, L¹ in each case is independently an amide. In some examples of Formula II, L¹ in each case is independently a carbamate ester. In some examples of Formula II, L¹ in each case is independently an amine. In some examples of Formula II, L¹ in each case is independently an ether. In some examples of Formula II, L¹ in each case is independently a carbonate ester. In some examples of Formula II, L¹ in each case is independently a thioether. In some examples of Formula II, L¹ in each case is independently a thioester. In some examples of Formula II, L¹ in each case is independently a urea. In some examples of Formula II having more than one L¹ groups, the compound includes a combination of the above (e.g., both a carbonate ester and a substituted or unsubstituted alkyl).

In some examples of Formula II, each L¹ is an ester represented by the formula —Z¹C(O)OZ²—, wherein Z¹ and Z² are each independently a substituted or unsubstituted C₁-C₁₀ alkyl. In some examples of Formula II, Z¹ and Z² are each independently a substituted or unsubstituted C₁-C₅ alkyl. In some examples of Formula II, Z¹ is —(C₂H₄)—. In some examples of Formula II, Z² is an unsubstituted C₁-C₇ alkyl. In some examples of Formula II, Z² is selected from:

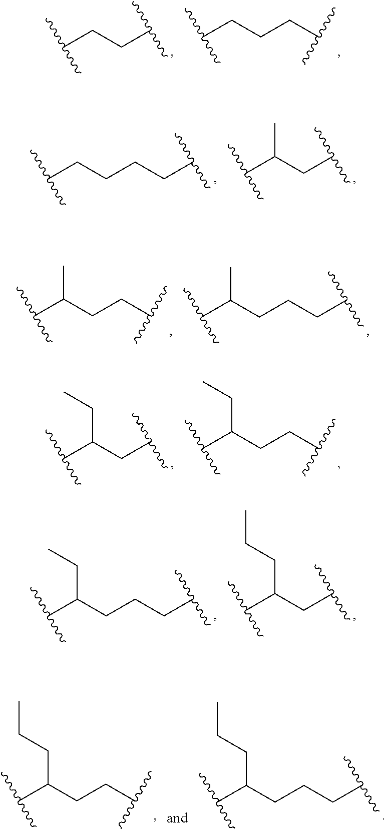

In some examples of Formula II, each R⁶ is independently an unsubstituted C₁-C₇ alkyl. In some examples of Formula II, each R⁶ is independently a linear C₁-C₇ alkyl. In some examples of Formula II, each R⁶ is independently a branched C₁-C₇ alkyl. In some examples of Formula II, each R⁶ is each independently a substituted or unsubstituted C₁-C₅ alkyl. In some examples of Formula II, each R⁶ is —(C₂H₄)—. In some examples of Formula II, each R⁶ is independently an unsubstituted C₁-C₇ alkyl. In some examples of Formula II, each R⁶ is independently selected from:

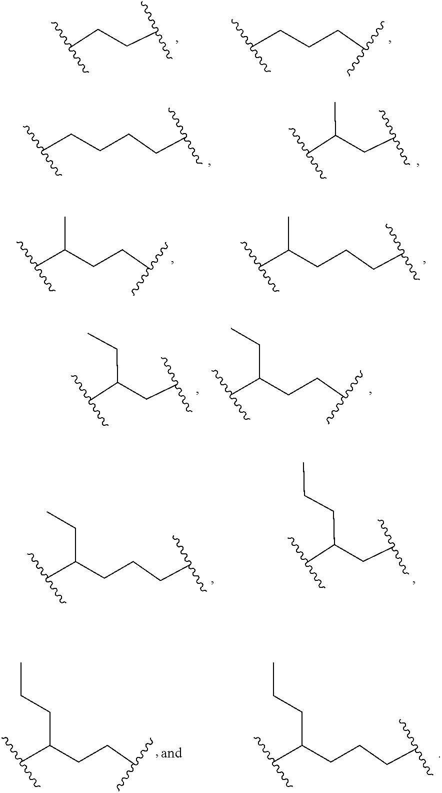

In some examples of Formula II, each R⁶ is the same. In some examples of Formula II, at least one R⁶ is different.

In some examples, the compound is defined by Formula III, or a pharmaceutically acceptable salt thereof:

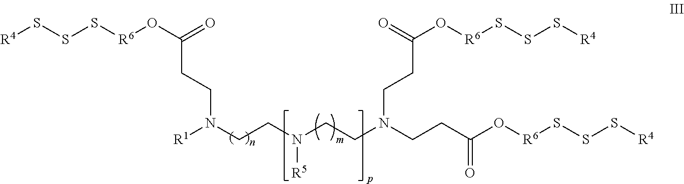

wherein
p is an integer from 0 to 5;
n is an integer from 1 to 10;
m, when present, is an integer from 1 to 10;
R¹ is OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl;
L¹, when present, is a substituted or unsubstituted alkyl, a carbonyl, an ester, an amide, a carbamate ester, an amine, an ether, a carbonate ester, a thioether, a thioester, or a urea; and each R⁶ is independently a substituted or unsubstituted C₁-C₇ alkyl.

In some examples of Formula III, n is from 1 to 7. In some examples of Formula III, n is from 1 to 6. In some examples of Formula III, n is from 1 to 5. In some examples of Formula III, n is from 1 to 4. In some examples of Formula III, n is from 1 to 3. In some examples of Formula III, n is 1.

In some examples of Formula III, m is from 1 to 7. In some examples of Formula III, m is from 1 to 6. In some examples of Formula III, m is from 1 to 5. In some examples of Formula III, m is from 1 to 4. In some examples of Formula III, m is from 1 to 3. In some examples of Formula III, m is 1. In some examples of Formula III, R⁵ is a substituted or unsubstituted C₁-C₅ alkyl. In some examples of Formula III, R⁵ is methyl. In some examples of Formula III, R⁵ is a substituted or unsubstituted C₁-C₅ alkyl alcohol. In some examples of Formula III, R⁵ is an unsubstituted C₁-C₅ alkyl alcohol. In some examples of Formula III, R⁵ is —CH₂OH. In some examples of Formula III, R⁵ is OH. In some examples of Formula III, R⁵ is hydrogen. In some examples of Formula III, R⁵ is -L¹-S—S—S—R^{a}.

In some examples of Formula III, each R⁴ is independently a substituted or unsubstituted C₈-C₁₈ alkyl (e.g., C₁₀-C₁₄ alkyl or C₁₂ alkyl). In some examples of Formula III, each R⁴ is independently a substituted C₈-C₁₈ alkyl (e.g., C₁₀-C₁₄ alkyl or C₁₂ alkyl). In some examples of Formula III, each R⁴ is independently a C₈-C₁₈ alkyl substituted with one or more substituents selected from the group consisting of amine, amide, ester, ether, and carbonate ester. In some examples of Formula III, each R⁴ is independently an unsubstituted C₈-C₁₈ alkyl (e.g., C₁₀-C₁₄ alkyl or C₁₂ alkyl). In some examples of Formula III, each R⁴ is independently an unsubstituted branched C₈-C₁₈ alkyl (e.g., C₁₀-C₁₄ alkyl or C₁₂ alkyl). In some examples of Formula III, each R⁴ is independently an unsubstituted linear C₈-C₁₈ alkyl (e.g., C₁₀-C₁₄ alkyl or C₁₂ alkyl). In some examples of Formula III, each R⁴ is the same. In some examples of Formula III, at least one R⁴ is different.

In some examples of Formula III, p is from 0 to 4. In some examples of Formula III, p is from 0 to 3. In some examples of Formula III, p is from 0 to 2. In some examples of Formula III, p is from 0 to 1. In some examples of Formula III, p is 0. In some examples of Formula III, p is 1. In some examples of Formula III, p is 2 or more (e.g., 2, 3, 4, 5). In some examples of Formula III, where p is 2 or more, each R⁵ is the same. In some examples of Formula III, where p is 2 or more, at least one R⁵ is different. In some examples of Formula III, where p is 2 or more, each m is the same. In some examples of Formula III, where p is 2 or more, at least one m is different.

In some examples of Formula III, R₁ is OH. In some examples of Formula III, R₁ is a substituted or unsubstituted C₁-C₅ alkyl. In some examples of Formula III, R₁ is a substituted C₁-C₅ alkyl. In some examples of Formula III, R₁ is an unsubstituted C₁-C₅ alkyl. In some examples of Formula III, R₁ is a substituted or unsubstituted C₁-C₅ alkyl alcohol. In some examples of Formula III, R₁ is a substituted C₁-C₅ alkyl alcohol. In some examples of Formula III, R₁ is an unsubstituted C₁-C₅ alkyl alcohol. In some examples of Formula III, R₁ is -L¹-S—S—S—R^{a}.

In some examples of Formula III, each R^{a}, when present, is independently a substituted or unsubstituted C₈-C₁₈ alkyl (e.g., C₁₀-C₁₄ alkyl or C₁₂ alkyl). In some examples of Formula III, each R^{a}, when present, is independently a substituted C₈-C₁₈ alkyl. In some examples of Formula III, each R^{a}, when present, is an unsubstituted C₈-C₁₈ alkyl.

In some examples of Formula III, L¹ is a substituted or unsubstituted alkyl. In some examples of Formula III, L¹ is a carbonyl. In some examples of Formula III, L¹ is an ester. In some examples of Formula III, L¹ is an amide. In some examples of Formula III, L¹ is a carbamate ester. In some examples of Formula III, L¹ is an amine. In some examples of Formula III, L¹ is an ether. In some examples of Formula III, L¹ is a carbonate ester. In some examples of Formula III, L¹ is a thioether. In some examples of Formula III, L¹ is a thioester. In some examples of Formula III, L¹ is a urea.

In some examples of Formula III, L¹ is an ester represented by the formula —Z¹C(O)OZ²—, wherein Z¹ and Z² are each independently a substituted or unsubstituted C₁-C₁₀ alkyl. In some examples of Formula III, Z¹ and Z² are each independently a substituted or unsubstituted C₁-C₅ alkyl. In some examples of Formula III, Z is —(C₂H₄)—. In some examples of Formula III, Z² is an unsubstituted C₁-C₇ alkyl. In some examples of Formula III, Z² is selected from;

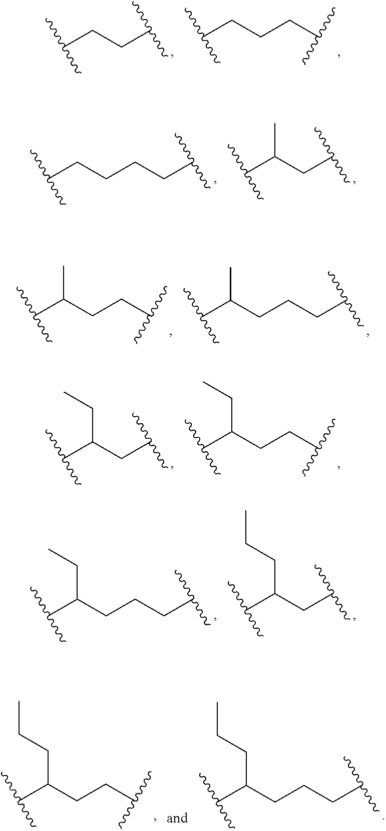

In some examples of Formula III, each R⁶ is independently an unsubstituted C₁-C₇ alkyl. In some examples of Formula III, each R⁶ is independently a linear C₁-C₇ alkyl. In some examples of Formula III, each R⁶ is independently a branched C₁-C₇ alkyl. In some examples of Formula III, each R⁶ is each independently a substituted or unsubstituted C₁-C₅ alkyl. In some examples of Formula III, each R⁶ is —(C₂H₄)—. In some examples of Formula III, each R⁶ is independently an unsubstituted C₁-C₇ alkyl. In some examples of Formula III, each R⁶ is independently selected from:

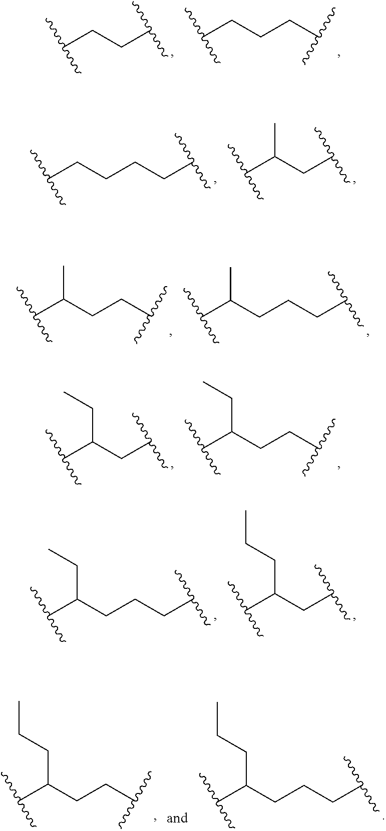

In some examples of Formula III, each R⁶ is the same. In some examples of Formula III, at least one R⁶ is different.

In some examples, the compound is defined by Formula IV, or a pharmaceutically acceptable salt thereof:

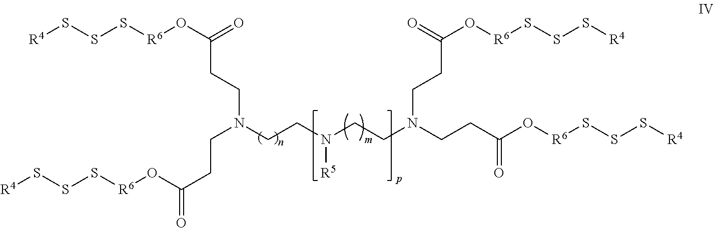

wherein
p is an integer from 0 to 5;
n is an integer from 1 to 10;
m, when present, is an integer from 1 to 10;
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl; and
each R⁶ is independently a substituted or unsubstituted C₁-C₇ alkyl.

In some examples of Formula IV, n is from 1 to 7. In some examples of Formula IV, n is from 1 to 6. In some examples of Formula IV, n is from 1 to 5. In some examples of Formula IV, n is from 1 to 4. In some examples of Formula IV, n is from 1 to 3. In some examples of Formula IV, n is 1.

In some examples of Formula IV, m is from 1 to 7. In some examples of Formula IV, m is from 1 to 6. In some examples of Formula IV, m is from 1 to 5. In some examples of Formula IV, m is from 1 to 4. In some examples of Formula IV, m is from 1 to 3. In some examples of Formula IV, m is 1. In some examples of Formula IV, R⁵ is a substituted or unsubstituted C₁-C₅ alkyl. In some examples of Formula IV, R⁵ is methyl. In some examples of Formula IV, R⁵ is a substituted or unsubstituted C₁-C₅ alkyl alcohol. In some examples of Formula IV, R⁵ is an unsubstituted C₁-C₅ alkyl alcohol. In some examples of Formula IV, R⁵ is —CH₂OH. In some examples of Formula IV, R⁵ is OH. In some examples of Formula IV, R⁵ is hydrogen. In some examples of Formula IV, R⁵ is -L¹-S—S—S—R^{a}.

In some examples of Formula IV, each R⁴ is independently a substituted or unsubstituted C₈-C₁₈ alkyl (e.g., C₁₀-C₁₄ alkyl or C₁₂ alkyl). In some examples of Formula IV, each R⁴ is independently a substituted C₈-C₁₈ alkyl (e.g., C₁₀-C₁₄ alkyl or C₁₂ alkyl). In some examples of Formula IV, each R⁴ is independently a C₈-C₁₈ alkyl substituted with one or more substituents selected from the group consisting of amine, amide, ester, ether, and carbonate ester. In some examples of Formula IV, each R⁴ is independently an unsubstituted C₈-C₁₈ alkyl (e.g., C₁₀-C₁₄ alkyl or C₁₂ alkyl). In some examples of Formula IV, each R⁴ is independently an unsubstituted branched C₈-C₁₈ alkyl (e.g., C₁₀-C₁₄ alkyl or C₁₂ alkyl). In some examples of Formula IV, each R⁴ is independently an unsubstituted linear C₈-C₁₈ alkyl (e.g., C₁₀-C₁₄ alkyl or C₁₂ alkyl). In some examples of Formula IV, each R⁴ is the same. In some examples of Formula IV, at least one R⁴ is different.

In some examples of Formula IV, p is from 0 to 4. In some examples of Formula IV, p is from 0 to 3. In some examples of Formula IV, p is from 0 to 2. In some examples of Formula IV, p is from 0 to 1. In some examples of Formula IV, p is 0. In some examples of Formula IV, p is 1. In some examples of Formula IV, p is 2 or more (e.g., 2, 3, 4, 5). In some examples of Formula IV, where p is 2 or more, each R⁵ is the same. In some examples of Formula IV, where p is 2 or more, at least one R⁵ is different. In some examples of Formula IV, where p is 2 or more, each m is the same. In some examples of Formula IV, where p is 2 or more, at least one m is different.

In some examples of Formula IV, each R^{a}, when present, is independently a substituted or unsubstituted C₈-C₁₈ alkyl (e.g., C₁₀-C₁₄ alkyl or C₁₂ alkyl). In some examples of Formula IV, each R^{a}, when present, is independently a substituted C₈-C₁₈ alkyl. In some examples of Formula IV, each R^{a}, when present, is independently an unsubstituted C₈-C₁₈ alkyl.

In some examples of Formula IV, each R⁶ is independently an unsubstituted C₁-C₇ alkyl. In some examples of Formula IV, each R⁶ is independently a linear C₁-C₇ alkyl. In some examples of Formula IV, each R⁶ is independently a branched C₁-C₇ alkyl. In some examples of Formula IV, each R⁶ is each independently a substituted or unsubstituted C₁-C₅ alkyl. In some examples of Formula IV, each R⁶ is —(C₂H₄)—. In some examples of Formula IV, each R⁶ is independently an unsubstituted C₁-C₇ alkyl. In some examples of Formula IV, each R⁶ is independently selected from.

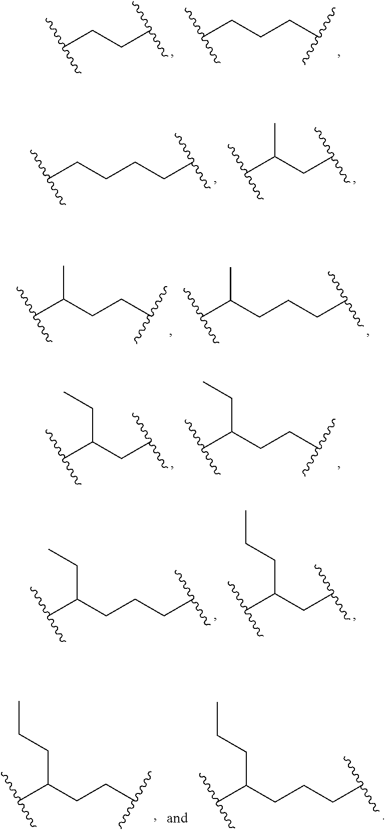

In some examples of Formula IV, each R⁶ is the same. In some examples of Formula IV, at least one R⁶ is different.

In some examples, the compound is defined by Formula V, or a pharmaceutically acceptable salt thereof:

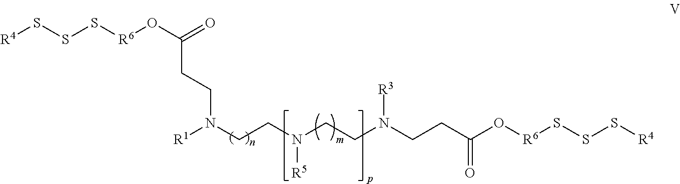

wherein
p is an integer from 0 to 5;
n is an integer from 1 to 10;
each m, when present, is independently an integer from 1 to 10;
R¹ and R³ are independently OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

In some examples of Formula V, n is from 1 to 7. In some examples of Formula V, n is from 1 to 6. In some examples of Formula V, n is from 1 to 5. In some examples of Formula V, n is from 1 to 4. In some examples of Formula V, n is from 1 to 3. In some examples of Formula V, n is 1.

In some examples of Formula V, m is from 1 to 7. In some examples of Formula V, m is from 1 to 6. In some examples of Formula V, m is from 1 to 5. In some examples of Formula V, m is from 1 to 4. In some examples of Formula V, m is from 1 to 3. In some examples of Formula V, m is 1. In some examples of Formula V, R⁵ is a substituted or unsubstituted C₁-C₅ alkyl. In some examples of Formula V, R⁵ is methyl. In some examples of Formula V, R⁵ is a substituted or unsubstituted C₁-C₅ alkyl alcohol. In some examples of Formula V, R⁵ is an unsubstituted C₁-C₅ alkyl alcohol. In some examples of Formula V, R⁵ is —CH₂OH. In some examples of Formula V, R⁵ is OH. In some examples of Formula V, R⁵ is hydrogen. In some examples of Formula V, R⁵ is -L¹-S—S—S—R^{a}.

In some examples of Formula V, R₁ is OH. In some examples of Formula V, R₁ is a substituted or unsubstituted C₁-C₅ alkyl. In some examples of Formula V, R₁ is a substituted C₁-C₅ alkyl. In some examples of Formula V, R₁ is an unsubstituted C₁-C₅ alkyl. In some examples of Formula V, R₁ is a substituted or unsubstituted C₁-C₅ alkyl alcohol. In some examples of Formula V, R₁ is a substituted C₁-C₅ alkyl alcohol. In some examples of Formula V, R₁ is an unsubstituted C₁-C₅ alkyl alcohol. In some examples of Formula V, R₁ is -L¹-S—S—S—R^{a};

In some examples of Formula V, R₃ is OH. In some examples of Formula V, R₃ is a substituted or unsubstituted C₁-C₅ alkyl. In some examples of Formula V, R₃ is a substituted C₁-C₅ alkyl. In some examples of Formula V, R₃ is an unsubstituted C₁-C₅ alkyl. In some examples of Formula V, R₃ is a substituted or unsubstituted C₁-C₅ alkyl alcohol. In some examples of Formula V, R₃ is a substituted C₁-C₅ alkyl alcohol. In some examples of Formula V, R₃ is an unsubstituted C₁-C₅ alkyl alcohol. In some examples of Formula V, R₃ is -L¹-S—S—S—R^{a}.

In some examples of Formula V, each R⁴ is independently a substituted or unsubstituted C₈-C₁₈ alkyl (e.g., C₁₀-C₁₄ alkyl or C₁₂ alkyl). In some examples of Formula V, each R⁴ is independently a substituted C₈-C₁₈ alkyl (e.g., C₁₀-C₁₄ alkyl or C₁₂ alkyl). In some examples of Formula V, each R⁴ is independently a C₈-C₁₈ alkyl substituted with one or more substituents selected from the group consisting of amine, amide, ester, ether, and carbonate ester. In some examples of Formula V, each R⁴ is independently an unsubstituted C₈-C₁₈ alkyl (e.g., C₁₀-C₁₄ alkyl or C₁₂ alkyl). In some examples of Formula V, each R⁴ is independently an unsubstituted branched C₈-C₁₈ alkyl (e.g., C₁₀-C₁₄ alkyl or C₁₂ alkyl). In some examples of Formula V, each R⁴ is independently an unsubstituted linear C₈-C₁₈ alkyl (e.g., C₁₀-C₁₄ alkyl or C₁₂ alkyl). In some examples of Formula V, each R⁴ is the same. In some examples of Formula V, at least one R⁴ is different.

In some examples of Formula V, p is from 0 to 4. In some examples of Formula V, p is from 0 to 3. In some examples of Formula V, p is from 0 to 2. In some examples of Formula V, p is from 0 to 1. In some examples of Formula V, p is 0. In some examples of Formula V, p is 1. In some examples of Formula V, p is 2 or more (e.g., 2, 3, 4, 5). In some examples of Formula V, where p is 2 or more, each R⁵ is the same. In some examples of Formula V, where p is 2 or more, at least one R⁵ is different. In some examples of Formula V, where p is 2 or more, each m is the same. In some examples of Formula V, where p is 2 or more, at least one m is different.

In some examples of Formula V, each R^{a}, when present, is independently a substituted or unsubstituted C₈-C₁₈ alkyl (e.g., C₁₀-C₁₄ alkyl or C₁₂ alkyl). In some examples of Formula V, each R^{a}, when present, is independently a substituted C₈-C₁₈ alkyl. In some examples of Formula V, each R^{a}, when present, is independently an unsubstituted C₈-C₁₈ alkyl.

In some examples of Formula V, L¹ in each case is independently a substituted or unsubstituted alkyl. In some examples of Formula V, L¹ in each case is independently a carbonyl. In some examples of Formula V, L¹ in each case is independently an ester. In some examples of Formula V, L¹ in each case is independently an amide. In some examples of Formula V, L¹ in each case is independently a carbamate ester. In some examples of Formula V, L¹ in each case is independently an amine. In some examples of Formula V, L¹ in each case is independently an ether. In some examples of Formula V, L¹ in each case is independently a carbonate ester. In some examples of Formula V, L¹ in each case is independently a thioether. In some examples of Formula V, L¹ in each case is independently a thioester. In some examples of Formula V, L¹ in each case is independently a urea. In some examples of Formula V having more than one L¹ groups, the compound includes a combination of the above (e.g., both a carbonate ester and a substituted or unsubstituted alkyl).

In some examples of Formula V, each L¹ is an ester represented by the formula —Z¹C(O)OZ²—, wherein Z¹ and Z² are each independently a substituted or unsubstituted C₁-C₁₀ alkyl. In some examples of Formula V, Z¹ and Z² are each independently a substituted or unsubstituted C₁-C₅ alkyl. In some examples of Formula V, Z¹ is —(C₂H₄)—. In some examples of Formula V, Z² is an unsubstituted C₁-C₇ alkyl. In some examples of Formula V, Z² is selected from:

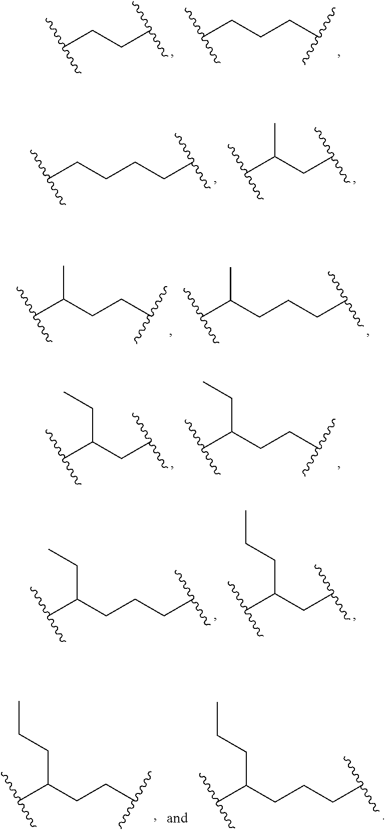

In some examples of Formula V, each R⁶ is independently an unsubstituted C₁-C₇ alkyl. In some examples of Formula V, each R⁶ is independently a linear C₁-C₇ alkyl. In some examples of Formula V, each R⁶ is independently a branched C₁-C₇ alkyl. In some examples of Formula V, each R⁶ is each independently a substituted or unsubstituted C₁-C₅ alkyl. In some examples of Formula V, each R⁶ is —(C₂H₄)—. In some examples of Formula V, each R⁶ is independently an unsubstituted C₁-C₇ alkyl. In some examples of Formula V, each R⁶ is independently selected from:

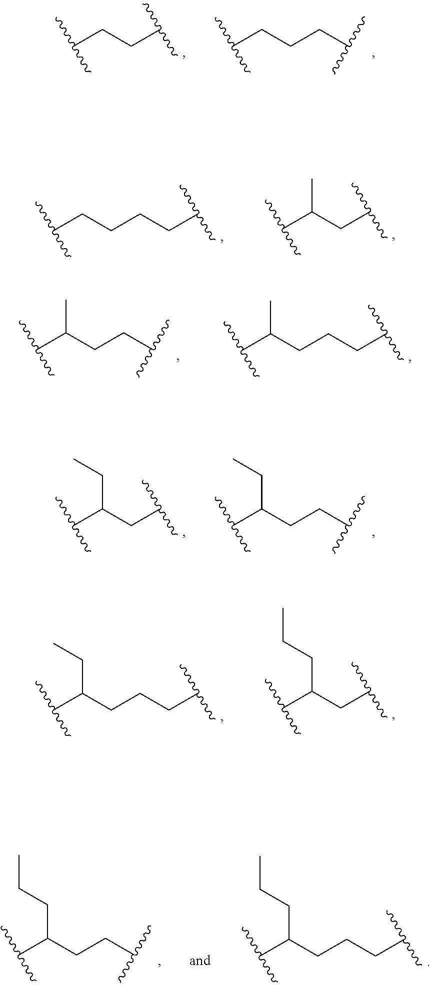

In some examples of Formula V, each R⁶ is the same. In some examples of Formula V, at least one R⁶ is different.

In some examples, the compound is selected from the group consisting of:

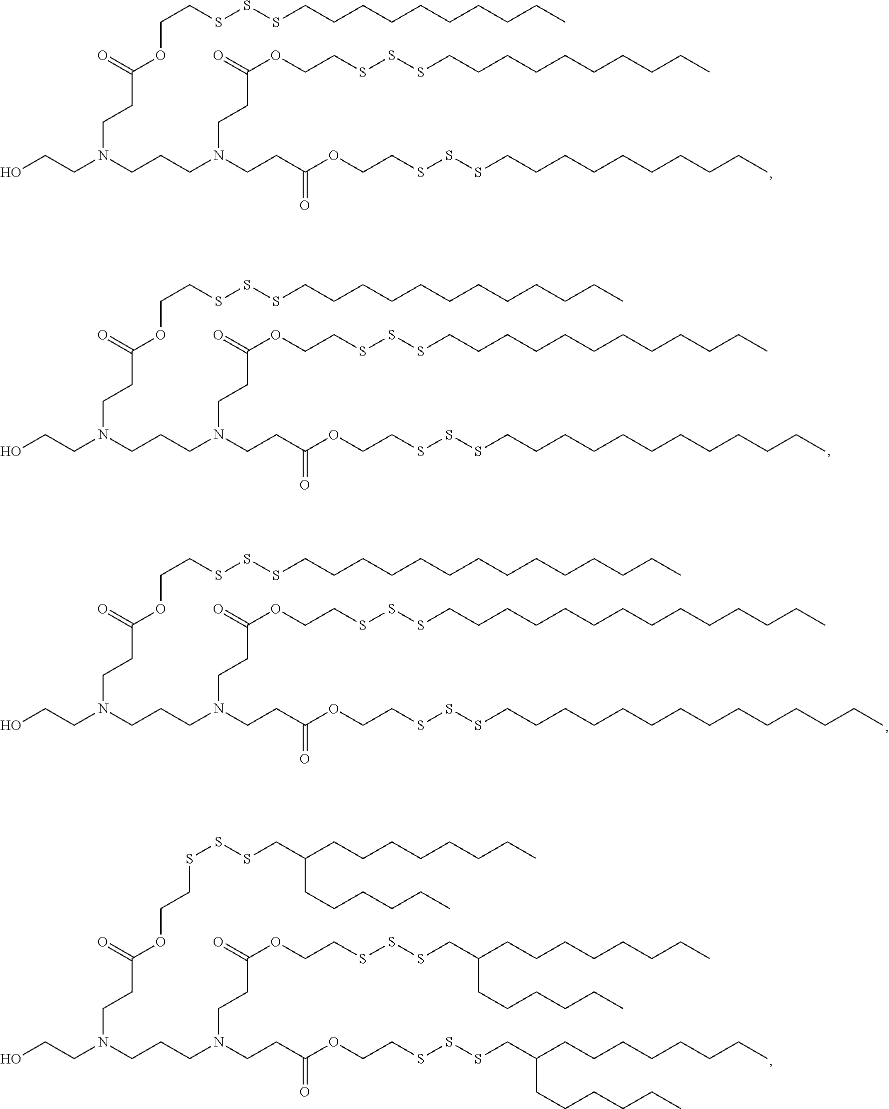 [SMILES: CCCCCCCCCCSSSCCOC(=O)CCN(CCO)CCCN(CCC(=O)OCCSSSCCCCCCCCCC)CCC(=O)OCCSSSCCCCCCCCCC ; CCCCCCCCCCCCSSSCCOC(=O)CCN(CCO)CCCN(CCC(=O)OCCSSSCCCCCCCCCCCC)CCC(=O)OCCSSSCCCCCCCCCCCC ; CCCCCCCCCCCCCCSSSCCOC(=O)CCN(CCO)CCCN(CCC(=O)OCCSSSCCCCCCCCCCCCCC)CCC(=O)OCCSSSCCCCCCCCCCCCCC ; CCCCCCCCC(CCCCCC)CSSSCCOC(=O)CCN(CCO)CCCN(CCC(=O)OCCSSSCC(CCCCCC)CCCCCCCC)CCC(=O)OCCSSSCC(CCCCCC)CCCCCCCC]

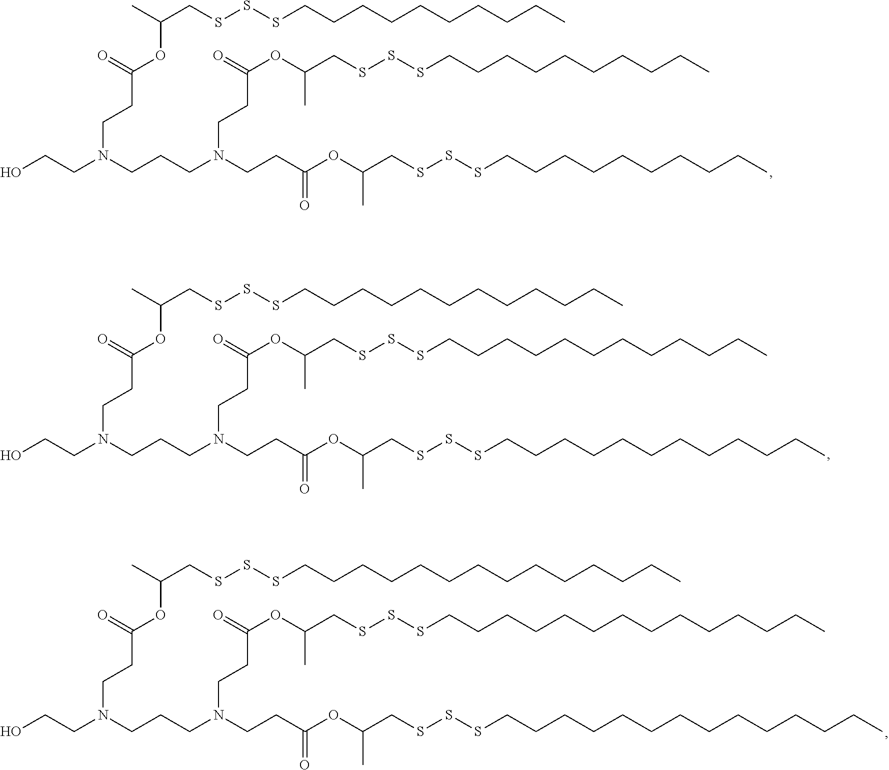 [SMILES: CCCCCCCCCCSSSCC(C)OC(=O)CCN(CCO)CCCN(CCC(=O)OC(C)CSSSCCCCCCCCCC)CCC(=O)OC(C)CSSSCCCCCCCCCC ; CCCCCCCCCCCCSSSCC(C)OC(=O)CCN(CCO)CCCN(CCC(=O)OC(C)CSSSCCCCCCCCCCCC)CCC(=O)OC(C)CSSSCCCCCCCCCCCC ; CCCCCCCCCCCCCCSSSCC(C)OC(=O)CCN(CCO)CCCN(CCC(=O)OC(C)CSSSCCCCCCCCCCCCCC)CCC(=O)OC(C)CSSSCCCCCCCCCCCCCC]

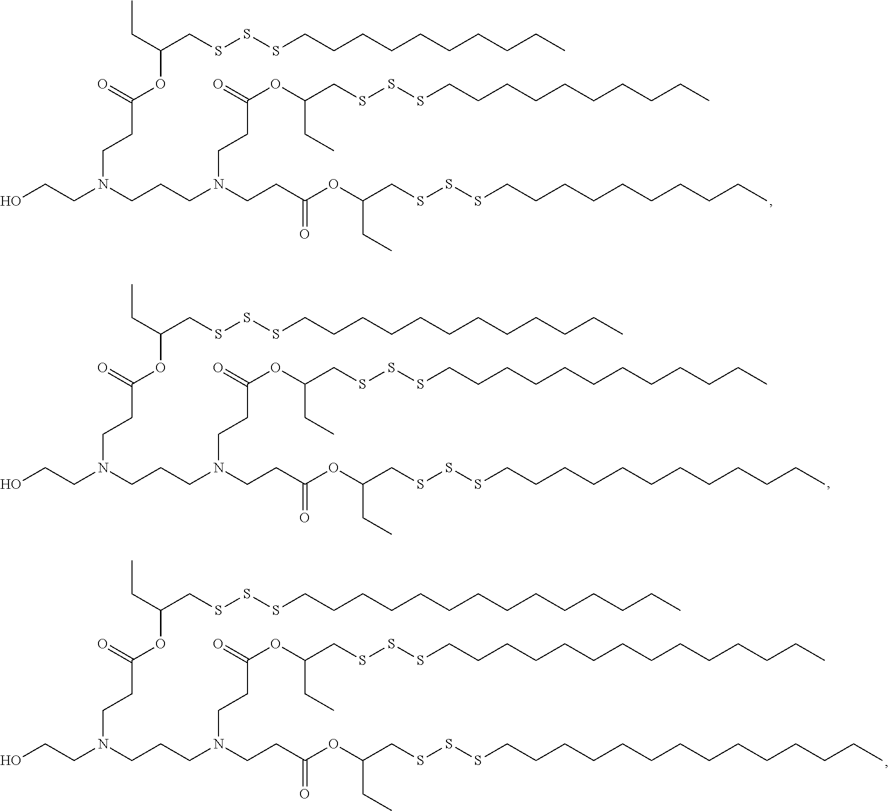 [SMILES: CCCCCCCCCCSSSCC(CC)OC(=O)CCN(CCO)CCCN(CCC(=O)OC(CC)CSSSCCCCCCCCCC)CCC(=O)OC(CC)CSSSCCCCCCCCCC ; CCCCCCCCCCCCSSSCC(CC)OC(=O)CCN(CCO)CCCN(CCC(=O)OC(CC)CSSSCCCCCCCCCCCC)CCC(=O)OC(CC)CSSSCCCCCCCCCCCC ; CCCCCCCCCCCCCCSSSCC(CC)OC(=O)CCN(CCO)CCCN(CCC(=O)OC(CC)CSSSCCCCCCCCCCCCCC)CCC(=O)OC(CC)CSSSCCCCCCCCCCCCCC]

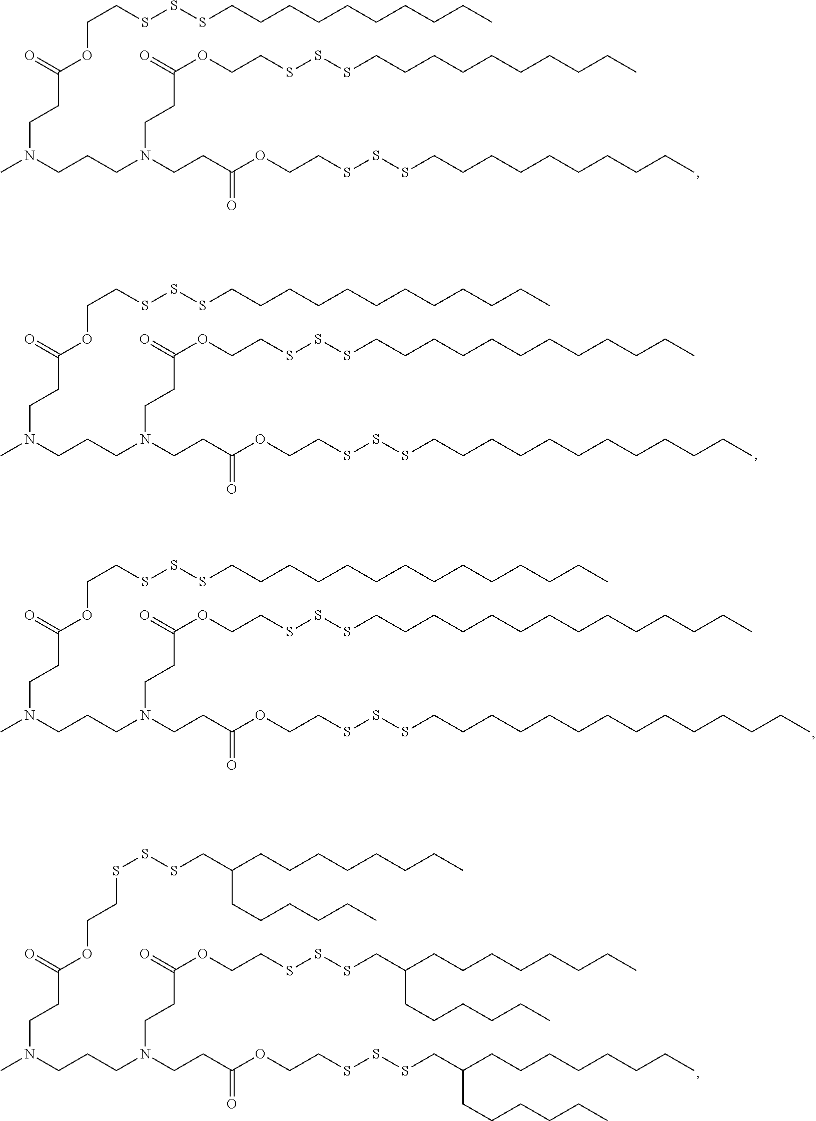

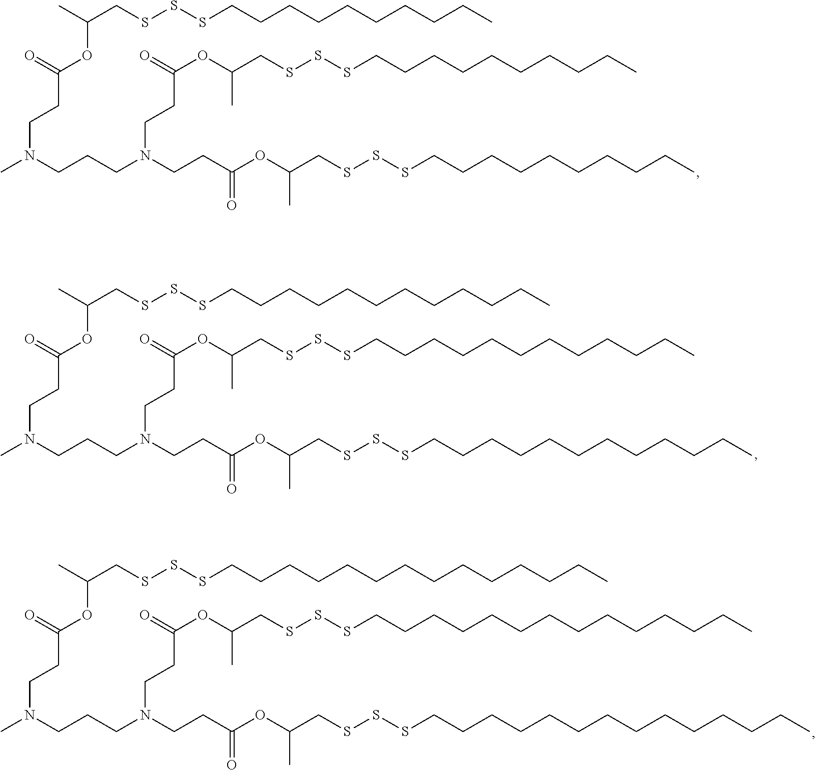

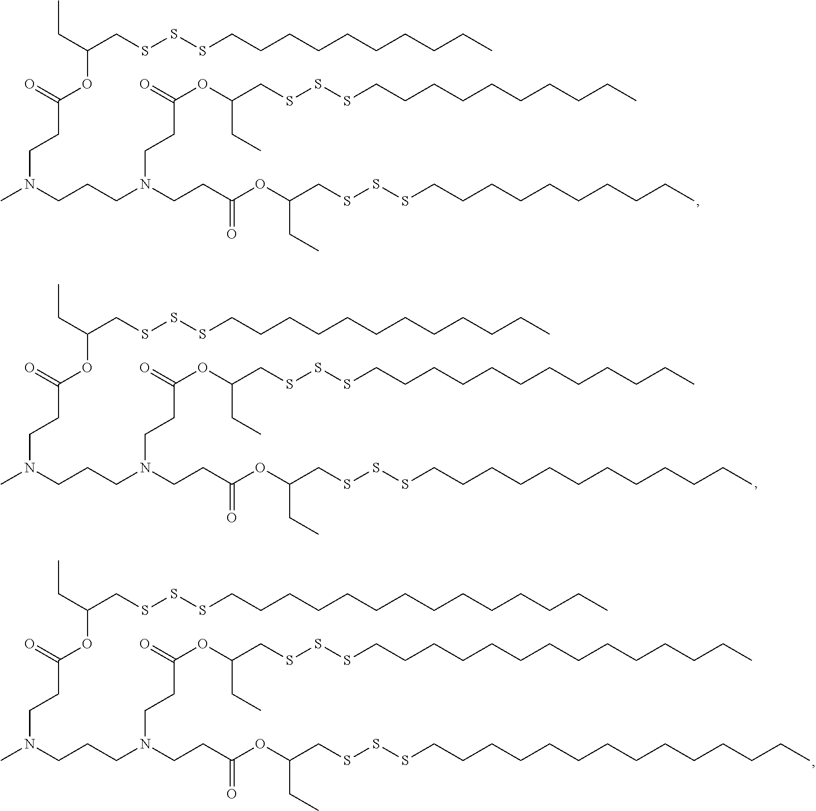

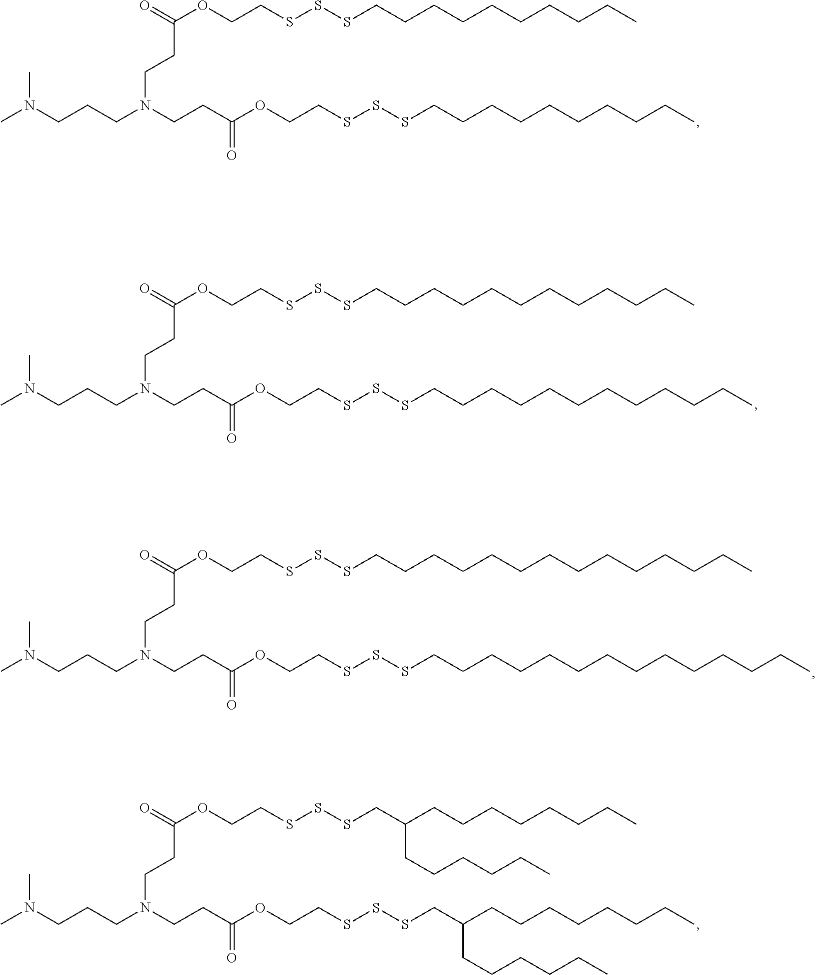

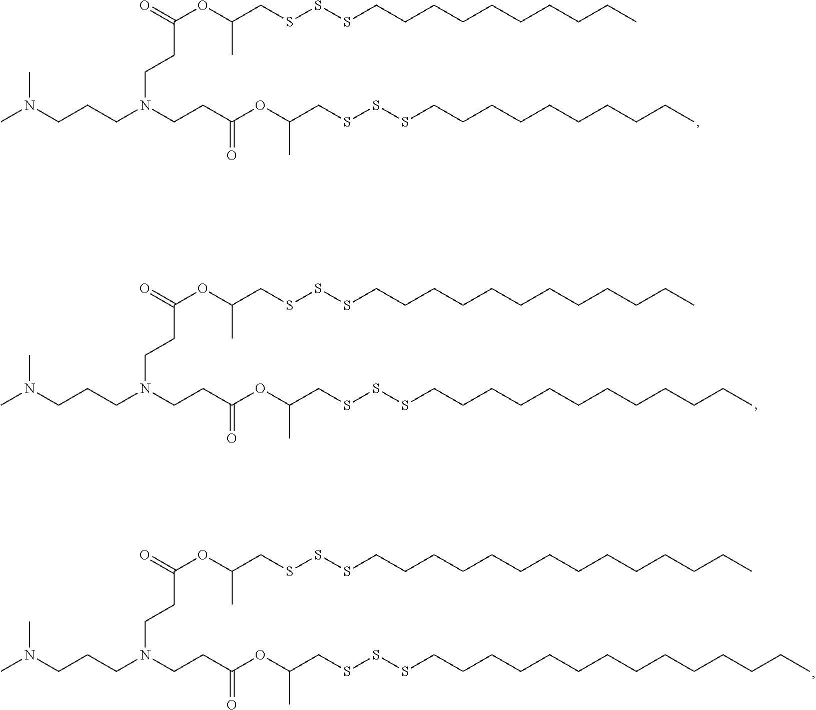

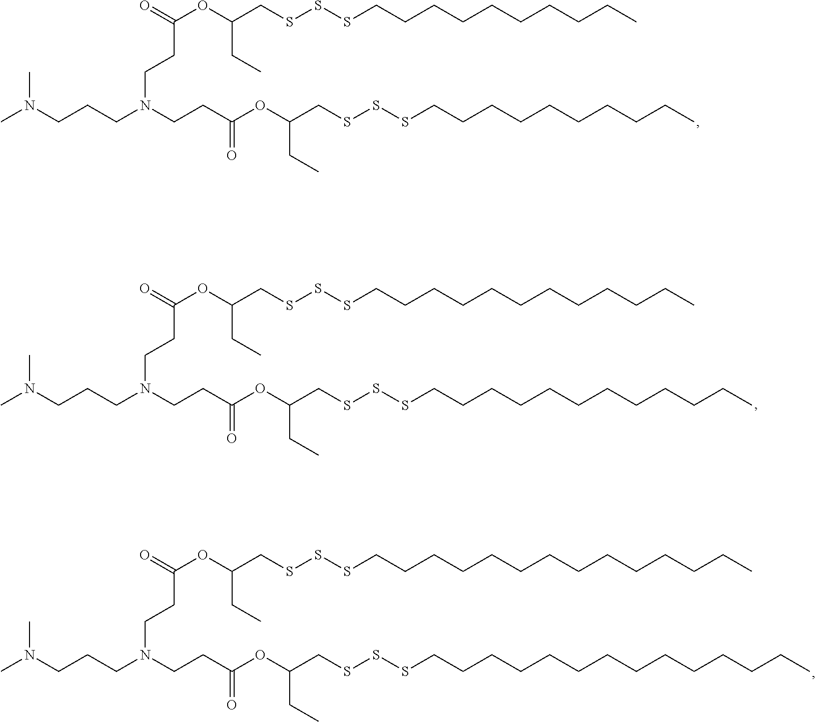

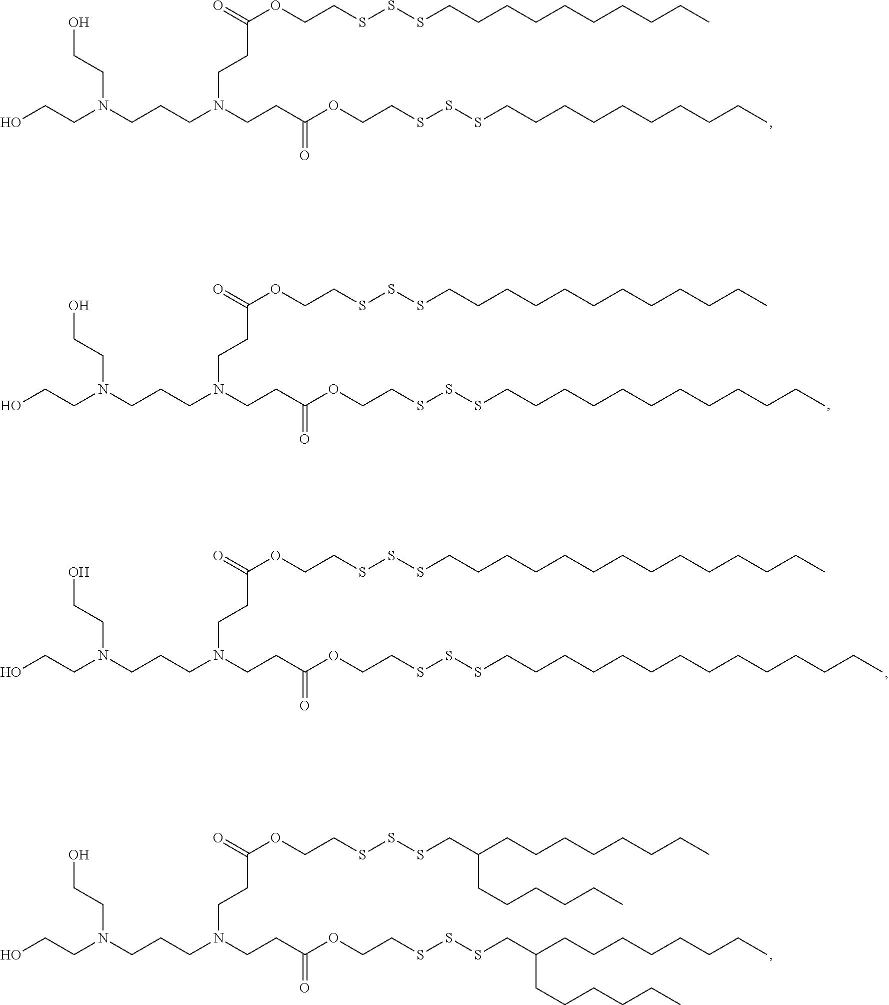 [SMILES: CCCCCCCCCCSSSCCOC(=O)CCN(CCCN(CCO)CCO)CCC(=O)OCCSSSCCCCCCCCCC ; CCCCCCCCCCCCSSSCCOC(=O)CCN(CCCN(CCO)CCO)CCC(=O)OCCSSSCCCCCCCCCCCC ; CCCCCCCCCCCCCCSSSCCOC(=O)CCN(CCCN(CCO)CCO)CCC(=O)OCCSSSCCCCCCCCCCCCCC ; CCCCCCCCC(CCCCCC)CSSSCCOC(=O)CCN(CCCN(CCO)CCO)CCC(=O)OCCSSSCC(CCCCCC)CCCCCCCC]

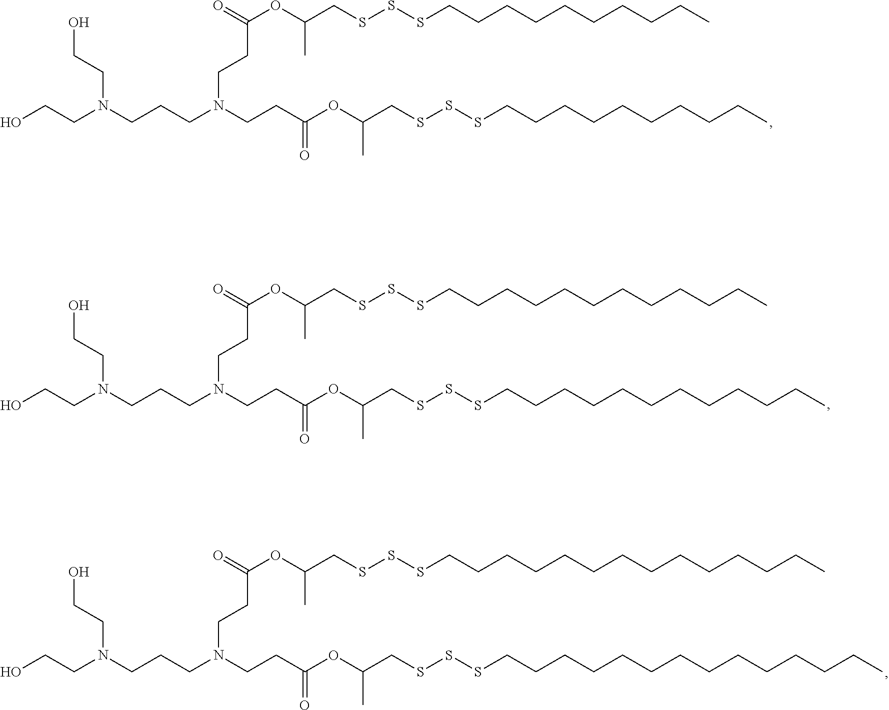 [SMILES: CCCCCCCCCCSSSCC(C)OC(=O)CCN(CCCN(CCO)CCO)CCC(=O)OC(C)CSSSCCCCCCCCCC ; CCCCCCCCCCCCSSSCC(C)OC(=O)CCN(CCCN(CCO)CCO)CCC(=O)OC(C)CSSSCCCCCCCCCCCC ; CCCCCCCCCCCCCCSSSCC(C)OC(=O)CCN(CCCN(CCO)CCO)CCC(=O)OC(C)CSSSCCCCCCCCCCCCCC]

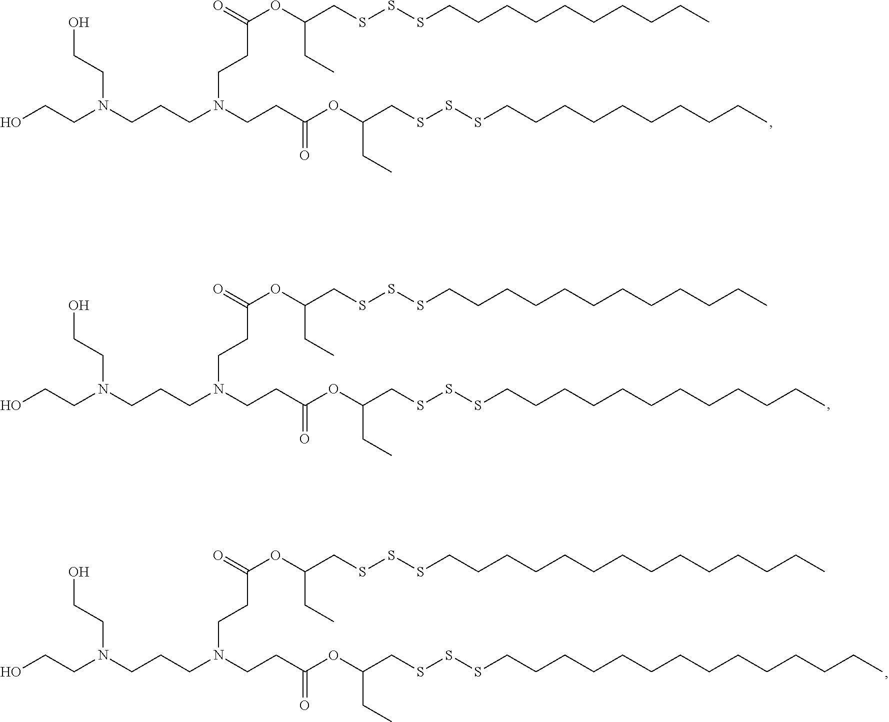 [SMILES: CCCCCCCCCCSSSCC(CC)OC(=O)CCN(CCCN(CCO)CCO)CCC(=O)OC(CC)CSSSCCCCCCCCCC ; CCCCCCCCCCCCSSSCC(CC)OC(=O)CCN(CCCN(CCO)CCO)CCC(=O)OC(CC)CSSSCCCCCCCCCCCC ; CCCCCCCCCCCCCCSSSCC(CC)OC(=O)CCN(CCCN(CCO)CCO)CCC(=O)OC(CC)CSSSCCCCCCCCCCCCCC]

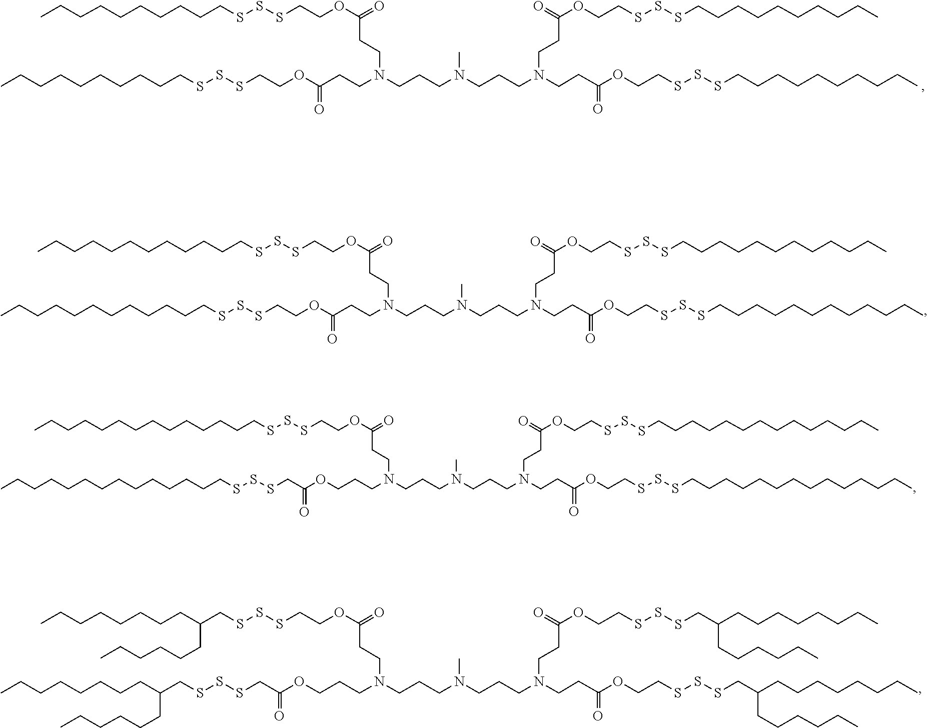

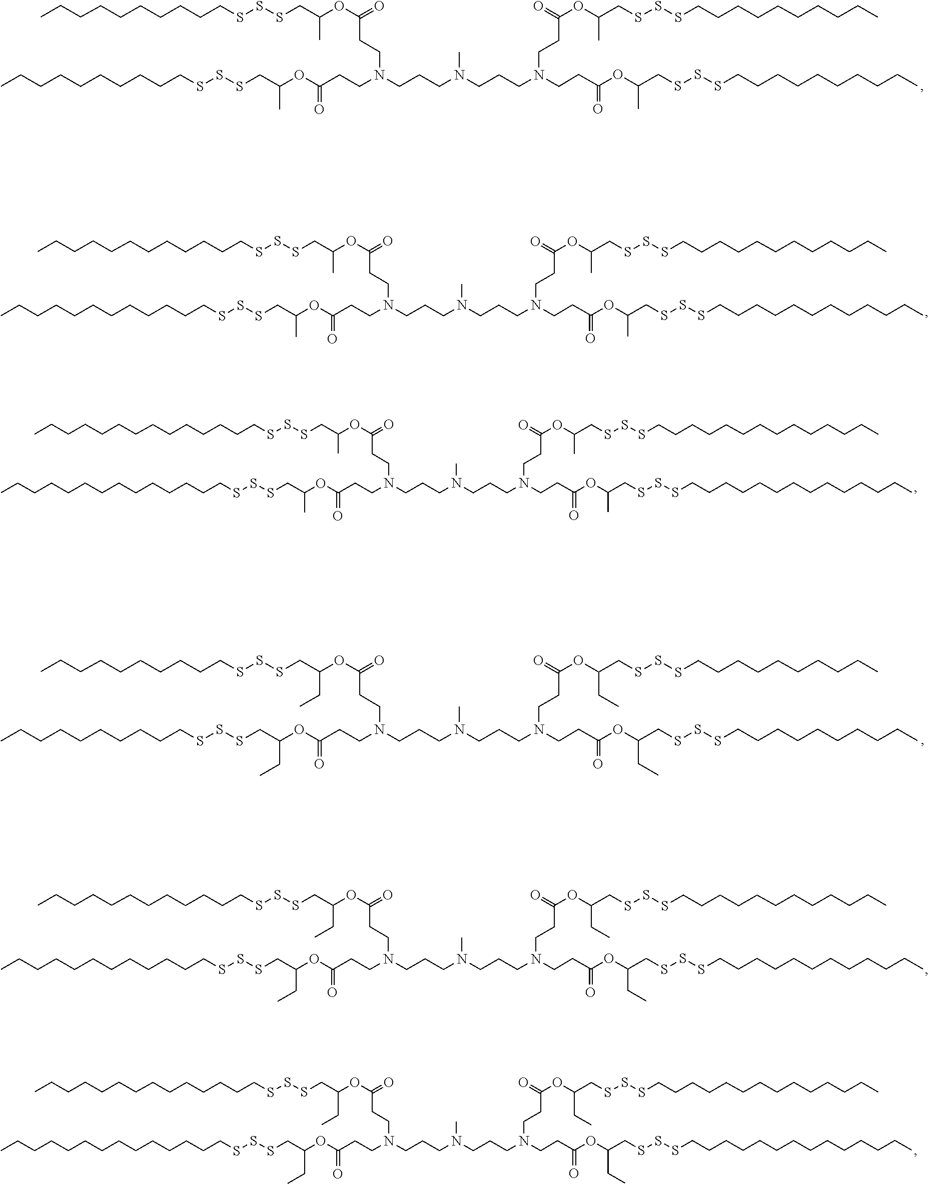

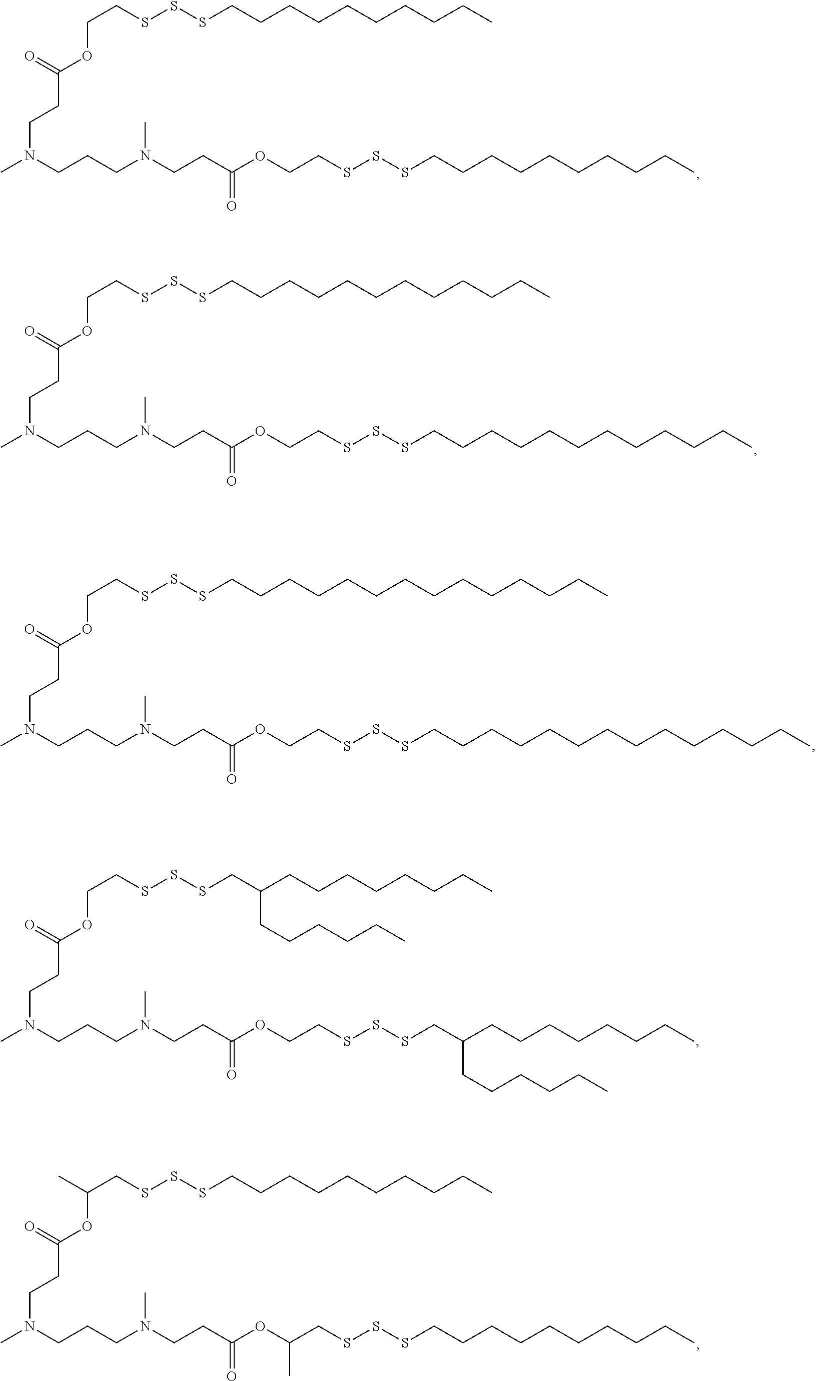

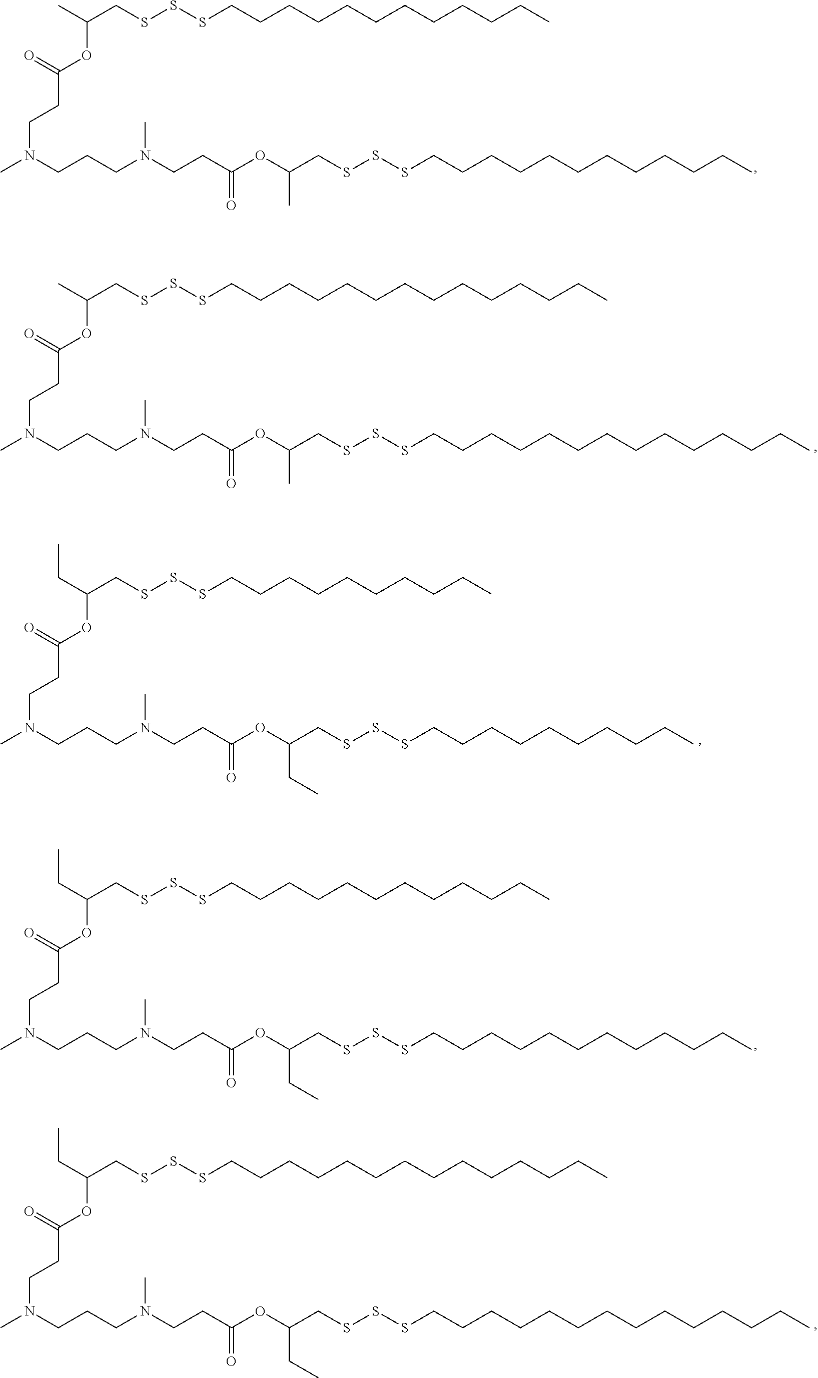

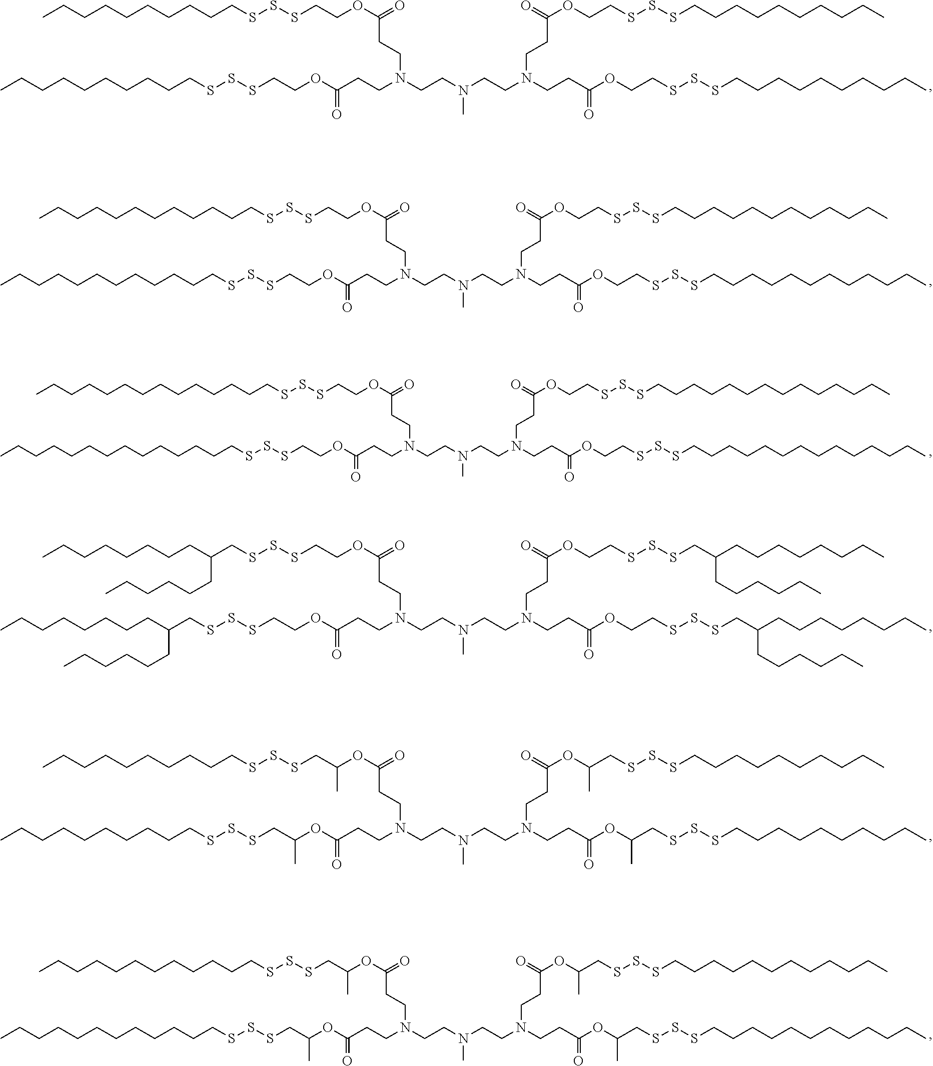

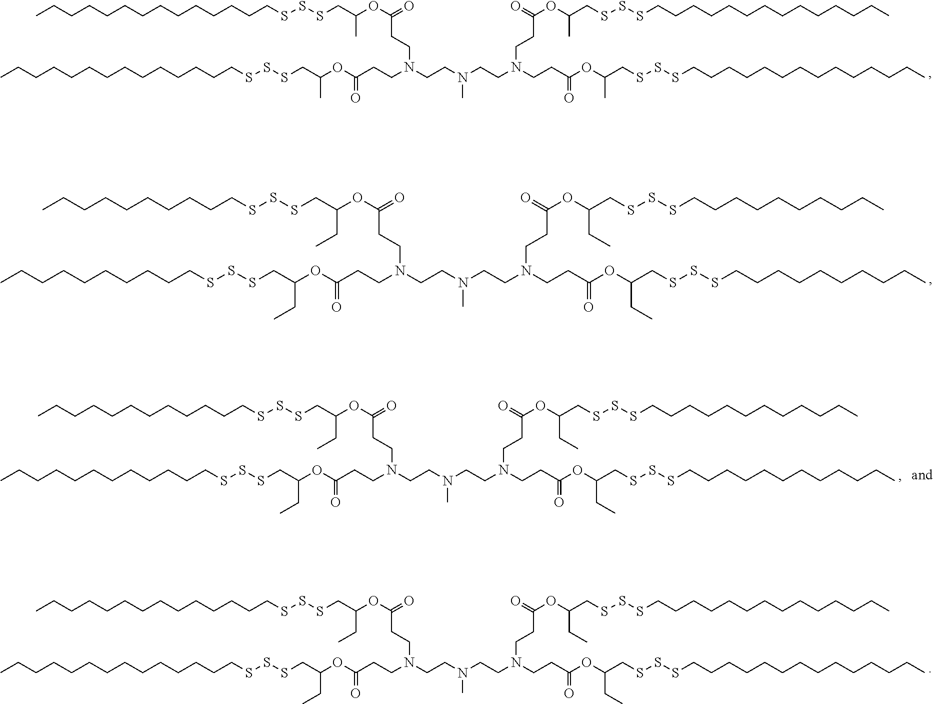

In some examples, the compound is selected from the group consisting of:

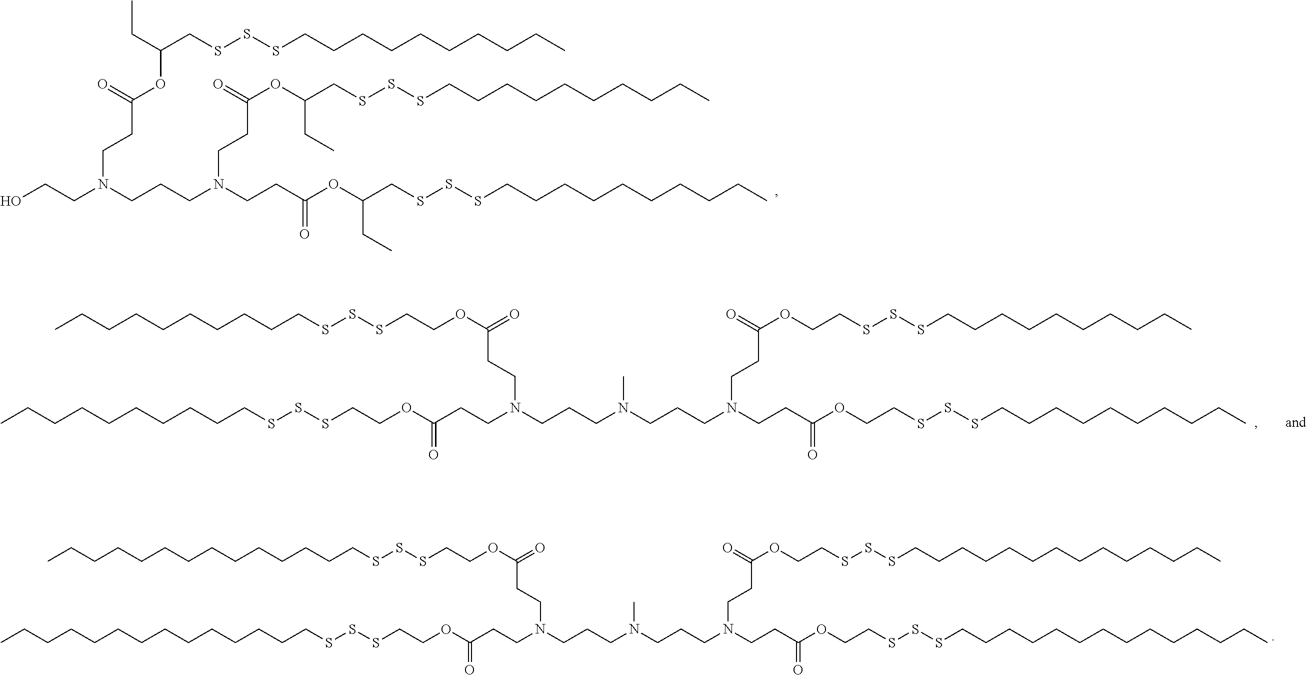

In some examples, the compound is selected from the group consisting of:

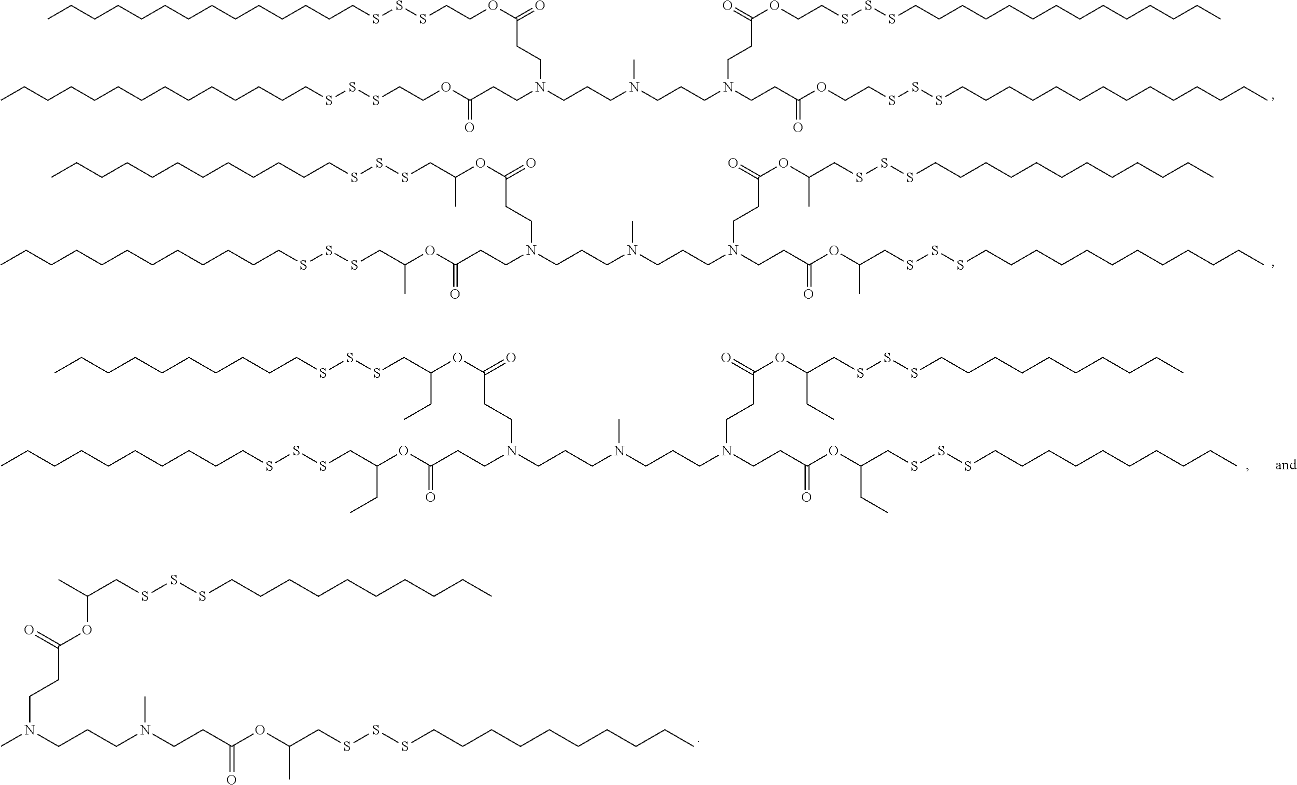

In some examples, the compound comprises:

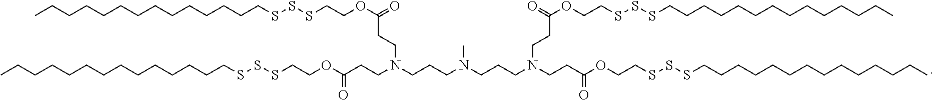 [SMILES: CCCCCCCCCCCCCCSSSCCOC(=O)CCN(CCCN(C)CCCN(CCC(=O)OCCSSSCCCCCCCCCCCCCC)CCC(=O)OCCSSSCCCCCCCCCCCCCC)CCC(=O)OCCSSSCCCCCCCCCCCCCC]

In some examples, the compound comprises:

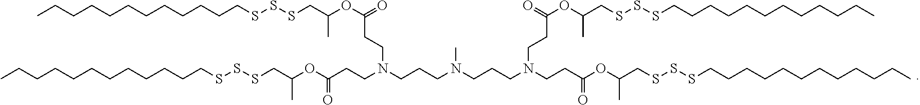 [SMILES: CCCCCCCCCCCCSSSCC(C)OC(=O)CCN(CCCN(C)CCCN(CCC(=O)OC(C)CSSSCCCCCCCCCCCC)CCC(=O)OC(C)CSSSCCCCCCCCCCCC)CCC(=O)OC(C)CSSSCCCCCCCCCCCC]

In some examples, the compound comprises:

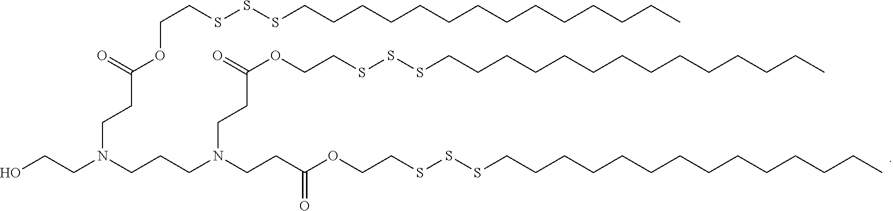 [SMILES: CCCCCCCCCCCCCCSSSCCOC(=O)CCN(CCO)CCCN(CCC(=O)OCCSSSCCCCCCCCCCCCCC)CCC(=O)OCCSSSCCCCCCCCCCCCCC]

### Lipid Nanoparticles

Also disclosed herein is a lipid nanoparticle (e.g., one or more nanoparticles) comprising any of the compounds disclosed herein.

In one aspect, the disclosure provides a nanoparticle comprising:
a compound of any one of Formulas I-V;
a non-cationic lipid;
a polyethylene glycol-lipid; and
a sterol.

In one aspect, the disclosure provides a nanoparticle comprising: a compound of Formula I; a non-cationic lipid; a polyethylene glycol-lipid; and a sterol.

In one aspect, the disclosure provides a nanoparticle comprising: a compound of Formula II; a non-cationic lipid; a polyethylene glycol-lipid; and a sterol.

In one aspect, the disclosure provides a nanoparticle comprising: a compound of Formula III; a non-cationic lipid; a polyethylene glycol-lipid; and a sterol.

In one aspect, the disclosure provides a nanoparticle comprising: a compound of Formula IV; a non-cationic lipid; a polyethylene glycol-lipid; and a sterol.

In one aspect, the disclosure provides a nanoparticle comprising: a compound of Formula V; a non-cationic lipid; a polyethylene glycol-lipid; and a sterol.

In one aspect, the disclosure provides a nanoparticle comprising: a compound of Formula

The various compounds of Formulas I-V are described in the Compounds section above.

In some embodiments, the nanoparticle comprises a compound of Formulas I-V in a molar ratio of about 10% to about 40%. In some embodiments, the nanoparticle comprises a compound of Formulas I-V in a molar ratio of about 10%, about 15%, about 20%, about 25%, about 30%, about 35%, or about 40%. In one embodiment, the nanoparticle comprises a compound of Formulas I-V in a molar ratio of about 20%. In some examples, a molar ratio of the compound is from 5% to 60%.

In some embodiments, the nanoparticle comprises a non-cationic lipid. In some embodiments, the non-cationic lipid interacts with the lipids as a helper lipid. In some embodiments, the non-cationic lipid can include, but is not limited to, 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In one embodiment, the non-cationic lipid is 1,2-dioleoyl-sn-glycero-3-phosphoethanolamine (DOPE). In one embodiment, the non-cationic lipid is 1-palmitoyl-2-oleoyl-sn-glycero-3-phosphoethanolamine (POPE), In one embodiment, the non-cationic lipid is 1,2-distearoyl-sn-glycero-3-phosphocholine (DSPC). In one embodiment, the non-cationic lipid is 1-stearoyl-2-oleoyl-sn-glycero-3-phosphoethanolamine (SOPE). While several non-cationic lipids are described here, additional non-cationic lipids can be used in combination with the compounds disclosed herein.

In some examples, a molar ratio of the non-cationic lipid is from 20% to 50%. In some embodiments, the nanoparticle comprises a non-cationic lipid in a molar ratio of about 10% to about 40%. In some embodiments, the nanoparticle comprises a non-cationic lipid in a molar ratio of about 10%, about 15%, about 20%, about 25%, about 30%, about 35%, or about 40%. In one embodiment, the nanoparticle comprises a non-cationic lipid in a molar ratio of about 30%.

In some embodiments, the nanoparticle includes a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines, PEG-modified diacylglycerols, and PEG-modified dialkylglycerols. Representative polyethylene glycol-lipids include DMG-PEG, DLPE-PEGs, DMPE-PEGs, DPPC-PEGs, and DSPE-PEGs. In one embodiment, the polyethylene glycol-lipid is 1,2-dimyristoyl-sn-glycerol, methoxypolyethylene glycol (DMG-PEG). In one embodiment, the polyethylene glycol-lipid is 1,2-dimyristoyl-sn-glycerol, methoxypolyethylene glycol-2000 (DMG-PEG2000). DMG-PEGXXXX means 1,2-dimyristoyl-sn-glycerol, methoxypolyethylene glycol-XXXX, wherein XXXX signifies the molecular weight of the polyethylene glycol moiety, e.g. DMG-PEG2000 or DMG-PEG5000.

In some embodiments, the nanoparticle comprises a polyethylene glycol-lipid in a molar ratio of about 0% to about 5%. In some examples, a molar ratio of the PEG-lipid is from 0.1% to 2%. In some embodiments, the nanoparticle comprises a polyethylene glycol-lipid in a molar ratio of about 0%, about 0.25%, about 0.5%, about 0.75%, about 1%, about 1.5%, about 2%, about 3%, about 4%, or about 5%. In one embodiment, the nanoparticle comprises a polyethylene glycol-lipid in a molar ratio of about 0.75%.

In some embodiments, the nanoparticle includes a sterol. Sterols are well known to those skilled in the art and generally refers to those compounds having a perhydrocyclopentanophenanthrene ring system and having one or more OH substituents. Examples of sterols include, but are not limited to, cholesterol, campesterol, ergosterol, sitosterol, and the like.

In some embodiments, the sterol is selected from a cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl)piperazine, or combinations thereof.

The sterol can be used to tune the particle permeability and fluidity base on its function in cell membranes. In one embodiment, the sterol is cholesterol.

In some embodiments, the nanoparticle comprises a sterol in a molar ratio of about 25% to about 50%. In some examples, a molar ratio of the sterol is from 20% to 50%. In some embodiments, the nanoparticle comprises a sterol in a molar ratio of about 25%, about 30%, about 35%, about 40%, about 45%, or about 50%. In one embodiment, the nanoparticle comprises a sterol in a molar ratio of about 40%.

In one embodiment, the disclosure provides a nanoparticle comprising:
a compound of Formulas I-V;
1,2-dioleoyl-sn-glycero-3-phosphoethanolamine (DOPE);
1,2-dimyristoyl-sn-glycerol, methoxypolyethylene glycol (DMG-PEG₂₀₀₀); and cholesterol.

In one embodiment, the disclosure provides a nanoparticle comprising:
a compound of Formulas I-V;
1-palmitoyl-2-oleoyl-sn-glycero-3-phosphoethanolamine (POPE);
1,2-dimyristoyl-sn-glycerol, methoxypolyethylene glycol (DMG-PEG₂₀₀₀); and cholesterol.

In one embodiment, the disclosure provides a nanoparticle comprising:
a compound of Formulas I-V;
1,2-distearoyl-sn-glycero-3-phosphocholine (DSPC);
1,2-dimyristoyl-sn-glycerol, methoxypolyethylene glycol (DMG-PEG₂₀₀₀); and cholesterol.

In some examples, a molar ratio of the compound is from 20% to 30%, a molar ratio of the non-cationic lipid is from 35% to 45%, a molar ratio of the sterol is from 35% to 45%, and a molar ratio of the polyethylene glycol-lipid is from 0.1% to 1%.

In one embodiment, the nanoparticle further comprises an agent. In one embodiment, the nanoparticle further comprises a therapeutic agent. In one embodiment, the nanoparticle further comprises a diagnostic agent.

The agents delivered into cells can be a polynucleotide. Polynucleotides or oligonucleotides that can be introduced according to the methods herein include DNA, cDNA, and RNA sequences of all types. For example, the polynucleotide can be double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), antisense RNA (asRNA) and combinations thereof. The polynucleotides can also be DNA constructs, such as expression vectors, expression vectors encoding a desired gene product (e.g., a gene product homologous or heterologous to the subject into which it is to be introduced), and the like. In one embodiment, the agent is an mRNA.

The nanoparticle can be of any shape. (e.g., a sphere, a rod, a quadrilateral, an ellipse, a triangle, a polygon, etc.). In some examples, the nanoparticle can have a regular shape, an irregular shape, an isotropic shape, an anisotropic shape, or a combination thereof. In some examples, the lipid particles are substantially spherical in shape.

The lipid particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The lipid particles can, for example, have an average particle size of 30 nanometers (nm) or more (e.g., 40 nm or more, 50 nm or more, 60 nm or more, 70 nm or more, 80 nm or more, 90 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 375 nm or more, 400 nm or more, 425 nm or more, 450 nm or more, 475 nm or more, 500 nm or more, 550 nm or more, 600 nm or more, 650 nm or more, 700 nm or more, or 750 nm or more). In some examples, the lipid particles can have an average particle size of 800 nm or less (e.g., 750 nm or less, 700 nm or less, 650 nm or less, 600 nm or less, 550 nm or less, 500 nm or less, 475 nm or less, 450 nm or less, 425 nm or less, 400 nm or less, 375 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 90 nm or less, 80 nm or less, 70 nm or less, 60 nm or less, 50 nm or less, or 40 nm or less). The average particle size of the lipid particles can range from any of the minimum values described above to any of the maximum values described above. For example, the lipid particles can have an average particle size of from 30 nm to 800 nm (e.g., from 30 nm to 425 nm, from 425 nm to 800 nm, from 30 nm to 200 nm, from 200 nm to 400 nm, from 400 nm to 600 nm, from 600 nm to 800 nm, from 50 nm to 800 nm, from 30 nm to 750 nm, or from 50 nm to 750 nm).

With respect to particle size distribution characterization, a parameter used to define the size range of the lipid particles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the lipid particles can have a polydispersity index of 0.5 or less (e.g., 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the lipid particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

### Compositions

Compositions, as described herein, comprising an active compound and an excipient of some sort may be useful in a variety of medical and non-medical applications. For example, pharmaceutical compositions comprising an active compound and an excipient may be useful in the delivery of an effective amount of an agent to a subject in need thereof. Nutraceutical compositions comprising an active compound and an excipient may be useful in the delivery of an effective amount of a nutraceutical, e.g., a dietary supplement, to a subject in need thereof. Cosmetic compositions comprising an active compound and an excipient may be formulated as a cream, ointment, balm, paste, film, or liquid, etc., and may be useful in the application of make-up, hair products, and materials useful for personal hygiene, etc. Compositions comprising an active compound and an excipient may be useful for non-medical applications, e.g., such as an emulsion or emulsifier, useful, for example, as a food component, for extinguishing fires, for disinfecting surfaces, for oil cleanup, etc.

In certain embodiments, the composition further comprises an agent, as described herein. For example, in certain embodiments, the agent is a small molecule, organometallic compound, nucleic acid, protein, peptide, polynucleotide, metal, targeting agent, an isotopically labeled chemical compound, drug, vaccine, immunological agent, or an agent useful in bioprocessing. In certain embodiments, the agent is a polynucleotide. In certain embodiments, the polynucleotide is DNA or RNA. In certain embodiments, the RNA is RNAi, dsRNA, siRNA, shRNA, miRNA, or antisense RNA. In certain embodiments, the polynucleotide and the one or more active compounds are not covalently attached. In some examples, a weight fraction of the agent is from 5% to 20%.

In one aspect, the disclosure provides a composition comprising:
a compound of Formulas I-V; and
an agent.

In one aspect, the disclosure provides a composition comprising:
a nanoparticle, comprising a compound of Formulas I-V; and
an agent.

In another aspect, disclosed herein is a composition comprising:
a nanoparticle, comprising a compound of Formulas I-V; and an agent,
wherein the agent comprises an mRNA encoding at least one antigenic polypeptide or an immunogenic fragment thereof capable of inducing an immune response to the antigenic polypeptide.

In some embodiments, the mRNA encoding at least one antigenic polypeptide or an immunogenic fragment thereof capable of inducing an immune response to the antigenic polypeptide is encapsulated by the nanoparticle.

In some aspects, disclosed herein is a pharmaceutical composition comprising a pharmaceutically acceptable carrier and a nanoparticle comprising an mRNA at least one antigenic polypeptide or an immunogenic fragment thereof capable of inducing an immune response to the antigenic polypeptide.

In some aspects, disclosed herein is a hydrogel matrix encapsulating the lipid nanoparticle and the agents described herein. The terms “hydrogel” and “hydrogel matrix” are used interchangeably and typically refer to cross-linked, water-insoluble, and water-containing materials. In the context of drug delivery systems, hydrogels can include biocompatible and non-toxic materials.

### Agents

Agents to be delivered by the compounds, compositions, and systems described herein may be therapeutic, diagnostic, or prophylactic agents. Any chemical compound to be administered to a subject may be delivered using the particles or nanoparticles described herein. The agent may be an organic molecule (e.g., a therapeutic agent, a drug), inorganic molecule, nucleic acid, protein, amino acid, peptide, polypeptide, polynucleotide, targeting agent, isotopically labeled organic or inorganic molecule, vaccine, immunological agent, etc.

In certain embodiments, the agents are organic molecules with pharmaceutical activity, e.g., a drug. In certain embodiments, the drug is an antibiotic, anti-viral agent, anesthetic, steroidal agent, anti-inflammatory agent, anti-neoplastic agent, anti-cancer agent, antigen, vaccine, antibody, decongestant, antihypertensive, sedative, birth control agent, progestational agent, anti-cholinergic, analgesic, anti-depressant, anti-psychotic, f3-adrenergic blocking agent, diuretic, cardiovascular active agent, vasoactive agent, non-steroidal anti-inflammatory agent, nutritional agent, etc.

In certain embodiments of the present disclosure, the agent to be delivered may be a mixture of agents.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Therapeutic and prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, cell extracts, and RNA encoding at least one antigenic polypeptide or an immunogenic fragment thereof (e.g., an immunogenic fragment capable of inducing an immune response to the antigenic polypeptide). Therapeutic and prophylactic agents may be combined with interleukins, interferon, cytokines, and adjuvants such as cholera toxin, alum, Freund's adjuvant, etc. Prophylactic agents include antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In some examples, the agent comprises an RNA. In some examples, the agent comprises an mRNA. In some examples, the agent comprises a polynucleotide encoding a macrophage polarizing factor, such as interleukin-4 (IL-4) or interleukin-13 (IL-13). In some examples, the agent comprises a polynucleotide encoding TNF-α, IL-1β, IL-6, IL-12, IL-23, CXCL1, CXCL3, CXCL5, CXCL8, CXCL9, CXCL10, CXCL11, CXCL13, CXCL16, CXCR3; CCL2, CCL3, CCL4, CCL5, CCL8, CCL11, CCL15, CCL19, CCL20, IL-10, TGF-β, IL-IRA, IL-4, IL-13, CCL1, CCL2, CCL5, CXCL10, CCL13, CCL14, CCL17, CCL18, CCL22, CCL23, CCL24, CCL26, CXCL16, CCR2, CCR3, CCR4, or nitric oxide. In some examples, the agent comprises a polynucleotide encoding interleukin-4. In some examples, the agent is encapsulated by the nanoparticle. In some aspects, the agent is a ribonucleic acid (RNA) (e.g., mRNA) polynucleotide having an open reading frame encoding at least one (e.g., at least 2, 3, 4 or 5) antigenic polypeptide or an immunogenic fragment thereof (e.g., an immunogenic fragment capable of inducing an immune response to the antigenic polypeptide). The sequences above are known in the art and can be found, for example, at ncbi.nlm.nih.gov.

In some embodiments, the nucleic acids disclosed herein comprise at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (ScamU), pseudouridine (Ψ), N¹-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me¹Ψ).

The structures of these modified nucleobases are shown below:

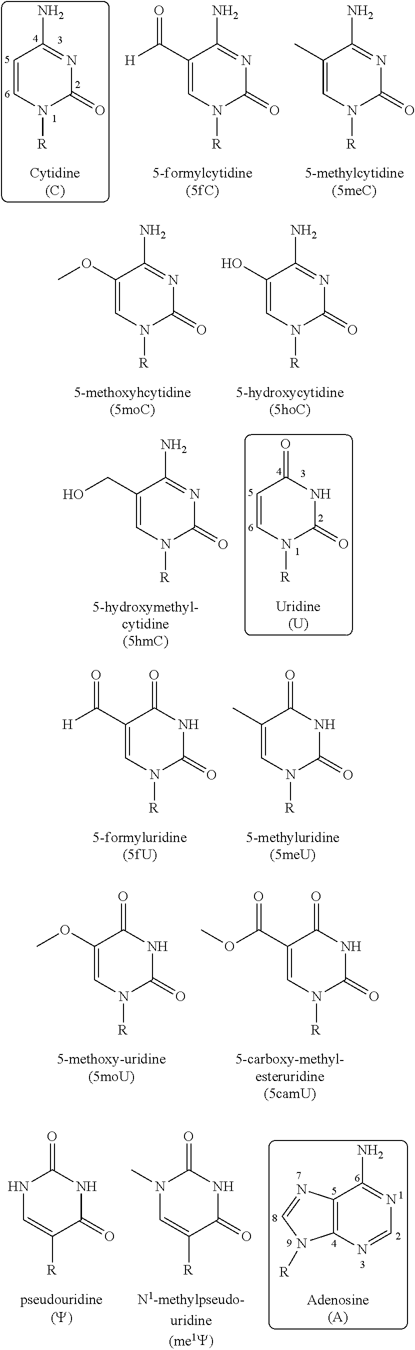

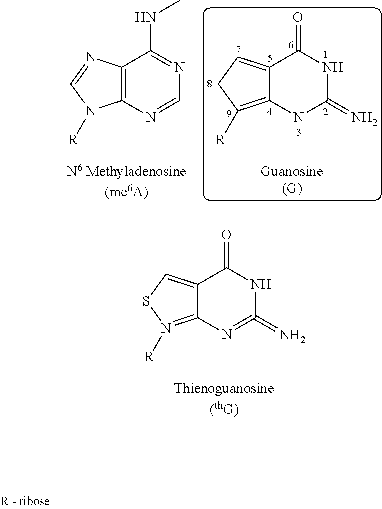

In one embodiment, the at least one chemically modified nucleotide is a chemically modified ribose.

In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-G-methyl (2′-G-Me). In one embodiment, the chemically modified ribose is 2′-Fluoro (2′-F).

The structures of these modified riboses are shown below:

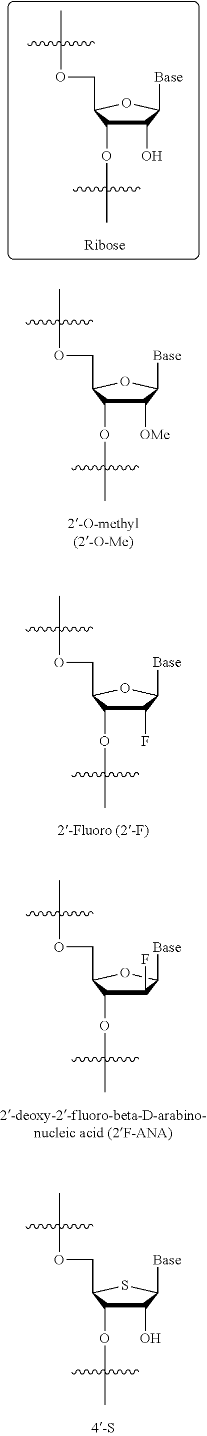

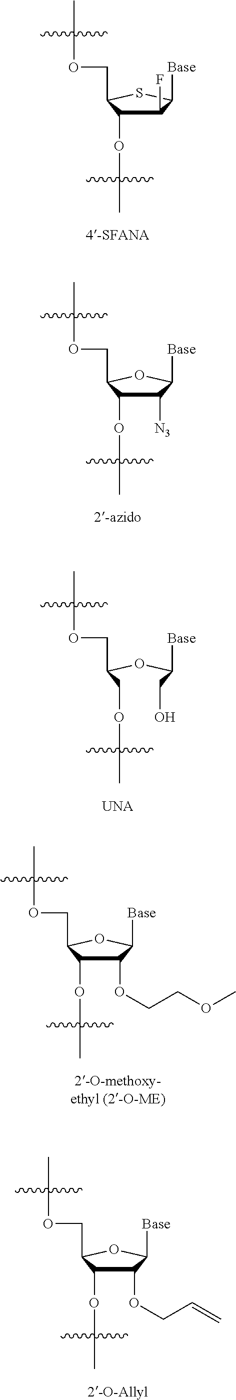

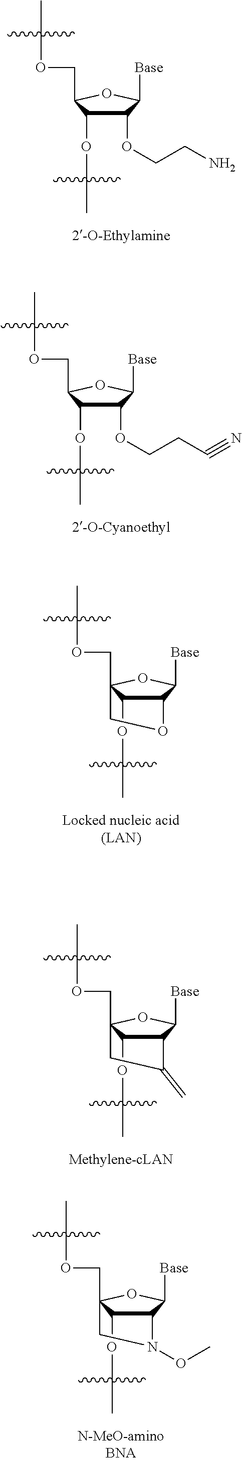

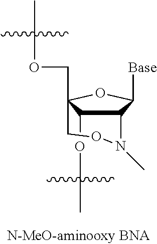

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

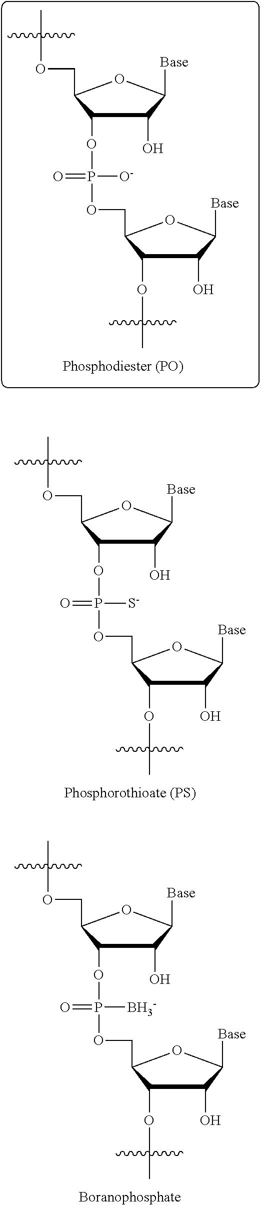

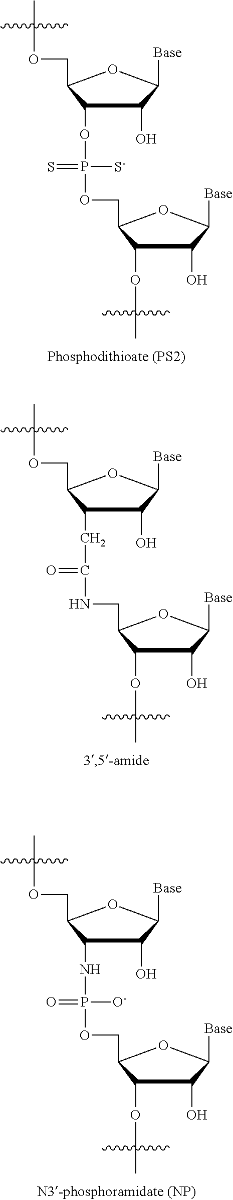

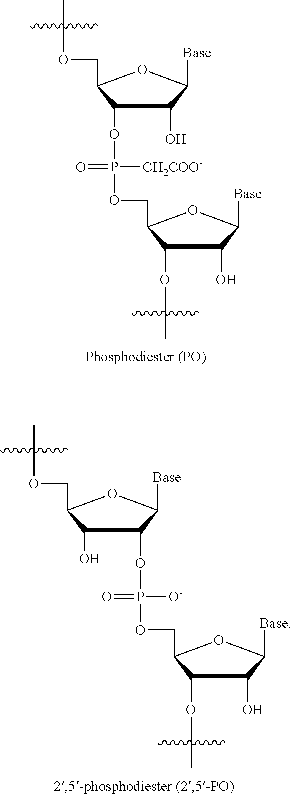

### Methods of Making

Also disclosed herein are methods of making any of the compounds or compositions disclosed herein. Also disclosed herein are methods of making any of the lipid particles disclosed herein. Also disclosed herein are methods of making any of the pharmaceutical compositions disclosed herein.

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

### Methods of Use

Also disclosed herein are methods of use of any of the compounds or compositions disclosed herein.

In one aspect, provided herein is a method for the delivery of an agent (for example, a polynucleotide) into a cell comprising;
introducing into the cell a composition comprising;
a nanoparticle, comprising;
   a compound of any one of Formulas I-V;
   a non-cationic lipid;
   a polyethylene glycol-lipid;
   a sterol; and
an agent.

In one aspect, disclosed herein is a method for the delivery of an agent into a cell comprising;
introducing into the cell a composition comprising;
a nanoparticle comprising;
a compound of Formula I, or a pharmaceutically acceptable salt thereof:

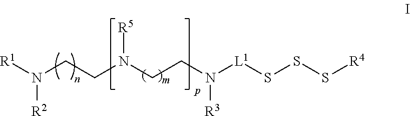

wherein
p is an integer from 0 to 5;
n is an integer from 1 to 10;
each m, when present, is independently an integer from 1 to 10;
R₁, R², and R³ are independently OH, a substituted or unsubstituted C₁-C₅ alkyl, substituted or unsubstituted C₁-C₅ alkyl alcohol, or -L¹-S—S—S—R^{a};
R⁴ is a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl; and
L¹ in each case is independently a substituted or unsubstituted alkyl, a carbonyl, an ester, an amide, a carbamate ester, an amine, an ether, a carbonate ester, a thioether, a thioester, or a urea.
a polyethylene glycol-lipid;
a sterol; and
an agent.

In some embodiments, a nanoparticle comprising any compound as described in the Compounds section above, is used in the methods herein, for delivery of an agent into a cell.

In some embodiments, the agent is a polynucleotide. In some embodiments, the agent is an RNA. In some embodiments, the agent is an mRNA. In some embodiments, the agent is a therapeutic agent, diagnostic agent, or prophylactic agent.

In some embodiments, provided herein are methods for the delivery of polynucleotides. In some embodiments, provided herein are methods for the delivery of polynucleotides (for example, mRNA) to correct a mutation in a genome. For example, mRNAs can be delivered to correct mutations that cause hemophilia (due to mutations in the genes encoding Factor VIII (F8; hemophilia A) or Factor IX (F9; hemoglobin B). In some embodiments, provided herein are methods for the delivery of polynucleotides. In some embodiments, provided herein are methods for the delivery of polynucleotides (for example, mRNA) to provide expression of the mRNA (and translation to produce a protein) in a cell. In some embodiments, provided herein are methods for the delivery of polynucleotides (for example, mRNA) to induce an immune response in a subject. In some embodiments, the RNA (e.g., mRNA) polynucleotide having an open reading frame encoding at least one (e.g., at least 2, 3, 4 or 5) hMPV, PIV, RSV, MeV, and/or a BetaCoV (e.g., MERS-CoV, SARS-CoV, SARS-CoV2, HCoV-OC43, HCoV-229E, HCoV-NL63, HCoV-NL, HCoV-NH, HCoV-HKU1) antigenic polypeptide, or any combination of two or more of the antigenic polypeptides.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the compositions and methods described herein are useful in treating or preventing a cancer. In some cases, the cancer is a circulating cancer cell (circulating tumor cell). In some cases, the cancer is a metastatic cancer cell.

In some aspects, the methods described herein are useful for promoting wound repair in a subject. For example, the administration of any of the compositions, nanoparticles, pharmaceutically acceptable compositions, or hydrogel matrices described herein can be used to promoting wound repair of a diabetic wound in the subject. Diabetic wounds can include acute diabetic wounds and chronic diabetic wounds. Acute diabetic wounds typically refer to wounds that undergoes normal healing over time. Chronic diabetic wounds do not generally follow the same progression of healing as acute diabetic wounds. For example, chronic diabetic wounds can include, but are not limited to, pressure ulcers, diabetic ulcers, venous ulcers, and arterial ulcers.

In some embodiments, the subject is a mammal. In some embodiments, the mammal is a human.

In some embodiments, the antibody or antigen binding fragment thereof and the nanoparticle are administered by intramuscularly injection or systematically.

In some embodiments, the method further comprises administering an additional therapeutic agent. In some embodiments, the additional therapeutic agent comprises an additional immunotherapeutic agent. In some embodiments, the immunotherapeutic agent is selected from an anti-CD40 antibody, an anti-PDL1 antibody, an anti-PD1 antibody, an anti-CTLA4 antibody, or a combination thereof.

In one embodiment, the immunotherapeutic agent is an anti-PDL1 antibody. In one embodiment, the anti-PDL1 antibody is selected from atezolizumab, durvalumab, or avelumab. In one embodiment, the anti-PDL1 antibody is atezolizumab (MPDL3280A)(Roche). In one embodiment, the anti-PDL1 antibody is durvalumab (MEDI4736). In one embodiment, the anti-PDL1 antibody is avelumab (MS0010718C).

In one embodiment, the immunotherapeutic agent is a programmed death protein 1 (PD-1) inhibitor or programmed death protein ligand 1 or 2 inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech).

In one embodiment, the immunotherapeutic agent is an anti-PD1 antibody. In one embodiment, the anti-PD1 antibody is nivolumab. In one embodiment, the anti-PD1 antibody is pembrolizumab.

In one embodiment, the immunotherapeutic agent is an anti-CTLA4 antibody. In one embodiment, the anti-CTLA4 antibody is ipilimumab.

In some embodiments, the additional therapeutic agent is an anti-neoplastic agent. For example, the anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeTP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

In one embodiment, provided herein is a method of treating an inflammation disorder, including autoimmune diseases in a subject. The method comprises administering to said subject a therapeutically effective amount of a compound, a combination of compounds, or a composition provided herein, or a pharmaceutically acceptable form thereof, or a pharmaceutical composition as provided herein. Examples of autoimmune diseases include but are not limited to acute disseminated encephalomyelitis (ADEM), Addison's disease, antiphospholipid antibody syndrome (APS), aplastic anemia, autoimmune hepatitis, autoimmune skin disease, coeliac disease, Crohn's disease, Diabetes mellitus (type 1), Goodpasture's syndrome, Graves' disease, Guillain-Barre syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis (also known as “giant cell arteritis”), warm autoimmune hemolytic anemia, Wegener's granulomatosis, alopecia universalis (e.g., inflammatory alopecia), Chagas disease, chronic fatigue syndrome, dysautonomia, endometriosis, hidradenitis suppurativa, interstitial cystitis, neuromyotonia, sarcoidosis, scleroderma, ulcerative colitis, vitiligo, and vulvodynia. Other disorders include bone-resorption disorders and thrombosis.

Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gout flare, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, polymyalgia rheumatic, reperfusion injury, regional enteritis, rheumatic fever, systemic lupus erythematosus, scleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds can also be useful in treating inflammation associated with trauma and non-inflammatory myalgia.

Immune disorders, such as auto-immune disorders include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), relapsing polychondritis (e.g., atrophic polychondritis and systemic polychondromalacia), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In some examples, the compound or composition can be administered to the subject in an amount of 1 microgram (μg) per kilogram (kg) of body weight of the subject per day (μg/kg/day) or more (e.g., 2 μg/kg/day or more, 3 μg/kg/day or more, 4 μg/kg/day or more, 5 μg/kg/day or more, 10 μg/kg/day or more, 15 μg/kg/day or more, 20 μg/kg/day or more, 25 μg/kg/day or more, 30 μg/kg/day or more, 35 μg/kg/day or more, 40 μg/kg/day or more, 45 μg/kg/day or more, 50 μg/kg/day or more, 60 μg/kg/day or more, 70 μg/kg/day or more, 80 μg/kg/day or more, 90 μg/kg/day or more, 100 μg/kg/day or more, 125 μg/kg/day or more, 150 μg/kg/day or more, 175 μg/kg/day or more, 200 μg/kg/day or more, 225 μg/kg/day or more, 250 μg/kg/day or more, 300 μg/kg/day or more, 350 μg/kg/day or more, 400 μg/kg/day or more, 450 μg/kg/day or more, 500 μg/kg/day or more, 600 μg/kg/day or more, 700 μg/kg/day or more, 800 μg/kg/day or more, 900 μg/kg/day or more, 1 milligram (mg)/kg/day or more, 2 mg/kg/day or more, 3 mg/kg/day or more, 4 mg/kg/day or more, 5 mg/kg/day or more, 6 mg/kg/day or more, 7 mg/kg/day or more, 8 mg/kg/day or more, or 9 mg/kg/day or more). In some examples, the compound or composition can be administered to the subject in an amount of 10 milligrams (mg) per kilogram (kg) of body weight of the subject per day (mg/kg/day) or less (e.g., 9 mg/kg/day or less, 8 mg/kg/day or less, 7 mg/kg/day or less, 6 mg/kg/day or less, 5 mg/kg/day or less, 4 mg/kg/day or less, 3 mg/kg/day or less, 2 mg/kg/day or less, 1 mg/kg/day or less, 900 μg/kg/day or less, 800 μg/kg/day or less, 700 μg/kg/day or less, 600 μg/kg/day or less, 500 μg/kg/day or less, 450 μg/kg/day or less, 400 μg/kg/day or less, 350 μg/kg/day or less, 300 μg/kg/day or less, 250 μg/kg/day or less, 225 μg/kg/day or less, 200 μg/kg/day or less, 175 μg/kg/day or less, 150 μg/kg/day or less, 125 μg/kg/day or less, 100 μg/kg/day or less, 90 μg/kg/day or less, 80 μg/kg/day or less, 70 μg/kg/day or less, 60 μg/kg/day or less, 50 μg/kg/day or less, 45 μg/kg/day or less, 40 μg/kg/day or less, 35 μg/kg/day or less, 30 μg/kg/day or less, 25 μg/kg/day or less, 20 μg/kg/day or less, 15 μg/kg/day or less, 10 μg/kg/day or less, 5 μg/kg/day or less, 4 μg/kg/day or less, 3 μg/kg/day or less, or 2 μg/kg/day or less).

The amount of the compound or composition administered to the subject can range from any of the minimum values described above to any of the maximum values described above. For example, the compound or composition can be administered to the subject in an amount of from 1 microgram (μg) per kilogram (kg) of body weight of the subject per day to 10 milligrams (mg)/kg/day (e.g., from 1 μg/kg/day to 100 μg/kg/day, from 100 μg/kg/day to 10 mg/kg/day, from 1 μg/kg/day to 10 μg/kg/day, from 10 μg/kg/day to 100 μg/kg/day, from 100 μg/kg/day to 1 mg/kg/day, from 1 mg/kg/day to 10 mg/kg/day, from 5 μg/kg/day to 10 mg/kg/day, from 1 μg/kg/day to 5 mg/kg/day, or from 5 to 5 mg/kg/day).

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

### Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1. Lipid Derivatives

Efficient delivery of mRNA is a key step and challenge for the applicant of mRNA therapeutics. Despite promising data from ongoing clinical trials, the clinical use of mRNA requires the discovery and development of more efficient delivery systems.

Described herein are lipid derivatives for vaccine, gene therapy, and drug delivery applications. Synthetic routes and characterizations are shown below.

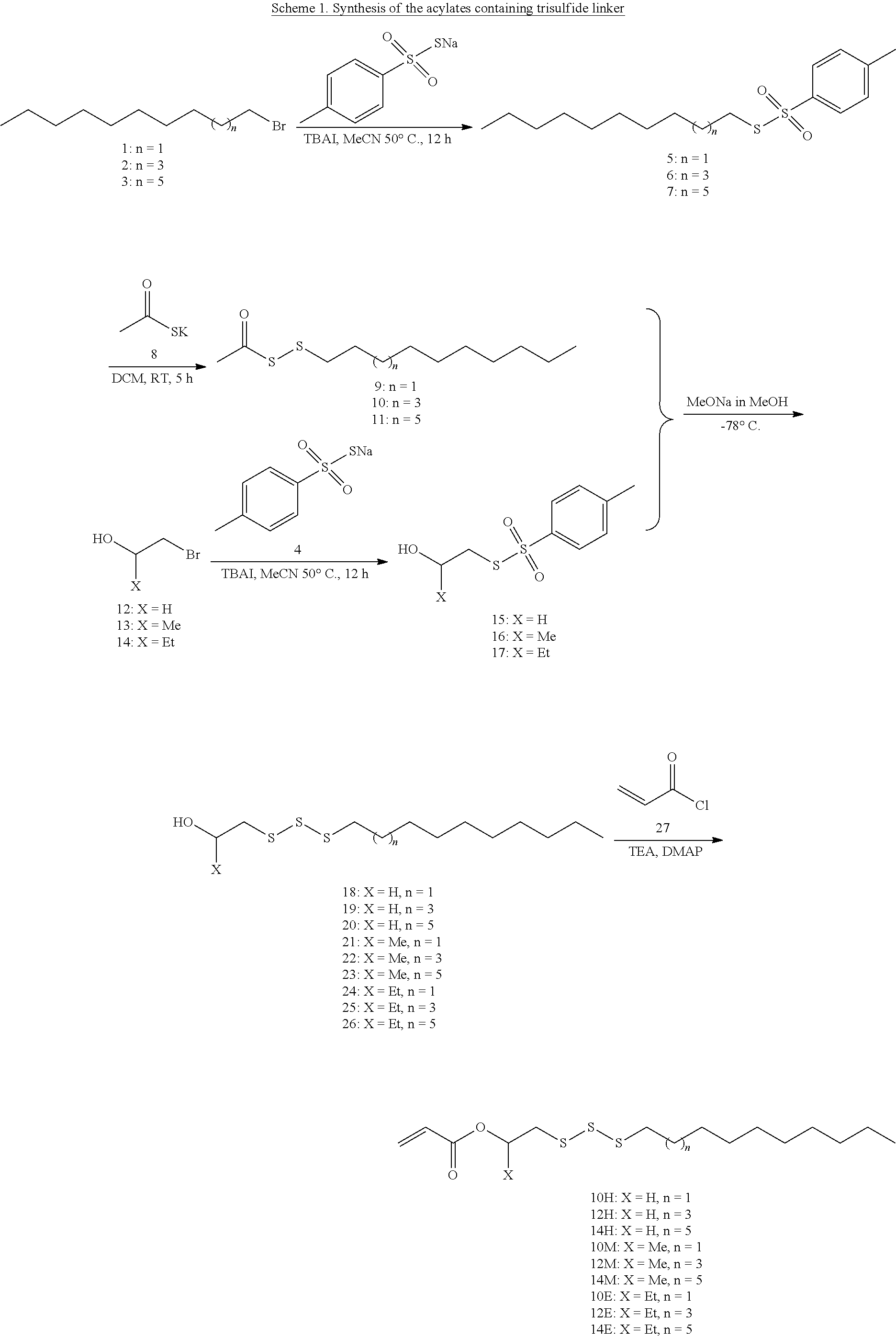

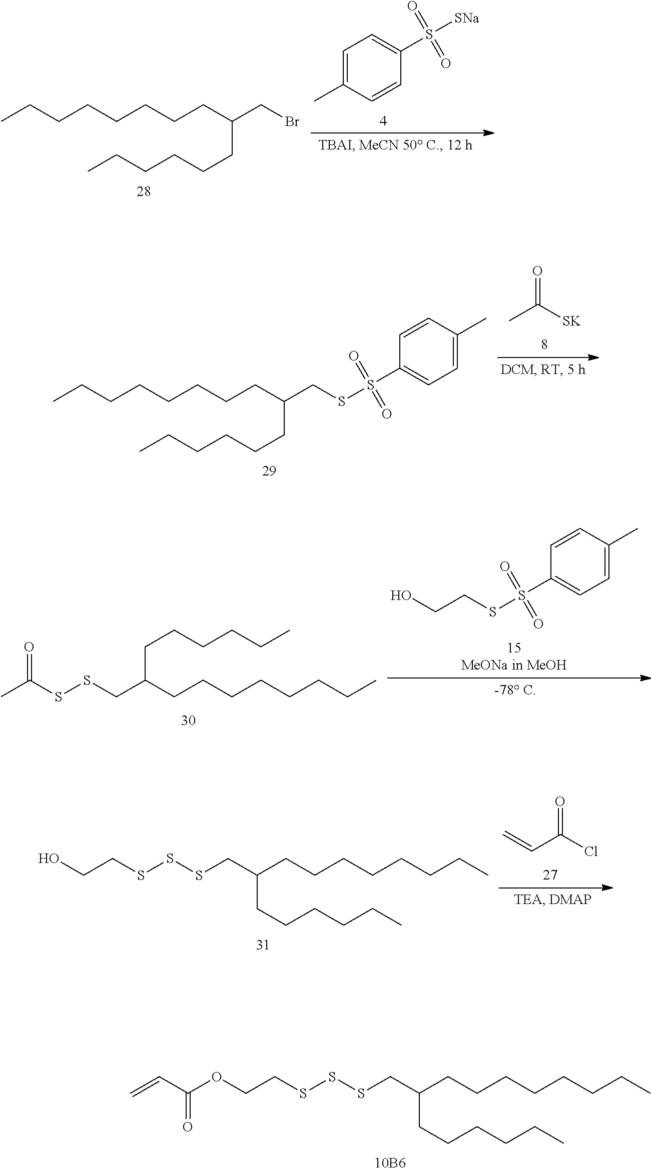

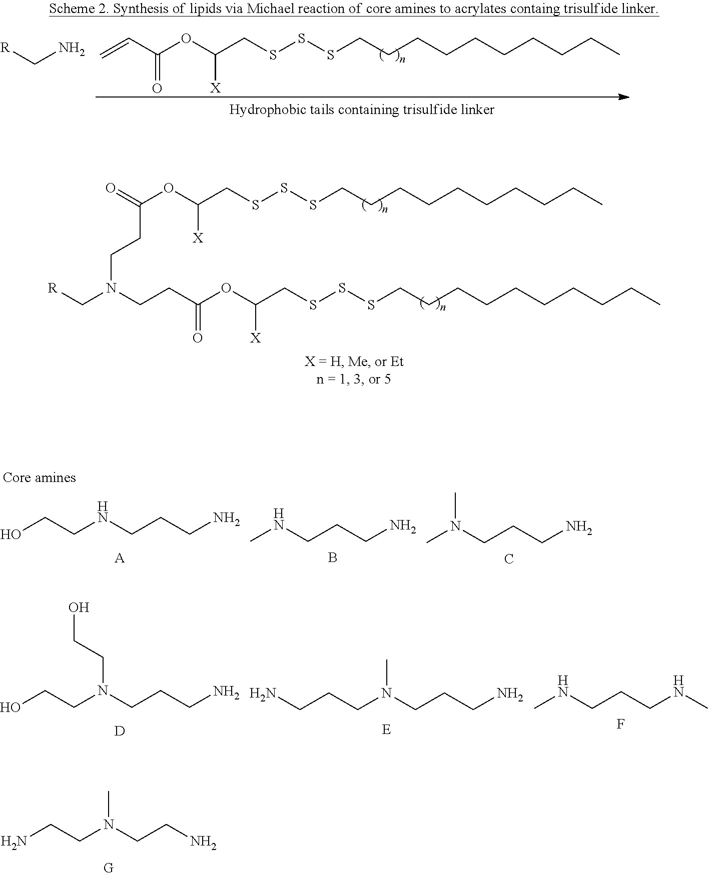

TABLE 1
Orthogonal Michael addition of core amines to alkyl acrylate

| | Core | | | | | | |
|---|---|---|---|---|---|---|---|
| | Amine | Amine | Amine | Amine | Amine | Amine | Amine |
| Tail | A | B | C | D | E | F | G |
| 10H | A-10H | B-10H | C-10H | D-10H | E-10H | F-10H | G-10H |
| 12H | A-12H | B-12H | C-12H | D-12H | E-12H | F-12H | G-12H |
| 14H | A-14H | B-14H | C-14H | D-14H | E-14H | F-14H | G-14H |
| 10B6 | A-10B6 | B-10B6 | C-10B6 | D-10B6 | E-10B6 | F-10B6 | G-10B6 |
| 10M | A-10M | B-10M | C-10M | D-10M | E-10M | F-10M | G-10M |
| 12M | A-12M | B-12M | C-12M | D-12M | E-12M | F-12M | G-12M |
| 14M | A-14M | B-14M | C-14M | D-14M | E-14M | F-14M | G-14M |
| 10E | A-10E | B-10E | C-10E | D-10E | E-10E | F-10E | G-10E |
| 12E | A-12E | B-12E | C-12E | D-12E | E-12E | F-12E | G-12E |
| 14E | A-14E | B-14E | C-14E | D-14E | E-14E | F-14E | G-14E |

TABLE 2
Alternative Names for the Compounds in Table 1

| | Amines | | | | | | |
|---|---|---|---|---|---|---|---|
| | Amine | Amine | Amine | Amine | Amine | Amine | Amine |
| Tails | A | B | C | D | E | F | G |
| 10H | TS1 | TS11 | TS21 | TS31 | TS41 | TS51 | TS61 |
| 12H | TS2 | TS12 | TS22 | TS32 | TS42 | TS52 | TS62 |
| 14H | TS3 | TS13 | TS23 | TS33 | TS43 | TS53 | TS63 |
| 10B6 | TS4 | TS14 | TS24 | TS34 | TS44 | TS54 | TS64 |
| 10M | TS5 | TS15 | TS25 | TS35 | TS45 | TS55 | TS65 |
| 12M | TS6 | TS16 | TS26 | TS36 | TS46 | TS56 | TS66 |
| 14M | TS7 | TS17 | TS27 | TS37 | TS47 | TS57 | TS67 |
| 10E | TS8 | TS18 | TS28 | TS38 | TS48 | TS58 | TS68 |
| 12E | TS9 | TS19 | TS29 | TS39 | TS49 | TS59 | TS69 |
| 14E | TS10 | TS20 | TS30 | TS40 | TS50 | TS60 | TS70 |

TABLE 3
List of trisulfide lipids

| Compound | |
|---|---|
| Code | Chemical Structure |
| A-10H | 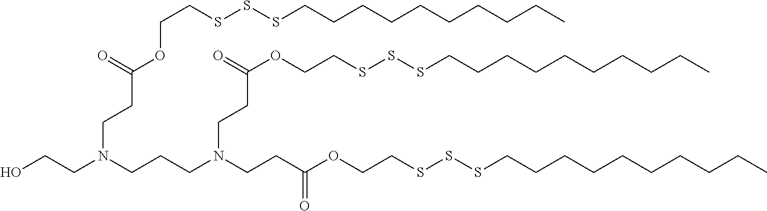 [SMILES: CCCCCCCCCCSSSCCOC(=O)CCN(CCO)CCCN(CCC(=O)OCCSSSCCCCCCCCCC)CCC(=O)OCCSSSCCCCCCCCCC] |
| A-12H | 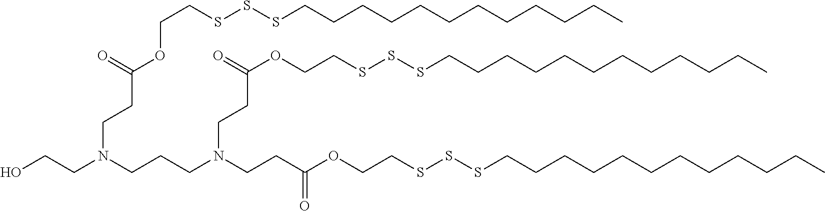 [SMILES: CCCCCCCCCCCCSSSCCOC(=O)CCN(CCO)CCCN(CCC(=O)OCCSSSCCCCCCCCCCCC)CCC(=O)OCCSSSCCCCCCCCCCCC] |
| A-14H | 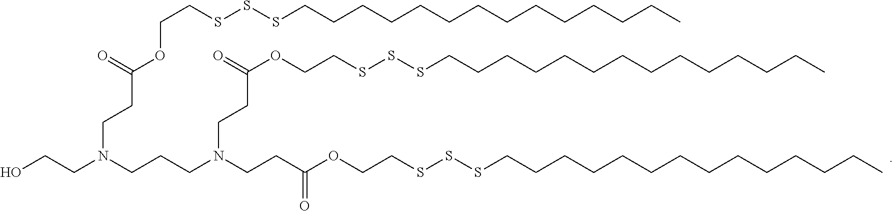 [SMILES: CCCCCCCCCCCCCCSSSCCOC(=O)CCN(CCO)CCCN(CCC(=O)OCCSSSCCCCCCCCCCCCCC)CCC(=O)OCCSSSCCCCCCCCCCCCCC] |
| A-10B6 | 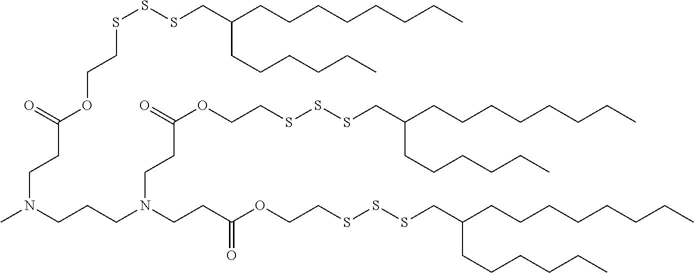 [SMILES: CCCCCCCCC(CCCCCC)CSSSCCOC(=O)CCN(C)CCCN(CCC(=O)OCCSSSCC(CCCCCC)CCCCCCCC)CCC(=O)OCCSSSCC(CCCCCC)CCCCCCCC] |
| A-10M | 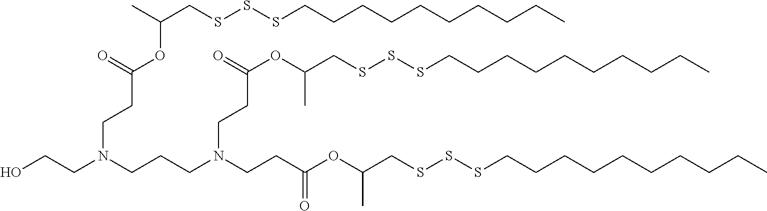 [SMILES: CCCCCCCCCCSSSCC(C)OC(=O)CCN(CCO)CCCN(CCC(=O)OC(C)CSSSCCCCCCCCCC)CCC(=O)OC(C)CSSSCCCCCCCCCC] |
| A-12M | 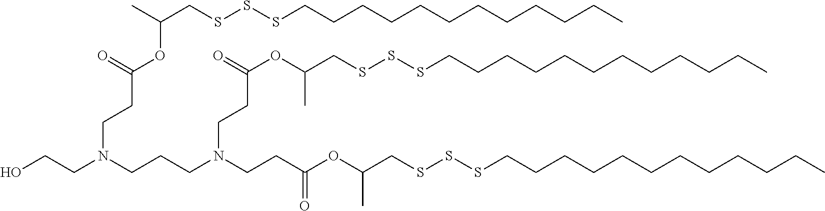 [SMILES: CCCCCCCCCCCCSSSCC(C)OC(=O)CCN(CCO)CCCN(CCC(=O)OC(C)CSSSCCCCCCCCCCCC)CCC(=O)OC(C)CSSSCCCCCCCCCCCC] |
| A-14M | 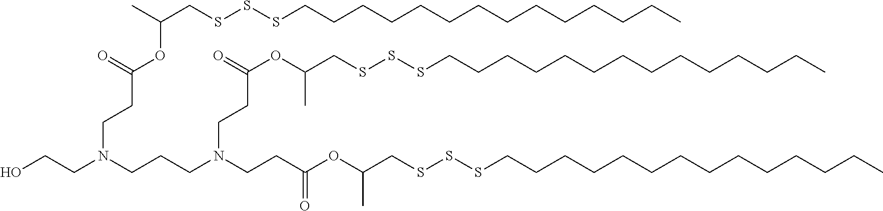 [SMILES: CCCCCCCCCCCCCCSSSCC(C)OC(=O)CCN(CCO)CCCN(CCC(=O)OC(C)CSSSCCCCCCCCCCCCCC)CCC(=O)OC(C)CSSSCCCCCCCCCCCCCC] |
| A-10E | 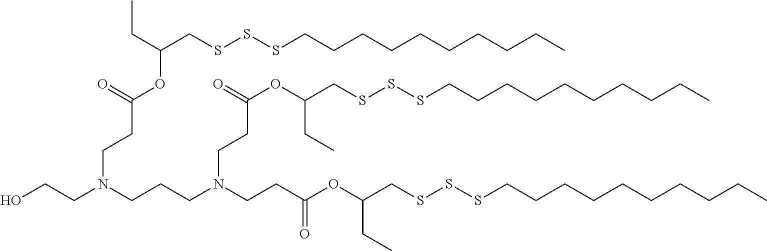 [SMILES: CCCCCCCCCCSSSCC(CC)OC(=O)CCN(CCO)CCCN(CCC(=O)OC(CC)CSSSCCCCCCCCCC)CCC(=O)OC(CC)CSSSCCCCCCCCCC] |
| A-12E | 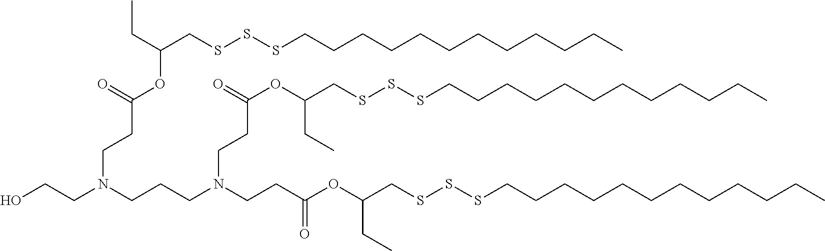 [SMILES: CCCCCCCCCCCCSSSCC(CC)OC(=O)CCN(CCO)CCCN(CCC(=O)OC(CC)CSSSCCCCCCCCCCCC)CCC(=O)OC(CC)CSSSCCCCCCCCCCCC] |
| A-14E | 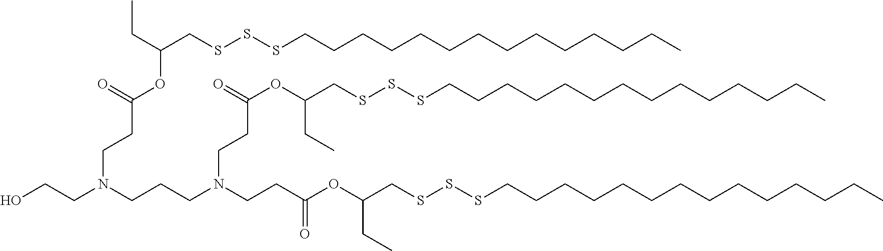 [SMILES: CCCCCCCCCCCCCCSSSCC(CC)OC(=O)CCN(CCO)CCCN(CCC(=O)OC(CC)CSSSCCCCCCCCCCCCCC)CCC(=O)OC(CC)CSSSCCCCCCCCCCCCCC] |
| B-10H | 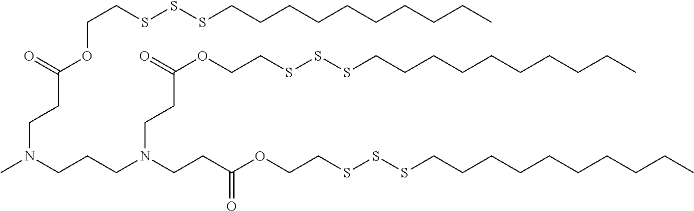 [SMILES: CCCCCCCCCCSSSCCOC(=O)CCN(C)CCCN(CCC(=O)OCCSSSCCCCCCCCCC)CCC(=O)OCCSSSCCCCCCCCCC] |
| B-12H | 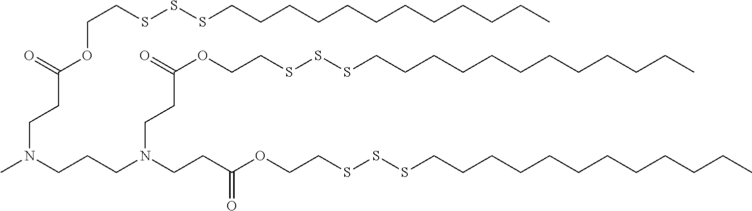 [SMILES: CCCCCCCCCCCCSSSCCOC(=O)CCN(C)CCCN(CCC(=O)OCCSSSCCCCCCCCCCCC)CCC(=O)OCCSSSCCCCCCCCCCCC] |
| B-14H | 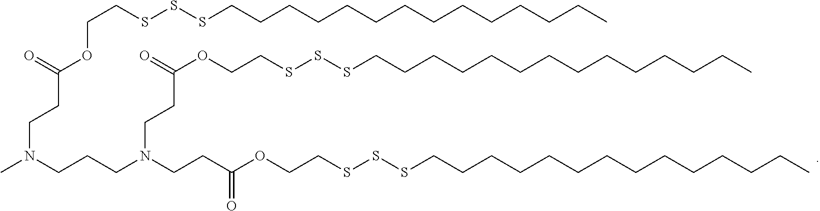 [SMILES: CCCCCCCCCCCCCCSSSCCOC(=O)CCN(C)CCCN(CCC(=O)OCCSSSCCCCCCCCCCCCCC)CCC(=O)OCCSSSCCCCCCCCCCCCCC] |
| B-10B6 | 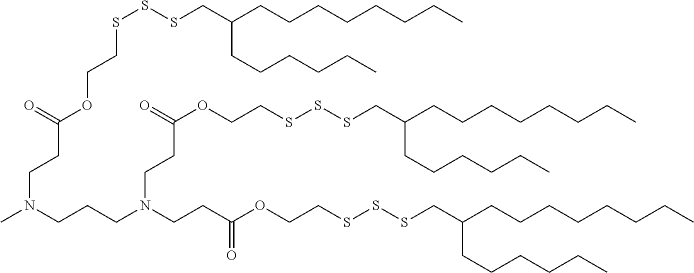 [SMILES: CCCCCCCCC(CCCCCC)CSSSCCOC(=O)CCN(C)CCCN(CCC(=O)OCCSSSCC(CCCCCC)CCCCCCCC)CCC(=O)OCCSSSCC(CCCCCC)CCCCCCCC] |
| B-10M | 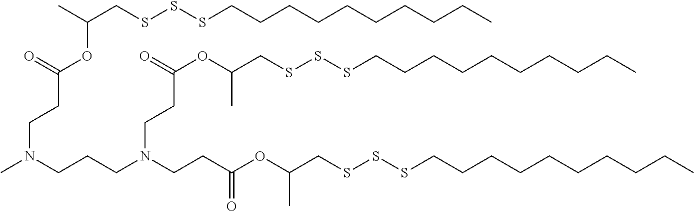 [SMILES: CCCCCCCCCCSSSCC(C)OC(=O)CCN(C)CCCN(CCC(=O)OC(C)CSSSCCCCCCCCCC)CCC(=O)OC(C)CSSSCCCCCCCCCC] |
| B-12M | 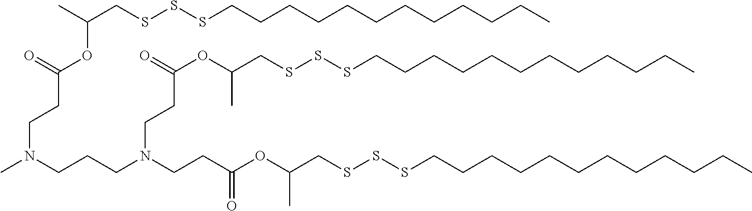 [SMILES: CCCCCCCCCCCCSSSCC(C)OC(=O)CCN(C)CCCN(CCC(=O)OC(C)CSSSCCCCCCCCCCCC)CCC(=O)OC(C)CSSSCCCCCCCCCCCC] |
| B-14M | 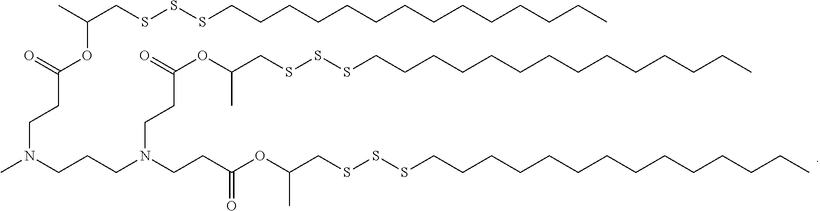 [SMILES: CCCCCCCCCCCCCCSSSCC(C)OC(=O)CCN(C)CCCN(CCC(=O)OC(C)CSSSCCCCCCCCCCCCCC)CCC(=O)OC(C)CSSSCCCCCCCCCCCCCC] |
| B-10E | 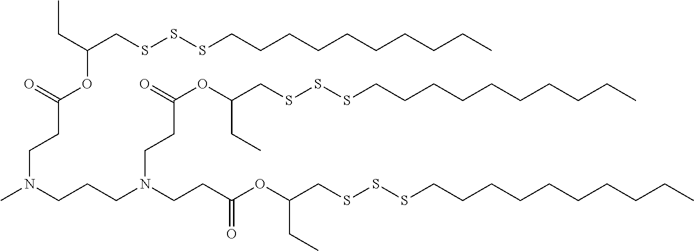 [SMILES: CCCCCCCCCCSSSCC(CC)OC(=O)CCN(C)CCCN(CCC(=O)OC(CC)CSSSCCCCCCCCCC)CCC(=O)OC(CC)CSSSCCCCCCCCCC] |
| B-12E | 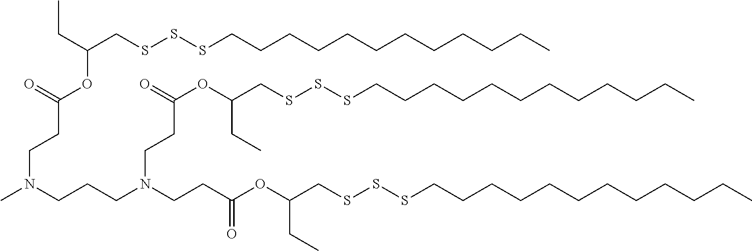 [SMILES: CCCCCCCCCCCCSSSCC(CC)OC(=O)CCN(C)CCCN(CCC(=O)OC(CC)CSSSCCCCCCCCCCCC)CCC(=O)OC(CC)CSSSCCCCCCCCCCCC] |
| B-14E | 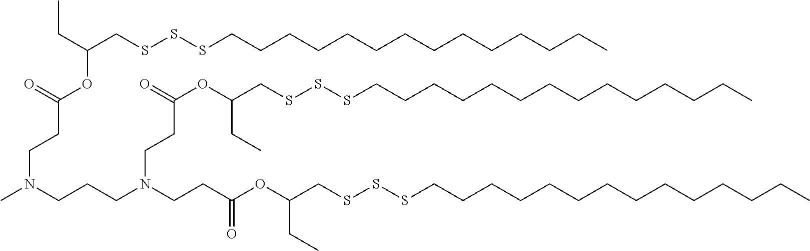 [SMILES: CCCCCCCCCCCCCCSSSCC(CC)OC(=O)CCN(C)CCCN(CCC(=O)OC(CC)CSSSCCCCCCCCCCCCCC)CCC(=O)OC(CC)CSSSCCCCCCCCCCCCCC] |
| C-10H | 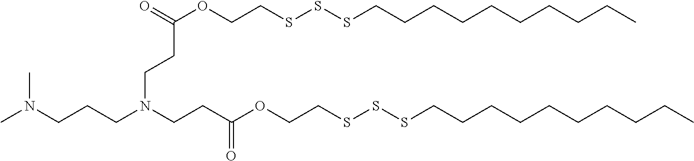 [SMILES: CCCCCCCCCCSSSCCOC(=O)CCN(CCCN(C)C)CCC(=O)OCCSSSCCCCCCCCCC] |
| C-12H | 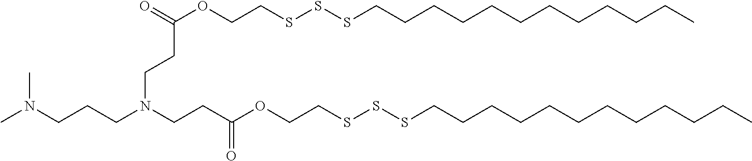 [SMILES: CCCCCCCCCCCCSSSCCOC(=O)CCN(CCCN(C)C)CCC(=O)OCCSSSCCCCCCCCCCCC] |
| C-14H | 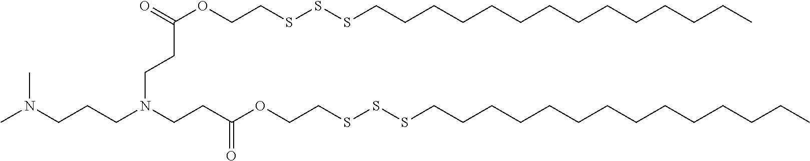 [SMILES: CCCCCCCCCCCCCCSSSCCOC(=O)CCN(CCCN(C)C)CCC(=O)OCCSSSCCCCCCCCCCCCCC] |
| C-10B6 | 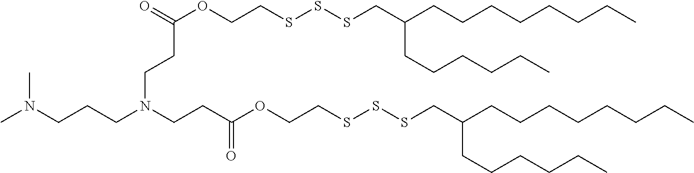 [SMILES: CCCCCCCCC(CCCCCC)CSSSCCOC(=O)CCN(CCCN(C)C)CCC(=O)OCCSSSCC(CCCCCC)CCCCCCCC] |
| C-10M | 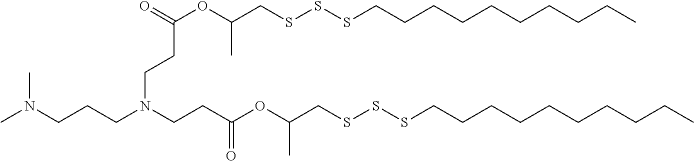 [SMILES: CCCCCCCCCCSSSCC(C)OC(=O)CCN(CCCN(C)C)CCC(=O)OC(C)CSSSCCCCCCCCCC] |
| C-12M | 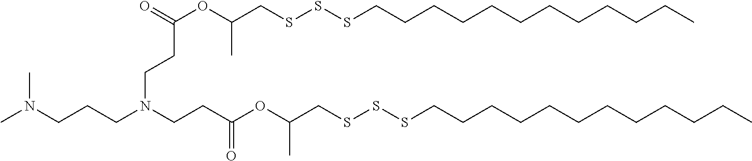 [SMILES: CCCCCCCCCCCCSSSCC(C)OC(=O)CCN(CCCN(C)C)CCC(=O)OC(C)CSSSCCCCCCCCCCCC] |
| C-14M | 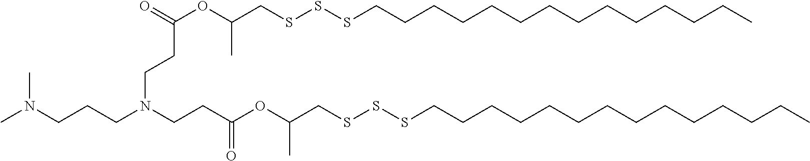 [SMILES: CCCCCCCCCCCCCCSSSCC(C)OC(=O)CCN(CCCN(C)C)CCC(=O)OC(C)CSSSCCCCCCCCCCCCCC] |
| C-10E | 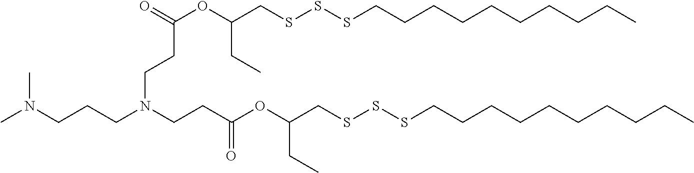 [SMILES: CCCCCCCCCCSSSCC(CC)OC(=O)CCN(CCCN(C)C)CCC(=O)OC(CC)CSSSCCCCCCCCCC] |
| C-12E | 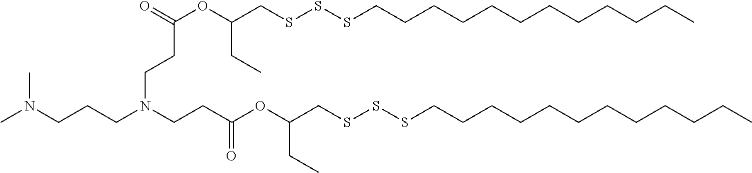 [SMILES: CCCCCCCCCCCCSSSCC(CC)OC(=O)CCN(CCCN(C)C)CCC(=O)OC(CC)CSSSCCCCCCCCCCCC] |
| C-14E | 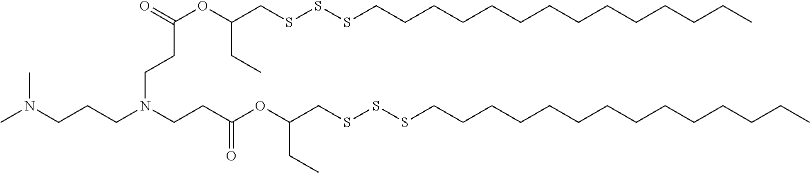 [SMILES: CCCCCCCCCCCCCCSSSCC(CC)OC(=O)CCN(CCCN(C)C)CCC(=O)OC(CC)CSSSCCCCCCCCCCCCCC] |
| D-10H | 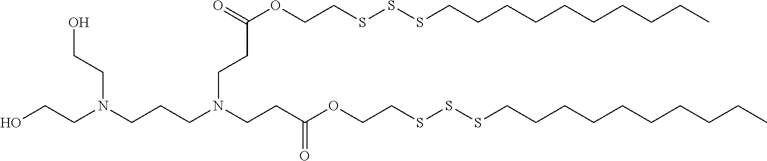 [SMILES: CCCCCCCCCCSSSCCOC(=O)CCN(CCCN(CCO)CCO)CCC(=O)OCCSSSCCCCCCCCCC] |
| D-12H | 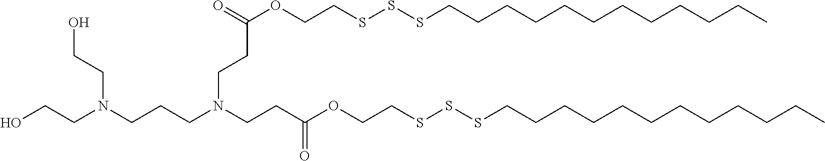 [SMILES: CCCCCCCCCCCCSSSCCOC(=O)CCN(CCCN(CCO)CCO)CCC(=O)OCCSSSCCCCCCCCCCCC] |
| D-14H | 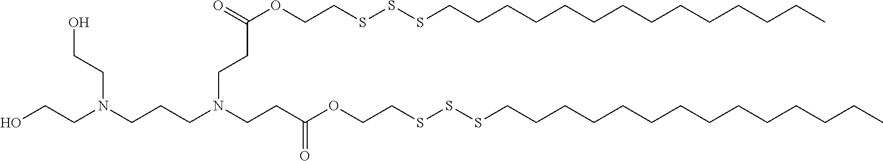 [SMILES: CCCCCCCCCCCCCCSSSCCOC(=O)CCN(CCCN(CCO)CCO)CCC(=O)OCCSSSCCCCCCCCCCCCCC] |
| D-10B6 | 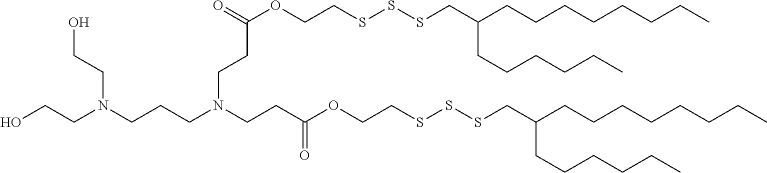 [SMILES: CCCCCCCCC(CCCCCC)CSSSCCOC(=O)CCN(CCCN(CCO)CCO)CCC(=O)OCCSSSCC(CCCCCC)CCCCCCCC] |
| D-10M | 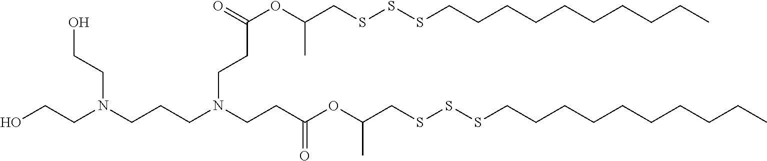 [SMILES: CCCCCCCCCCSSSCC(C)OC(=O)CCN(CCCN(CCO)CCO)CCC(=O)OC(C)CSSSCCCCCCCCCC] |
| D-12M | 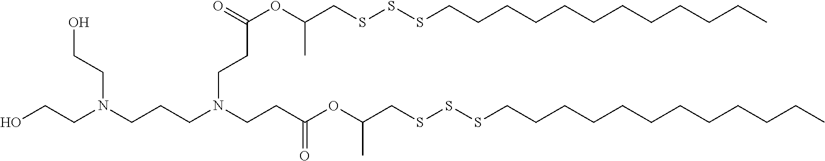 [SMILES: CCCCCCCCCCCCSSSCC(C)OC(=O)CCN(CCCN(CCO)CCO)CCC(=O)OC(C)CSSSCCCCCCCCCCCC] |
| D-14M | 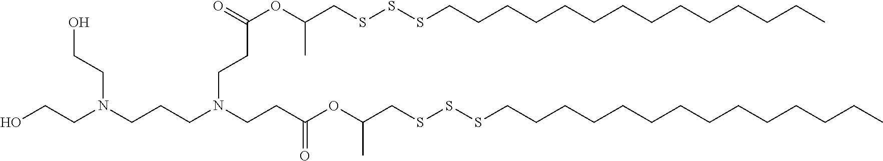 [SMILES: CCCCCCCCCCCCCCSSSCC(C)OC(=O)CCN(CCCN(CCO)CCO)CCC(=O)OC(C)CSSSCCCCCCCCCCCCCC] |
| D-10E | 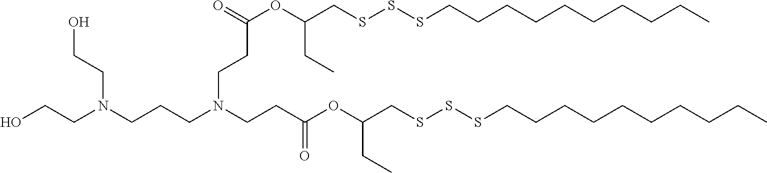 [SMILES: CCCCCCCCCCSSSCC(CC)OC(=O)CCN(CCCN(CCO)CCO)CCC(=O)OC(CC)CSSSCCCCCCCCCC] |
| D-12E | 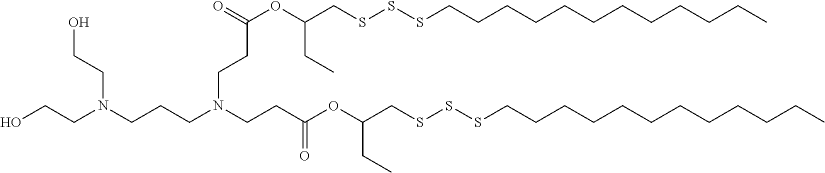 [SMILES: CCCCCCCCCCCCSSSCC(CC)OC(=O)CCN(CCCN(CCO)CCO)CCC(=O)OC(CC)CSSSCCCCCCCCCCCC] |
| D-14E | 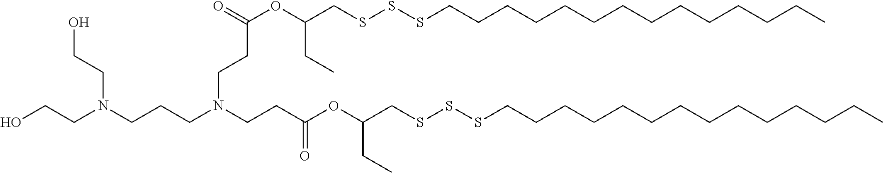 [SMILES: CCCCCCCCCCCCCCSSSCC(CC)OC(=O)CCN(CCCN(CCO)CCO)CCC(=O)OC(CC)CSSSCCCCCCCCCCCCCC] |
| E-10H | 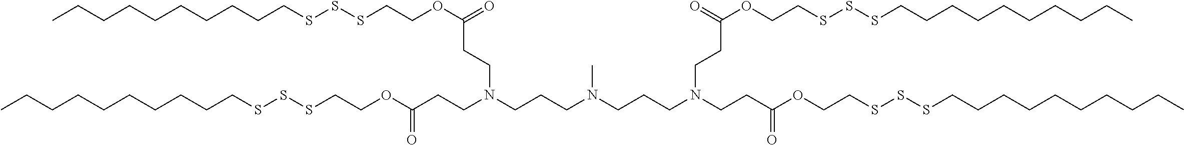 [SMILES: CCCCCCCCCCSSSCCOC(=O)CCN(CCCN(C)CCCN(CCC(=O)OCCSSSCCCCCCCCCC)CCC(=O)OCCSSSCCCCCCCCCC)CCC(=O)OCCSSSCCCCCCCCCC] |
| E-12H | 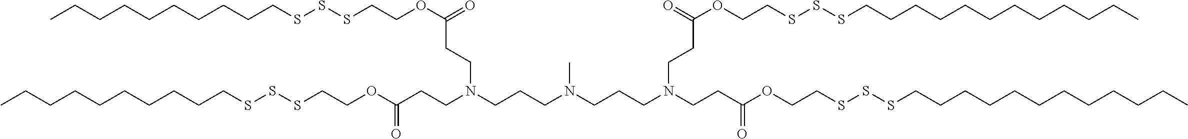 [SMILES: CCCCCCCCCCCCSSSCCOC(=O)CCN(CCCN(C)CCCN(CCC(=O)OCCSSSCCCCCCCCCC)CCC(=O)OCCSSSCCCCCCCCCC)CCC(=O)OCCSSSCCCCCCCCCCCC] |
| E-14H | 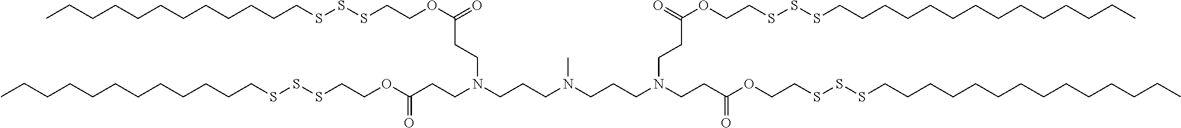 [SMILES: CCCCCCCCCCCCCCSSSCCOC(=O)CCN(CCCN(C)CCCN(CCC(=O)OCCSSSCCCCCCCCCCCC)CCC(=O)OCCSSSCCCCCCCCCCCC)CCC(=O)OCCSSSCCCCCCCCCCCCCC] |
| E-10B6 | 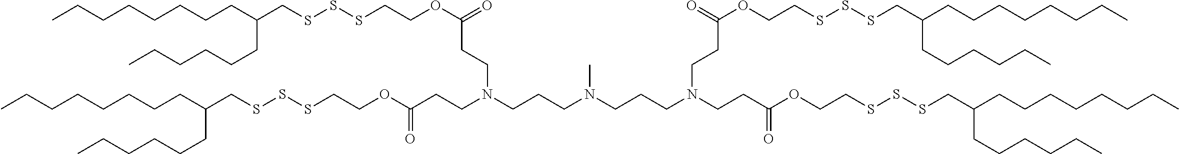 [SMILES: CCCCCCCCC(CCCCCC)CSSSCCOC(=O)CCN(CCCN(C)CCCN(CCC(=O)OCCSSSCC(CCCCCC)CCCCCCCC)CCC(=O)OCCSSSCC(CCCCCC)CCCCCCCC)CCC(=O)OCCSSSCC(CCCCCC)CCCCCCCC] |
| E-10M | 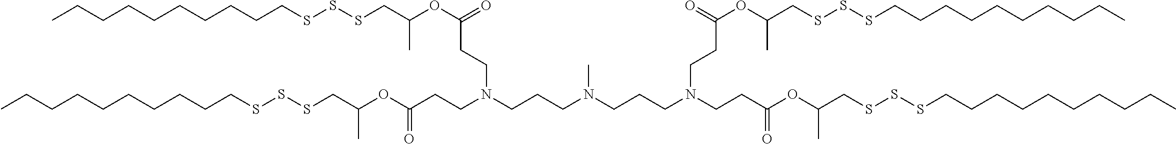 [SMILES: CCCCCCCCCCSSSCC(C)OC(=O)CCN(CCCN(C)CCCN(CCC(=O)OC(C)CSSSCCCCCCCCCC)CCC(=O)OC(C)CSSSCCCCCCCCCC)CCC(=O)OC(C)CSSSCCCCCCCCCC] |
| E-12M | 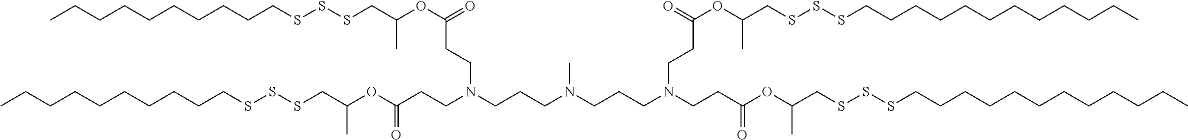 [SMILES: CCCCCCCCCCCCSSSCC(C)OC(=O)CCN(CCCN(C)CCCN(CCC(=O)OC(C)CSSSCCCCCCCCCC)CCC(=O)OC(C)CSSSCCCCCCCCCC)CCC(=O)OC(C)CSSSCCCCCCCCCCCC] |
| E-14M | 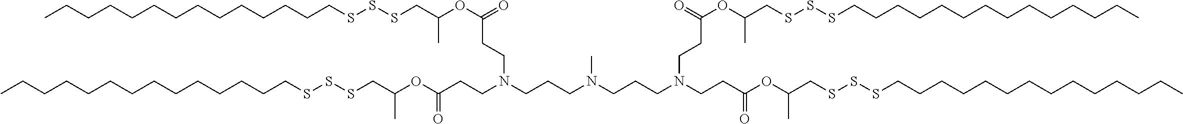 [SMILES: CCCCCCCCCCCCCCSSSCC(C)OC(=O)CCN(CCCN(C)CCCN(CCC(=O)OC(C)CSSSCCCCCCCCCCCCCC)CCC(=O)OC(C)CSSSCCCCCCCCCCCCCC)CCC(=O)OC(C)CSSSCCCCCCCCCCCCCC] |
| E-10E | 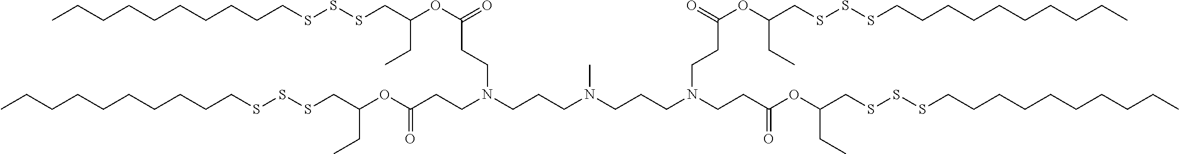 [SMILES: CCCCCCCCCCSSSCC(CC)OC(=O)CCN(CCCN(C)CCCN(CCC(=O)OC(CC)CSSSCCCCCCCCCC)CCC(=O)OC(CC)CSSSCCCCCCCCCC)CCC(=O)OC(CC)CSSSCCCCCCCCCC] |
| E-12E | 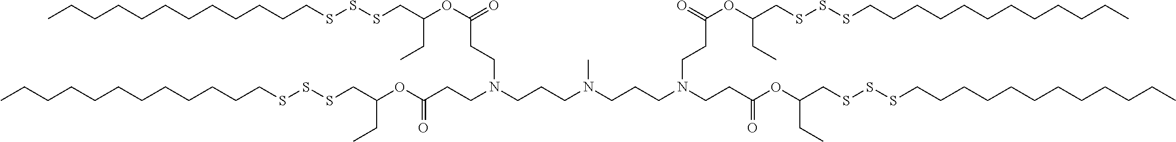 [SMILES: CCCCCCCCCCCCSSSCC(CC)OC(=O)CCN(CCCN(C)CCCN(CCC(=O)OC(CC)CSSSCCCCCCCCCCCC)CCC(=O)OC(CC)CSSSCCCCCCCCCCCC)CCC(=O)OC(CC)CSSSCCCCCCCCCCCC] |
| E-14E | 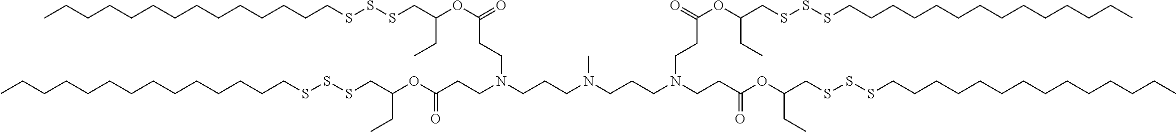 [SMILES: CCCCCCCCCCCCCCSSSCC(CC)OC(=O)CCN(CCCN(C)CCCN(CCC(=O)OC(CC)CSSSCCCCCCCCCCCCCC)CCC(=O)OC(CC)CSSSCCCCCCCCCCCCCC)CCC(=O)OC(CC)CSSSCCCCCCCCCCCCCC] |
| F-10H | 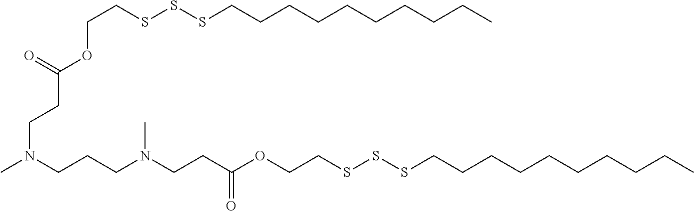 [SMILES: CCCCCCCCCCSSSCCOC(=O)CCN(C)CCCN(C)CCC(=O)OCCSSSCCCCCCCCCC] |
| F-12H | 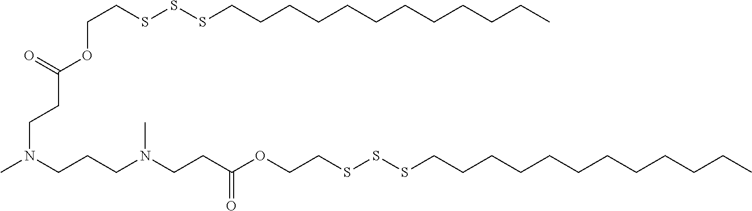 [SMILES: CCCCCCCCCCCCSSSCCOC(=O)CCN(C)CCCN(C)CCC(=O)OCCSSSCCCCCCCCCCCC] |
| F-14H | 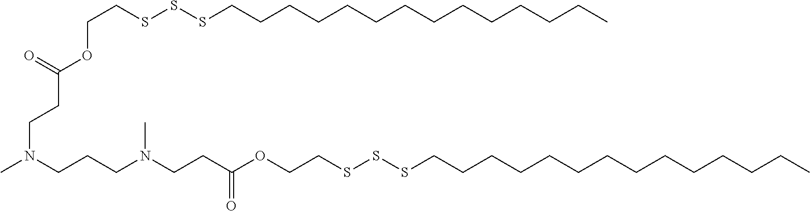 [SMILES: CCCCCCCCCCCCCCSSSCCOC(=O)CCN(C)CCCN(C)CCC(=O)OCCSSSCCCCCCCCCCCCCC] |
| F-10B6 | 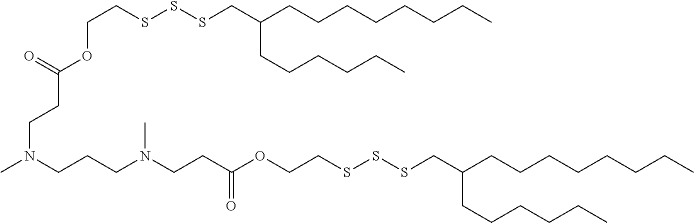 [SMILES: CCCCCCCCC(CCCCCC)CSSSCCOC(=O)CCN(C)CCCN(C)CCC(=O)OCCSSSCC(CCCCCC)CCCCCCCC] |
| F-10M | 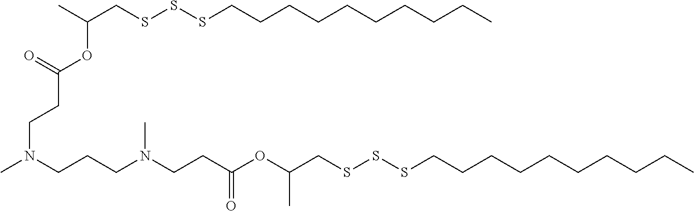 [SMILES: CCCCCCCCCCSSSCC(C)OC(=O)CCN(C)CCCN(C)CCC(=O)OC(C)CSSSCCCCCCCCCC] |
| F-12M | 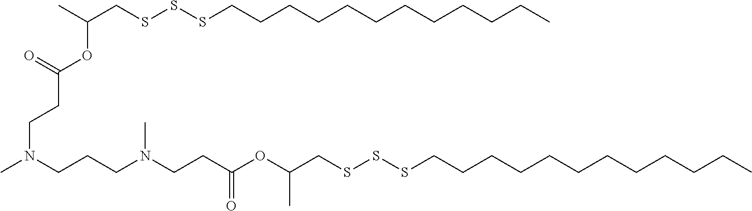 [SMILES: CCCCCCCCCCCCSSSCC(C)OC(=O)CCN(C)CCCN(C)CCC(=O)OC(C)CSSSCCCCCCCCCCCC] |
| F-14M | 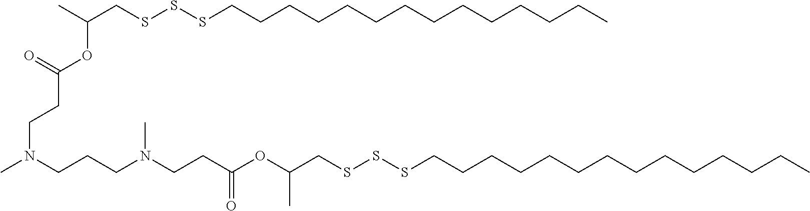 [SMILES: CCCCCCCCCCCCCCSSSCC(C)OC(=O)CCN(C)CCCN(C)CCC(=O)OC(C)CSSSCCCCCCCCCCCCCC] |
| F-10E | 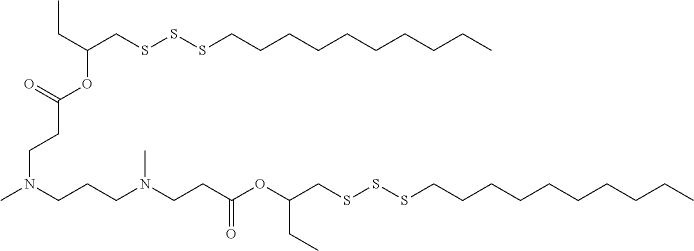 [SMILES: CCCCCCCCCCSSSCC(CC)OC(=O)CCN(C)CCCN(C)CCC(=O)OC(CC)CSSSCCCCCCCCCC] |
| F-12E | 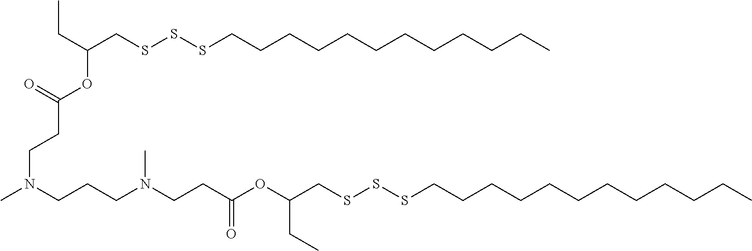 [SMILES: CCCCCCCCCCCCSSSCC(CC)OC(=O)CCN(C)CCCN(C)CCC(=O)OC(CC)CSSSCCCCCCCCCCCC] |
| F-14E | 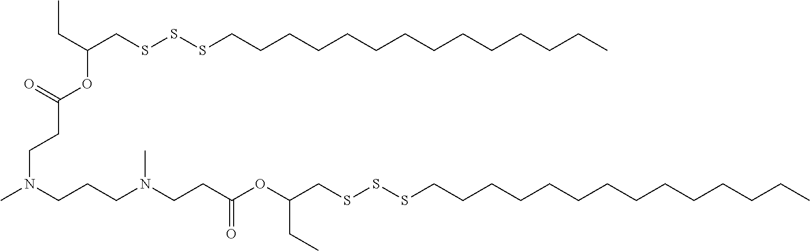 [SMILES: CCCCCCCCCCCCCCSSSCC(CC)OC(=O)CCN(C)CCCN(C)CCC(=O)OC(CC)CSSSCCCCCCCCCCCCCC] |
| G-10H | 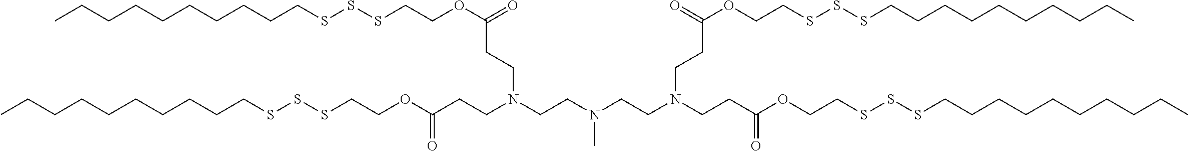 [SMILES: CCCCCCCCCCSSSCCOC(=O)CCN(CCC(=O)OCCSSSCCCCCCCCCC)CCN(C)CCN(CCC(=O)OCCSSSCCCCCCCCCC)CCC(=O)OCCSSSCCCCCCCCCC] |
| G-12H | 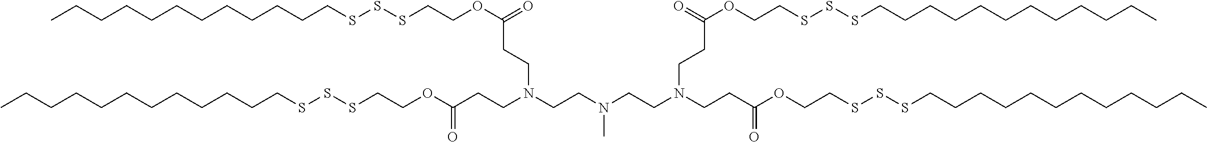 [SMILES: CCCCCCCCCCCCSSSCCOC(=O)CCN(CCC(=O)OCCSSSCCCCCCCCCCCC)CCN(C)CCN(CCC(=O)OCCSSSCCCCCCCCCCCC)CCC(=O)OCCSSSCCCCCCCCCCCC] |
| G-14H | 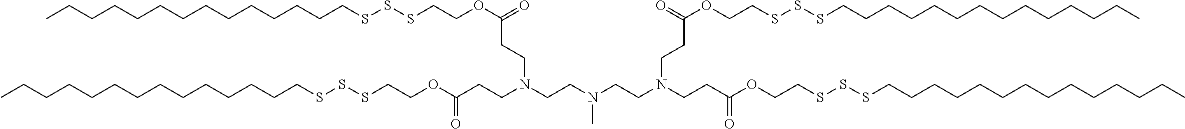 [SMILES: CCCCCCCCCCCCCCSSSCCOC(=O)CCN(CCC(=O)OCCSSSCCCCCCCCCCCCCC)CCN(C)CCN(CCC(=O)OCCSSSCCCCCCCCCCCCCC)CCC(=O)OCCSSSCCCCCCCCCCCCCC] |
| G-10B6 | 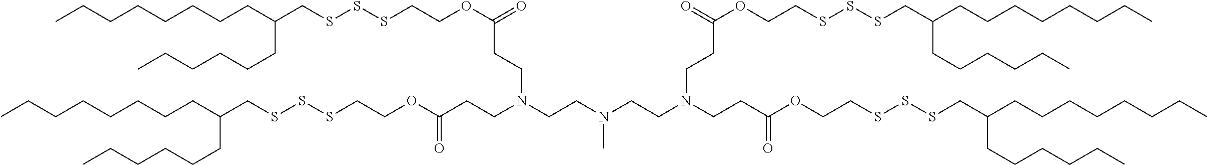 [SMILES: CCCCCCCCC(CCCCCC)CSSSCCOC(=O)CCN(CCC(=O)OCCSSSCC(CCCCCC)CCCCCCCC)CCN(C)CCN(CCC(=O)OCCSSSCC(CCCCCC)CCCCCCCC)CCC(=O)OCCSSSCC(CCCCCC)CCCCCCCC] |
| G-10M | 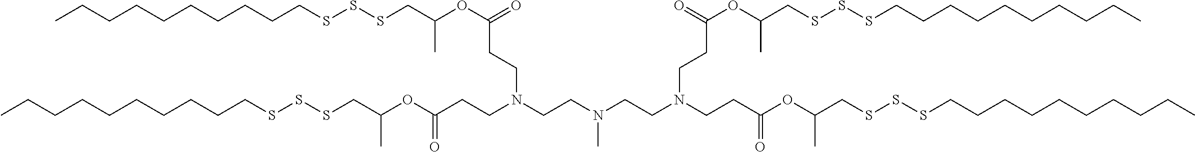 [SMILES: CCCCCCCCCCSSSCC(C)OC(=O)CCN(CCC(=O)OC(C)CSSSCCCCCCCCCC)CCN(C)CCN(CCC(=O)OC(C)CSSSCCCCCCCCCC)CCC(=O)OC(C)CSSSCCCCCCCCCC] |
| G-12M | 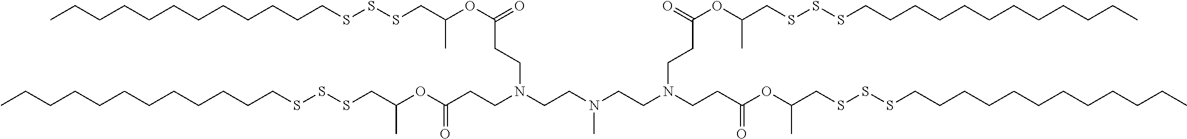 [SMILES: CCCCCCCCCCCCSSSCC(C)OC(=O)CCN(CCC(=O)OC(C)CSSSCCCCCCCCCCCC)CCN(C)CCN(CCC(=O)OC(C)CSSSCCCCCCCCCCCC)CCC(=O)OC(C)CSSSCCCCCCCCCCCC] |
| G-14M | 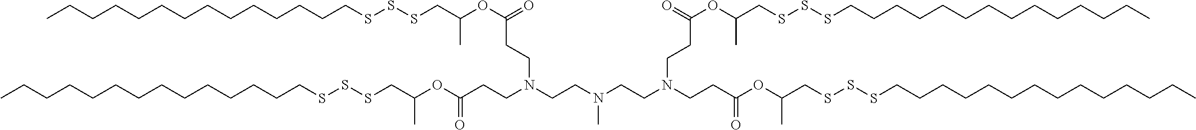 [SMILES: CCCCCCCCCCCCCCSSSCC(C)OC(=O)CCN(CCC(=O)OC(C)CSSSCCCCCCCCCCCCCC)CCN(C)CCN(CCC(=O)OC(C)CSSSCCCCCCCCCCCCCC)CCC(=O)OC(C)CSSSCCCCCCCCCCCCCC] |
| G-10E | 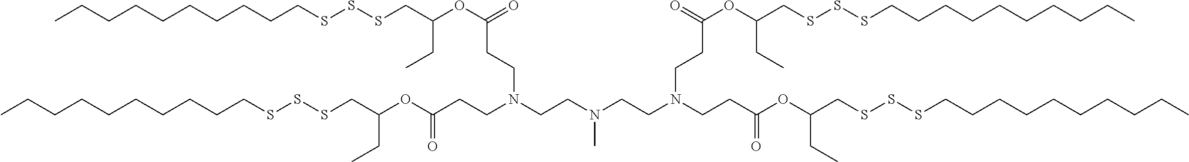 [SMILES: CCCCCCCCCCSSSCC(CC)OC(=O)CCN(CCC(=O)OC(CC)CSSSCCCCCCCCCC)CCN(C)CCN(CCC(=O)OC(CC)CSSSCCCCCCCCCC)CCC(=O)OC(CC)CSSSCCCCCCCCCC] |
| G-12E | 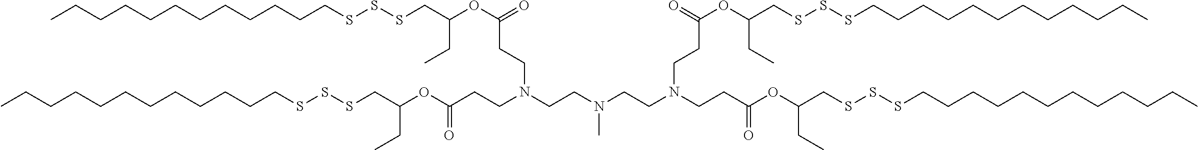 [SMILES: CCCCCCCCCCCCSSSCC(CC)OC(=O)CCN(CCC(=O)OC(CC)CSSSCCCCCCCCCCCC)CCN(C)CCN(CCC(=O)OC(CC)CSSSCCCCCCCCCCCC)CCC(=O)OC(CC)CSSSCCCCCCCCCCCC] |
| G-14E | 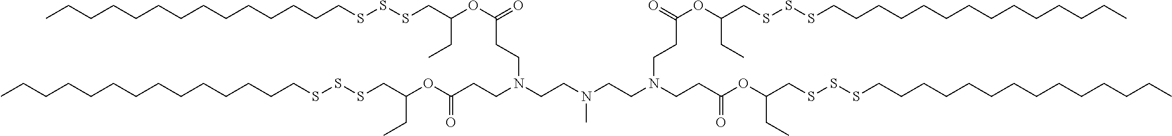 [SMILES: CCCCCCCCCCCCCCSSSCC(CC)OC(=O)CCN(CCC(=O)OC(CC)CSSSCCCCCCCCCCCCCC)CCN(C)CCN(CCC(=O)OC(CC)CSSSCCCCCCCCCCCCCC)CCC(=O)OC(CC)CSSSCCCCCCCCCCCCCC] |

### General Procedure for the Synthesis of Thiotosylates 5-7, 15-17, and 29

To a dry round bottom flask alkyl halide (1.0 eq, 10 mmol) was added in dry acetonitrile (50 mL). Then sodium 4-methylbenzenesulfonotioate (1.0 eq, 10.5 mmol) and TBAI (1.0 mmol, 0.1 eq.) was added, reaction was refluxed for 3 h. Solvent was evaporated after starting material was consumed totally, residue was dissolved in 200 mL of EA, washed with 50 mL of water, then the organic layers were dried over anhydrous Na2SO4, filtrated and the solvent was evaporated. The residue was purified by column chromatography.

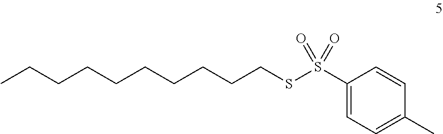 [SMILES: CCCCCCCCCCSS(=O)(=O)c1ccc(C)cc1]

Yield: 90%. ¹H NMR (300 MHz, CDCl3) δ 7.81 (d, J=8.4 Hz, 2H), 7.34 (d, J=8.1 Hz, 2H), 2.97 (t, J=7.5 Hz, 2H), 2.45 (s, 3H), 1.66-1.51 (m, 2H), 1.36-1.13 (m, 14H), 0.88 (t, J 6.9 Hz, 3H).

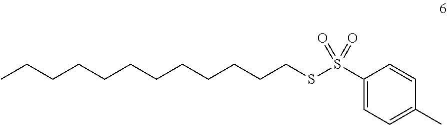 [SMILES: CCCCCCCCCCCCSS(=O)(=O)c1ccc(C)cc1]

Yield: 92%. ¹H NMR (300 MHz, CDCl3) δ 7.81 (dd, J=8.4, 1.8 Hz, 2H), 7.34 (d, J 7.8 Hz, 2H), 2.97 (t, J=7.5 Hz, 2H), 2.45 (s, 3H), 1.57 (h, J=7.2 Hz, 2H), 1.31-1.17 (m, 18H), 0.88 (t, J=6.9 Hz, 3H).

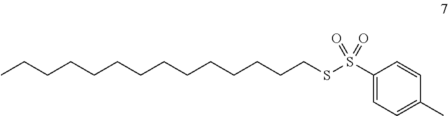 [SMILES: CCCCCCCCCCCCCCSS(=O)(=O)c1ccc(C)cc1]

Yield: 89%. ¹H NMR (300 MHz, CDCl3) δ 7.81 (d, J=8.4 Hz, 2H), 7.34 (d, J=8.1 Hz, 2H), 2.98 (t, J=7.5 Hz, 2H), 2.45 (s, 3H), 1.59 (q, J=7.2 Hz, 2H), 1.23 (d, J=13.1 Hz, 22H), 0.88 (t, J=6.6 Hz, 3H).

### General Procedure for the Synthesis of Disulfanyl Acetates 9-11 and 30

Sulfonothioate (3 mmol, 1 equiv.) and KSAc (3.9 mmol, 1.3 equiv.) were dissolved in DCM (20 mL) at r.t. and stirred for 6 h. Then the mixture was diluted with DCM (100 mL) and washed with saturated NaCl aqueous solution. The organic phase was dried over anhydrous Na2SO4, filtered, the solvent was evaporated. The residue was purified by column chromatography.

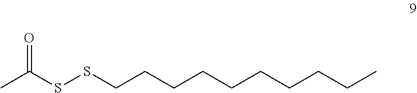

Yield: 89%. ¹H NMR (300 MHz, CDCl3) δ 2.71 (t, J=7.5 Hz, 2H), 2.44 (s, 3H), 1.62 (p, J=7.2 Hz, 2H), 1.42-1.25 (m, 14H), 0.88 (t, J=6.6 Hz, 3H).

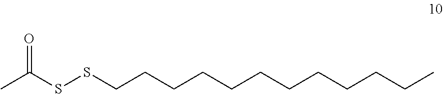 [SMILES: CCCCCCCCCCCCSSC(C)=O]

Yield: 86%. ¹H NMR (400 MHz, CDCl3) δ 2.72 (t, J=7.2 Hz, 2H), 2.44 (s, 3H), 1.62 (p, J=7.2 Hz, 2H), 1.38 (t, J=7.2 Hz, 2H), 1.30-1.20 (m, 18H), 0.88 (t, J=6.8 Hz, 3H).

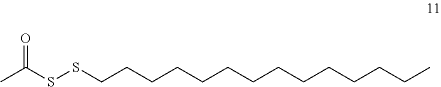 [SMILES: CCCCCCCCCCCCCCSSC(C)=O]

Yield: 88%. ¹H NMR (300 MHz, CDCl3) δ 2.71 (t, J=7.2 Hz, 2H), 2.44 (s, 3H), 1.67-1.59 (m, 2H), 1.42-1.20 (m, 22H), 0.88 (t, J=6.6 Hz, 3H).

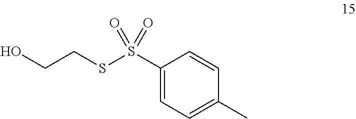

Yield: 90%. ¹H NMR (400 MHz, CDCl3) δ 7.83 (d, J=8.4 Hz, 2H), 7.36 (d, J=8.0 Hz, 2H), 3.86 (q, J=6.0 Hz, 2H), 3.17 (t, J=6.0 Hz, 2H), 2.46 (s, 3H).

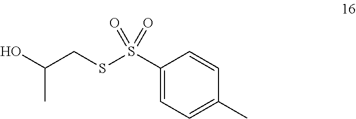

Yield: 86%. ¹H NMR (300 MHz, CDCl3) δ 7.81 (d, J=8.4 Hz, 2H), 7.35 (d, J=8.4 Hz, 2H), 4.10-3.96 (m, 1H), 3.13 (dd, J=14.0, 4.0 Hz, 1H), 3.00 (dd, J=14.0, 7.5 Hz, 1H), 2.45 (s, 3H), 2.12 (br. s, 1H), 1.23 (d, J=6.3 Hz, 3H). MS (ESI, m/z): [M+H]+ calcd. For C10H15O3S2, 247.0; found: 247.1.

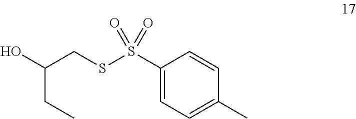 [SMILES: CCC(O)CSS(=O)(=O)c1ccc(C)cc1]

Yield: 82%. ¹H NMR (300 MHz, CDCl3) δ 7.81 (d, J=8.4 Hz, 2H), 7.35 (d, J=8.7 Hz, 2H), 3.82-3.69 (m, 1H), 3.15 (dd, J=14.0, 3.6 Hz, 1H), 2.99 (dd, J=14.0, 8.0 Hz, 1H), 2.45 (s, 3H), 2.13 (br. s, 1H), 1.60-1.43 (m, 2H), 0.92 (t, J=7.5 Hz, 3H). MS (ESI, m/z): [M+H]+ calcd. For C₁₁H17O3S2, 261.1; found: 261.1.

### General Procedure for the Synthesis of Alcohols Containing Trisulfide Linker (18-26, 31)

A mixture of thiosulfonate (6.0 mmol, 1.2 equiv) and disulfanyl acetate (5.0 mmol, 1.0 equiv) were dissolved in THE (50 mL) and the resulting solution was cooled to −78° C. for 10 min under N2. Sodium methoxide (5 mL, 1 M solution in MeOH, 1.0 equiv) was added dropwise, and the resultant solution was stirred rapidly for 5 min. saturated aqueous ammonium chloride (50 mL) was added to quench the reaction, THE was removed under reduced pressure and the product was extracted with EtOAc (3×50 mL). Following drying over anhydrous Na2SO4 and evaporation of solvent, the residue was purified via silica column chromatography using hexane/ethyl acetate as an eluent.

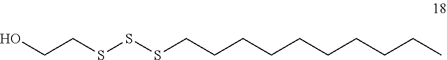 [SMILES: CCCCCCCCCCSSSCCO]

Yield: 68%. ¹H NMR (300 MHz, CDCl3) δ 3.96 (t, J=5.7 Hz, 2H), 3.05 (t, J=5.7 Hz, 2H), 2.95-2.85 (m, 2H), 1.86 (s, 1H), 1.81-1.68 (m, 2H), 1.46-1.21 (m, 14H), 0.88 (t, J 6.6 Hz, 3H).

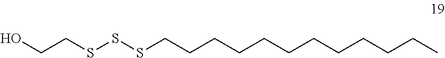 [SMILES: CCCCCCCCCCCCSSSCCO]

Yield: 65%. ¹H NMR (300 MHz, CDCl3) δ 3.96 (q, J=5.9 Hz, 2H), 3.05 (t, J=5.7 Hz, 2H), 2.90 (t, J=7.3 Hz, 2H), 2.06 (td, J=6.6, 1.9 Hz, 1H), 1.75 (p, J=7.2 Hz, 2H), 1.45-1.35 (m 2H), 1.35-1.15 (m, 16H), 0.88 (t, J=6.6 Hz, 3H).

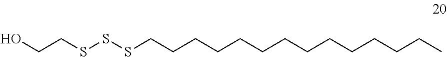 [SMILES: CCCCCCCCCCCCCCSSSCCO]

Yield: 58%. ¹H NMR (300 MHz, CDCl3) δ 3.96 (t, J=5.7 Hz, 2H), 3.05 (t, J=5.7 Hz, 2H), 2.90 (t, J=7.2 Hz, 2H), 1.90 (br. s, 1H), 1.80-1.68 (m, 2H), 1.46-1.18 (m, 22H), 0.88 (t, J=6.6 Hz, 3H).

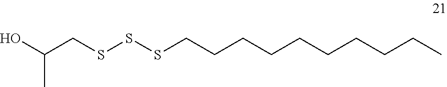 [SMILES: CCCCCCCCCCSSSCC(C)O]

Yield: 55%. H NMR (300 MHz, CDCl3) δ 4.16 (dqd, J=8.4, 6.3, 3.6 Hz, 1H), 3.07 (dd, J=13.8, 3.6 Hz, 1H), 2.90 (t, J=7.2 Hz, 2H), 2.80 (dd, J=13.8, 8.7 Hz, 1H), 2.11 (s, 1H), 1.79-1.68 (m, 2H), 1.44-1.23 (m, 17H), 0.88 (t, J=6.6 Hz, 3H).

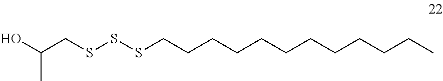 [SMILES: CCCCCCCCCCCCSSSCC(C)O]

Yield: 54%. ¹H NMR (300 MHz, CDCl3) δ 4.16 (dqd, J=8.4, 6.3, 3.6 Hz, 1H), 3.07 (dd, J=13.8, 3.6 Hz, 1H), 2.90 (t, J=7.2 Hz, 2H), 2.80 (dd, J=13.8, 8.6 Hz, 1H), 1.98 (s, 1H), 1.79-1.68 (m, 2H), 1.46-1.21 (m, 21H), 0.88 (t, J=6.6 Hz, 3H).

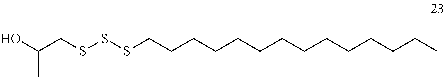 [SMILES: CCCCCCCCCCCCCCSSSCC(C)O]

Yield: 55%. ¹H NMR (300 MHz, CDCl3) δ 4.16 (dqd, J=8.4, 6.3, 3.6 Hz, 1H), 3.07 (dd, J=13.8, 3.6 Hz, 1H), 2.90 (d, J=7.2 Hz, 2H), 2.80 (dd, J=13.8, 8.7 Hz, 1H), 2.03 (s, 1H), 1.81-1.68 (m, 2H), 1.45-1.20 (m, 26H), 0.88 (t, J=6.6 Hz, 3H).

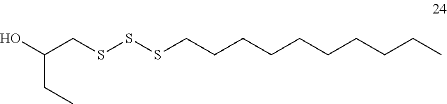 [SMILES: CCCCCCCCCCSSSCC(O)CC]

Yield: 58%. ¹H NMR (300 MHz, CDCl3) δ 3.89 (dddd, J=8.7, 6.6, 5.7, 3.3 Hz, 1H), 3.11 (dd, J=13.8, 3.3 Hz, 1H), 2.90 (t, J=7.5 Hz, 2H), 2.80 (dd, J=13.8, 8.8 Hz, 1H), 2.11 (s, 1H), 1.74 (ddd, J=14.8, 8.3, 6.9 Hz, 2H), 1.66-1.54 (m, 2H), 1.45-1.21 (m, 14H), 1.01 (t, J 7.4 Hz, 3H), 0.88 (t, J=6.6 Hz, 3H).

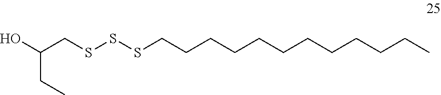 [SMILES: CCCCCCCCCCCCSSSCC(O)CC]

Yield: 51%. ¹H NMR (300 MHz, CDCl3) δ 3.89 (dddd, J=8.7, 6.9, 5.7, 3.3 Hz, 1H), 3.11 (dd, J=13.8, 3.3 Hz, 1H), 2.90 (t, J=7.5 Hz, 2H), 2.80 (dd, J=13.8, 8.7 Hz, 1H), 2.10 (s, 1H), 1.82-1.68 (m, 2H), 1.60 (pd, J=6.3, 1.5 Hz, 2H), 1.45-1.20 (m, 18H), 1.01 (t, J=7.4 Hz, 3H), 0.88 (t, J=6.6 Hz, 3H).

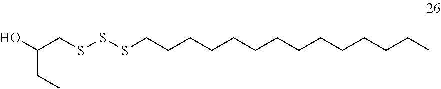 [SMILES: CCCCCCCCCCCCCCSSSCC(O)CC]

Yield: 56%. ¹H NMR (300 MHz, CDCl3) δ 3.89 (dddd, J=8.7, 6.9, 5.7, 3.3 Hz, 1H), 3.11 (dd, J=13.8, 3.3 Hz, 1H), 2.90 (t, J=7.5 Hz, 2H), 2.80 (dd, J=13.8, 8.7 Hz, 1H), 2.04 (s, 1H), 1.81-1.68 (m, 2H), 1.60 (pd, J=6.4, 1.3 Hz, 2H), 1.45-1.21 (m, 22H), 1.01 (t, J=7.4 Hz, 3H), 0.88 (t, J=6.6 Hz, 3H).

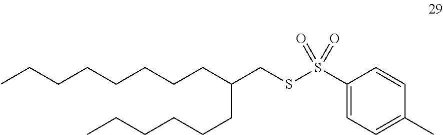 [SMILES: CCCCCCCCC(CCCCCC)CSS(=O)(=O)c1ccc(C)cc1]

Yield: 85%. ¹H NMR (300 MHz, CDCl3) δ 7.81 (d, J=8.1 Hz, 2H), 7.30 (d, J=8.1 Hz, 2H), 2.97 (d, J=6.0 Hz, 2H), 2.45 (s, 3H), 1.64-1.54 (m, 1H), 1.33-1.08 (m, 24H), 0.88 (td, J 6.9, 3.0 Hz, 6H).

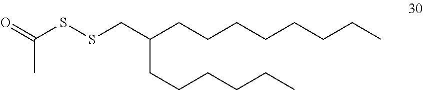 [SMILES: CCCCCCCCC(CCCCCC)CSSC(C)=O]

Yield: 83%. ¹H NMR (300 MHz, CDCl3) δ 2.72 (d, J=6.3 Hz, 2H), 2.43 (s, 3H), 1.66-1.55 (m, 1H), 1.38-1.22 (m, 24H), 0.88 (t, J=6.6 Hz, 6H)

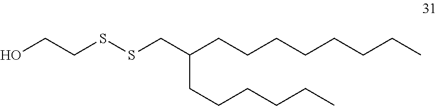 [SMILES: CCCCCCCCC(CCCCCC)CSSCCO]

Yield: 56%. ¹H NMR (300 MHz, CDCl3) δ 3.96 (t, J=5.7 Hz, 2H), 3.05 (t, J=5.7 Hz, 2H), 2.92 (d, J=6.3 Hz, 2H), 1.95 (s, 1H), 1.81-1.69 (m, 1H), 1.44-1.20 (m, 24H), 0.95-0.83 (m, 6H).

### General Procedure for the Synthesis of Acrylates Containing Trisulfide Linker (10H, 12H, 14H, 10B6, 10M, 12M, 14M, 10E, 12E, and 14E).

Alcohol containing trisulfide linker (3.0 mmol, 1.0 eq.) and Et3N (6.0 mmol, 2.0 eq.) were dissolved in 30 mL of CH2Cl2 and the solution was cooled to 0° C. under an argon atmosphere; then a solution of acryloyl chloride (3.6 mmol, 1.2 eq.) was added dropwise. The resulting mixture was stirred for 2 h, then quenched with water (10 mL), and extracted with CH2Cl2 (3×20 mL). The combined organic phases were dried over Na2SO4. The solvent was removed under reduced pressure, and the residue was purified on a silica gel column eluted with Hexane and Ethyl Acetate to afford the desired acrylates as oils.

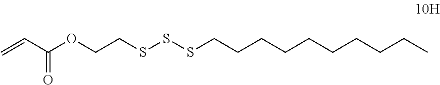 [SMILES: C=CC(=O)OCCSSSCCCCCCCCCC]

Yield: 92%. ¹H NMR (300 MHz, CDCl3) δ 6.44 (dd, J=17.4, 1.5 Hz, 1H), 6.14 (dd, J 17.4, 10.5 Hz, 1H), 5.85 (dd, J=10.5, 1.5 Hz, 1H), 4.49 (t, J=6.6 Hz, 2H), 3.14 (t, J=6.6 Hz, 2H), 2.96-2.84 (m, 2H), 1.82-1.66 (m, 2H), 1.47-1.18 (m, 14H), 0.88 (t, J=6.6 Hz, 3H).

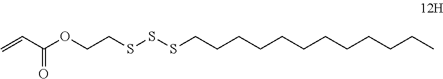 [SMILES: C=CC(=O)OCCSSSCCCCCCCCCCCC]

Yield: 90%. ¹H NMR (400 MHz, CDCl3) δ 6.44 (dd, J=17.2, 1.6 Hz, 1H), 6.14 (dd, J 17.2, 10.6 Hz, 1H), 5.86 (dd, J=10.6, 1.6 Hz, 1H), 4.49 (t, J=6.8 Hz, 2H), 3.14 (t, J=6.8 Hz, 2H), 2.88 (t, J=7.2 Hz, 2H), 1.73 (p, J=7.2 Hz, 2H), 1.44-1.35 (m, 2H), 1.35-1.19 (m, 16H), 0.88 (t, J=6.8 Hz, 3H).

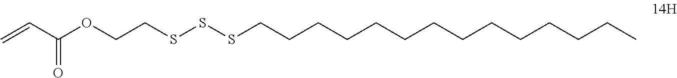 [SMILES: C=CC(=O)OCCSSSCCCCCCCCCCCCCC]

Yield: 91%. ¹H NMR (300 MHz, CDCl3) δ 6.44 (dd, J=17.4, 1.5 Hz, 1H), 6.14 (dd, J 17.4, 10.5 Hz, 1H), 5.85 (dd, J=10.5, 1.5 Hz, 1H), 4.49 (t, J=6.6 Hz, 2H), 3.14 (t, J=6.6 Hz, 2H), 2.94-2.83 (m, 2H), 1.80-1.66 (m, 2H), 1.46-1.20 (m, 22H), 0.88 (t, J=6.6 Hz, 3H).

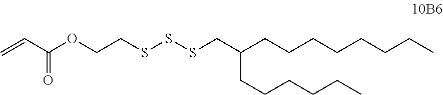 [SMILES: C=CC(=O)OCCSSSCC(CCCCCC)CCCCCCCC]

Yield: 93%. ¹H NMR (300 MHz, CDCl3) δ 6.44 (dd, J=17.4, 1.5 Hz, 1H), 6.14 (dd, J 17.4, 10.5 Hz, 1H), 5.86 (dd, J=10.5, 1.5 Hz, 1H), 4.49 (t, J=6.6 Hz, 2H), 3.14 (t, J=6.6 Hz, 2H), 2.90 (d, J=6.3 Hz, 2H), 1.73 (h, J=6.0 Hz, 1H), 1.41-1.22 (m, 24H), 0.94-0.83 (m, 6H).

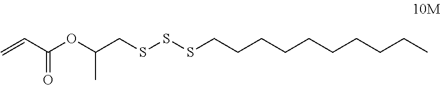 [SMILES: C=CC(=O)OC(C)CSSSCCCCCCCCCC]

Yield: 94%. ¹H NMR (300 MHz, CDCl3) δ 6.41 (dd, J=17.4, 1.5 Hz, 1H), 6.11 (dd, J 17.4, 10.5 Hz, 1H), 5.83 (dd, J=10.5, 1.5 Hz, 1H), 5.35-5.20 (m, 1H), 3.26-2.98 (m, 2H), 2.87 (t, J=7.2 Hz, 2H), 1.79-1.65 (m, 2H), 1.43-1.22 (m, 17H), 0.88 (t, J=6.6 Hz, 3H).

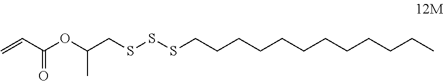 [SMILES: C=CC(=O)OC(C)CSSSCCCCCCCCCCCC]

Yield: 90%. ¹H NMR (300 MHz, CDCl3) δ 6.42 (dd, J=17.4, 1.5 Hz, 1H), 6.11 (dd, J 17.4, 10.5 Hz, 1H), 5.83 (dd, J=10.5, 1.5 Hz, 1H), 5.34-5.23 (m, 1H), 3.19-3.00 (m, 2H), 2.93-2.83 (m, 2H), 1.80-1.65 (m, 2H), 1.45-1.21 (m, 21H), 0.88 (t, J=6.6 Hz, 3H).

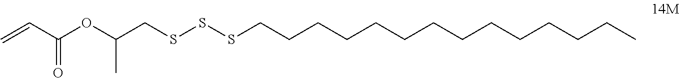 [SMILES: C=CC(=O)OC(C)CSSSCCCCCCCCCCCCCC]

Yield: 91%. ¹H NMR (300 MHz, CDCl3) δ 6.42 (dd, J=17.4, 1.5 Hz, 1H), 6.11 (dd, J 17.4, 10.4 Hz, 1H), 5.83 (dd, J=10.4, 1.5 Hz, 1H), 5.29 (h, J=6.3 Hz, 1H), 3.20-2.99 (m, 2H), 2.92-2.82 (m, 2H), 1.73 (ddd, J=14.8, 8.2, 6.8 Hz, 2H), 1.47-1.18 (m, 26H), 0.88 (t, J 6.6 Hz, 3H).

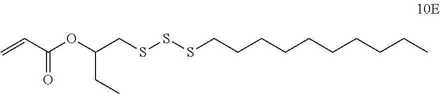 [SMILES: C=CC(=O)OC(CC)CSSSCCCCCCCCCC]

Yield: 88%. ¹H NMR (300 MHz, CDCl3) δ 6.42 (dd, J=17.4, 1.5 Hz, 1H), 6.13 (dd, J 17.4, 10.5 Hz, 1H), 5.84 (dd, J=10.5, 1.5 Hz, 1H), 5.18 (dq, J=7.2, 5.7 Hz, 1H), 3.11 (d, J 6.0 Hz, 2H), 2.93-2.81 (m, 2H), 1.89-1.65 (m, 4H), 1.45-1.21 (m, 14H), 0.95 (t, J=7.4 Hz, 3H), 0.88 (t, J=6.6 Hz, 3H).

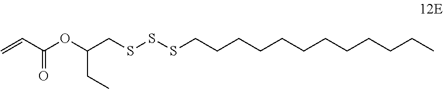 [SMILES: C=CC(=O)OC(CC)CSSSCCCCCCCCCCCC]

Yield: 89%. ¹H NMR (300 MHz, CDCl3) δ 6.42 (dd, J=17.4, 1.5 Hz, 1H), 6.13 (dd, J 17.4, 10.5 Hz, 1H), 5.84 (dd, J=10.5, 1.5 Hz, 1H), 5.19 (dq, J=7.2, 5.9 Hz, 1H), 3.11 (d, J 6.0 Hz, 2H), 2.87 (t, J=7.2 Hz, 2H), 1.88-1.64 (m, 4H), 1.45-1.20 (m, 18H), 0.95 (t, J=7.4 Hz, 3H), 0.88 (t, J=6.6 Hz, 3H).

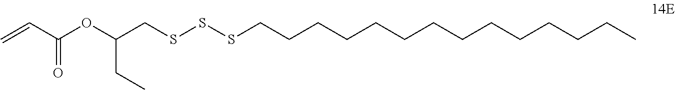 [SMILES: C=CC(=O)OC(CC)CSSSCCCCCCCCCCCCCC]

Yield: 91%. ¹H NMR (300 MHz, CDCl3) δ 6.42 (dd, J=17.4, 1.5 Hz, 1H), 6.13 (dd, J 17.4, 10.5 Hz, 1H), 5.83 (dd, J=10.5, 1.5 Hz, 1H), 5.18 (dq, J=7.2, 6.0 Hz, 1H), 3.11 (d, J 6.0 Hz, 2H), 2.92-2.82 (m, 2H), 1.90-1.65 (m, 4H), 1.45-1.19 (m, 23H), 0.95 (t, J=7.4 Hz, 3H), 0.88 (t, J=6.6 Hz, 3H).

### General Procedure for Michael Addition of Core Amines to Acrylates Containing Trisulfide Linker

A mixture of core amine and acylate (1.2 eq.) was warmed to 90° C. and kept stirring for 12 h. The reaction mixture was purified via CombiFlash system eluted with dichloromethane and DCM/MeOH/NH3·H2O (75:22:3) to afford the desired acrylates as oils

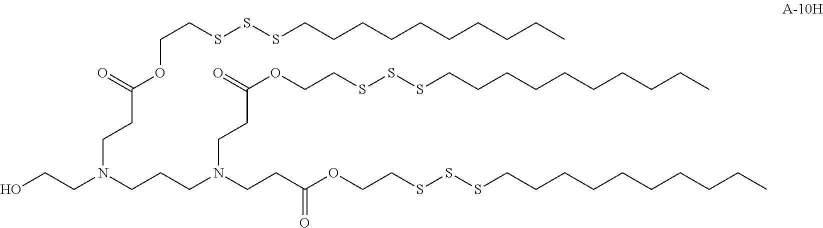 [SMILES: CCCCCCCCCCSSSCCOC(=O)CCN(CCO)CCCN(CCC(=O)OCCSSSCCCCCCCCCC)CCC(=O)OCCSSSCCCCCCCCCC]

¹H NMR (300 MHz, CDCl3) δ 4.41 (q, J=6.6 Hz, 6H), 3.60 (s, 2H), 3.10 (td, J=6.6, 2.1 Hz, 6H), 2.88 (t, J=7.2 Hz, 8H), 2.76 (t, J=7.2 Hz, 4H), 2.72-2.30 (m, 12H), 1.81-1.60 (m, 8H), 1.42-1.20 (m, 42H), 0.88 (t, J=6.9 Hz, 9H).

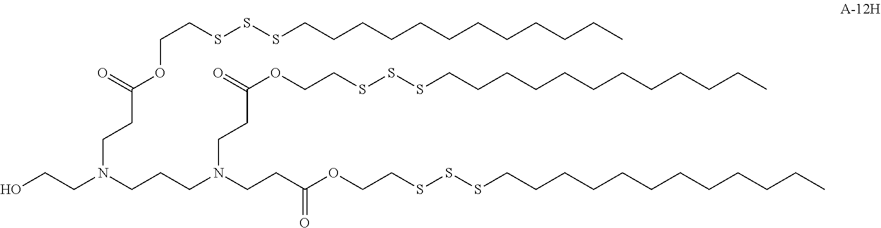 [SMILES: CCCCCCCCCCCCSSSCCOC(=O)CCN(CCO)CCCN(CCC(=O)OCCSSSCCCCCCCCCCCC)CCC(=O)OCCSSSCCCCCCCCCCCC]

¹H NMR (300 MHz, CDCl3) δ 4.40 (q, J=6.9 Hz, 6H), 3.75-3.50 (m, 2H), 3.10 (td, J 6.6, 2.1 Hz, 6H), 2.88 (t, J=7.2 Hz, 8H), 2.76 (t, J=6.9 Hz, 6H), 2.66-2.34 (m, 10H), 1.85-1.57 (m, 8H), 1.45-1.20 (m, 54H), 0.88 (t, J=6.6 Hz, 9H).

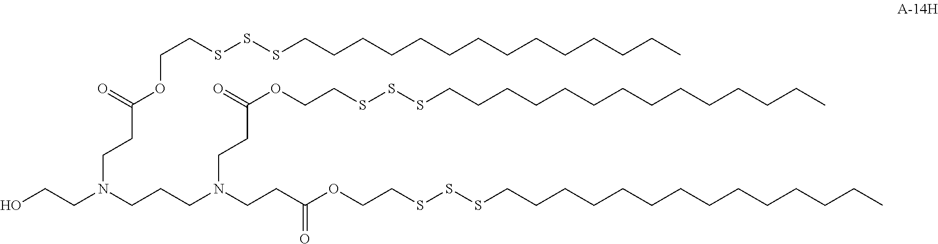 [SMILES: CCCCCCCCCCCCCCSSSCCOC(=O)CCN(CCO)CCCN(CCC(=O)OCCSSSCCCCCCCCCCCCCC)CCC(=O)OCCSSSCCCCCCCCCCCCCC]

¹H NMR (300 MHz, CDCl3) δ 4.40 (q, J=6.6 Hz, 6H), 3.58 (t, J=5.1 Hz, 2H), 3.10 (td, J=6.6, 2.1 Hz, 6H), 2.87 (t, J=7.2 Hz, 8H), 2.76 (t, J=7.1 Hz, 4H), 2.65-2.56 (m, 2H), 2.56-2.35 (m, 10H), 1.79-1.67 (m, 6H), 1.66-1.54 (m, 2H), 1.45-1.19 (m, 66H), 0.88 (t, J 6.6 Hz, 9H).

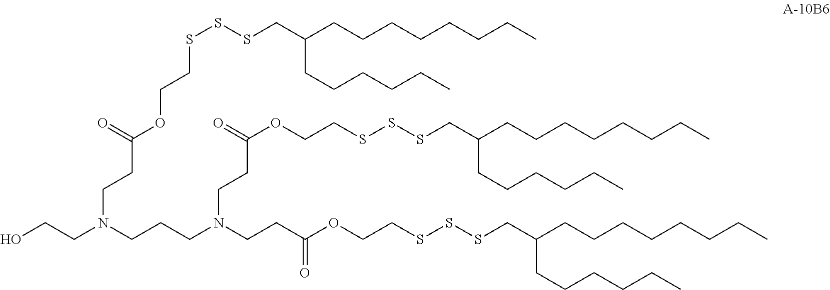 [SMILES: CCCCCCCCC(CCCCCC)CSSSCCOC(=O)CCN(CCO)CCCN(CCC(=O)OCCSSSCC(CCCCCC)CCCCCCCC)CCC(=O)OCCSSSCC(CCCCCC)CCCCCCCC]

¹H NMR (300 MHz, CDCl3) δ 4.41 (q, J=6.9 Hz, 6H), 3.60 (s, 2H), 3.10 (td, J=6.6, 2.2 Hz, 6H), 2.89 (d, J=6.3 Hz, 6H), 2.76 (t, J=6.9 Hz, 6H), 2.70-2.30 (m, 12H), 1.81-1.57 (m, 5H), 1.27 (s, 72H), 0.93-0.81 (m, 18H).

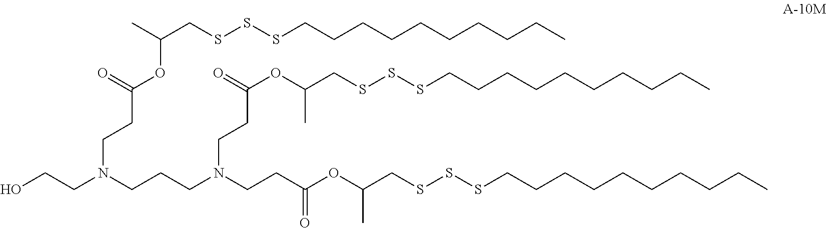 [SMILES: CCCCCCCCCCSSSCC(C)OC(=O)CCN(CCO)CCCN(CCC(=O)OC(C)CSSSCCCCCCCCCC)CCC(=O)OC(C)CSSSCCCCCCCCCC]

¹H NMR (300 MHz, CDCl3) δ 5.22 (p, J=6.3 Hz, 3H), 3.62 (s, 2H), 3.13-2.96 (m, 6H), 2.93-2.84 (m, 6H), 2.77 (t, J=7.1 Hz, 6H), 2.69-2.02 (m, 12H), 1.81-1.53 (m, 9H), 1.48-1.16 (m, 51H), 0.96-0.78 (m, 9H).

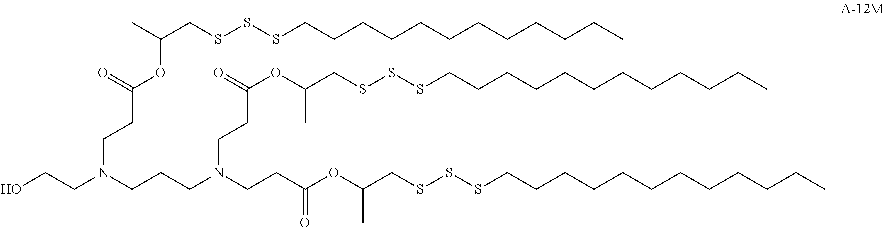 [SMILES: CCCCCCCCCCCCSSSCC(C)OC(=O)CCN(CCO)CCCN(CCC(=O)OC(C)CSSSCCCCCCCCCCCC)CCC(=O)OC(C)CSSSCCCCCCCCCCCC]

¹H NMR (300 MHz, CDCl3) δ 5.22 (hept, J=6.6 Hz, 3H), 3.61 (s, 2H), 3.14-2.97 (m, 6H), 2.96-2.80 (m, 8H), 2.77 (t, J=7.2 Hz, 4H), 2.72-2.18 (m, 12H), 1.84-1.52 (m, 8H), 1.48-0.99 (m, 63H), 0.96-0.79 (m, 9H).

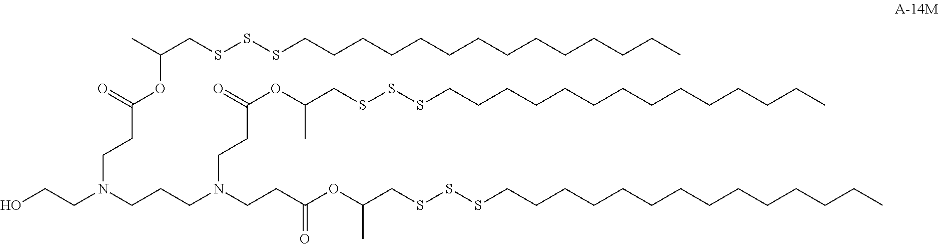 [SMILES: CCCCCCCCCCCCCCSSSCC(C)OC(=O)CCN(CCO)CCCN(CCC(=O)OC(C)CSSSCCCCCCCCCCCCCC)CCC(=O)OC(C)CSSSCCCCCCCCCCCCCC]

¹H NMR (300 MHz, CDCl3) δ 5.21 (hept, J=6.3 Hz, 3H), 3.59 (s, 2H), 3.13-2.96 (m 6H), 2.87 (t, J=7.2 Hz, 8H), 2.77 (t, J=7.2 Hz, 4H), 2.46 (q, J=10.8 Hz, 12H), 1.80-1.56 (m, 8H), 1.47-1.18 (m, 75H), 0.96-0.81 (m, 9H).

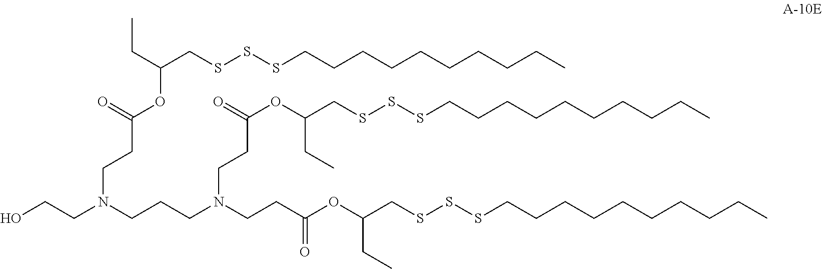 [SMILES: CCCCCCCCCCSSSCC(CC)OC(=O)CCN(CCO)CCCN(CCC(=O)OC(CC)CSSSCCCCCCCCCC)CCC(=O)OC(CC)CSSSCCCCCCCCCC]

¹H NMR (300 MHz, CDCl3) δ 5.20-5.05 (m, 3H), 3.60 (s, 2H), 3.12-3.00 (m, 6H), 2.87 (t, J=7.2 Hz, 8H), 2.78 (t, J=7.2 Hz, 4H), 2.73-2.52 (m, 4H), 2.47 (t, J=7.2 Hz, 8H), 1.84-1.58 (m, 14H), 1.48-1.21 (m, 42H), 0.99-0.83 (m, 18H).

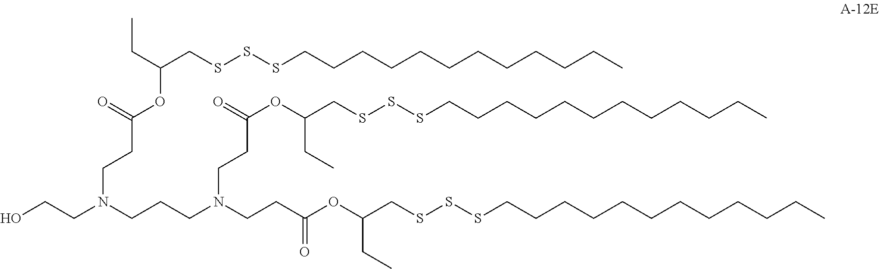 [SMILES: CCCCCCCCCCCCSSSCC(CC)OC(=O)CCN(CCO)CCCN(CCC(=O)OC(CC)CSSSCCCCCCCCCCCC)CCC(=O)OC(CC)CSSSCCCCCCCCCCCC]

¹H NMR (300 MHz, CDCl3) δ 5.20-5.01 (m, 3H), 3.60 (s, 2H), 3.14-3.00 (m, 6H), 2.87 (t, J=7.2 Hz, 7H), 2.78 (t, J=7.2 Hz, 5H), 2.71-2.32 (m 12H), 1.90-1.55 (m, 14H), 1.55-1.00 (m, 54H), 0.91 (dt, J=16.5, 7.2 Hz, 18H).

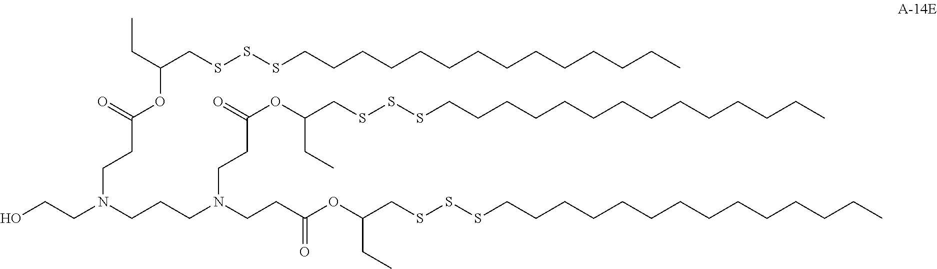 [SMILES: CCCCCCCCCCCCCCSSSCC(CC)OC(=O)CCN(CCO)CCCN(CCC(=O)OC(CC)CSSSCCCCCCCCCCCCCC)CCC(=O)OC(CC)CSSSCCCCCCCCCCCCCC]

¹H NMR (300 MHz, CDCl3) δ 5.17-5.02 (m, 3H), 3.60 (s, 1H), 3.14-3.00 (m, 6H), 2.98-2.72 (m, 12H), 2.47 (t, J=7.2 Hz, 12H), 1.82-1.58 (m, 14H), 1.50-1.15 (m, 66H), 0.98-0.80 (m, 18H).

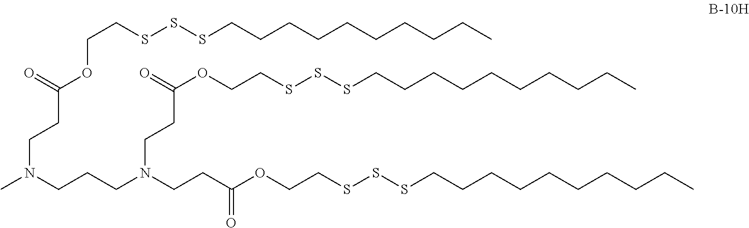 [SMILES: CCCCCCCCCCSSSCCOC(=O)CCN(C)CCCN(CCC(=O)OCCSSSCCCCCCCCCC)CCC(=O)OCCSSSCCCCCCCCCC]

¹H NMR (300 MHz, CDCl3) δ 4.40 (td, J=6.6, 4.2 Hz, 6H), 3.09 (t, J=6.6 Hz, 6H), 2.94-2.82 (m, 6H), 2.76 (t, J=7.2 Hz, 6H), 2.62-2.50 (m, 2H), 2.50-2.33 (m, 8H), 2.25 (s, 3H), 1.81-1.60 (m, 8H), 1.45-1.19 (m, 42H), 0.88 (t, J=6.6 Hz, 9H).

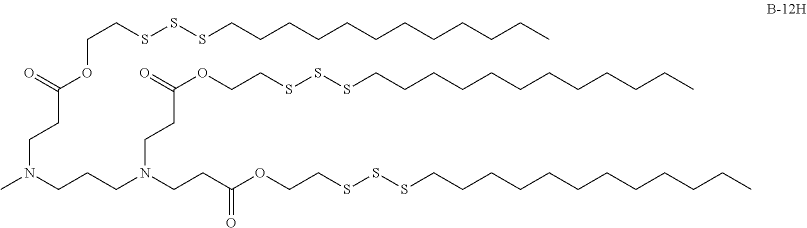 [SMILES: CCCCCCCCCCCCSSSCCOC(=O)CCN(C)CCCN(CCC(=O)OCCSSSCCCCCCCCCCCC)CCC(=O)OCCSSSCCCCCCCCCCCC]

¹H NMR (300 MHz, CDCl3) δ 4.39 (t, J=6.6 Hz, 6H), 3.09 (t, J=6.6 Hz, 6H), 2.92-2.84 (m, 6H), 2.75 (t, J=6.9 Hz, 6H), 2.67-2.53 (m, 2H), 2.46 (t, J=6.9 Hz, 8H), 2.29 (s, 3H), 1.83-1.56 (m, 8H), 1.44-1.19 (m, 54H), 0.88 (t, J=6.6 Hz, 9H).

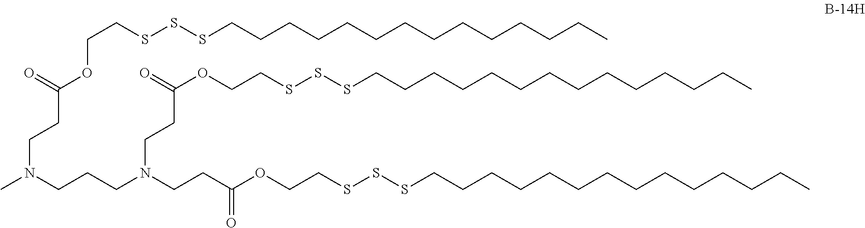 [SMILES: CCCCCCCCCCCCCCSSSCCOC(=O)CCN(C)CCCN(CCC(=O)OCCSSSCCCCCCCCCCCCCC)CCC(=O)OCCSSSCCCCCCCCCCCCCC]

¹H NMR (300 MHz, CDCl3) δ 4.40 (td, J=6.6, 3.9 Hz, 6H), 3.10 (t, J=6.6 Hz, 6H), 2.93-2.82 (m, 6H), 2.76 (t, J=7.2 Hz, 6H), 2.62-2.50 (m, 2H), 2.50-2.32 (m, 8H), 2.25 (s, 3H), 1.78-1.68 (m, 6H), 1.66-1.57 (m, 2H), 1.45-1.20 (m, 66H), 0.88 (d, J=6.6 Hz, 9H).

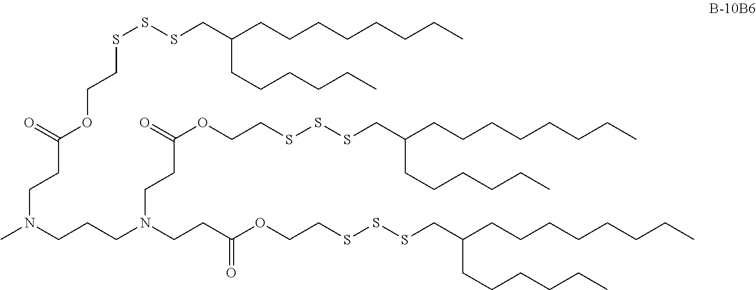 [SMILES: CCCCCCCCC(CCCCCC)CSSSCCOC(=O)CCN(C)CCCN(CCC(=O)OCCSSSCC(CCCCCC)CCCCCCCC)CCC(=O)OCCSSSCC(CCCCCC)CCCCCCCC]

¹H NMR (300 MHz, CDCl3) δ 4.43 (td, J=6.6, 4.5 Hz, 6H), 3.11 (t, J=6.6 Hz, 6H), 2.91 (d, J=6.4 Hz, 6H), 2.82-2.62 (m, 8H), 2.62-2.38 (m, 8H), 2.26 (s, 3H), 1.76 (p, J=6.0 Hz, 5H), 1.40-1.20 (m, 72H), 0.93-0.86 (m, 18H).

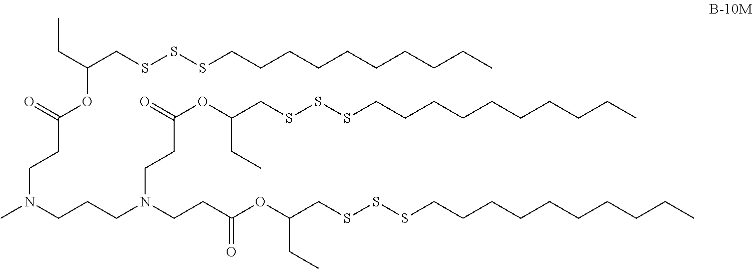 [SMILES: CCCCCCCCCCSSSCC(CC)OC(=O)CCN(C)CCCN(CCC(=O)OC(CC)CSSSCCCCCCCCCC)CCC(=O)OC(CC)CSSSCCCCCCCCCC]

¹H NMR (300 MHz, CDCl3) δ 5.21 (hd, J=6.0, 3.6 Hz, 3H), 3.13-2.95 (m, 6H), 2.87 (d, J=7.2 Hz, 6H), 2.76 (t, J=7.2 Hz, 6H), 2.43 (t, J=7.2 Hz, 9H), 2.26 (s, 3H), 1.78-1.56 (m, 8H), 1.46-1.16 (m, 51H), 0.87 (t, J=6.6 Hz, 9H).

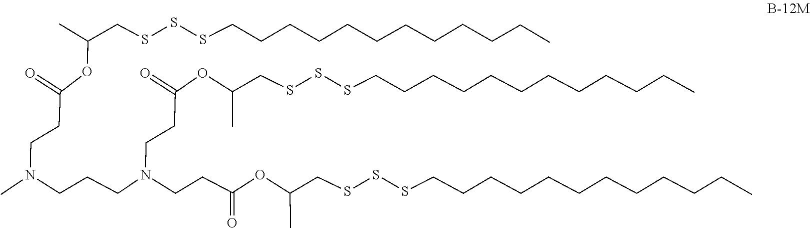 [SMILES: CCCCCCCCCCCCSSSCC(C)OC(=O)CCN(C)CCCN(CCC(=O)OC(C)CSSSCCCCCCCCCCCC)CCC(=O)OC(C)CSSSCCCCCCCCCCCC]

¹H NMR (300 MHz, CDCl3) δ 5.23 (h, J=6.0 Hz, 3H), 3.15-2.98 (m, 6H), 2.91 (d, J 7.2 Hz, 6H), 2.84-2.68 (m, 6H), 2.67-2.20 (m 12H), 1.81-1.59 (m, 8H), 1.51-1.19 (m, 63H), 0.96-0.82 (m, 9H).

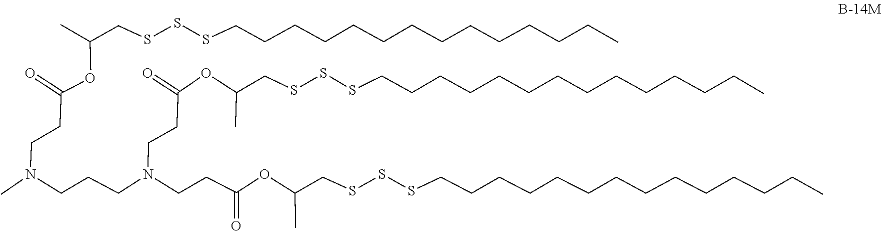 [SMILES: CCCCCCCCCCCCCCSSSCC(C)OC(=O)CCN(C)CCCN(CCC(=O)OC(C)CSSSCCCCCCCCCCCCCC)CCC(=O)OC(C)CSSSCCCCCCCCCCCCCC]

¹H NMR (300 MHz, CDCl3) δ 5.21 (qd, J=6.3, 3.6 Hz, 3H), 3.13-2.95 (m, 6H), 2.87 (t, J=7.2 Hz, 6H), 2.75 (q, J=7.8 Hz, 6H), 2.60-2.15 (m, 12H), 1.82-1.52 (m, 8H), 1.49-1.00 (m, 75H), 0.96-0.78 (m, 9H).

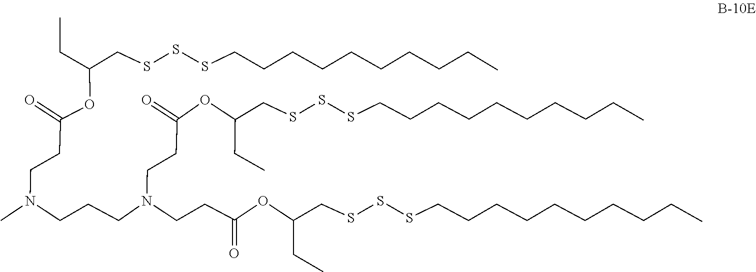 [SMILES: CCCCCCCCCCSSSCC(CC)OC(=O)CCN(C)CCCN(CCC(=O)OC(CC)CSSSCCCCCCCCCC)CCC(=O)OC(CC)CSSSCCCCCCCCCC]

¹H NMR (300 MHz, CDCl3) δ 5.11 (ddt, J=12.6, 6.9, 4.5 Hz, 3H), 3.14-2.99 (m, 6H), 2.95-2.65 (m, 12H), 2.65-2.38 (m, 9H), 2.38-2.00 (m, 4H), 1.84-1.55 (m, 14H), 1.47-1.17 (m, 42H), 1.00-0.81 (m, 18H).

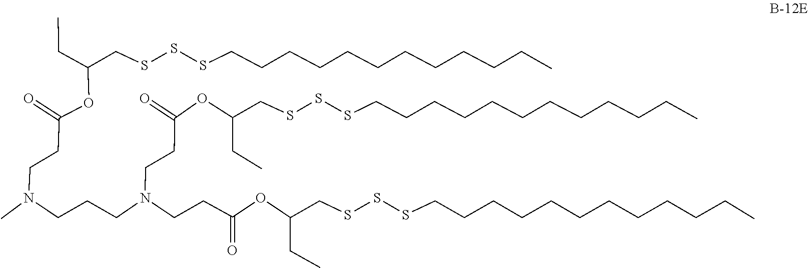 [SMILES: CCCCCCCCCCCCSSSCC(CC)OC(=O)CCN(C)CCCN(CCC(=O)OC(CC)CSSSCCCCCCCCCCCC)CCC(=O)OC(CC)CSSSCCCCCCCCCCCC]

¹H NMR (300 MHz, CDCl3) δ 5.25-4.92 (m, 3H), 3.17-2.97 (m, 6H), 2.87 (t, J=7.2 Hz, 6H), 2.83-2.66 (m, 6H), 2.65-2.07 (m, 13H), 1.84-1.58 (m, 14H), 1.48-1.17 (m, 54H), 1.01-0.80 (m, 18H).

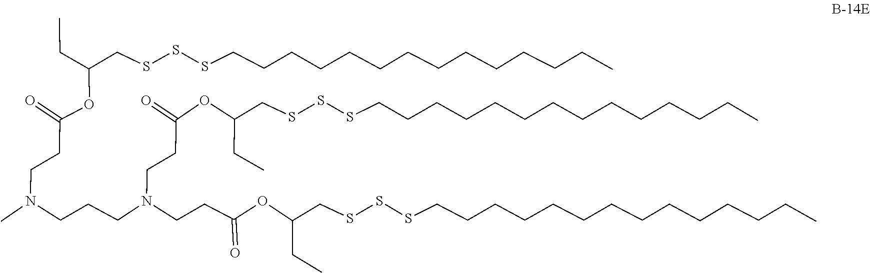 [SMILES: CCCCCCCCCCCCCCSSSCC(CC)OC(=O)CCN(C)CCCN(CCC(=O)OC(CC)CSSSCCCCCCCCCCCCCC)CCC(=O)OC(CC)CSSSCCCCCCCCCCCCCC]

¹H NMR (300 MHz, CDCl3) δ 5.21-5.07 (m, 3H), 3.16-2.98 (m, 9H), 2.97-2.80 (m, 6H), 2.80-2.63 (m, 6H), 2.62-2.01 (m, 10H), 1.87-1.60 (m, 14H), 1.51-1.09 (m, 54H), 1.00-0.81 (m, 18H).

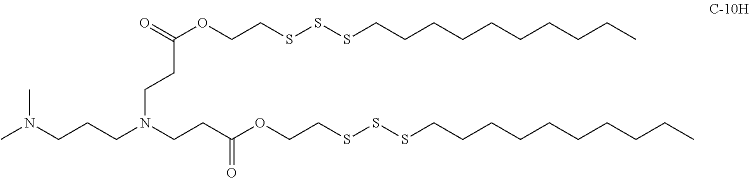 [SMILES: CCCCCCCCCCSSSCCOC(=O)CCN(CCCN(C)C)CCC(=O)OCCSSSCCCCCCCCCC]

¹H NMR (300 MHz, CDCl3) δ 4.38 (t, J=6.6 Hz, 4H), 3.08 (t, J=6.6 Hz, 4H), 2.87 (t, J 7.2 Hz, 4H), 2.75 (t, J=6.9 Hz, 4H), 2.45 (t, J=6.9 Hz, 6H), 2.38 (t, J=7.5 Hz, 2H), 2.31 (s, 6H), 1.81-1.57 (m, 6H), 1.51-1.13 (m, 28H), 0.87 (t, J=6.6 Hz, 6H).

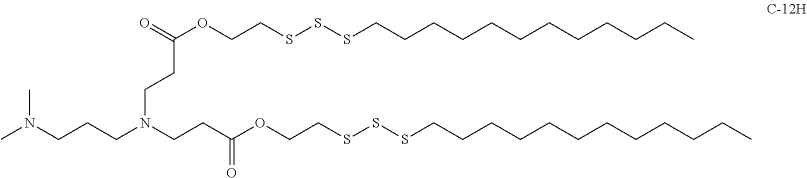 [SMILES: CCCCCCCCCCCCSSSCCOC(=O)CCN(CCCN(C)C)CCC(=O)OCCSSSCCCCCCCCCCCC]

¹H NMR (300 MHz, CDCl3) δ 4.39 (t, J=6.6 Hz, 4H), 3.09 (t, J=6.6 Hz, 4H), 2.87 (t, J 7.2 Hz, 4H), 2.76 (t, J=6.9 Hz, 4H), 2.46 (t, J=6.9 Hz, 6H), 2.42-2.28 (m, 8H), 1.70 (dt, J 14.4, 7.5 Hz, 6H), 1.50-1.12 (m, 36H), 0.87 (t, J=6.6 Hz, 6H).

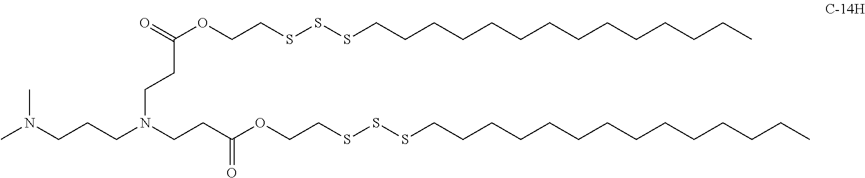 [SMILES: CCCCCCCCCCCCCCSSSCCOC(=O)CCN(CCCN(C)C)CCC(=O)OCCSSSCCCCCCCCCCCCCC]

¹H NMR (300 MHz, CDCl3) δ 4.38 (t, J=6.6 Hz, 4H), 3.09 (t, J=6.6 Hz, 4H), 2.87 (t, J 7.2 Hz, 4H), 2.75 (t, J=6.9 Hz, 4H), 2.53-2.36 (m, 8H), 2.33 (s, 6H), 1.79-1.61 (m, 6H), 1.49-1.17 (m, 44H), 0.87 (t, J=6.6 Hz, 6H).

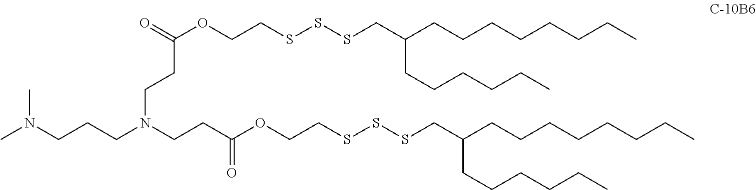 [SMILES: CCCCCCCCC(CCCCCC)CSSSCCOC(=O)CCN(CCCN(C)C)CCC(=O)OCCSSSCC(CCCCCC)CCCCCCCC]

¹H NMR (300 MHz, CDCl3) δ 4.39 (t, J=6.6 Hz, 4H), 3.09 (t, J=6.6 Hz, 4H), 2.89 (d, J 6.3 Hz, 4H), 2.76 (t, J=6.9 Hz, 4H), 2.46 (t, J=6.9 Hz, 6H), 2.41-2.21 (m, 6H), 1.83-1.57 (m 4H), 1.39-1.22 (m, 48H), 0.88 (t, J=6.6 Hz, 12H).

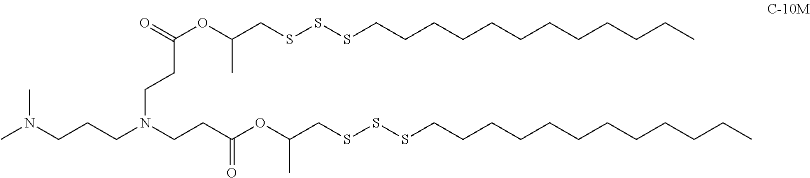 [SMILES: CCCCCCCCCCCCSSSCC(C)OC(=O)CCN(CCCN(C)C)CCC(=O)OC(C)CSSSCCCCCCCCCCCC]

¹H NMR (300 MHz, CDCl3) δ 5.20 (h, J=6.3 Hz, 2H), 3.03 (qd, J=13.8, 6.3 Hz, 4H), 2.91-2.81 (m, 4H), 2.76 (t, J=7.2 Hz, 4H), 2.44 (td, J=7.2, 4.2 Hz, 6H), 2.35-2.19 (m, 8H), 1.79-1.55 (m, 6H), 1.44-1.21 (m, 34H), 0.87 (t, J=6.6 Hz, 6H).

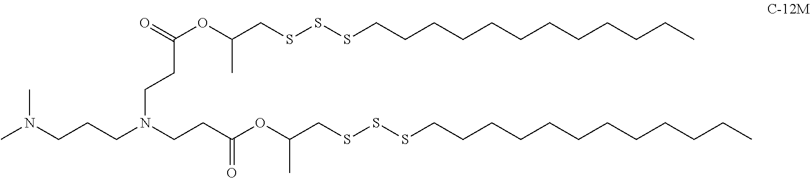 [SMILES: CCCCCCCCCCCCSSSCC(C)OC(=O)CCN(CCCN(C)C)CCC(=O)OC(C)CSSSCCCCCCCCCCCC]

¹H NMR (300 MHz, CDCl3) δ 5.20 (h, J=6.3 Hz, 2H), 3.04 (qd, J=13.8, 6.0 Hz, 4H), 2.87 (t, J=7.2 Hz, 4H), 2.75 (t, J=7.2 Hz, 4H), 2.45 (dt, J=11.2, 6.9 Hz, 8H), 2.36 (d, J=2.4 Hz, 6H), 1.78-1.63 (m, 6H), 1.46-1.18 (m, 42H), 0.87 (d, J=6.6 Hz, 6H).

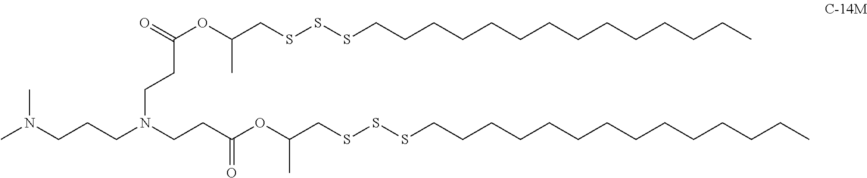 [SMILES: CCCCCCCCCCCCCCSSSCC(C)OC(=O)CCN(CCCN(C)C)CCC(=O)OC(C)CSSSCCCCCCCCCCCCCC]

¹H NMR (300 MHz, CDCl3) δ 5.19 (h, J=6.3 Hz, 2H), 3.03 (qd, J=13.8, 6.3 Hz, 4H), 2.86 (t, J=7.2 Hz, 4H), 2.76 (t, J=7.2 Hz, 4H), 2.54-2.36 (m, 6H), 2.36-2.09 (m, 8H), 1.83-1.51 (m, 6H), 1.42-1.18 (m, 50H), 0.87 (t, J=6.6 Hz, 6H).

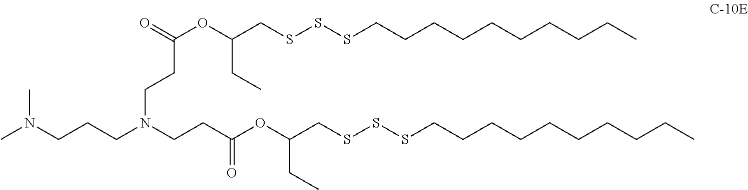 [SMILES: CCCCCCCCCCSSSCC(CC)OC(=O)CCN(CCCN(C)C)CCC(=O)OC(CC)CSSSCCCCCCCCCC]

¹H NMR (300 MHz, CDCl3) δ 5.21-5.02 (m, 2H), 3.14-2.99 (m, 4H), 2.86 (t, J=7.2 Hz, 4H), 2.78 (t, J=7.2 Hz, 4H), 2.46 (dd, J=8.1, 6.3 Hz, 6H), 2.40-2.15 (m, 8H), 1.83-1.56 (m, 10H), 1.47-1.17 (m, 28H), 0.90 (dt, J=15.6, 7.2 Hz, 12H).

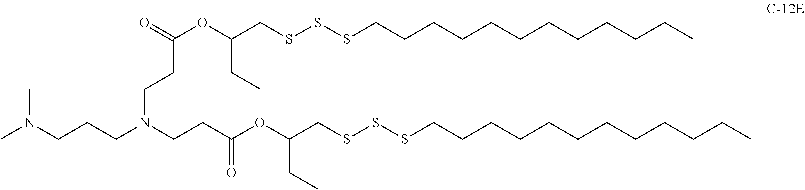 [SMILES: CCCCCCCCCCCCSSSCC(CC)OC(=O)CCN(CCCN(C)C)CCC(=O)OC(CC)CSSSCCCCCCCCCCCC]

¹H NMR (300 MHz, CDCl3) δ 5.10 (ddd, J=11.1, 6.9, 5.4 Hz, 2H), 3.14-2.98 (m, 4H), 2.87 (t, J=7.2 Hz, 4H), 2.77 (t, J=7.2 Hz, 4H), 2.69-2.25 (m, 14H), 1.90-1.55 (m, 10H), 1.50-1.13 (m, 36H), 1.00-0.79 (m, 12H).

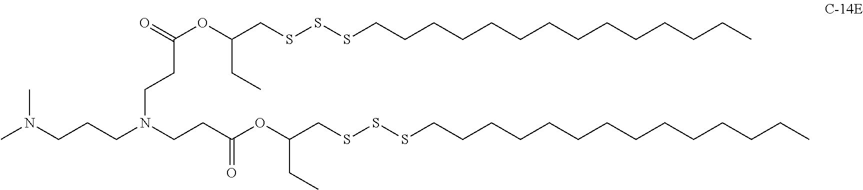 [SMILES: CCCCCCCCCCCCCCSSSCC(CC)OC(=O)CCN(CCCN(C)C)CCC(=O)OC(CC)CSSSCCCCCCCCCCCCCC]

¹H NMR (300 MHz, CDCl3) δ 5.20-4.98 (m, 2H), 3.11-2.98 (m, 4H), 2.86 (t, J=7.2 Hz, 4H), 2.78 (t, J=7.2 Hz, 4H), 2.45 (t, J=7.2 Hz, 6H), 2.37-2.12 (m, 8H), 1.82-1.55 (m, 10H), 1.25 (s, 44H), 0.90 (dt, J=15.9, 7.2 Hz, 12H).

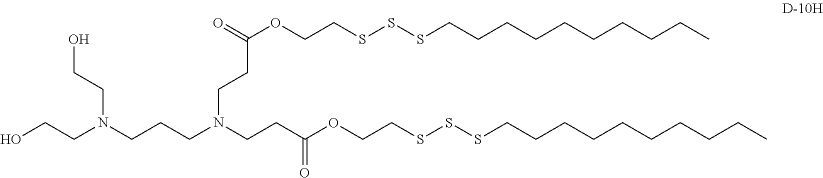 [SMILES: CCCCCCCCCCSSSCCOC(=O)CCN(CCCN(CCO)CCO)CCC(=O)OCCSSSCCCCCCCCCC]

¹H NMR (300 MHz, CDCl3) δ 4.39 (t, J=6.6 Hz, 4H), 3.72-3.60 (m, 4H), 3.09 (t, J 6.6 Hz, 4H), 2.91-2.84 (m, 4H), 2.76 (t, J=6.9 Hz, 4H), 2.72-2.58 (m, 6H), 2.48 (td, J=6.9, 3.0 Hz, 6H), 1.80-1.56 (m, 6H), 1.50-1.14 (m, 28H), 0.87 (t, J=6.6 Hz, 6H). MS (ESI, m/z): [M+H]+ calcd. For C37H75N2O6S6, 835.4; found: 835.7.

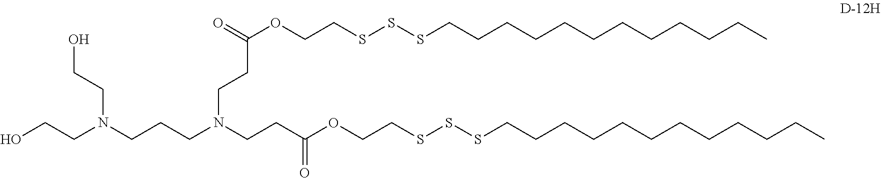 [SMILES: CCCCCCCCCCCCSSSCCOC(=O)CCN(CCCN(CCO)CCO)CCC(=O)OCCSSSCCCCCCCCCCCC]

¹H NMR (300 MHz, CDCl3) δ 4.39 (t, J=6.6 Hz, 4H), 3.65 (t, J=5.1 Hz, 4H), 3.09 (t, J 6.6 Hz, 4H), 2.87 (t, J=7.2 Hz, 4H), 2.76 (t, J=6.9 Hz, 4H), 2.73-2.57 (m, 6H), 2.48 (dq, J 7.1, 3.3 Hz, 6H), 1.71 (ddt, J=19.2, 12.0, 6.6 Hz, 6H), 1.44-1.21 (m, 36H), 0.87 (t, J 6.6 Hz, 6H).

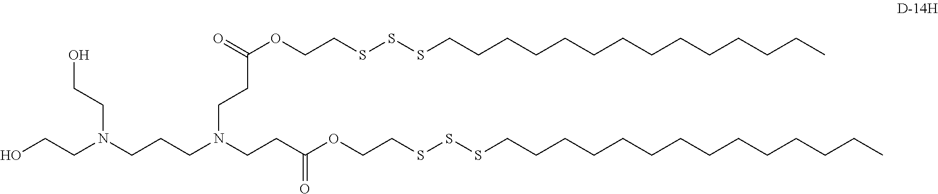 [SMILES: CCCCCCCCCCCCCCSSSCCOC(=O)CCN(CCCN(CCO)CCO)CCC(=O)OCCSSSCCCCCCCCCCCCCC]

¹H NMR (300 MHz, CDCl3) δ 4.40 (t, J=6.6 Hz, 4H), 3.66 (td, J=5.1, 2.4 Hz, 4H), 3.10 (t, J=6.6 Hz, 4H), 2.88 (t, J=7.2 Hz, 4H), 2.77 (t, J=6.9 Hz, 4H), 2.73-2.57 (m, 6H), 2.49 (td, J=6.9, 4.2 Hz, 6H), 1.79-1.62 (m, 6H), 1.45-1.20 (m, 44H), 0.88 (t, J=6.6 Hz, 6H).

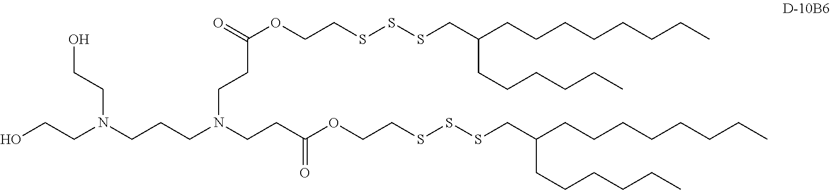 [SMILES: CCCCCCCCC(CCCCCC)CSSSCCOC(=O)CCN(CCCN(CCO)CCO)CCC(=O)OCCSSSCC(CCCCCC)CCCCCCCC]

¹H NMR (300 MHz, CDCl3) δ 4.40 (t, J=6.6 Hz, 4H), 3.67 (q, J=4.5 Hz, 4H), 3.09 (t, J 6.6 Hz, 4H), 2.89 (d, J=6.3 Hz, 4H), 2.80-2.59 (m, 10H), 2.54-2.41 (m, 6H), 1.71 (dp, J 13.2, 6.3 Hz, 4H), 1.41-1.21 (m, 48H), 0.88 (t, J=6.3 Hz, 12H).

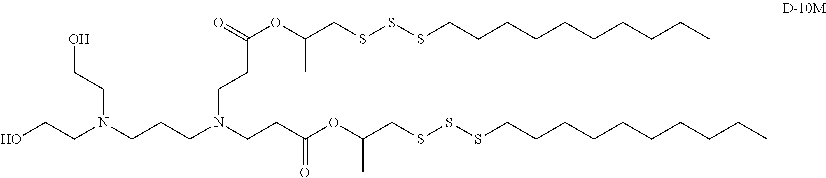 [SMILES: CCCCCCCCCCSSSCC(C)OC(=O)CCN(CCCN(CCO)CCO)CCC(=O)OC(C)CSSSCCCCCCCCCC]

¹H NMR (300 MHz, CDCl3) δ 5.20 (h, J=6.3 Hz, 2H), 3.70-3.59 (m, 4H), 3.12-2.96 (m, 4H), 2.86 (t, J=7.2 Hz, 4H), 2.77 (t, J=7.2 Hz, 4H), 2.71-2.57 (m, 6H), 2.47 (dt, J 10.8, 6.9 Hz, 6H), 1.79-1.57 (m, 6H), 1.44-1.19 (m, 34H), 0.87 (t, J=6.6 Hz, 6H).

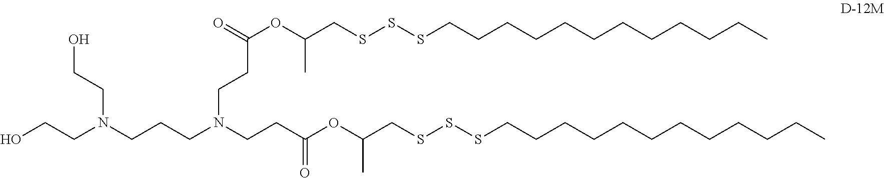 [SMILES: CCCCCCCCCCCCSSSCC(C)OC(=O)CCN(CCCN(CCO)CCO)CCC(=O)OC(C)CSSSCCCCCCCCCCCC]

¹H NMR (300 MHz, CDCl3) δ 5.21 (h, J=6.3 Hz, 2H), 3.68 (dq, J=5.4, 2.4 Hz, 4H), 3.13-2.97 (m, 4H), 2.88 (t, J=7.2 Hz, 4H), 2.83-2.60 (m, 12H), 2.48 (dt, J=14.1, 6.9 Hz, 6H), 1.72 (tt, J=11.1, 5.1 Hz, 6H), 1.45-1.18 (m, 42H), 0.88 (t, J=6.6 Hz, 6H).

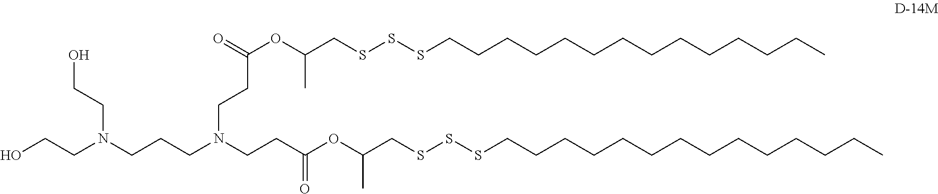 [SMILES: CCCCCCCCCCCCCCSSSCC(C)OC(=O)CCN(CCCN(CCO)CCO)CCC(=O)OC(C)CSSSCCCCCCCCCCCCCC]

¹H NMR (300 MHz, CDCl3) δ 5.21 (h, J=6.3 Hz, 2H), 3.66 (td, J=5.1, 2.4 Hz, 4H), 3.04 (qd, J=13.8, 6.3 Hz, 4H), 2.87 (t, J=7.2 Hz, 4H), 2.83-2.57 (m, 11H), 2.53-2.40 (m, 6H), 1.72 (tt, J=13.5, 6.3 Hz, 6H), 1.45-1.20 (m, 50H), 0.88 (t, J=6.6 Hz, 6H).

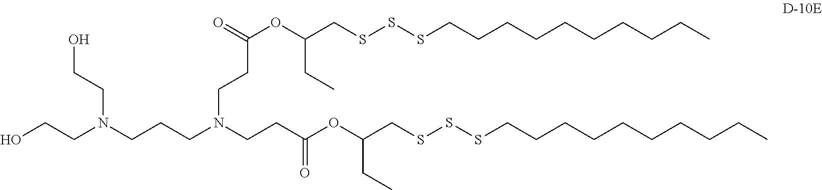 [SMILES: CCCCCCCCCCSSSCC(CC)OC(=O)CCN(CCCN(CCO)CCO)CCC(=O)OC(CC)CSSSCCCCCCCCCC]

¹H NMR (300 MHz, CDCl3) δ 5.10 (ddd, J=11.1, 6.9, 5.4 Hz, 2H), 3.69 (d, J=5.1 Hz, 4H), 3.06 (dd, J=6.0, 2.1 Hz, 4H), 2.87 (t, J=7.2 Hz, 4H), 2.77 (dt, J=10.5, 6.0 Hz, 10H), 2.49 (dt, J=9.6, 6.6 Hz, 6H), 1.85-1.59 (m, 10H), 1.49-1.13 (m, 28H), 0.98-0.83 (m, 12H).

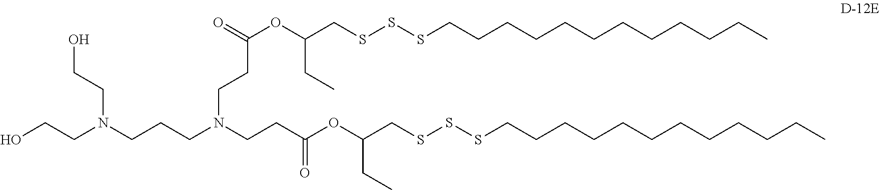 [SMILES: CCCCCCCCCCCCSSSCC(CC)OC(=O)CCN(CCCN(CCO)CCO)CCC(=O)OC(CC)CSSSCCCCCCCCCCCC]

¹H NMR (300 MHz, CDCl3) δ 5.20-5.02 (m, 2H), 3.66 (t, J=5.1 Hz, 4H), 3.13-3.00 (m, 4H), 2.86 (t, J=7.2 Hz, 4H), 2.79 (t, J=7.2 Hz, 4H), 2.75-2.60 (m, 6H), 2.49 (q, J=7.1 Hz, 6H), 1.85-1.57 (m, 10H), 1.52-1.12 (m, 36H), 0.98-0.80 (m, 12H).

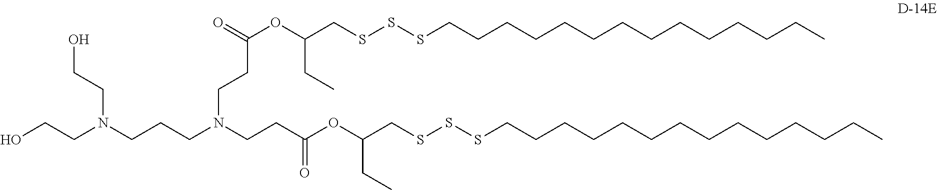 [SMILES: CCCCCCCCCCCCCCSSSCC(CC)OC(=O)CCN(CCCN(CCO)CCO)CCC(=O)OC(CC)CSSSCCCCCCCCCCCCCC]

¹H NMR (300 MHz, CDCl3) δ 5.17-5.04 (m, 2H), 3.68 (q, J=5.7 Hz, 4H), 3.13-3.00 (m, 4H), 2.87 (t, J=7.2 Hz, 4H), 2.84-2.58 (m, 11H), 2.49 (q, J=7.2 Hz, 6H), 1.84-1.61 (m. 10H), 1.26 (s, 44H), 1.00-0.82 (m, 12H).

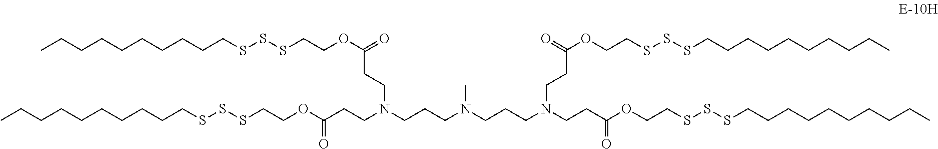 [SMILES: CCCCCCCCCCSSSCCOC(=O)CCN(CCCN(C)CCCN(CCC(=O)OCCSSSCCCCCCCCCC)CCC(=O)OCCSSSCCCCCCCCCC)CCC(=O)OCCSSSCCCCCCCCCC]

¹H NMR (300 MHz, CDCl3) δ 4.39 (t, J=6.6 Hz, 8H), 3.09 (t, J=6.6 Hz, 8H), 2.87 (t, J 7.2 Hz, 8H), 2.75 (t, J=6.9 Hz, 8H), 2.64-2.43 (m, 13H), 2.44-2.04 (m, 6H), 1.86-1.54 (m, 12H), 1.44-1.19 (m, 56H), 0.87 (t, J=6.6 Hz, 12H).

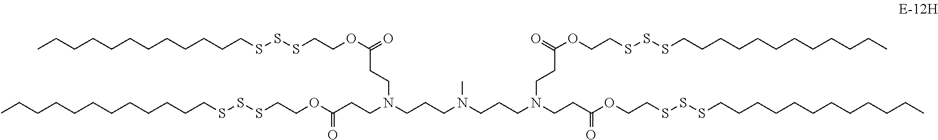 [SMILES: CCCCCCCCCCCCSSSCCOC(=O)CCN(CCCN(C)CCCN(CCC(=O)OCCSSSCCCCCCCCCCCC)CCC(=O)OCCSSSCCCCCCCCCCCC)CCC(=O)OCCSSSCCCCCCCCCCCC]

¹H NMR (300 MHz, CDCl3) δ 4.39 (t, J=6.6 Hz, 8H), 3.09 (t, J=6.6 Hz, 8H), 2.87 (t, J 7.2 Hz, 8H), 2.76 (t, J=6.9 Hz, 8H), 2.60-2.41 (m, 13H), 2.40-2.01 (m, 6H), 1.80-1.59 (m, 12H), 1.43-1.22 (m, 72H), 0.87 (t, J=6.6 Hz, 12H).

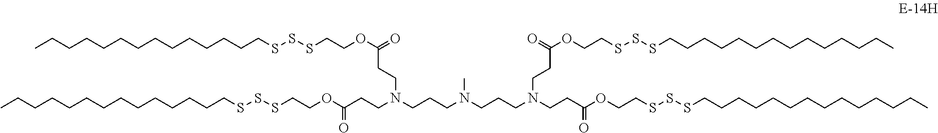 [SMILES: CCCCCCCCCCCCCCSSSCCOC(=O)CCN(CCCN(C)CCCN(CCC(=O)OCCSSSCCCCCCCCCCCCCC)CCC(=O)OCCSSSCCCCCCCCCCCCCC)CCC(=O)OCCSSSCCCCCCCCCCCCCC]

¹H NMR (300 MHz, CDCl3) δ 4.39 (t, J=6.6 Hz, 8H), 3.09 (t, J=6.6 Hz, 8H), 2.88 (t, J 7.2 Hz, 8H), 2.76 (t, J=6.9 Hz, 8H), 2.71-2.54 (m, 3H), 2.46 (t, J=6.9 Hz, 16H), 1.82-1.65 (m, 12H), 1.43-1.19 (m, 88H), 0.87 (t, J=6.6 Hz, 12H).

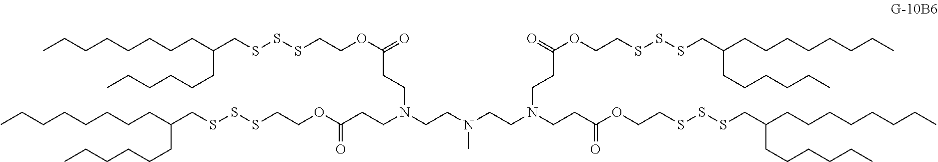 [SMILES: CCCCCCCCC(CCCCCC)CSSSCCOC(=O)CCN(CCC(=O)OCCSSSCC(CCCCCC)CCCCCCCC)CCN(C)CCN(CCC(=O)OCCSSSCC(CCCCCC)CCCCCCCC)CCC(=O)OCCSSSCC(CCCCCC)CCCCCCCC]

¹H NMR (300 MHz, CDCl3) δ 4.38 (t, J=6.6 Hz, 8H), 3.08 (t, J=6.6 Hz, 8H), 2.88 (d, J 6.3 Hz, 8H), 2.75 (t, J=6.9 Hz, 8H), 2.45 (t, J=6.9 Hz, 13H), 2.43-2.20 (m, 6H), 1.71 (tt, J 17.1, 8.4 Hz, 8H), 1.41-1.20 (m, 96H), 0.98-0.74 (m, 24H).

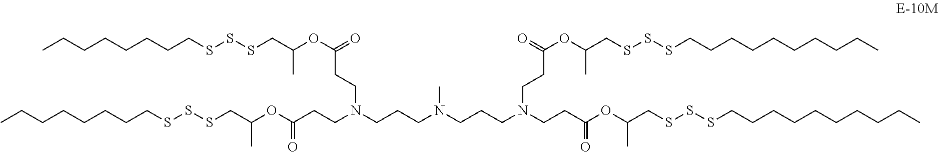 [SMILES: CCCCCCCCCCSSSCC(C)OC(=O)CCN(CCCN(C)CCCN(CCC(=O)OC(C)CSSSCCCCCCCC)CCC(=O)OC(C)CSSSCCCCCCCC)CCC(=O)OC(C)CSSSCCCCCCCCCC]

¹H NMR (300 MHz, CDCl3) δ 5.20 (h, J=6.3 Hz, 4H), 3.04 (qd, J=13.8, 6.3 Hz, 8H), 2.86 (t, J=7.2 Hz, 8H), 2.77 (t, J=7.2 Hz, 8H), 2.43 (t, J=7.2 Hz, 13H), 2.36-1.96 (m, 6H), 1.80-1.51 (m, 12H), 1.46-1.16 (m, 68H), 0.87 (t, J=6.6 Hz, 12H).

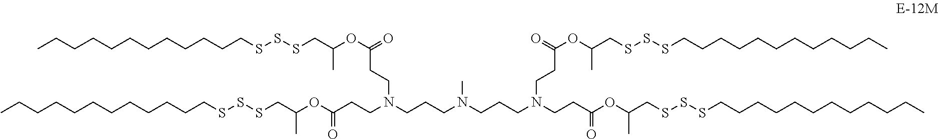 [SMILES: CCCCCCCCCCCCSSSCC(C)OC(=O)CCN(CCCN(C)CCCN(CCC(=O)OC(C)CSSSCCCCCCCCCCCC)CCC(=O)OC(C)CSSSCCCCCCCCCCCC)CCC(=O)OC(C)CSSSCCCCCCCCCCCC]

¹H NMR (300 MHz, CDCl3) δ 5.20 (h, J=6.3 Hz, 4H), 3.04 (qd, J=13.8, 6.0 Hz, 8H), 2.89 (t, J=7.2 Hz, 8H), 2.76 (t, J=7.2 Hz, 10H), 2.65-2.14 (m, 17H), 1.91-1.53 (m, 12H), 1.46-1.18 (m, 84H), 0.87 (t, J=6.6 Hz, 12H).

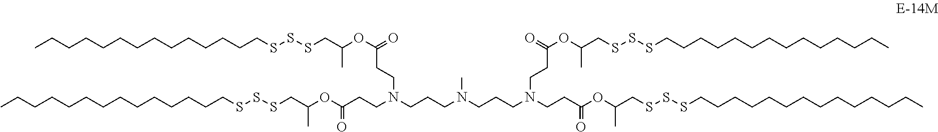 [SMILES: CCCCCCCCCCCCCCSSSCC(C)OC(=O)CCN(CCCN(C)CCCN(CCC(=O)OC(C)CSSSCCCCCCCCCCCCCC)CCC(=O)OC(C)CSSSCCCCCCCCCCCCCC)CCC(=O)OC(C)CSSSCCCCCCCCCCCCCC]

¹H NMR (300 MHz, CDCl3) δ 5.20 (h, J=6.3 Hz, 4H), 3.13-2.96 (m, 8H), 2.87 (t, J 7.2 Hz, 8H), 2.76 (t, J=6.9 Hz, 10H), 2.64-2.26 (m, 15H), 1.85-1.51 (m, 12H), 1.46-1.17 (m, 100H), 0.88 (t, J=6.6 Hz, 12H).

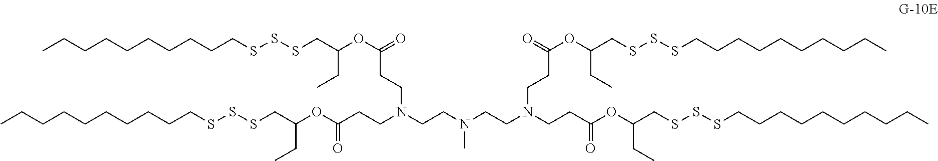 [SMILES: CCCCCCCCCCSSSCC(CC)OC(=O)CCN(CCC(=O)OC(CC)CSSSCCCCCCCCCC)CCN(C)CCN(CCC(=O)OC(CC)CSSSCCCCCCCCCC)CCC(=O)OC(CC)CSSSCCCCCCCCCC]

¹H NMR (300 MHz, CDCl3) δ 5.09 (dt, J=7.2, 5.7 Hz, 4H), 3.14-2.98 (m, 8H), 2.86 (t, J=7.2 Hz, 8H), 2.78 (t, J=7.2 Hz, 8H), 2.45 (t, J=7.2 Hz, 13H), 2.38-2.04 (m, 6H), 1.81-1.56 (m, 20H), 1.44-1.20 (m, 56H), 0.99-0.81 (m, 24H).

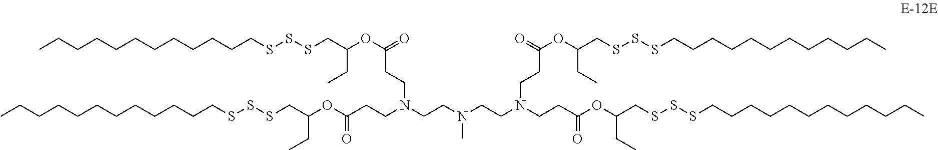 [SMILES: CCCCCCCCCCCCSSSCC(CC)OC(=O)CCN(CCC(=O)OC(CC)CSSSCCCCCCCCCCCC)CCN(C)CCN(CCC(=O)OC(CC)CSSSCCCCCCCCCCCC)CCC(=O)OC(CC)CSSSCCCCCCCCCCCC]

¹H NMR (300 MHz, CDCl3) δ 5.09 (dt, J=6.9, 5.7 Hz, 4H), 3.13-2.99 (m, 8H), 2.87 (t, J=7.2 Hz, 8H), 2.77 (t, J=7.2 Hz, 8H), 2.73-2.38 (m, 15H), 2.38-2.01 (m, 4H), 1.91-1.52 (m, 20H), 1.44-1.18 (m, 72H), 1.00-0.80 (m, 24H).

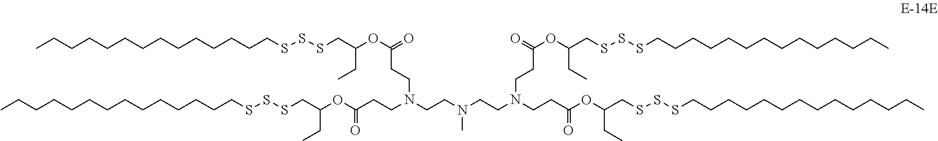 [SMILES: CCCCCCCCCCCCCCSSSCC(CC)OC(=O)CCN(CCC(=O)OC(CC)CSSSCCCCCCCCCCCCCC)CCN(C)CCN(CCC(=O)OC(CC)CSSSCCCCCCCCCCCCCC)CCC(=O)OC(CC)CSSSCCCCCCCCCCCCCC]

¹H NMR (300 MHz, CDCl3) δ 5.09 (dt, J=6.9, 5.4 Hz, 4H), 3.11-3.01 (m, 8H), 2.87 (t, J=7.2 Hz, 8H), 2.78 (t, J=7.2 Hz, 8H), 2.46 (t, J=7.2 Hz, 13H), 2.40-2.12 (m, 6H), 1.79-1.58 (m, 20H), 1.53-1.08 (m, 88H), 0.90 (dt, J=15.9, 7.2 Hz, 24H).

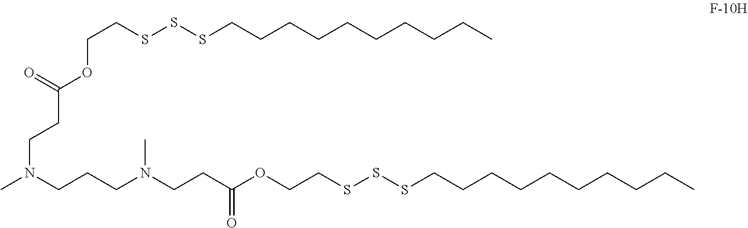 [SMILES: CCCCCCCCCCSSSCCOC(=O)CCN(C)CCCN(C)CCC(=O)OCCSSSCCCCCCCCCC]

¹H NMR (300 MHz, CDCl3) δ 4.42 (t, J=6.6 Hz, 4H), 3.11 (t, J=6.6 Hz, 4H), 2.89 (t, J 7.2 Hz, 4H), 2.72 (t, J=7.2 Hz, 4H), 2.52 (t, J=7.2 Hz, 4H), 2.39 (t, J=7.5 Hz, 4H), 2.25 (s, 6H), 1.82-1.58 (m, 6H), 1.46-1.19 (m, 28H), 0.89 (t, J=6.6 Hz, 6H). MS (ESI, m/z): [M+H]+ calcd. For C35H71N2O4S6, 775.4; found: 775.8.

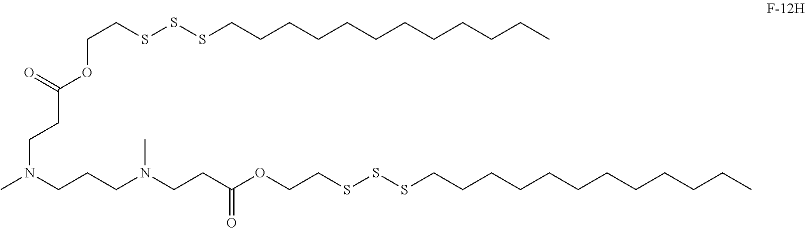 [SMILES: CCCCCCCCCCCCSSSCCOC(=O)CCN(C)CCCN(C)CCC(=O)OCCSSSCCCCCCCCCCCC]

¹H NMR (300 MHz, CDCl3) δ 4.40 (t, J=6.6 Hz, 4H), 3.09 (t, J=6.6 Hz, 4H), 2.87 (t, J 7.2 Hz, 4H), 2.71 (t, J=7.2 Hz, 4H), 2.51 (t, J=7.2 Hz, 4H), 2.38 (t, J=7.2 Hz, 4H), 2.23 (s, 6H), 1.80-1.57 (m, 6H), 1.47-1.16 (m, 36H), 0.87 (t, J=6.6 Hz, 6H).

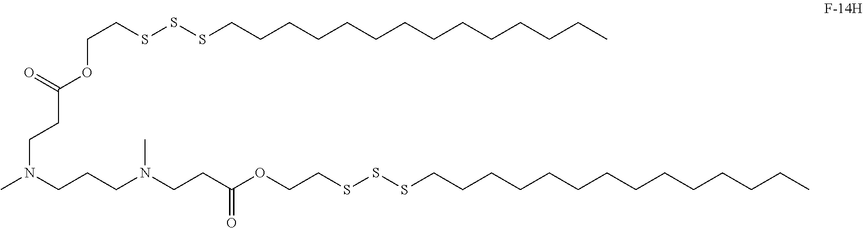 [SMILES: CCCCCCCCCCCCCCSSSCCOC(=O)CCN(C)CCCN(C)CCC(=O)OCCSSSCCCCCCCCCCCCCC]

¹H NMR (300 MHz, CDCl3) δ 4.40 (t, J=6.6 Hz, 4H), 3.09 (t, J=6.6 Hz, 4H), 2.87 (t, J 7.2 Hz, 4H), 2.71 (t, J=7.2 Hz, 4H), 2.50 (t, J=7.2 Hz, 4H), 2.44-2.32 (m, 4H), 2.23 (d, J 0.9 Hz, 6H), 1.80-1.56 (m, 6H), 1.47-1.20 (m, 44H), 0.87 (t, J=6.6 Hz, 6H).

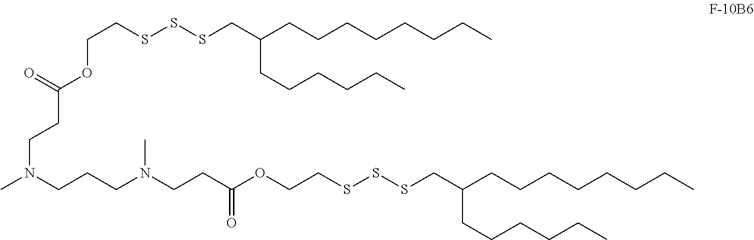 [SMILES: CCCCCCCCC(CCCCCC)CSSSCCOC(=O)CCN(C)CCCN(C)CCC(=O)OCCSSSCC(CCCCCC)CCCCCCCC]

¹H NMR (300 MHz, CDCl3) δ 4.40 (t, J=6.6 Hz, 4H), 3.09 (t, J=6.6 Hz, 4H), 2.89 (d, J 6.3 Hz, 4H), 2.71 (t, J=7.2 Hz, 4H), 2.50 (t, J=7.2 Hz, 4H), 2.37 (t, J=7.5 Hz, 4H), 2.23 (s, 6H), 1.68 (dp, J=32.5, 6.6 Hz, 4H), 1.38-1.19 (m, 48H), 0.93-0.82 (m, 12H).

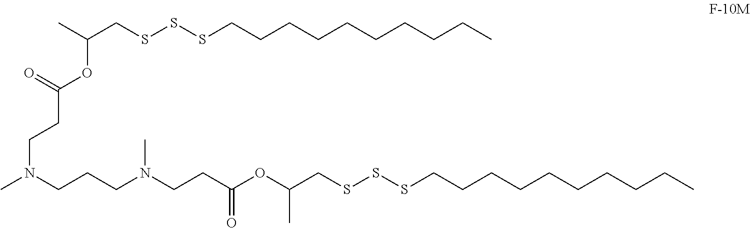 [SMILES: CCCCCCCCCCSSSCC(C)OC(=O)CCN(C)CCCN(C)CCC(=O)OC(C)CSSSCCCCCCCCCC]

¹H NMR (300 MHz, CDCl3) δ 5.28-5.15 (m, 2H), 3.14-2.96 (m, 4H), 2.96-2.82 (m, 4H), 2.82-2.72 (m, 4H), 2.60-2.42 (m, 8H), 2.29 (s, 6H), 1.70 (dp, J=13.2, 7.2 Hz, 6H), 1.45-1.19 (m, 34H), 0.88 (t, J=6.6 Hz, 6H).

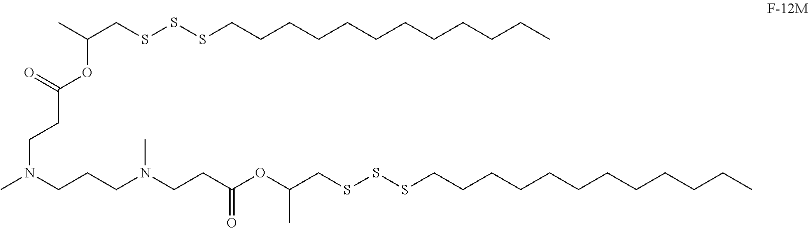 [SMILES: CCCCCCCCCCCCSSSCC(C)OC(=O)CCN(C)CCCN(C)CCC(=O)OC(C)CSSSCCCCCCCCCCCC]

¹H NMR (300 MHz, CDCl3) δ 5.21 (h, J=6.3 Hz, 2H), 3.12-2.96 (m, 4H), 2.86 (t, J 7.2 Hz, 4H), 2.70 (t, J=7.5 Hz, 4H), 2.50-2.43 (m, 4H), 2.37 (t, J=7.2 Hz, 4H), 2.22 (s, 6H), 1.79-1.56 (m, 6H), 1.45-1.17 (m, 42H), 0.87 (t, J=6.6 Hz, 6H).

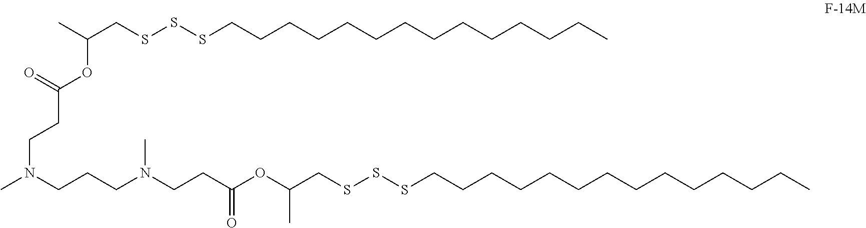 [SMILES: CCCCCCCCCCCCCCSSSCC(C)OC(=O)CCN(C)CCCN(C)CCC(=O)OC(C)CSSSCCCCCCCCCCCCCC]

¹H NMR (300 MHz, CDCl3) δ 5.21 (h, J=6.3 Hz, 2H), 3.04 (qd, J=13.9, 6.3 Hz, 4H), 2.87 (t, J=7.2 Hz, 4H), 2.72 (t, J=7.2 Hz, 4H), 2.49 (t, J=7.5 Hz, 4H), 2.44-2.32 (m, 4H), 2.24 (d, J=1.3 Hz, 6H), 1.79-1.59 (m, 6H), 1.44-1.19 (m, 50H), 0.87 (t, J=6.6 Hz, 6H).

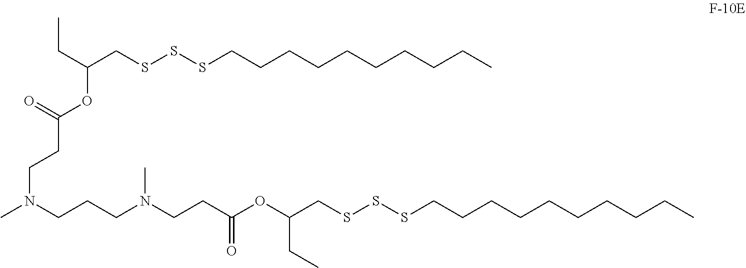 [SMILES: CCCCCCCCCCSSSCC(CC)OC(=O)CCN(C)CCCN(C)CCC(=O)OC(CC)CSSSCCCCCCCCCC]

¹H NMR (300 MHz, CDCl3) δ 5.18-5.01 (m, 2H), 3.12-3.01 (m, 4H), 2.86 (t, J=7.2 Hz, 4H), 2.71 (t, J=6.9 Hz, 4H), 2.49 (t, J=7.2 Hz, 4H), 2.37 (t, J=7.5 Hz, 4H), 2.23 (s, 6H), 1.86-1.53 (m, 10H), 1.53-1.01 (m, 28H), 0.99-0.80 (m, 12H).

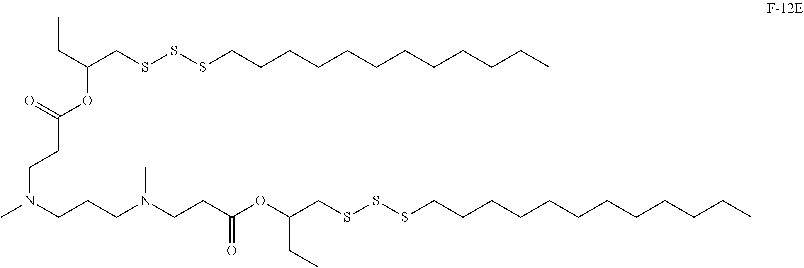 [SMILES: CCCCCCCCCCCCSSSCC(CC)OC(=O)CCN(C)CCCN(C)CCC(=O)OC(CC)CSSSCCCCCCCCCCCC]

¹H NMR (300 MHz, CDCl3) δ 5.17-5.05 (m, 2H), 3.14-2.99 (m, 4H), 2.87 (t, J=7.2 Hz, 4H), 2.80-2.68 (m, 4H), 2.51 (t, J=7.2 Hz, 4H), 2.39 (d, J=7.8 Hz, 4H), 2.24 (s, 6H), 1.85-1.54 (m, 10H), 1.46-1.17 (m, 36H), 0.97-0.81 (m, 12H).

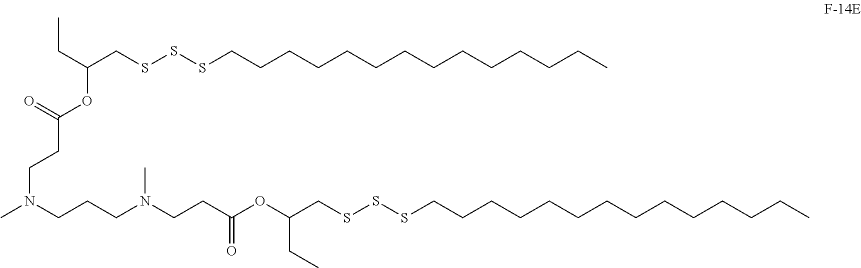 [SMILES: CCCCCCCCCCCCCCSSSCC(CC)OC(=O)CCN(C)CCCN(C)CCC(=O)OC(CC)CSSSCCCCCCCCCCCCCC]

¹H NMR (300 MHz, CDCl3) δ 5.16-5.05 (m, 2H), 3.14-2.98 (m, 4H), 2.87 (t, J=7.2 Hz, 4H), 2.81-2.68 (m, 4H), 2.51 (t, J=7.2 Hz, 4H), 2.41 (d, J=7.2 Hz, 4H), 2.24 (s, 6H), 1.84-1.59 (m, 10H), 1.49-1.14 (m, 44H), 0.97-0.83 (m, 12H).

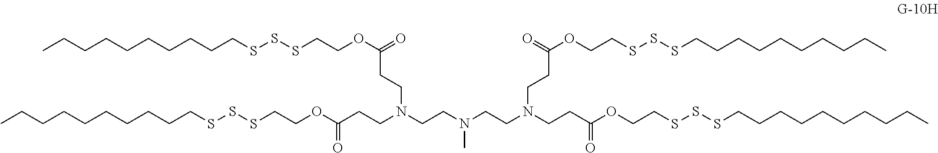 [SMILES: CCCCCCCCCCSSSCCOC(=O)CCN(CCC(=O)OCCSSSCCCCCCCCCC)CCN(C)CCN(CCC(=O)OCCSSSCCCCCCCCCC)CCC(=O)OCCSSSCCCCCCCCCC]

¹H NMR (300 MHz, CDCl3) δ 4.39 (t, J=6.6 Hz, 8H), 3.09 (t, J=6.6 Hz, 8H), 2.88 (t, J 7.2 Hz, 8H), 2.80 (t, J=7.2 Hz, 8H), 2.65-2.50 (m, 8H), 2.47 (t, J=7.2 Hz, 8H), 2.25 (s, 3H), 1.80-1.65 (m, 8H), 1.47-1.21 (m, 56H), 0.88 (t, J=6.6 Hz, 12H).

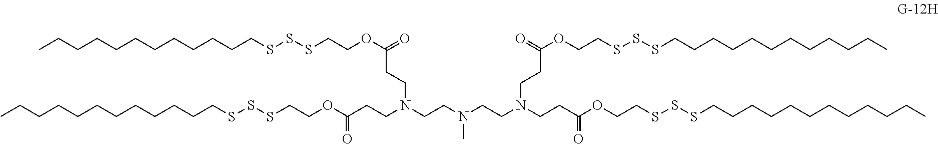 [SMILES: CCCCCCCCCCCCSSSCCOC(=O)CCN(CCC(=O)OCCSSSCCCCCCCCCCCC)CCN(C)CCN(CCC(=O)OCCSSSCCCCCCCCCCCC)CCC(=O)OCCSSSCCCCCCCCCCCC]

¹H NMR (300 MHz, CDCl3) δ 4.39 (t, J=6.6 Hz, 8H), 3.09 (t, J=6.6 Hz, 8H), 2.87 (t, J 7.2 Hz, 8H), 2.80 (t, J=7.2 Hz, 8H), 2.63-2.51 (m, 6H), 2.47 (t, J=6.9 Hz, 10H), 2.24 (s, 3H), 1.80-1.63 (m, 8H), 1.44-1.16 (m, 72H), 0.87 (t, J=6.6 Hz, 12H).

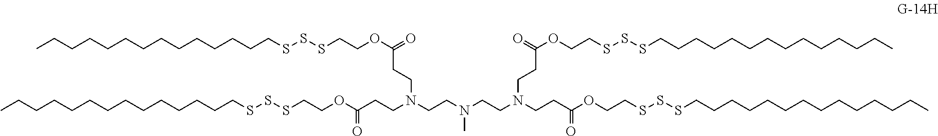 [SMILES: CCCCCCCCCCCCCCSSSCCOC(=O)CCN(CCC(=O)OCCSSSCCCCCCCCCCCCCC)CCN(C)CCN(CCC(=O)OCCSSSCCCCCCCCCCCCCC)CCC(=O)OCCSSSCCCCCCCCCCCCCC]

¹H NMR (300 MHz, CDCl3) δ 4.39 (t, J=6.6 Hz, 8H), 3.10 (t, J=6.6 Hz, 8H), 2.88 (t, J 7.2 Hz, 8H), 2.80 (t, J=7.2 Hz, 8H), 2.63-2.53 (m, 6H), 2.47 (t, J=6.9 Hz, 10H), 2.25 (s, 3H), 1.80-1.66 (m, 8H), 1.54-1.10 (m, 88H), 0.88 (t, J=6.6 Hz, 12H).

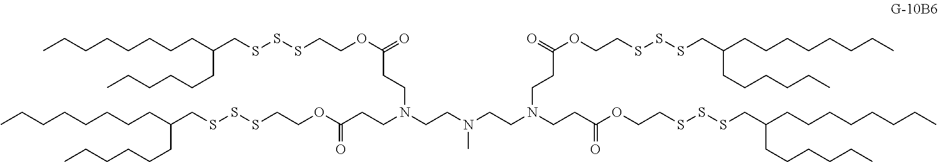 [SMILES: CCCCCCCCC(CCCCCC)CSSSCCOC(=O)CCN(CCC(=O)OCCSSSCC(CCCCCC)CCCCCCCC)CCN(C)CCN(CCC(=O)OCCSSSCC(CCCCCC)CCCCCCCC)CCC(=O)OCCSSSCC(CCCCCC)CCCCCCCC]

¹H NMR (300 MHz, CDCl3) δ 4.46-4.34 (m, 8H), 3.09 (t, J=6.6 Hz, 8H), 2.90 (d, J 6.3 Hz, 8H), 2.81 (td, J=6.9, 3.3 Hz, 8H), 2.74-2.44 (m, 16H), 2.28 (s, 3H), 1.73 (h, J=6.0 Hz, 4H), 1.44-1.18 (m, 96H), 1.06-0.74 (m, 24H).

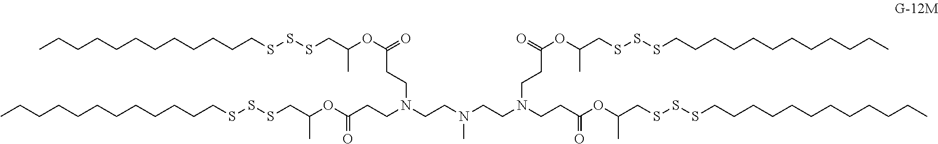 [SMILES: CCCCCCCCCCCCSSSCC(C)OC(=O)CCN(CCC(=O)OC(C)CSSSCCCCCCCCCCCC)CCN(C)CCN(CCC(=O)OC(C)CSSSCCCCCCCCCCCC)CCC(=O)OC(C)CSSSCCCCCCCCCCCC]

¹H NMR (300 MHz, CDCl3) δ 5.21 (qd, J=6.9, 4.5 Hz, 4H), 3.02 (ddd, J=15.8, 13.8, 6.3 Hz, 8H), 2.87 (t, J=7.2 Hz, 8H), 2.83-2.71 (m, 8H), 2.65-2.53 (m, 7H), 2.46 (q, J=7.8 Hz, 9H), 2.26 (d, J=4.5 Hz, 3H), 1.81-1.62 (m, 8H), 1.44-1.21 (m, 84H), 0.88 (t, J=6.6 Hz, 12H).

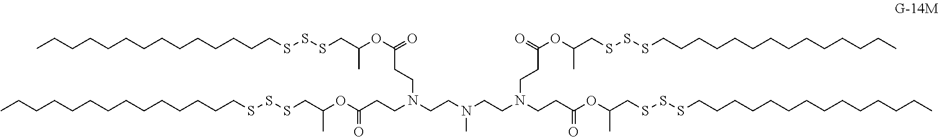 [SMILES: CCCCCCCCCCCCCCSSSCC(C)OC(=O)CCN(CCC(=O)OC(C)CSSSCCCCCCCCCCCCCC)CCN(C)CCN(CCC(=O)OC(C)CSSSCCCCCCCCCCCCCC)CCC(=O)OC(C)CSSSCCCCCCCCCCCCCC]

¹H NMR (300 MHz, CDCl3) δ 5.27-5.15 (m, 4H), 3.12-2.96 (m, 8H), 2.91-2.83 (m, 8H), 2.76 (dt, J=20.4, 6.6 Hz, 8H), 2.56 (dd, J=9.6, 5.1 Hz, 7H), 2.46 (q, J=7.2 Hz, 9H), 2.24 (s, 3H), 1.80-1.65 (m, 8H), 1.44-1.21 (m, 100H), 0.88 (t, J=6.6 Hz, 12H).

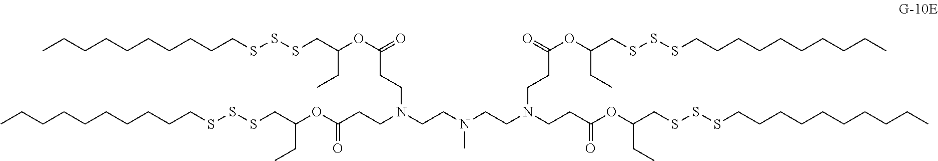 [SMILES: CCCCCCCCCCSSSCC(CC)OC(=O)CCN(CCC(=O)OC(CC)CSSSCCCCCCCCCC)CCN(C)CCN(CCC(=O)OC(CC)CSSSCCCCCCCCCC)CCC(=O)OC(CC)CSSSCCCCCCCCCC]

¹H NMR (300 MHz, CDCl3) δ 5.19-5.05 (m, 4H), 3.10-3.02 (m, 8H), 2.84 (dt, J 16.2, 7.5 Hz, 16H), 2.58 (s, 7H), 2.47 (t, J=6.9 Hz, 9H), 2.27 (d, J=7.2 Hz, 3H), 1.83-1.61 (m, 16H), 1.49-1.15 (m, 56H), 0.98-0.84 (m, 24H).

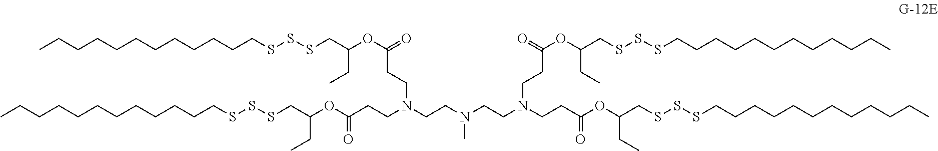 [SMILES: CCCCCCCCCCCCSSSCC(CC)OC(=O)CCN(CCC(=O)OC(CC)CSSSCCCCCCCCCCCC)CCN(C)CCN(CCC(=O)OC(CC)CSSSCCCCCCCCCCCC)CCC(=O)OC(CC)CSSSCCCCCCCCCCCC]

¹H NMR (300 MHz, CDCl3) δ 5.19-5.04 (m, 4H), 3.12-3.00 (m, 8H), 2.91-2.71 (m 16H), 2.57 (s, 7H), 2.47 (t, J=7.2 Hz, 9H), 2.26 (s, 3H), 1.83-1.61 (m, 16H), 1.26 (s, 72H), 0.99-0.83 (m, 24H).

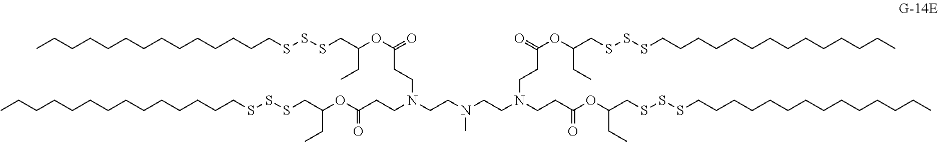 [SMILES: CCCCCCCCCCCCCCSSSCC(CC)OC(=O)CCN(CCC(=O)OC(CC)CSSSCCCCCCCCCCCCCC)CCN(C)CCN(CCC(=O)OC(CC)CSSSCCCCCCCCCCCCCC)CCC(=O)OC(CC)CSSSCCCCCCCCCCCCCC]

¹H NMR (300 MHz, CDCl3) δ 5.17-5.06 (m, 4H), 3.08-3.01 (m, 8H), 2.91-2.75 (m, 16H), 2.70-2.55 (m, 7H), 2.47 (t, J=7.2 Hz, 10H), 2.27 (s, 3H), 1.81-1.64 (m, 16H), 1.58-1.00 (m, 88H), 0.96-0.85 (m, 24H).

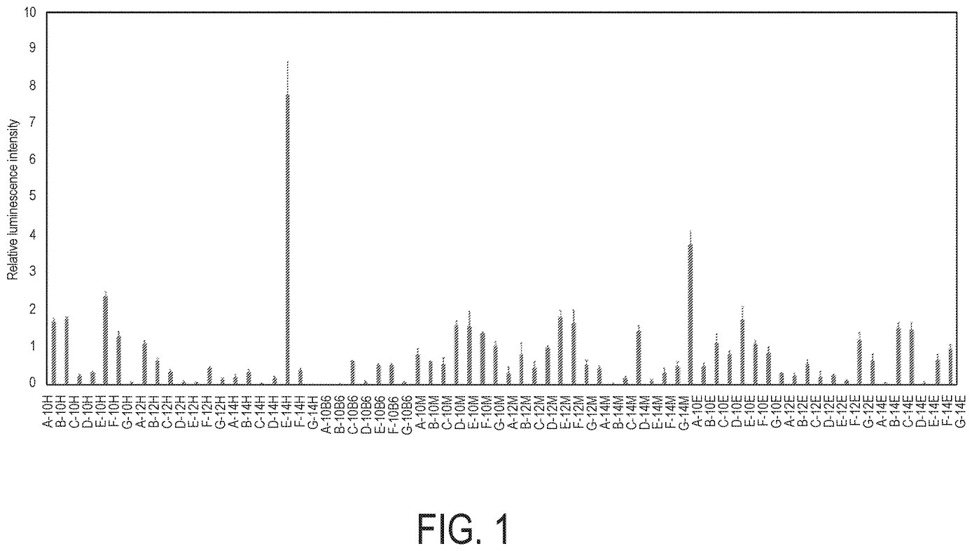

In vitro delivery efficiency of trisulfide lipid nanoparticles in Hep3B cells. 2×10⁴ Hep3B cells were seeded per well in white-bottom 96-well plates 1 day before the treatment. Cells were then incubated with firefly luciferase mRNA-trisulfide lipid nanoparticles for 18 hours (50 ng mRNA per well, in triplicate). The luminescence intensity was measured and normalized to that of Lipofectamine™ 3000 reagent treatment. (FIG. 1).

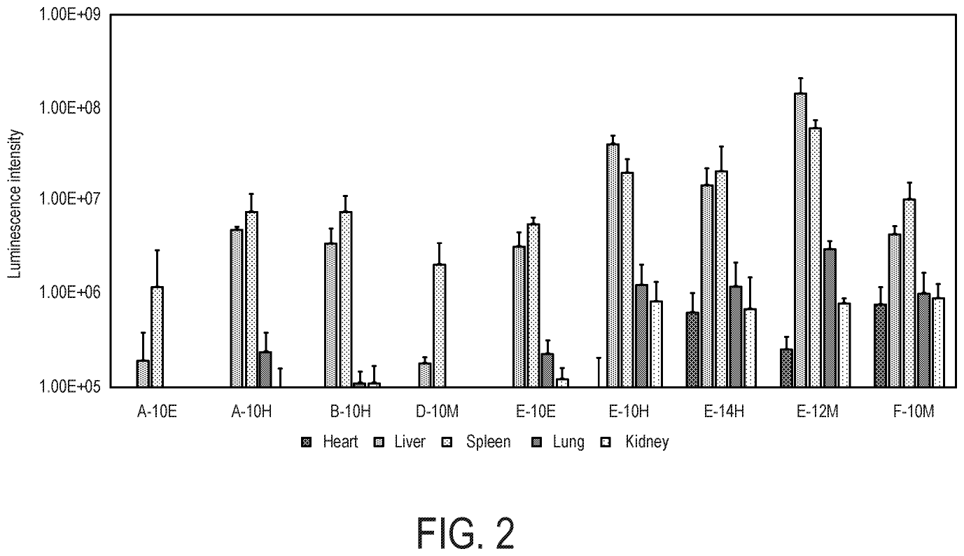

In vivo delivery efficacy of trisulfide lipid nanoparticles in C57BL/6 mice. C57BL/6 mice were injected intravenously with firefly luciferase mRNA-trisulfide lipid nanoparticles (mRNA dose of 0.3 mg/kg, three mice in each group). 6 hours after intravenous injection, 150 μl of XenoLight d-luciferin was injected to each mouse through intraperitoneal injection. 8 minutes after the injection of the substrate, mice were euthanized, and major organs were collected for imaging. (FIG. 2).

### Example 2. Lipid Nanoparticle (LNP) Delivery of mRNA for Modulating the Microenvironment to Treat Acute Diabetic Wounds

Diabetic wounds, a common complication in patients with hyperglycemia, are associated with high morbidity, mortality, and recurrence rates, leading to substantial global economic losses.³ Although wound off-loading or growth factor therapy has demonstrated significant reductions in healing time for chronic wounds in clinical trials, their widespread use is hindered by financial costs and potential side effects.⁴⁻⁶ There is an urgent need to develop a more effective, safe, and convenient method for managing diabetic wounds.

A great challenge for diabetic wound healing is due to its complex microenvironment.^{7, 8} The etiology of chronic, non-healing diabetic wounds is multi-faceted, involving the uncontrolled accumulation of reactive oxygen species (ROS) and sustained inflammation.⁹⁻¹¹ The abnormal accumulation of ROS in the pathological microenvironment in diabetic wounds is responsible for excessive oxidative stress and deficient angiogenesis, leading to delayed healing.^{12, 13} Persistent inflammation in wounds is due to the presence of M1 phenotype macrophages in the microenvironment.¹⁴⁻¹⁶ The M1 phenotype macrophages in diabetic wounds continue to release pro-inflammatory cytokines, such as tumor necrosis factor-alpha (TNF-α), while M2 phenotype macrophages can promote wound repair by anti-inflammatory responses.^{17, 18} Thus, modulating the wound inflammation microenvironment, including ROS mitigation and phenotype regulation, could be a useful approach for treating diabetic wound healing.

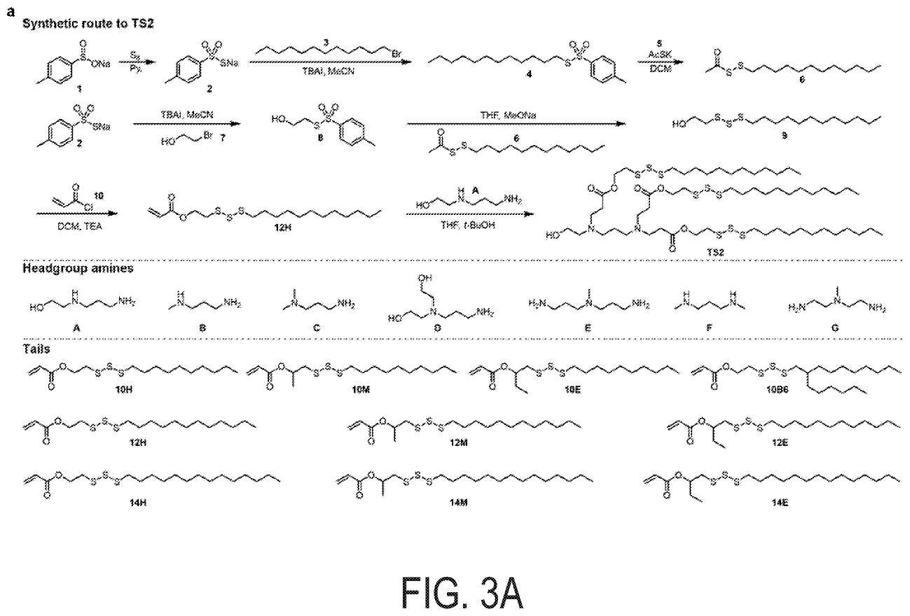

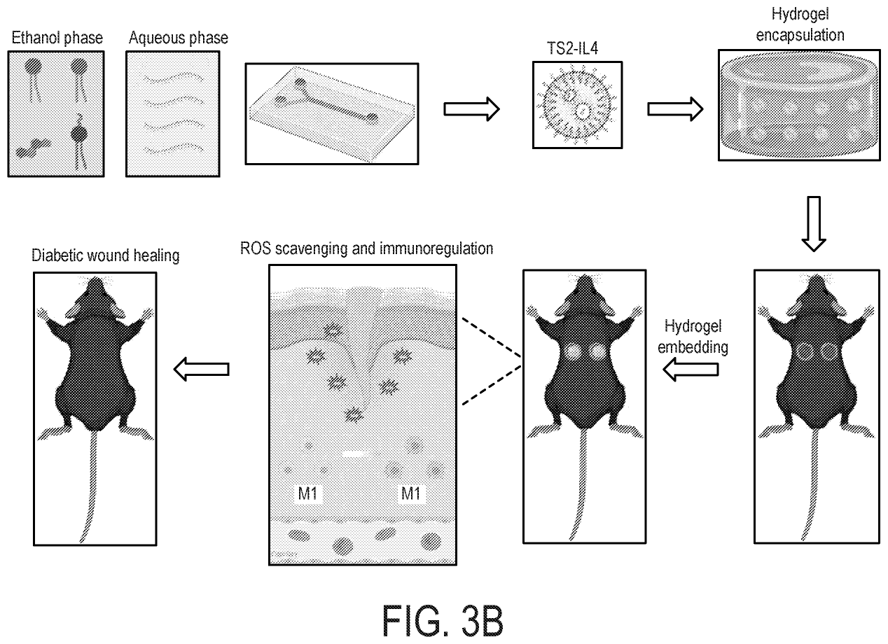

Lipid nanoparticle (LNP)-mRNA formulations have been developed and are under clinical evaluation for the prevention and treatment of virus infections, cancer, and genetic diseases based on their unique biocompatibility, biodegradability, and delivery efficiency.¹⁹⁻²² Hence, it was hypothesized that the development of a bioresponsive LNP-mRNA formulation capable of delivering specific therapeutic proteins may reshape the wound microenvironment for the treatment of diabetic wounds. Inspired by previous reports on ROS-triggered sulfur atom oxidation,²³⁻²⁵ a library of ionizable lipids was designed and synthesized containing a ROS-sensitive trisulfide linker using a combinatorial approach (FIG. 3A). The ROS-scavenging ability of these trisulfide-derived lipids formulated LNP (TS LNP) may restore the redox balance in the pathological microenvironment of diabetic wounds. Additionally, previous studies have shown that interleukin-4 (IL4) can promote macrophage polarization towards the M2 phenotype and have been evaluated in preclinical trials.²⁶⁻²⁸ Thus, the development of TS LNPs capable of delivering IL4 mRNA has the possibility to promote diabetic wound healing. Specifically, in this example, TS LNPs were applied to encapsulate IL4 mRNA. Then, the TS-IL4 mRNA formulation can be loaded into a hydrogel, which is then used to treat diabetic wounds. After ROS scavenging and immunoregulation of macrophage polarization by TS-IL4 LNP, the wound microenvironment is reshaped, ultimately achieving diabetic wound healing (FIG. 3B).

### Material and Methods

### Chemicals and Reagents

1,2-dioleoyl-sn-glycero-3-phosphoethanolamine (DOPE), cholesterol, 1,2-dimyristoyl-rac-glycero-3-methoxypolyethylene glycol-2000 (DMG-PEG_{2K}), D-Lin-MC3-DMA (MC3) and 1,2-Distearoyl-sn-glycero-3-phosphoethanolamine (DSPE) was purchased from Avanti Polar Lipids. All other chemicals and solvents were purchased from Sigma-Aldrich.

### Cell Culture

Murine cell line RAW264.7 cells was purchased from ATCC and cultured in Dulbecco's modified Eagle medium (DMEM) (Corning) supplemented with 10% FBS in a 5% CO₂ atmosphere at 37° C. Fibroblasts were purchased from ATCC and cultured in Eagle's Minimum Essential Medium (EMEM) supplemented with 10% FBS in a 5% CO₂ atmosphere.

### RNA Synthesis

The linear dsDNA of firefly luciferase (FLuc) and IL4 were obtained from Integrated DNA Technologies. The pUC19 vector was used for Golden Gate assembly to generate the corresponding plasmids. mRNAs were synthesized by previously reported methods.⁴⁶

### Preparation and Characterization of LNPs

The LNP-mRNA formulations were prepared by mixing an ethanol solution containing ionizable lipids, DOPE, cholesterol, and DMG-PEG₂ₖ, and a citrate solution (pH=3) containing mRNA via NanoAssemblr (Precision NanoSystems). Size, polydispersity index (PDI), and zeta potential were measured by NanoZS Zetasizer (Malvern). The encapsulation efficiency was quantified via a RiboGreen assay. The morphology was observed by Glacios cryo-TEM (Thermo Scientific). For the initial screening, the LNP-mRNA was prepared by mixing ionizable lipids, DOPE, cholesterol and DMG-PEG_{2K} at a molar ratio of 20:30:40:0.75, followed by the addition of FLuc mRNA into the lipid compounds of 10:1. In the first round of optimization study, the molar ratio of each lipid component was adjusted based on an L16 (4)⁴ orthogonal table. In the second round of optimization study, the mRNA to compound ratio was optimized based on an L12 (3)⁴ orthogonal table. The MC3, ALC-0315, and SM102 LNP-mRNA formulations were prepared.²⁰ mRNA delivery efficiency was measured 18 h post-treatment by adding Bright-Glo luciferase substrate (Promega) to cells and quantified using Cytation 5 (Biotek).

### Assessment of Macrophage Polarization In Vitro

To evaluate the TS-LNPs on the polarization of macrophages. RAW274.7 cells were pretreated with LPS (10 μg mL⁻¹) for 24 h. Then, the cells were seeded in a 6-well plate with 5×10⁴ cells per well. After overnight incubation, the cells were treated with TS2-IL4 LNP, MC3-IL4 LNP, and free IL4 mRNA for 48 h. Cells treated with LPS alone were used as the control. After all treatments, the cell was collected and harvested, and then stained with anti-mouse CD86 (Biolegend) and anti-mouse CD206 antibody (Biolegend) for 30 mins for flow cytometry analysis. Data were analyzed by FlowJo.

### Assessment of Intracellular ROS Scavenging

The intracellular ROS scavenging capacity of the TS2 LNP was evaluated on fibroblast cells.⁴⁷ Briefly, cells were seeded in a 12-well plate and cultured for 24 h. The cells were then co-incubated with H₂O₂ (200 μM) and either TS2 LNP or MC3 LNP for another 24 h. After being rinsed with PBS three times, cells were incubated with DCFDA (10 M) for 20 min. The intracellular ROS level was evaluated by flow cytometry. Meanwhile, the fluorescence in cells was photographed by a confocal fluorescence microscope. Similarly, cell viability was evaluated by a fluorescence-based Live/Dead kit and MTT Kit according to the manufacturer's instructions.

### Assessment of Wound Healing in Diabetic Mice

All male db/db mice (BKS.Cg-Dock7m+/+Leprdb/J, 00642, 12 weeks) were purchased from the Jackson Laboratory. All mouse studies were approved by the Institutional Animal Care and Use Committee (IACUC) at the Icahn School of Medicine at Mount Sinai. The full-thickness wound model was constructed as previously described. Briefly, db/db mice were anesthetized using isoflurane and then wounds were created on shaved back skin by using 7-mm punch biopsy punches (Integra Miltex).

To examine the delivery and expression of TS2-mRNA encoding Fluc, mice were randomly divided into three groups and treated with FLuc mRNA, MC3-FLuc LNP, and TS2-FLuc LNP. The hydrogel (Advanced BioMatrix) was prepared and loaded with FLuc, MC3-FLuc, and TS2-FLuc according to the manufacturer's instructions. Hydrogel was mixed with LNP at a 1:1 volume ratio for 15 minutes before treatment. Subsequently, 20 μL of the hydrogel mixture, containing 2 g FLuc, was administered to the wound. After 6 hours, the in vivo bioluminescence imaging was performed.

To evaluate the process of wound healing, the mice were applied with free IL4 mRNA, MC3-IL4 LNP, TS2-IL4 LNP, SM102-IL4 LNP, and TS2 LNP. The hydrogel mixture was prepared as described above, and 20 μL of the hydrogel solution, containing 2 g of IL4 mRNA was administered to the wound. After treatment, the wound area was affixed with a 7-mm donut-shaped silicone splint and interrupted using 6-0 nylon sutures (Ethicon). The wound area was measured and captured with a digital camera on the day of surgery, and every 3 days until the wound was completely healed.

### Histology and Immunofluorescence

After treatment, all the regenerated dorsal skins were collected, One-half was snap-frozen and stored at −80° C., while the other half was fixed in 4% paraformaldehyde and processed for paraffin embedding. Masson's trichrome staining and H&E were performed on 5 μm sections. A Nikon microscope was used to observe the stained sections and collect images while measuring the thickness of the epidermis.

For in vivo immunofluorescence staining, de-paraffinization and antigen retrieval (citrate buffer, 10 mM, 0.05% Tween 20, pH 6.0) of the paraffin-embedded mice wound sections were performed as previously described. The sections were incubated with rabbit mAB CD31 (1:200, Abcam ab222783), goat pAB α-SMA (1:200, Abcam ab7817), CD206 (1:100, Biolegend) and CD86 (1:100, Biolegend) at 4° C. overnight. Followed by PBS wash, secondary antibodies were added and incubated for 1 h at 37° C.: Alexa Fluor 594 donkey anti-rabbit IgG (1:200, Invitrogen, A21207) and Alexa Fluor 488 donkey anti-rabbit IgG (1:800, Invitrogen, A21206). Nuclear counterstaining was performed with 4′,6-diamidino-2-phenylindole (DAPI) (Invitrogen). Fluorescent images of the slices were taken using a fluorescence microscope and quantification was performed by ImageJ.

### Assessment of IL4 Expression

For in vitro assays, 1×10⁶ RAW264.7 cells were seeded into each well of a 6-well plate and treated with 0.2 μg TS2-IL4 LNP. At different time points, the cell supernatant was collected by centrifugation and the IL4 concentration was determined by ELISA kit (RayBiotech) according to the manufacturer's protocol.

### Skin Dissociation and Flow Cytometry

For the analysis of macrophages at the wound site, wound tissues were collected and analyzed by flow cytometry according to the previously reported method. Briefly, mouse skin composed of wound and approximately 0.5 mm of peri-wound tissue was excised and kept on ice-cold sterile PBS. Afterward, the skin tissues were finely minced and placed for 30 min at 37° C. in a shaker in a digesting enzyme cocktail of 2 mg ml⁻¹ Collagenase P (Roche), 2 mg ml⁻¹ Dispase (Sigma), and 1 mg ml⁻¹ DNase I (Stemcell Technologies) in DMEM (Gibco) with 10% FBS and 1% P/S. During the digestion, glass pipettes were used to break down the extracellular matrix every 10 min. Single-cell suspensions were passed through a 40 μm cell strainer. After PBS wash and cell counting, single-cell suspensions were incubated with fluorescence-labeled CD45, CD11b, F4/80, CD206, and CD86 antibodies. All samples were detected using the flow cytometer and analyzed with FlowJo.

### Statistics

Statistical analysis was performed using Graph Pad Prism software. In the statistical analysis for comparison between multiple data groups, one-way analysis of variance (ANOVA) followed by Tukey's comparison tests were conducted with thresholds of *P<0.05, **P<0.01, ***P<0.001 and ****P<0.0001. In the statistical analysis for comparison between two data groups, unpaired Student's t tests were used with thresholds of *P<0.05, **P<0.01, ***P<0.001 and ****P<0.0001.

### Results

Synthesis and Formulation of Trisulfide-Derived LNPs (TS LNP) for mRNA Delivery.

As shown in FIG. 3A, the synthetic process of trisulfide-derived ionizable lipids commenced with the preparation of ten hydrophobic tails featuring both ester and trisulfide linkers. Firstly, alkyl thiosulfonates 4 and 8 were prepared by substitution reactions of sodium 4-methylbenzenesulfonothioate 2 with alkyl bromide 3 and 7, respectively. Then, the replacement of alkyl thiosulfonothioate 4 with potassium thioacetate 5 in dichloromethane afforded disulfanyl acetate 6. The introduction of trisulfide linker in alcohol 9 was realized via reactions between disulfanyl acetates 6 and alkyl thiotosylate 8 in the presence of sodium methoxide according to a previously reported procedure.²⁴ Finally, acrylation of alcohol 9 gave acrylate 12H which underwent Michael addition reaction with amine A to give the final product TS2. Both linear and branched tails were incorporated to evaluate their effects on mRNA delivery efficiency. Acrylates tails were named according to the length of hydrocarbon domains and ester types. Tails 10H, 12H, and 14H, featured primary ester linkers, whereas tails 10M, 10B6, 12M, 14M, 10E, 12E, and 14E had the primary esters replaced with branched esters (M refers to methyl branch, E refers to ethyl branch). In total, a diverse library of 70 ROS-sensitive trisulfide lipids were obtained via a combinatorial Aza-Michael addition reaction involving seven aliphatic amines and ten acrylate hydrophobic tails (Table 3), following a modified procedure previously documented.²⁹ The crude product was purified via silica gel flash chromatography, and the chemical structures of lipids were confirmed by ¹H NMR spectra and mass spectroscopy.

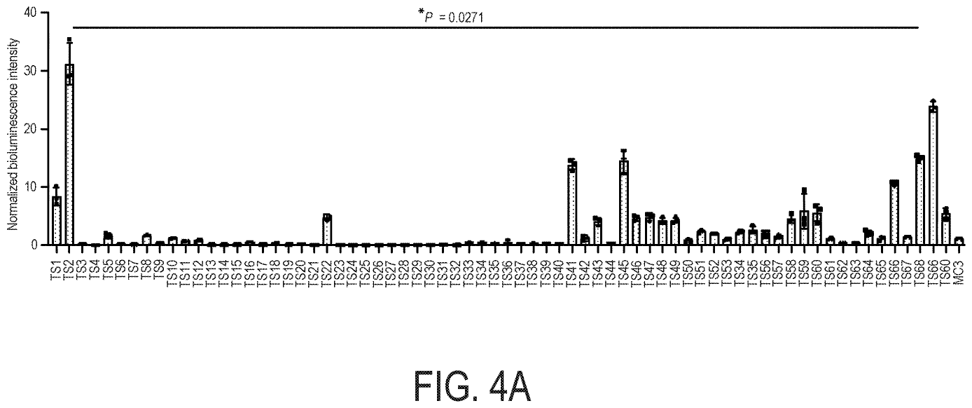

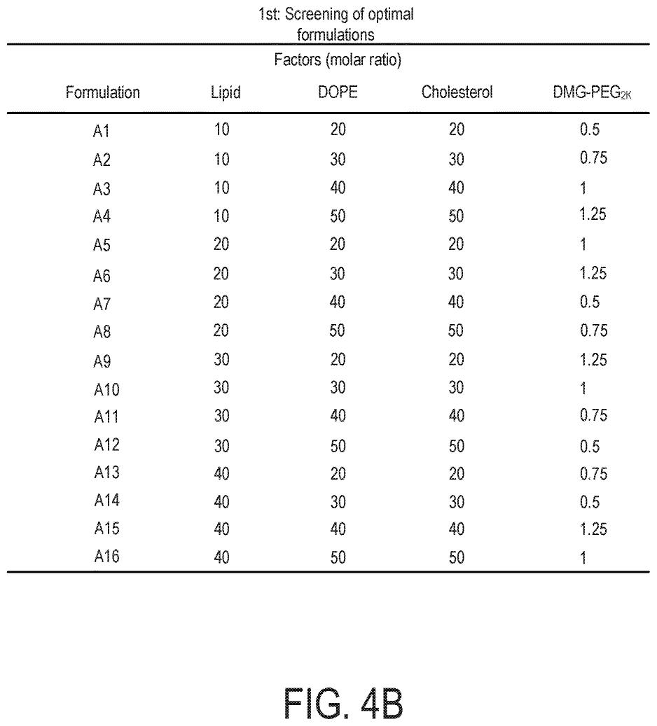

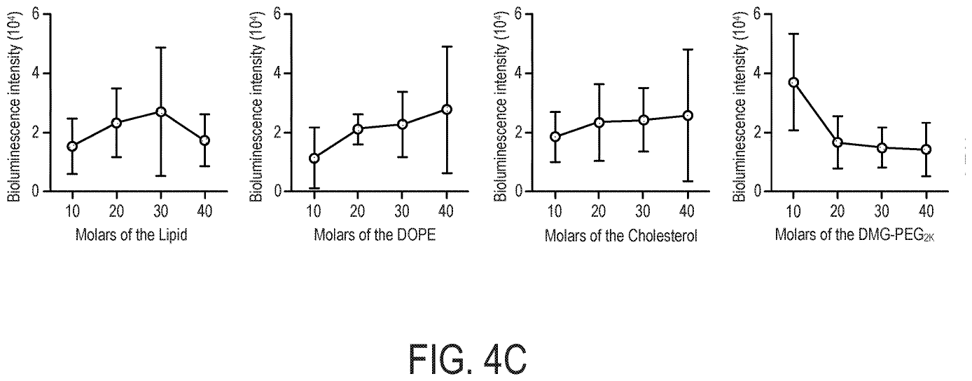

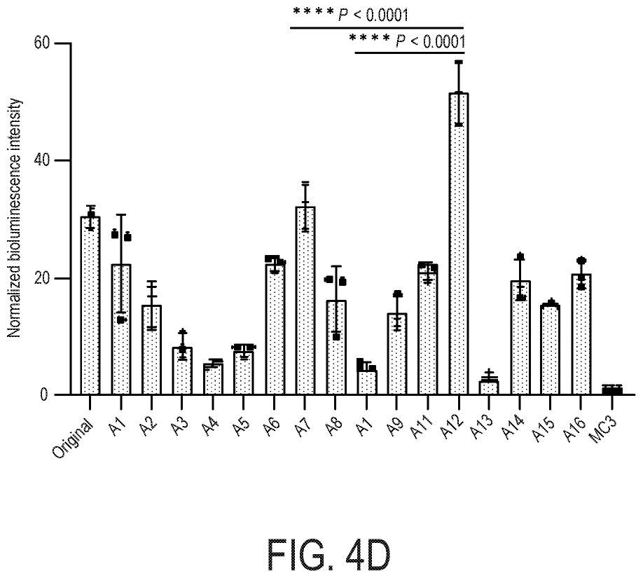

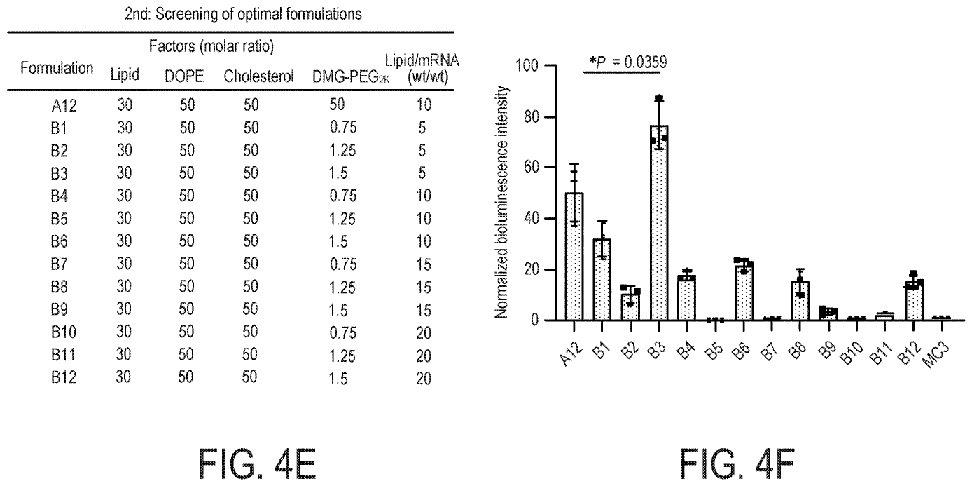

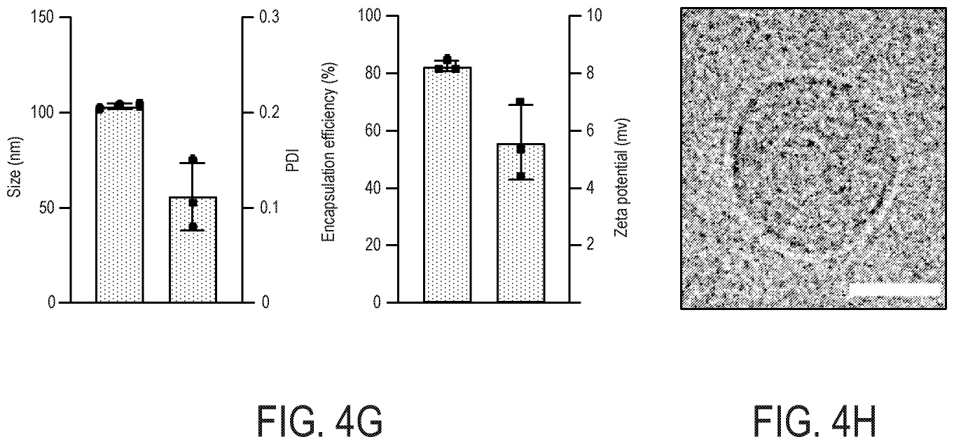

Trisulfide lipids were formulated with firefly luciferase (Fluc) mRNA and assessed the mRNA delivery efficiency in macrophages based on bioluminescence intensity. Among all these LNPs, TS2 LNP exhibited superior mRNA delivery efficiency compared to other TS LNPs. Moreover, the delivery efficiency of TS2 LNP increased by 30 folds compared to the clinically used MC3 LNP, at the same mRNA concentration. Besides, the majority of the LNPs incorporating TS lipids with amines E (TS41-50), F (TS51-60) or G (TS61-70) as the headgroups showed higher or comparable in vitro mRNA delivery efficiency as compared to MC3 LNP, while most TS lipids with amines B, C, or, D displayed low mRNA delivery efficiency (FIG. 4A).

To further optimize the formulation of TS2 LNP, an L 16(4) 4 orthogonal table was designed to determine the optimal molar ratios of each lipid component (FIG. 4B). Based on the bioluminescence intensity obtained from the 16 orthogonal formulations of TS2 LNP, the impact was systematically examined of various molar ratios of individual lipid components on mRNA delivery (FIGS. 4C and 4D). The optimal molar ratios, specifically for ionizable lipids: DOPE: Cholesterol: DMG-PEG₂ₖ, were determined to be in a ratio of 30:50:50:0.5. Compared to the original formulation, the mRNA delivery efficiency exhibited a 1.7-fold improvement using the optimal formulation. Subsequently, the ionizable lipid-to-mRNA mass ratios were further investigated and adjusted the DMG-PEG_{2K} molar ratios within the orthogonal-predicted formulation to enhance the mRNA delivery efficiency of TS2 LNP (FIG. 4E). After the second round of optimization, the lipid-to-mRNA mass ratio was determined to be 5 (wt/wt), and the DMG-PEG_{2K} molar ratio was determined as 1.5. The bioluminescence intensity of the TS2 LNP after the second round of optimization was 1.5 folds greater than that of the top orthogonal formulation obtained after the initial optimization (A12). Moreover, the delivery efficiency of TS2 LNP exceeded that of MC3 LNP by over 80-fold (FIG. 4F). This lead TS2 LNP formulation displayed a hydrodynamic diameter of approximately 100 nm, with a polydispersity index (PDI) of less than 0.15 (FIG. 4G). The encapsulation efficiency of mRNA was around 80%, and the particles exhibited a slight positive charge (FIG. 4G). Additionally, the resulting TS2 LNP showed a spherical morphology visualized by cryogenic transmission electron microscopy (cryo-TEM) (FIG. 4H). Consequently, the TS2 LNP (B3) formulation was selected for the ex vivo delivery of mRNA into macrophages in the following studies.

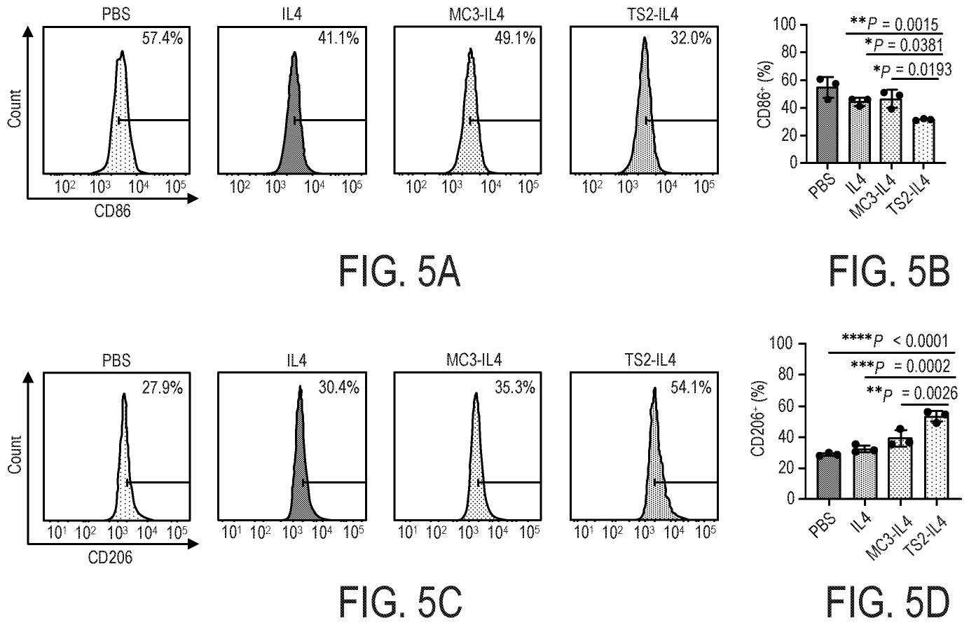

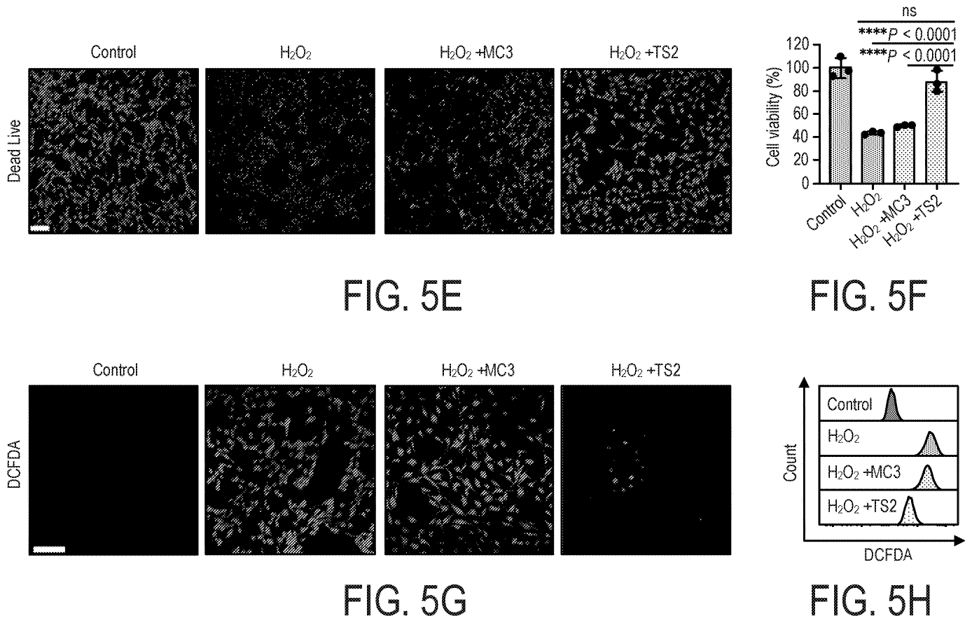

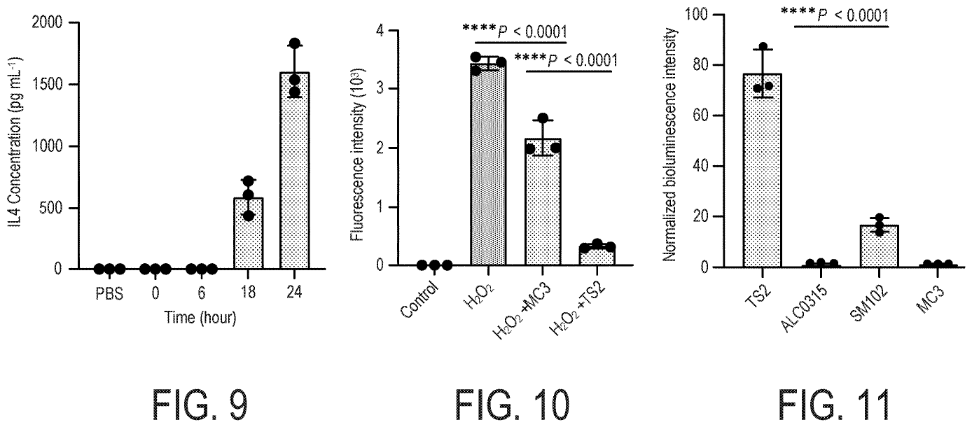

TS2-IL4 LNP induces macrophage polarization and scavenges intracellular ROS in vitro. In the pathological wounds of diabetes, wounds are frequently accompanied by dysregulation of inflammation, compromised macrophage invasion, aggregation, motility, and phenotypic transformation.³⁰ These abnormalities result in an augmented population of M1 phenotype macrophages and a continual release of inflammatory factors, thereby instigating persistent inflammation. The conversion of pro-inflammatory M1 phenotype macrophages to anti-inflammatory M2 phenotype macrophages becomes challenging, impeding the healing process and causing delayed wound healing.^{31, 32} In previous studies, IL4 was found to induce the polarization of macrophages towards an anti-inflammatory M2 phenotype.¹⁵ In the following experiment, TS2 LNP with IL4 mRNA was formulated to reprogram the macrophage phenotype. The results indicate that IL4 was expressed and secreted in macrophages as early as 6 hours following co-incubation with TS2-IL4 LNP (FIG. 9). Afterwards, the lipopolysaccharide (LPS)-stimulated RAW 264.7 cells were incubated with different treatments including PBS, free IL4 mRNA, MC3-IL4 LNP, and TS2-IL4 LNP. The cells were then collected and analyzed by flow cytometry. The results showed a downregulation of the M1 marker (CD86⁺) of the macrophage treated by TS2-IL4 LNP (approximately 31.4%), which was significantly lower than macrophages treated with free IL4 mRNA (approximately 44.4%) and MC3-IL4 LNP (approximately 46.7%), respectively (FIGS. 5A and 5B). Meanwhile, the marker of M2 macrophages (CD206⁺) was upregulated after treatment with TS2-IL4 LNP (approximately 53.0%), which was 1.6 folds and 1.3 folds higher than the groups treated with free IL4 mRNA (approximately 32.1%) and MC3-IL4 LNP (approximately 38.9%) (FIGS. 5C and 5D). These results indicated that the TS2-IL4 LNP was able to induce the phenotypic shift of macrophages from M1 to M2 by IL4 expression.

In the complex wound environment, the uncontrolled accumulation of ROS, including hydrogen peroxide (H₂O₂), induces oxidative stress.³³ This stress hampers the viability of endogenous fibroblasts, vascular endothelial cells, and immune cells, while also deactivating growth factors in the wound, significantly diminishing their healing potential.³⁴ To verify whether TS2 LNP can respond to and scavenge intracellular ROS at the cellular level, the protective capacity of TS2 LNP for fibroblast cells against excessive ROS was studied. Initially, H₂O₂ was used to induce oxidative stress to simulate the ROS microenvironment in vitro. Live/dead Confocal laser scanning microscopy (CLSM) imaging revealed that fibroblast cells co-incubated with H₂O₂ experienced significant cell death. However, following a co-incubation of TS2 LNP with H₂O₂, higher cell viability was observed, which was comparable to the PBS control group (FIG. 5E). It is noteworthy that the MC3 LNP treatment group also displayed a considerable number of dead cells. Subsequently, the MTT assay results were consistent with the fluorescence imaging. After 24 hours of exposure to H₂O₂, only 43.4% of cells survived. In contrast, cell viability increased to 88.1% after TS2 LNP treatment compared to the MC3-treated group (FIG. 5F). These results suggest that TS2 LNP has the capability to protect fibroblasts against oxidative stress and significantly enhance cell viability.

Furthermore, intracellular ROS levels were assessed using a ROS-responsive indicator 2′,7′-Dichlorodihydrofluorescein diacetate (DCFDA). As shown in FIG. 5G, fibroblasts in the TS2 LNP-treated groups exhibited significantly lower green fluorescence compared to those in the H₂O₂ and MC3 LNP-treated groups. Quantitatively, the fluorescence intensity in TS2 LNP-treated cells decreased by 7-fold and 5-fold in comparison to H2O2 and MC3 treatments, respectively (FIG. 10). Further flow cytometry presented similar results regarding TS2 LNP's capacity to scavenge ROS in fibroblasts (FIG. 5H). Altogether, the TS2 LNP proves its ability for efficient ROS responsiveness and antioxidant capabilities, thereby facilitating the healing process of wounds in diabetes.

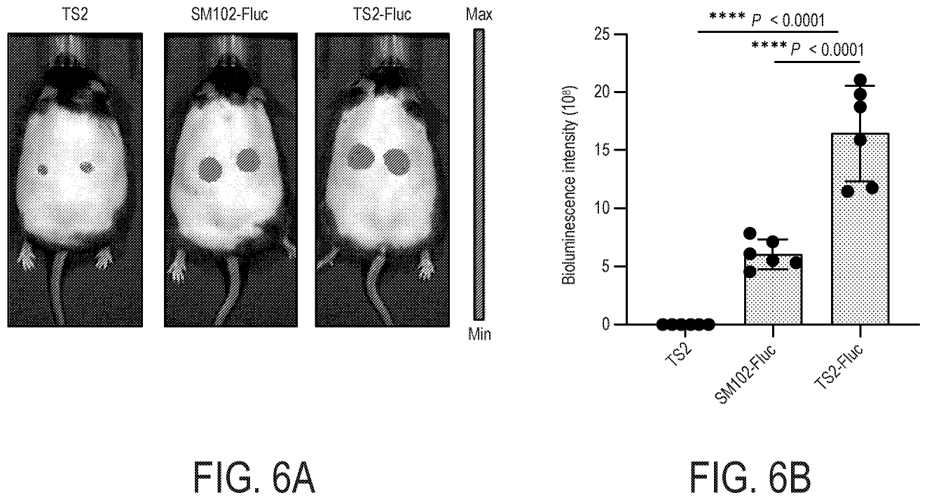

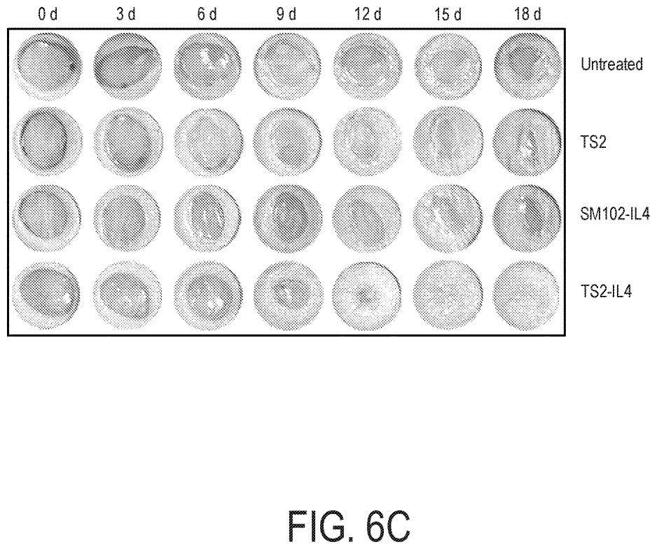

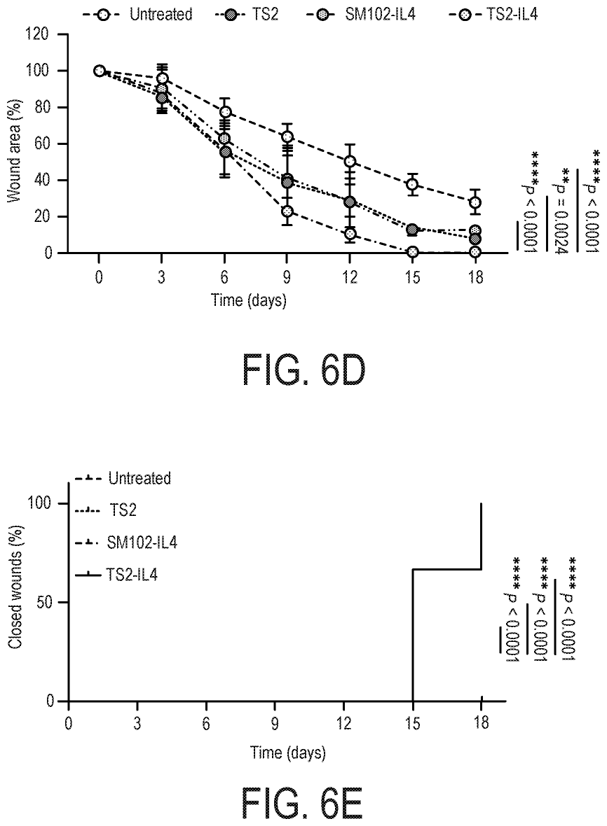

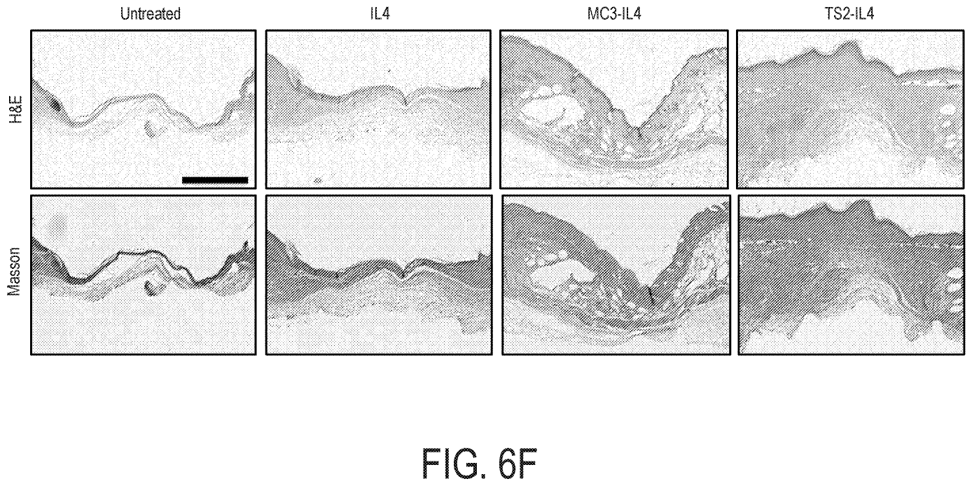

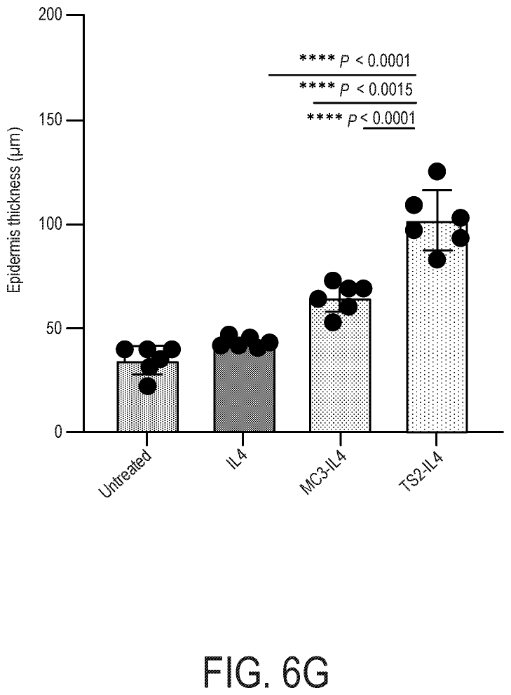

TS2-IL4 LNP loaded in a hydrogel accelerated the wound healing in db/db mice. Next, the study examined the delivery of mRNA by TS2 LNP at the diabetic wound site in db/db mice. The wound model was established by creating 7 mm round wounds on the back of db/db mice.³¹ To facilitate clinical applications, the TS2 LNP-Fluc mRNA was loaded in a hydrogel and embedded in the wound for LNP delivery. Bioluminescence imaging results revealed that TS2-Fluc LNP exhibited 5.1-fold and 1868.1-fold higher bioluminescence intensity than MC3-Fluc LNP and free Fluc mRNA, respectively (FIGS. 6A and 6B). Then, the therapeutic efficacy of TS2-IL4 LNP was evaluated. All mice with wounds were randomly divided into four groups (untreated, IL4 mRNA, MC3-4L4 LNP, and TS2-IL4 LNP). Subsequently, the hydrogel was embedded at the wound site as described above. The wound sites were monitored every three days over a total period of 18 days. As shown in FIG. 6C, the TS2-IL4 LNP treatment displayed an accelerated rate of wound healing compared to the other groups starting from day 9. Quantitative results indicated that approximately 80% wound closure was achieved by day 12 in the TS2-IL4 LN treatment group, while no significant differences were observed in the MC3-IL4 LNP and free IL4 mRNA treatment groups compared to the untreated group (FIG. 6D). Furthermore, the group treated with TS2-IL4 LNP exhibited complete closure starting from day 15, with all wounds closed after day 18 post-treatment. Notably, over half of the wounds achieved complete closure by day 15 after being treated with TS2-IL4 LNP, the time-to-closure function of which was significantly more efficient than other groups (FIG. 6E).

In order to further assess the chronic wound healing capability of TS2-IL4 LNP, hematoxylin and eosin (H&E) staining and Masson's trichrome staining were conducted. After 18 days of treatment, the TS2-IL4 L NP treated group exhibited the formation of a complete epidermal layer, in contrast to the partial epidermis observed in the MC3-IL4 LNP, free IL4 mRNA, and untreated groups (FIG. 6F). Moreover, the TS2-IL4 LNP treated group displayed the highest epidermal thickness values of 101.4 μm, compared to MC3-IL4 LNP (approximately 64.3 Pin), free IL4 mRNA (approximately 43.5 μm), and the untreated group (approximately 34.5 μm) showed 1.5-fold, 2.3-fold, and 2.9-fold increases, respectively (FIG. 6G). These results demonstrated that the TS2-IL4 LNP can accelerate diabetic wound healing.

Macrophage Polarization and Neovascularization at the Wound Site in db/db Mice

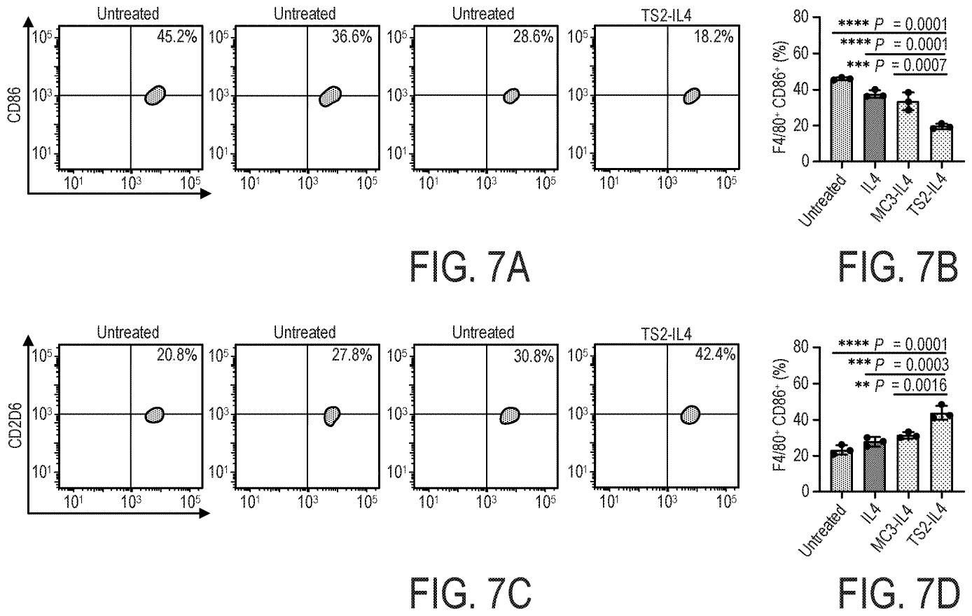

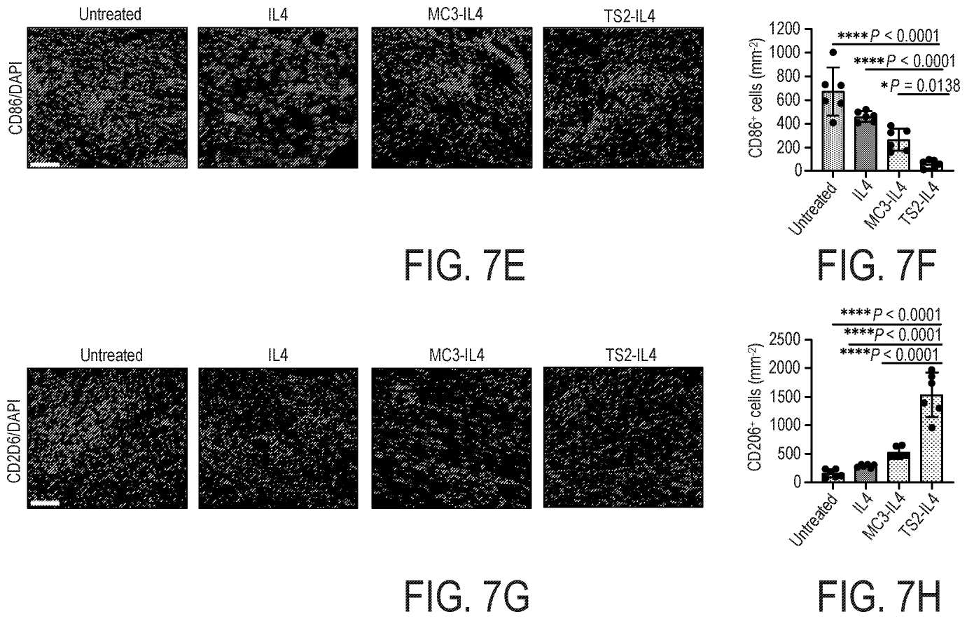

During the wound healing process, inflammation initiates the entire cascade that profoundly phenomenon influenced by macrophage polarity. M2 phenotype macrophages play a crucial role in inhibiting inflammation and facilitating tissue regeneration.¹⁷ Macrophage polarization was studied at the wound site in different treatment groups via flow cytometry and immunofluorescence staining. As shown in FIGS. 7A and 7B, a decrease in the M1 phenotype macrophage population was observed in free IL4 mRNA, MC3-IL4 LNP, and TS2-IL4 LNP treated groups. The percentage of M1 phenotype macrophage (F4/80+CD86+) in wounds treated with TS2-IL4 LNP (approximately 19.4%) was reduced by 2.3 folds compared to that of the untreated group, which was also lower than IL4 mRNA, or MC3-IL4 LNP treated group. In contrast, the percentage of M2 phenotype macrophage (F4/80⁺ CD206⁺) in the TS2-IL4 LNP treated group (approximately 43.8%) was 1.8 folds, 1.5 folds and 1.3 folds higher than those untreated, free IL4 mRNA, and MC3-IL4 LNP groups with increase, respectively (FIGS. 7C and 7D).

Next, the experiment characterized M2 macrophage polarization through CD206/CD86 immunofluorescence staining 18 days post-treatment. Within the subcutaneous layer of the wound site, the TS2-IL4 LNP treated group exhibited the lowest number of CD86-positive cells (green) compared to the untreated group, as well as the IL4 mRNA and MC3-IL4 LNP treated groups (FIGS. 7E and 7F). In contrast, a widespread distribution of CD206-positive cells (green) was observed in the TS2-IL4 LNP group. Additionally, when compared to the TS2-IL4 LNP treated group, both the MC3-TL4 LNP and free IL4 mRNA groups displayed a significant decrease in CD206 expression (FIGS. 7G and 711).

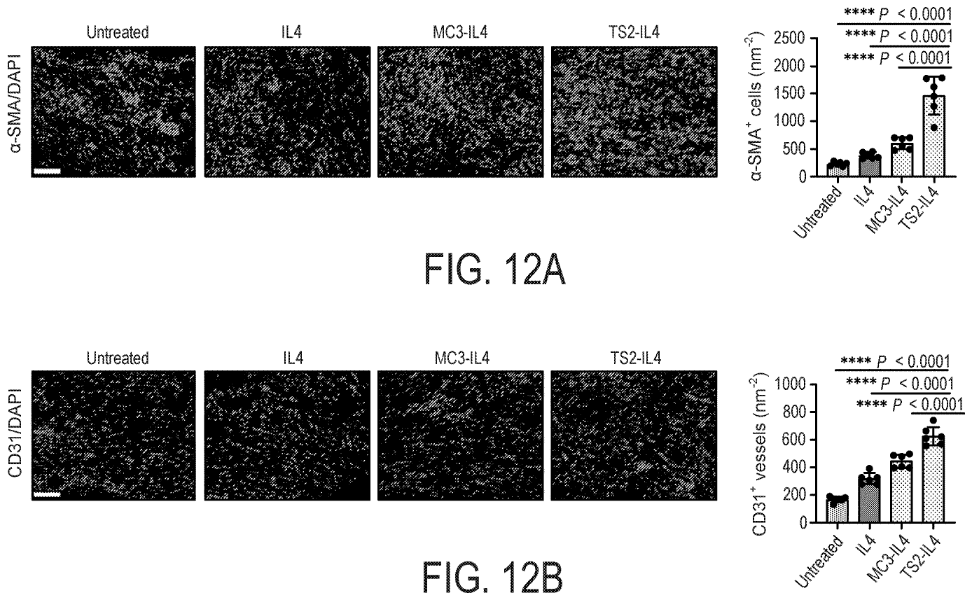

Finally, α-SMA and CD31 immunofluorescence staining was used to assess angiogenesis in wounds treated with TS2-IL4 LNP on day 18. Considering the well-established deficiency in wound contraction capacity and the dysregulated fibroblast-to-myofibroblast conversion observed in diabetic mice throughout the healing process,^{35, 36} α-SMA expression was specifically investigated to determine the potential impact of TS2-IL4 LNP on myofibroblast levels. Notably, the α-SMA positive cell count significantly increased in the TS2-IL4 LNP treatment group, which is higher than those in free IL4 mRNA and MC3-IL4 LNP groups (FIGS. 12A-12B). Besides, it has been widely reported that vascularization has a positive influence on wound closure.^{37, 38} The formation of new blood vessels (CD31 positive) was further examined. Compared with the free IL4 mRNA or MC3-IL4 treated group, there is an evidently higher number of CD31-positive cells in the wounds treated with TS2-IL4 LNP (FIGS. 12A-12B). Taken together, these results demonstrated that the TS2-IL4 LNP significantly induces M2 macrophage polarization and promotes angiogenesis, thereby greatly accelerating the wound closure rate.

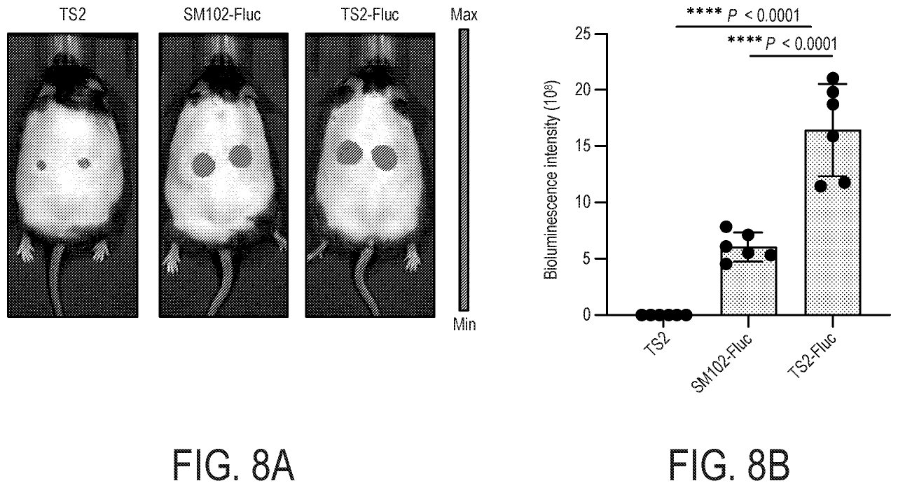

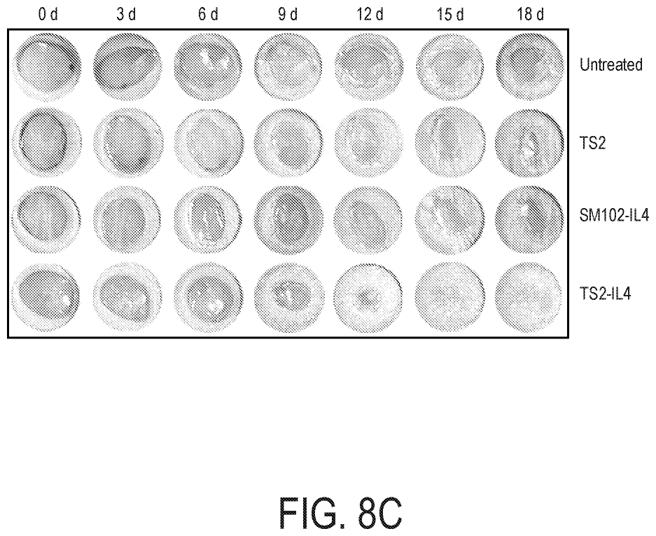

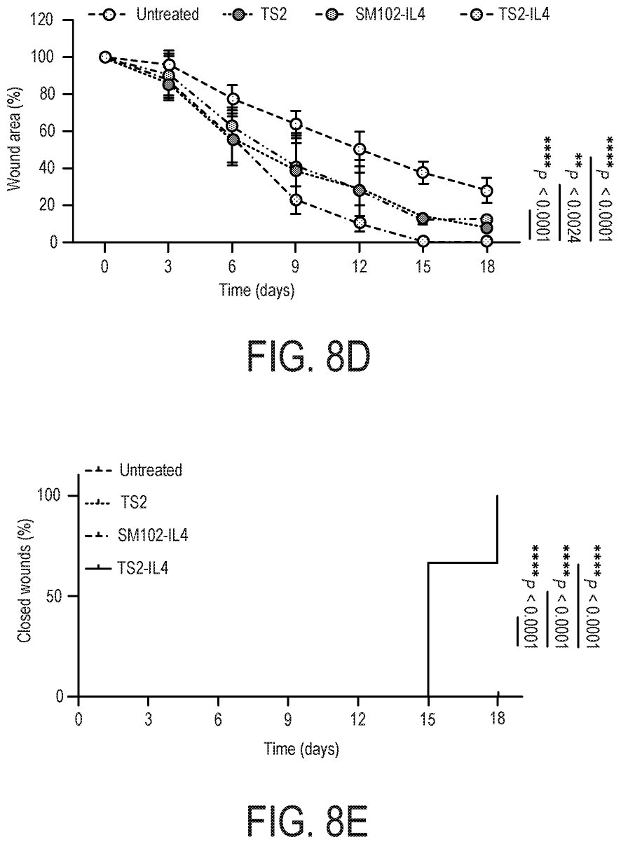

In a separate experiment, the delivery efficiency of TS2 LNP was studied together with ALC-0315 and SM102 LNP, two FDA-approved LNP formulations. The results showed that the order of delivery efficiency was TS2 LNP>SM102 LNP>ALC-0315 LNP in RAW264.7 (FIG. 11). The results revealed that the mRNA delivery efficiency of SM102 LNP in wound tissue was also lower than that of the TS2 LNP vehicle (FIGS. 8A and 8B). Then, the study compared the wound healing efficacy between the TS2-IL4 LNP and SM102-IL4 LNP in diabetic mice. After 18 days of treatment, while both the SM102-IL4 LNP group and the TS2 LNP group without loading IL4 mRNA accelerated wound healing compared to the untreated group, the TS2-IL4 LNP treatment group exhibited the fastest healing outcome (FIGS. 8C-8E). These results also indicate that the accelerated wound healing may be attributed to the combination effect of ROS scavenging and TL4-induced macrophage polarization.

### Discussion

A large number of diabetic wounds can develop into chronic wounds because of the complex microenvironment, characterized by the presence of excess ROS and sustained inflammation. Despite the FDA's approval of recombinant growth factor-based products, they do not fundamentally address the challenges posed by the diabetic wound microenvironment.⁴ Moreover, there is a potential risk of cancer associated with their repeated administration.³⁹ Additionally, ongoing clinical studies on cell-based therapies for wound healing face challenges such as a shorter half-life, difficulties in storage conditions, and higher costs without conclusive superiority in fastening the healing.⁶ To facilitate the healing of diabetic wounds, it is imperative to modulate the wound microenvironment by scavenging excessive ROS, alleviating inflammation, and promoting vascularization to accelerate wound healing.

The utilization of LNP-mRNA formulation for treating diabetic wounds represents a hopeful therapeutic strategy. There have been reports of repeated injection of LNP delivering VEGF mRNA for the treatment of chronic wounds.⁴⁰ Although expressing VEGF would be beneficial for wound healing by enhancing angiogenesis, uncontrolled angiogenesis resulting from VEGF overexpression might lead to the formation of abnormal blood vessels, contributing to complications such as keloid formation.⁴¹⁻⁴⁴ Currently reported LNP formulations have not addressed the challenges of the wound microenvironment, which may impede the wound-healing performance. Consequently, there is an urgent need to develop new strategies to modulate the wound microenvironment and promote wound healing.

In this example, it was hypothesized that the development of a ROS scavenging LNP-mRNA formulation may reshape the wound microenvironment to treat diabetic wounds. To test this approach, ROS-responsive TS LNP were synthesized and formulated. By developing a diverse library of 70 ROS-sensitive trisulfide ionizable lipids through a combinatorial approach, hydrophobic tails were systematically explored for mRNA delivery to macrophages, thereby modulating the wound microenvironment. Researchers previously reported that the reaction of trisulfide with ROS results in the generation of H₂S, a potent antioxidant that neutralizes ROS and alleviates oxidative stress.⁴⁵ This trisulfide group effectively clears excessive ROS within cells, providing cellular protection and potential anti-inflammatory effects. Furthermore, in the highly oxidizing microenvironment, the trisulfide linker plays a crucial role in lipid degradation and subsequent decomposition of LNP. In cell assays, it was shown that TS2 LNP exhibited superior ROS scavenging capabilities in comparison to the clinically approved MC3 LNP. This protective mechanism shielded fibroblasts from oxidative stress, leading to a significant 50% increase in cell viability in the high ROS microenvironment (FIG. 5F). It is noteworthy that, in the diabetic wound model, TS2 LNP without loaded mRNA also effectively promotes wound healing to some extent, which is attributed to the ROS scavenging capacity of the trisulfide group (FIG. 8C).

To induce the macrophage polarization at the wound, TS LNP was formulated with IL4 mRNA, as IL4 typically activates the signal transducer and activator of transcription 6, promoting M2-associated gene transcription while decreasing M1-associated genes.²⁸ In cellular experiments, it was observed that TS2-IL4 LNP can express and secrete IL4 in macrophages, facilitating M1 macrophage polarization into the M2 phenotype. Compared to MC3-IL4 LNP and free IL4 mRNA groups, the percentage of M2 macrophages in TS2-IL4 LNP groups elevated by 1.3 and 1.6-fold, respectively (FIG. 5D). Similarly, at the wound sites, an increase in M2 macrophages percentage was observed in TS2-IL4 LNP group in comparison to MC3-IL4 LNP and free IL4 mRNA (FIG. 7D). However, in the absence of IL4 mRNA, there was a decrease in wound healing efficacy (FIG. 8C). Benefiting from the polarization of macrophages into the M2 phenotype, immunofluorescence staining for CD31 and α-SMA revealed an increased density of newly formed and more mature blood vessels after TS2-IL4 LNP treatment (FIGS. 12A-12B).

In the db/db diabetic wound model, the TS2-IL4 LNP treatment group displayed an accelerated healing rate compared to the MC3-IL4 LNP, SM102-IL4 LNP, free IL4 mRNA, and TS2 LNP treatment groups (FIGS. 6C and 8C). This outcome is primarily attributed to the combined effects of ROS scavenging at the wound site and the increased presence of M2 macrophages, resulting in the promoted formation of an intact epidermis, blood vessels, and myofibroblasts.

### CONCLUSION

In summary, this example developed and investigated a formulation based on ROS-sensitive TS LNP for IL4 mRNA delivery. The designed formulation exhibited robust ROS-scavenging capabilities, protecting fibroblasts against oxidative stress and significantly enhancing cell viability. Additionally, the TS2-IL4 LNP demonstrated the ability to reprogram M1 macrophages into the anti-inflammatory M2 phenotype. In the diabetic mouse wound model, upon a single administration of TS2-IL4 LNP at the wound site, the TS2-IL4 LNP exhibited accelerated wound healing efficacy by scavenging ROS and inducing M2 macrophage polarization. Collectively, this formulation can be used not only for diabetic wounds but also for other types of chronic or acute wound treatment.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A compound defined by Formula I, or a pharmaceutically acceptable salt thereof: 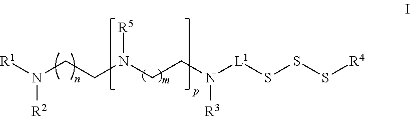
wherein
p is an integer from 0 to 5;
n is an integer from 1 to 10;
m, when present, is an integer from 1 to 10;
R¹, R², and R³ are independently OH, substituted or unsubstituted C₁-C₅ alkyl, substituted or unsubstituted C₁-C₅ alkyl alcohol, or -L¹-S—S—S—R^{a};
R⁴ is a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl; and
L¹ in each case is independently a substituted or unsubstituted alkyl, a carbonyl, an ester, an amide, a carbamate ester, an amine, an ether, a carbonate ester, a thioether, a thioester, or a urea.

2. The compound of claim 1, wherein each L¹ is an ester represented by the formula —Z¹C(O)OZ²—, wherein Z¹ and Z² are each independently a substituted or unsubstituted C₁-C₁₀ alkyl.

3. (canceled)

4. (canceled)

5. (canceled)

6. (canceled)

7. (canceled)

8. The compound of claim 1, wherein the compound is further defined by Formula II, or a pharmaceutically acceptable salt thereof: 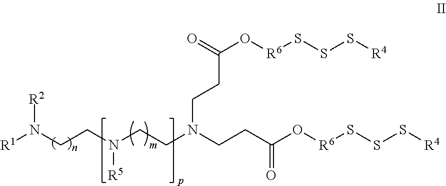
wherein
p is an integer from 0 to 5;
n is an integer from 1 to 10;
m, when present, is an integer from 1 to 10;
R¹ and R² are independently OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

9. The compound of claim 1, wherein the compound is further defined by Formula III, or a pharmaceutically acceptable salt thereof: 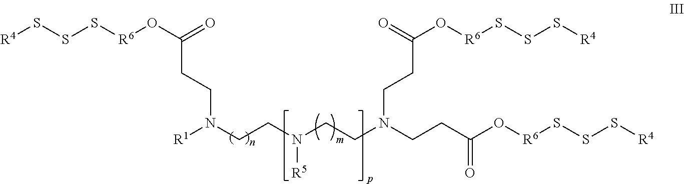
wherein
p is an integer from 0 to 5;
n is an integer from 1 to 10;
m, when present, is an integer from 1 to 10;
R¹ is OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl;
L¹, when present, is a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

10. The compound of claim 1, wherein the compound is further defined by Formula VI, or a pharmaceutically acceptable salt thereof: 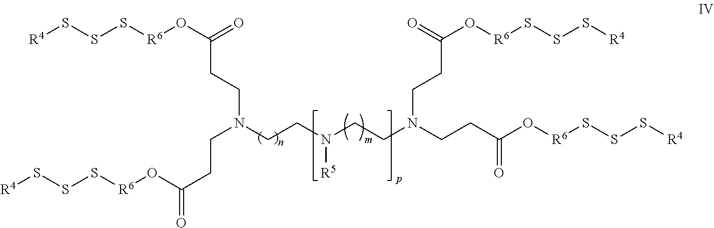
wherein
p is an integer from 0 to 5;
n is an integer from 1 to 10;
m, when present, is an integer from 1 to 10;
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl; and
each R⁶ is independently a substituted or unsubstituted C₁-C₇ alkyl.

11. The compound of claim 1, wherein the compound is further defined by Formula V, or a pharmaceutically acceptable salt thereof: 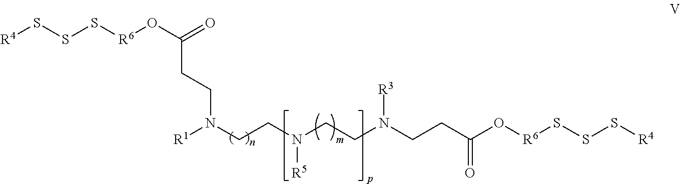
wherein
p is an integer from 0 to 5;
n is an integer from 1 to 10;
each m, when present, is independently an integer from 1 to 10;
R¹ and R³ are independently OH, substituted or unsubstituted C₁-C₅ alkyl, substituted or unsubstituted C₁-C₅ alkyl alcohol, or -L¹-S—S—S—R^{a};
each R⁴ is independently a substituted or unsubstituted C₈-C₁₈ alkyl;
each R⁵, when present, is independently hydrogen, OH, a substituted or unsubstituted C₁-C₅ alkyl, a substituted or unsubstituted C₁-C₅ alkyl alcohol, or -L¹-S—S—S—R^{a};
each R^{a}, when present, is independently a substituted or unsubstituted C₈-C₁₈ alkyl;
L¹ in each case is independently a substituted or unsubstituted alkyl, a carbonyl, an ester, an amide, a carbamate ester, an amine, an ether, a carbonate ester, a thioether, a thioester, or a urea; and
each R⁶ is independently a substituted or unsubstituted C₁-C₇ alkyl.

12. The compound of claim 1, wherein p is from 1 to 5.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The compound of claim 1, wherein the compound is selected from the group consisting of:

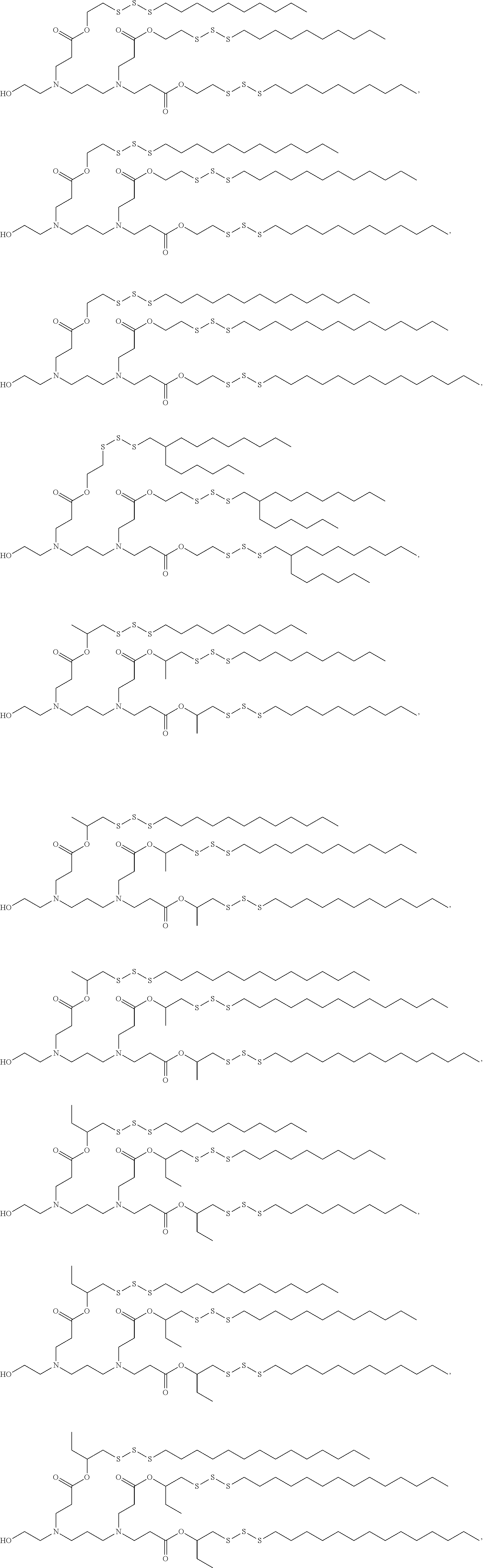

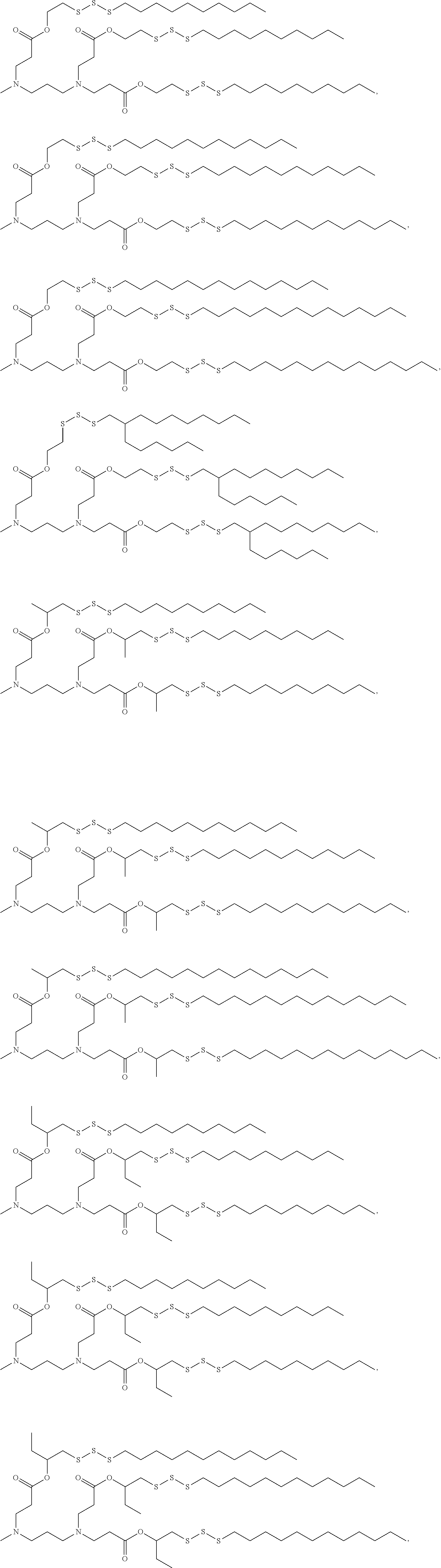

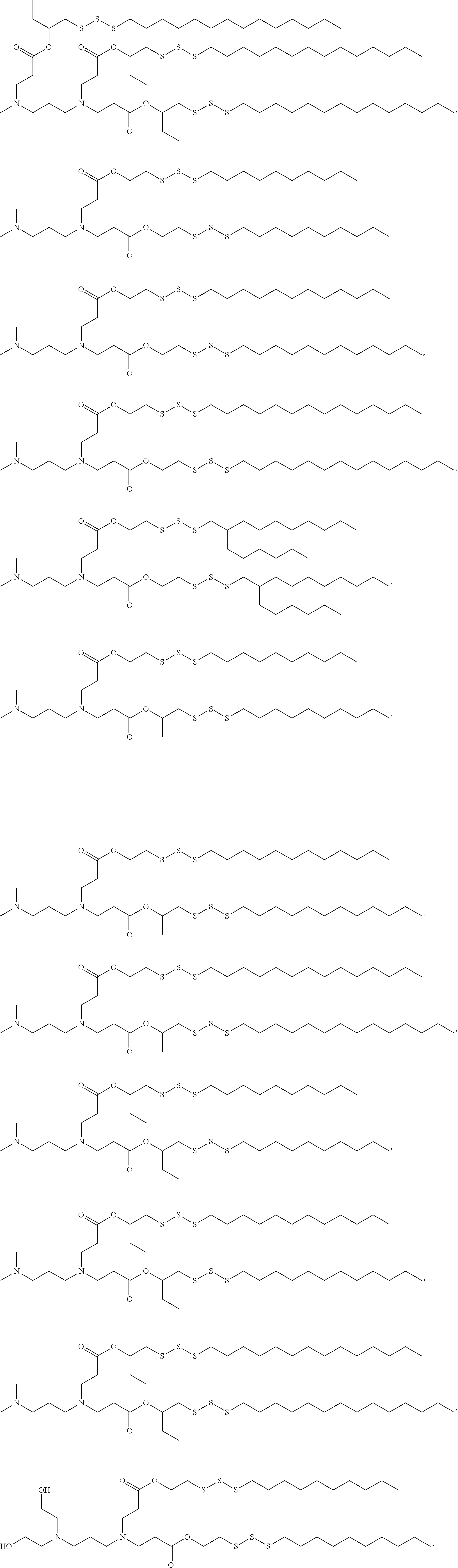

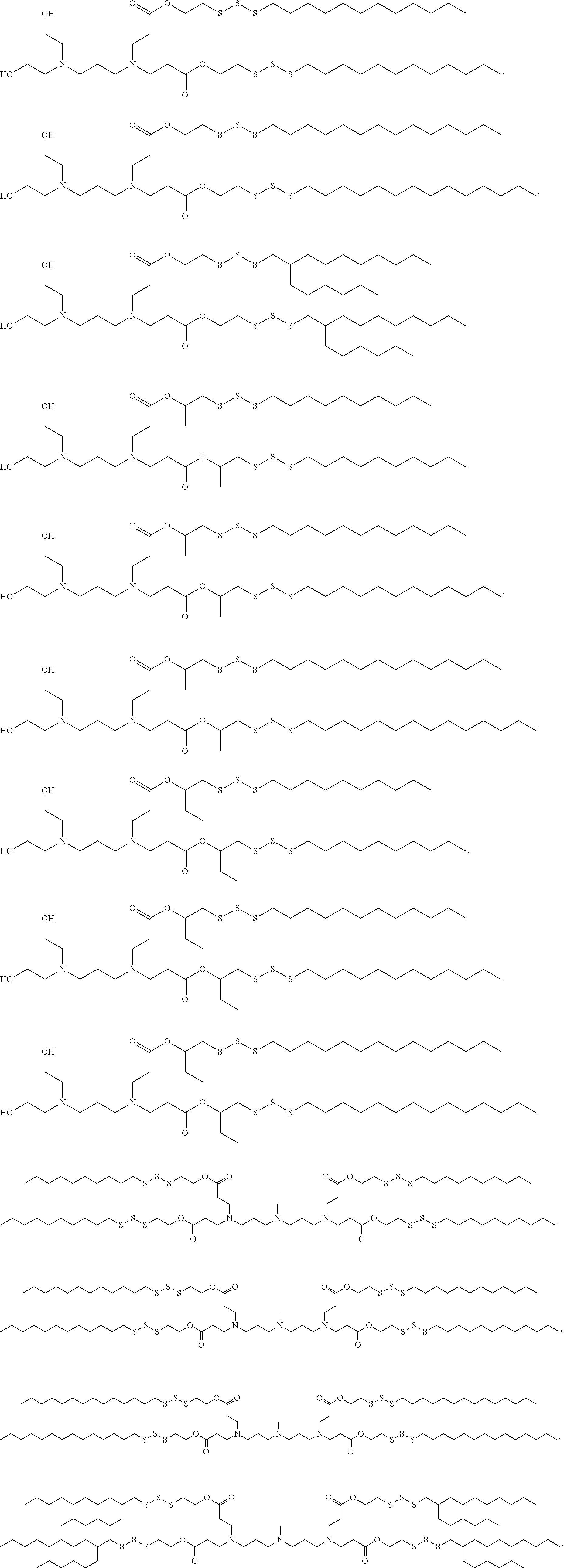

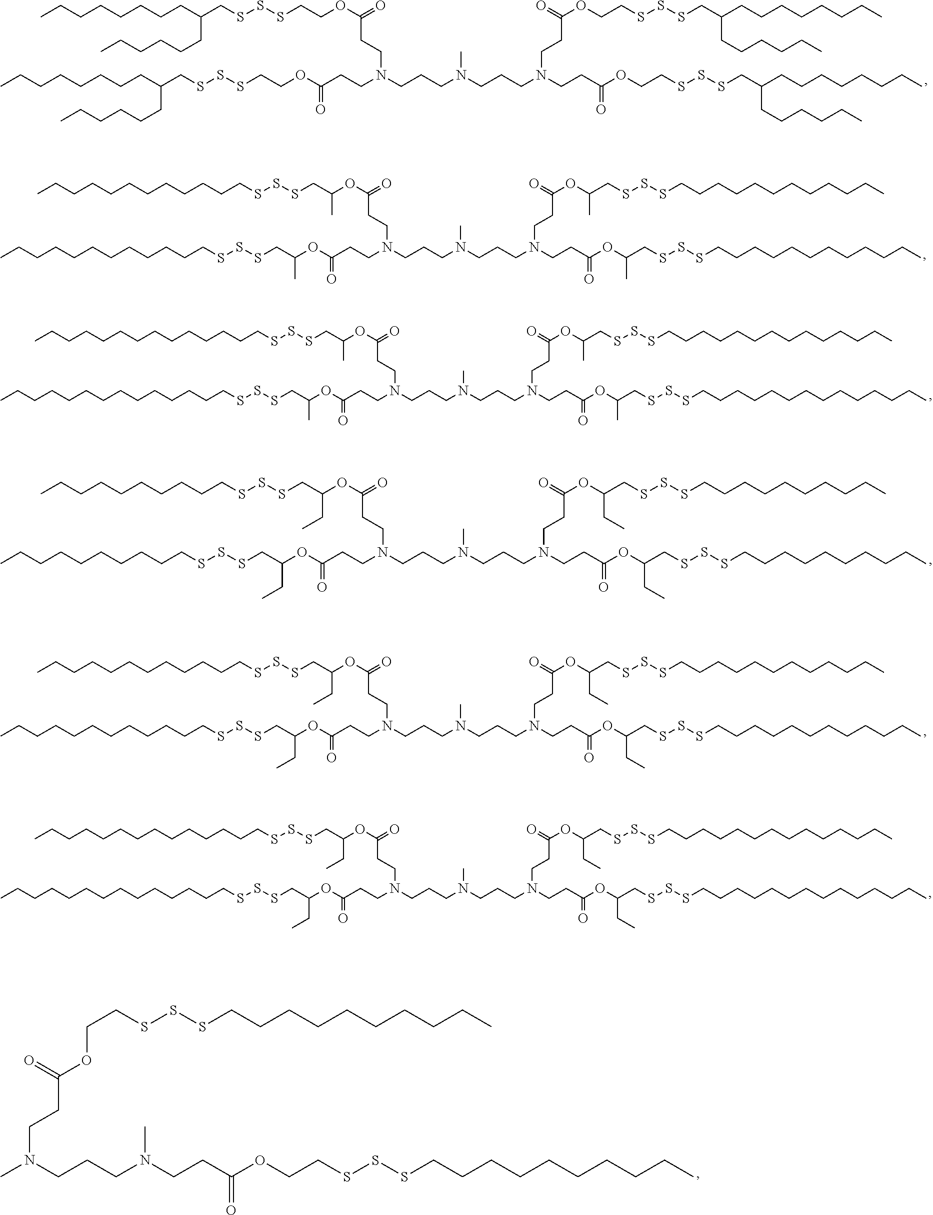

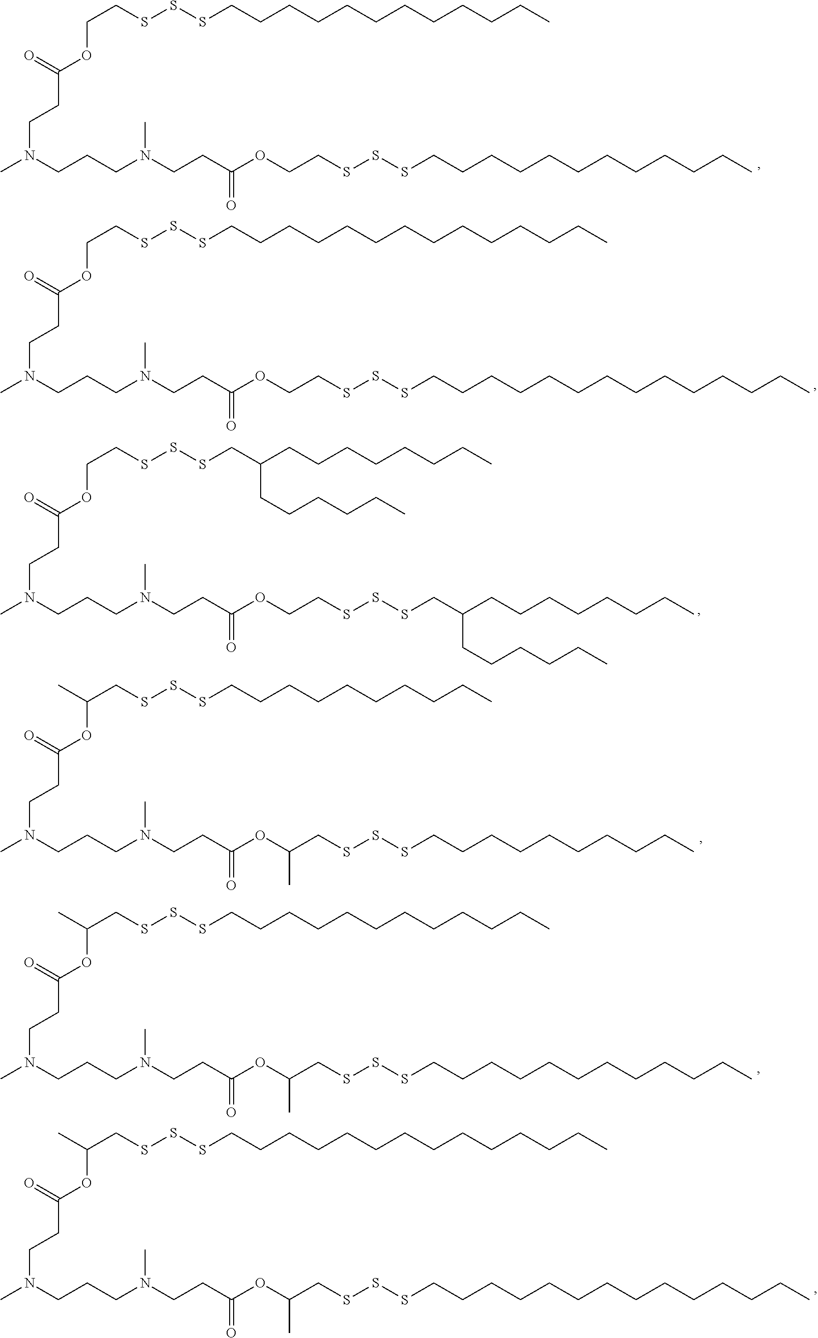

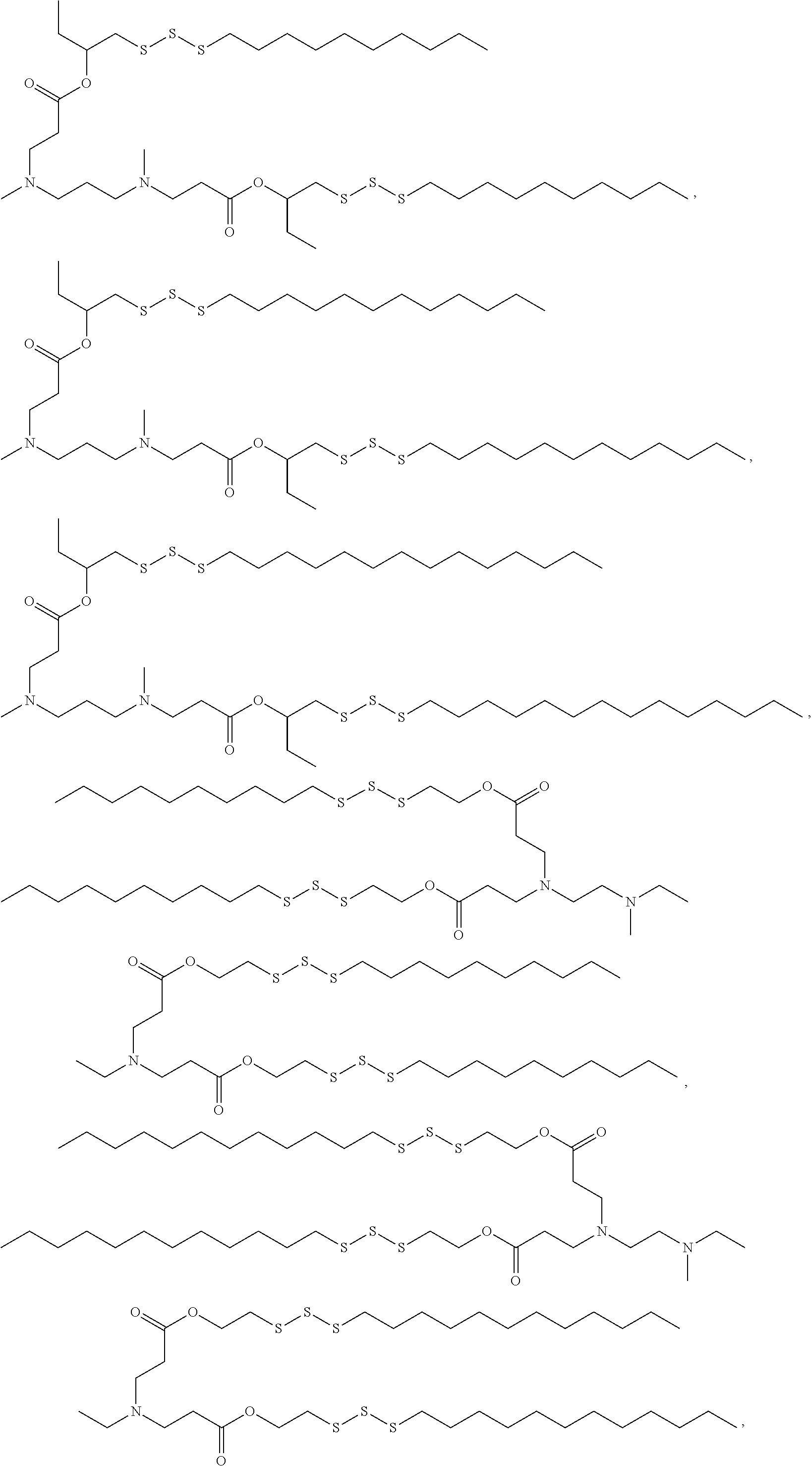

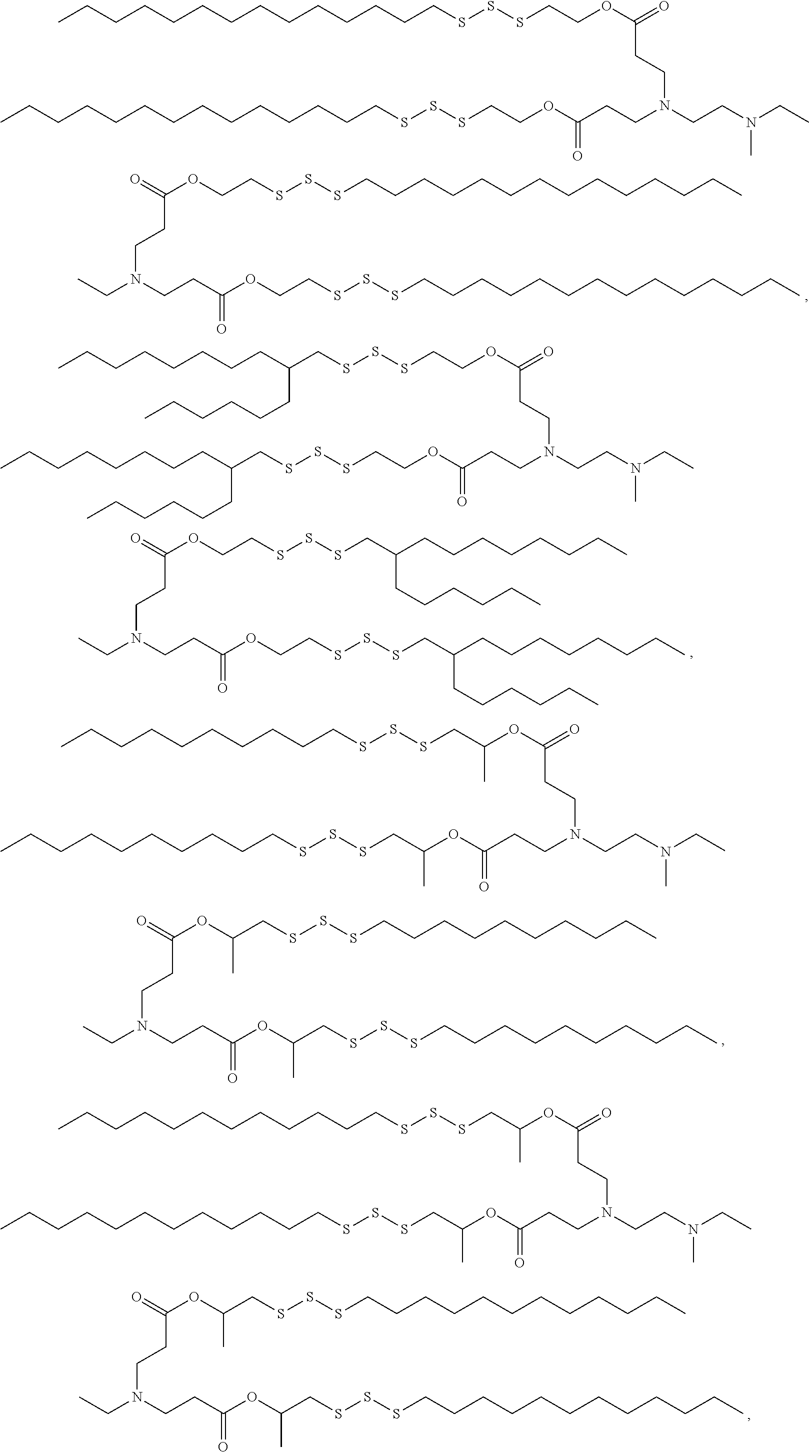

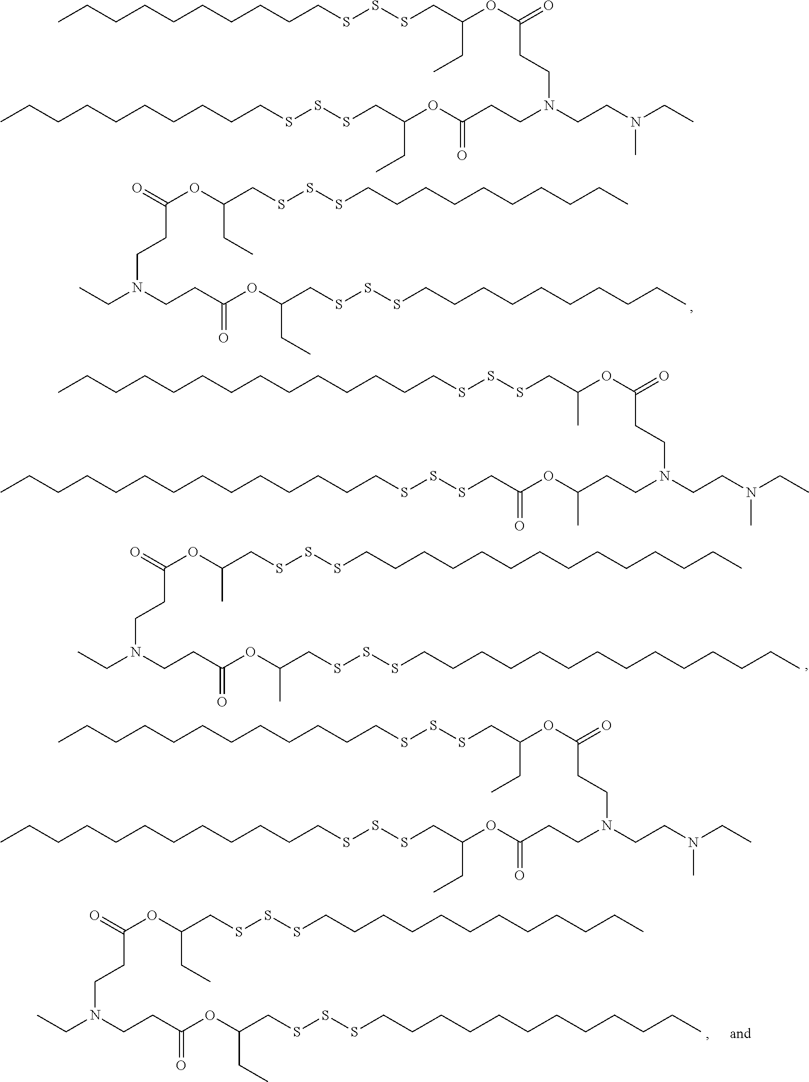

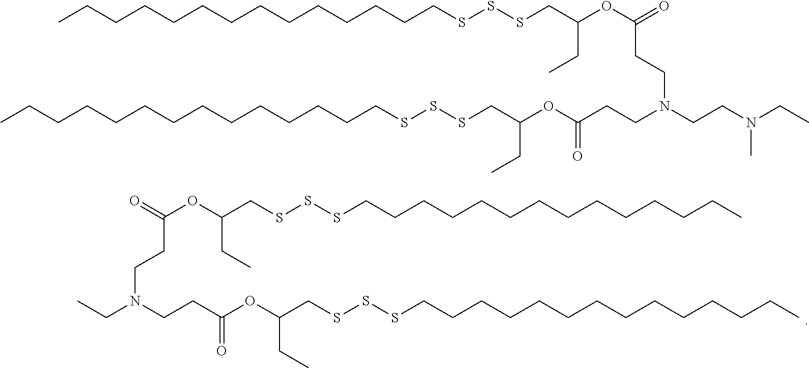

34. The compound of claim 1, wherein the compound is selected from the group consisting of:

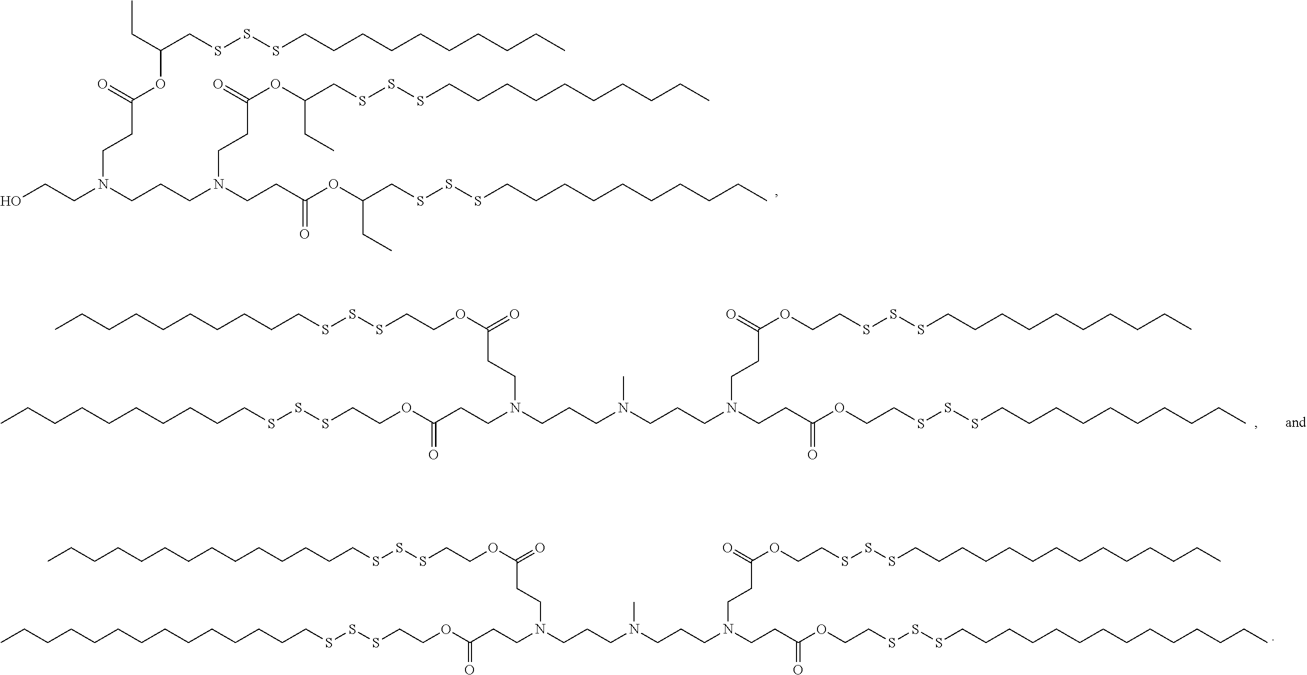

35. The compound of claim 1, wherein the compound is selected from the group consisting of:

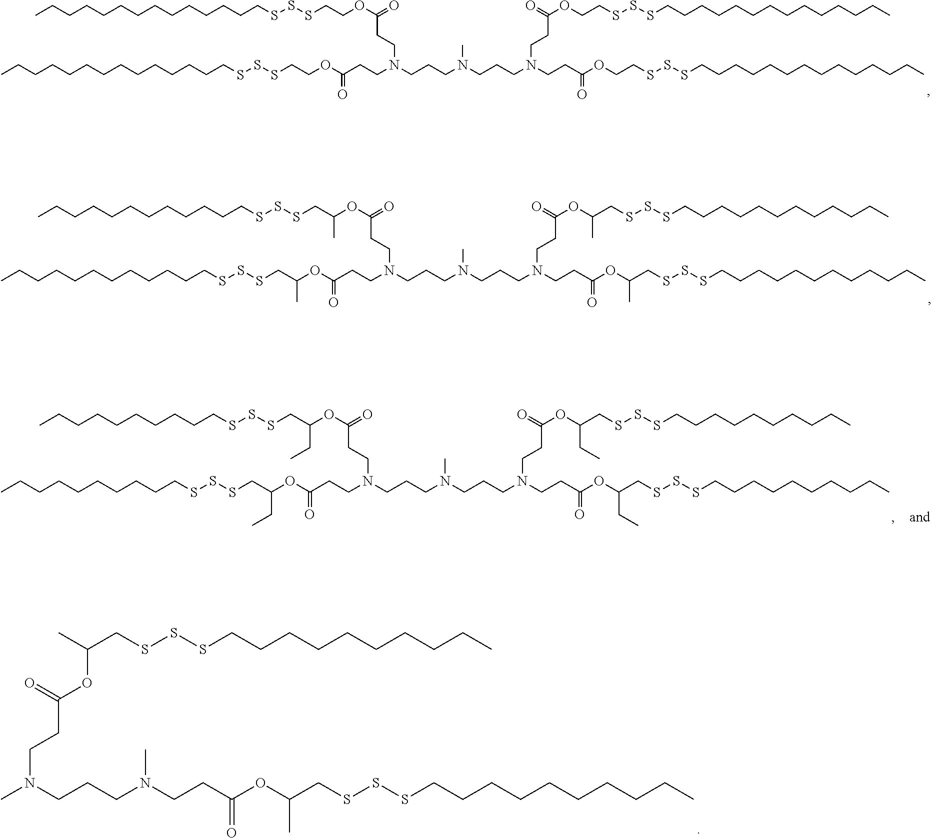

36. (canceled)

37. (canceled)

38. A composition comprising:
the compound of claim 1; and
an agent, wherein the agent comprises mRNA.

39. (canceled)

40. (canceled)

41. (canceled)

42. (canceled)

43. (canceled)

44. (canceled)

45. A lipid nanoparticle comprising:
the compound of claim 1;
a non-cationic lipid;
a polyethylene glycol (PEG)-lipid; and
a sterol.

46. The lipid nanoparticle of claim 45, further comprising an agent.

47. The lipid nanoparticle of claim 46, wherein the agent comprises a polynucleotide.

48. (canceled)

49. (canceled)

50. The lipid nanoparticle of claim 46, wherein the agent comprises a polynucleotide encoding a macrophage polarizing factor.

51. The lipid nanoparticle of claim 46, wherein the agent comprises a polynucleotide encoding interleukin-4.

52. The lipid nanoparticle of claim 46, wherein the agent is encapsulated by the nanoparticle.

53. (canceled)

54. (canceled)

55. (canceled)

56. (canceled)

57. (canceled)

58. (canceled)

59. (canceled)

60. (canceled)

61. (canceled)

62. A hydrogel matrix encapsulating the lipid nanoparticle and the agent according to claim 46.

63. A method for delivering an agent into a cell, comprising:
introducing into the cell a therapeutically effective amount of the lipid nanoparticle of claim 46.

64. A method for promoting wound repair in a subject, the method comprising:
administering to the subject a therapeutically effective amount of the lipid nanoparticle of claim 46.
